(19) United States
(12) Patent Application Publication
(10) Pub. No.: US 2004/0063673 A1
(43) Pub. Date: Apr. 1, 2004

(54) Cyclic compounds containing zinc binding groups as matrix metalloproteinase inhibitors

(76) Inventor: Adam Richard Johnson, Ann Arbor, MI (US)

Correspondence Address: WARNER-LAMBERT COMPANY, 2800 PLYMOUTH RD, ANN ARBOR, MI, 48105, US

(21) Appl. No.: 10/634,531

(22) Filed: Aug. 5, 2003

Related U.S. Application Data
(60) Provisional application No. 60/403,255, filed on Aug. 13, 2002.

(51) Int. Cl. A61K 31/66; A61K 31/515; A61K 31/433; A61K 31/426; A61K 31/4196; A61K 31/4162

(52) U.S. Cl. 514/114; 514/270; 514/362; 514/365; 514/381; 514/383; 514/400; 514/438; 514/408; 514/561; 514/575; 514/406; 544/301; 548/136; 548/254; 548/267.2; 548/335.5; 548/373.1; 548/577; 549/72

(57) ABSTRACT

This invention provides compounds defined by Formula I

Z—L—R¹—Q—D—(V¹)ₘ—R² I

or a pharmaceutically acceptable salt thereof,

wherein Z, L, R¹, Q, D, V¹, m, and R² are as defined in the specification. The invention also provides pharmaceutical compositions comprising a compound of Formula I, or a pharmaceutically acceptable salt thereof, as defined in the specification, together with a pharmaceutically acceptable carrier, diluent, or excipient. The invention also provides methods of inhibiting an MMP-13 enzyme in an animal, comprising administering to the animal a compound of Formula I, or a pharmaceutically acceptable salt thereof. The invention also provides methods of treating a disease mediated by an MMP-13 enzyme in a patient, comprising administering to the patient a compound of Formula I, or a pharmaceutically acceptable salt thereof, either alone or in a pharmaceutical composition. The invention also provides methods of treating diseases such as heart disease, multiple sclerosis, osteo- and rheumatoid arthritis, arthritis other than osteo- or rheumatoid arthritis, cardiac insufficiency, inflammatory bowel disease, heart failure, age-related macular degeneration, chronic obstructive pulmonary disease, asthma, periodontal diseases, psoriasis, atherosclerosis, and osteoporosis in a patient, comprising administering to the patient a compound of Formula I, or a pharmaceutically acceptable salt thereof, either alone or in a pharmaceutical composition. The invention also provides combinations, comprising a compound of Formula I, or a pharmaceutically acceptable salt thereof, together with another pharmaceutically active component as described in the specification.

### CROSS-REFERENCE TO RELATED APPLICATIONS

This application claims benefit of priority from U.S. Provisional Patent Application No. 60/403,255, filed Aug. 13, 2002.

### INVENTION OF THE INVENTION

This invention relates to cyclic compounds containing zinc binding groups which inhibit matrix metalloproteinase enzymes and thus are useful for treating diseases resulting from MMP-mediated tissue breakdown such as heart disease, cardiac insufficiency, inflammatory bowel disease, multiple sclerosis, osteo- and rheumatoid arthritis, arthritis other than osteo- or rheumatoid arthritis, heart failure, age-related macular degeneration, chronic obstructive pulmonary disease, asthma, periodontal diseases, psoriasis, atherosclerosis, and osteoporosis.

### BACKGROUND OF THE INVENTION

Matrix metalloproteinases (sometimes referred to as MMPs) are naturally occurring enzymes found in most mammals. Over-expression and activation of MMPs, or an imbalance between MMPs and inhibitors of MMPs, have been suggested as factors in the pathogenesis of diseases characterized by the breakdown of extracellular matrix or connective tissues.

Stromelysin-1 and gelatinase A are members of the MMP family. Other members include fibroblast collagenase (MMP-1), neutrophil collagenase (MMP-8), gelatinase B (92 kDa gelatinase) (MMP-9), stromelysin-2 (MMP-10), stromelysin-3 (MMP-11), matrilysin (MMP-7), collagenase 3 (MMP-13), TNF-alpha converting enzyme (TACE), and other newly discovered membrane-associated matrix metalloproteinases (Sato H., Takino T., Okada Y., Cao J., Shinagawa A., Yamamoto E., and Seiki M., Nature, 1994;370:61-65). These enzymes have been implicated with a number of diseases which result from breakdown of connective tissue, including such diseases as rheumatoid arthritis, osteoarthritis, osteoporosis, periodontitis, multiple sclerosis, gingivitis, corneal epidermal and gastric ulceration, atherosclerosis, neointimal proliferation which leads to restenosis and ischemic heart failure, and tumor metastasis. A method for preventing and treating these and other diseases is now recognized to be by inhibiting matrix metalloproteinase enzymes, thereby curtailing and/or eliminating the breakdown of connective tissues that results in the disease states.

There is a catalytic zinc domain in matrix metalloproteinases that is typically the focal point for inhibitor design. The modification of substrates by introducing zinc-chelating groups has generated potent inhibitors such as peptide hydroxamates and thiol-containing peptides. Peptide hydroxamates and the natural endogenous inhibitors of MMPs (TIMPs) have been used successfully to treat animal models of cancer and inflammation. MMP inhibitors have also been used to prevent and treat congestive heart failure and other cardiovascular diseases, U.S. Pat. No. 5,948,780.

A major limitation on the use of currently known MMP inhibitors is their lack of specificity for any particular enzyme. Recent data has established that specific MMP enzymes are associated with some diseases, with no effect on others. The MMPs are generally categorized based on their substrate specificity, and indeed the collagenase subfamily of MMP-1, MMP-8, and MMP-13 selectively cleave native interstitial collagens, and thus are associated mainly with diseases linked to such interstitial collagen tissue. This is evidenced by the recent discovery that MMP-13 alone is over expressed in breast carcinoma, while MMP-1 alone is over expressed in papillary carcinoma (see Chen et al., J. Am. Chem. Soc., 2000;122:9648-9654).

Selective inhibitors of MMP-13 include a compound named WAY-170523, which has been reported by Chen et al., supra., 2000, and other compounds are reported in PCT International Patent Application Publication numbers WO 01/63244; WO 00/09485; WO 01/12611; WO 02/34726; and WO 02/34753, and European Patent Application numbers EP 935,963 and EP 1,138,680. Further, U.S. Pat. No. 6,008,243 discloses inhibitors of MMP-13. However, no selective or nonselective inhibitor of MMP-13 has been approved and marketed for the treatment of any disease in any mammal.

The need continues to find new low molecular weight compounds that are potent and selective MMP inhibitors, and that have an acceptable therapeutic index of toxicity/potency for use clinically in the treatment of disease states. An inhibitor that does not possess a zinc binding group and that effectively binds allosterically to an MMP enzyme would be expected to be a potent and highly selective, if not specific, inhibitor of the MMP versus other MMP enzymes. Unfortunately, such allosterically binding inhibitors have not been disclosed. Nevertheless, if such an inhibitor were then derivatized by attaching a zinc binding group at the right location on the inhibitor, a super potent inhibitor that retains high selectivity for the MMP enzyme would result. An object of this invention is to provide a group of selective MMP-13 inhibitor compounds characterized as being cyclic compounds containing zinc binding groups.

### SUMMARY OF THE INVENTION

This invention provides cyclic compounds containing zinc binding groups defined by Formula I.

Accordingly, embodiments of the invention include:

1. A compound of Formula I

Z—L—R¹—Q—D—(V¹)ₘ—R² I

or a pharmaceutically acceptable salt thereof,

wherein:

Z is selected from:

HO₂C;

HO(H)N(O)C;

H(O)C—N(OH);

CH₃(O)C—N(OH);

CH₃(H)N(O)C—N(OH);

HS;

H₂N(O)₂S;

CH₃(H)N(O)₂S;

HO(O)P;

(HO)₂(O)P;

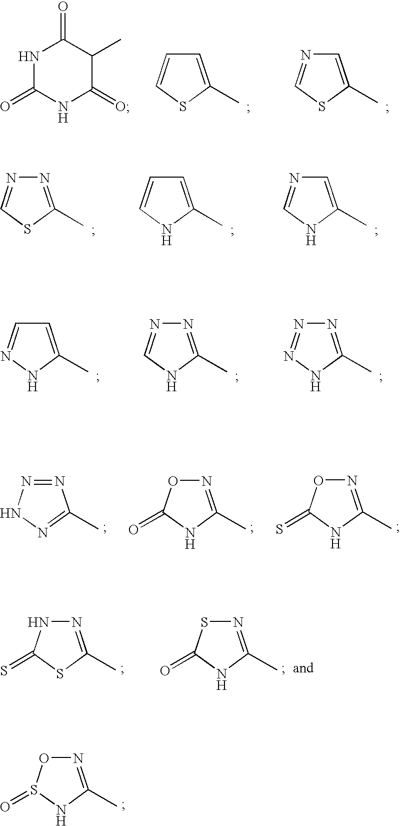

L is selected from:

C₃-C₅ alkylenyl;

Substituted C₃-C₅ alkylenyl;

3- to 5-membered heteroalkylenyl; and

Substituted 3- to 5-membered heteroalkylenyl;

Substituted L groups contain 1 or 2 substituents on a carbon atom or nitrogen atom independently selected from:

HO;

CN; and

CF₃;

wherein each substituent on a carbon atom may further be independently F, and wherein 2 substituents may be taken together with a carbon atom to which they are both bonded to form the group C═O;

R¹ is independently selected from:

C₅ or C₆ cycloalkylenyl-(C₁-C₈ alkylenyl);

Substituted C₅ or C₆ cycloalkylenyl-(C₁-C₈ alkylenyl);

5- or 6-membered heterocycloalkylenyl-(C₁-C₈ alkylenyl);

Substituted 5- or 6-membered heterocycloalkylenyl-(C₁-C₈ alkylenyl);

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl);

Phenyl;

Substituted phenyl;

Naphthyl;

Substituted naphthyl;

5- or 6-membered heteroaryl;

Substituted 5- or 6-membered heteroaryl;

8- to 10-membered heterobiaryl; and

Substituted 8- to 10-membered heterobiaryl;

R² is independently selected from:

H;

C₁-C₆ alkyl;

Phenyl-(C₁-C₈ alkylenyl);

Substituted phenyl-(C₁-C₈ alkylenyl);

Naphthyl-(C₁-C₈ alkylenyl);

Substituted naphthyl-(C₁-C₈ alkylenyl);

5- or 6-membered heteroaryl-(C₁-C₈ alkylenyl);

Substituted 5- or 6-membered heteroaryl-(C₁-C₈ alkylenyl);

8- to 10-membered heterobiaryl-(C₁-C₈ alkylenyl);

Substituted 8- to 10-membered heterobiaryl-(C₁-C₈ alkylenyl);

Phenyl-O—(C₁-C₈ alkylenyl);

Substituted phenyl-O—(C₁-C₈ alkylenyl);

Phenyl-S—(C₁-C₈ alkylenyl);

Substituted phenyl-S—(C₁-C₈ alkylenyl);

Phenyl-S(O)—(C₁-C₈ alkylenyl);

Substituted phenyl-S(O)—(C₁-C₈ alkylenyl);

Phenyl-S(O)₂—(C₁-C₈ alkylenyl); and

Substituted phenyl-S(O)₂—(C₁-C₈ alkylenyl);

Each substituted R¹ group contains from 1 to 3 substituents, and each substituted R² group contains from 1 to 4 substituents, wherein each substituent is independently on a carbon or nitrogen atom, independently selected from:

C₁-C₆ alkyl;

CN;

CF₃;

HO;

(C₁-C₆ alkyl)-O;

(C₁-C₆ alkyl)-S(O)₂;

H₂N;

(C₁-C₆ alkyl)-N(H);

(C₁-C₆ alkyl)₂-N;

(C₁-C₆ alkyl)-C(O)O—(C₁-C₈ alkylenyl)ₘ;

(C₁-C₆ alkyl)-C(O)O-(1- to 8-membered heteroalkylenyl)ₘ;

(C₁-C₆ alkyl)-C(O)N(H)—(C₁-C₈ alkylenyl)ₘ;

(C₁-C₆ alkyl)-C(O)N(H)-(1- to 8-membered heteroalkylenyl)ₘ;

H₂NS(O)₂—(C₁-C₈ alkylenyl);

(C₁-C₆ alkyl)-N(H)S(O)₂—(C₁-C₈ alkylenyl)ₘ;

(C₁-C₆ alkyl)₂—NS(O)₂—(C₁-C₈ alkylenyl)ₘ;

3- to 6-membered heterocycloalkyl-(G)ₘ;

Substituted 3- to 6-membered heterocycloalkyl-(G)ₘ;

5- or 6-membered heteroaryl-(G)ₘ;

Substituted 5- or 6-membered heteroaryl-(G)ₘ;

(C₁-C₆ alkyl)-S(O)₂—N(H)—C(O)—(C₁-C₈ alkylenyl)ₘ; and

(C₁-C₆ alkyl)-C(O)—N(H)—S(O)₂—(C₁-C₈ alkylenyl)ₘ;

wherein each substituent on a carbon atom may further be independently selected from:

Halo; and

HO₂C;

wherein 2 substituents may be taken together with a carbon atom to which they are both bonded to form the group C═O;

wherein two adjacent, substantially sp² carbon atoms may be taken together with a diradical substituent to form a cyclic diradical selected from:

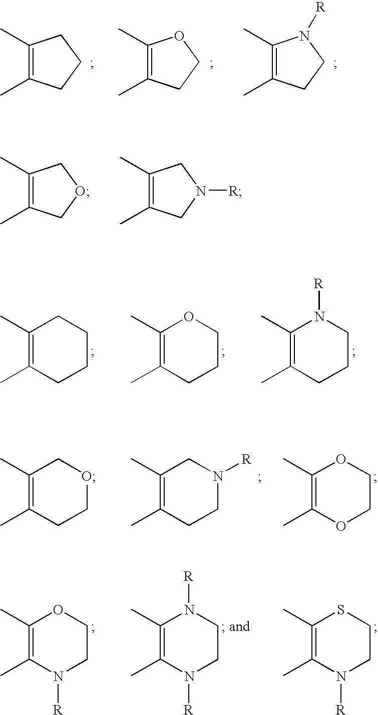

R is H or C₁-C₆ alkyl;

G is CH₂; O, S, S(O); or S(O)₂;

Each m is an integer of 0 or 1;

Q, when bonded to a nitrogen atom in group D, is selected from:

OC(O);

CH(R⁶)C(O);

OC(NR⁶);

CH(R⁶)C(NR⁶);

N(R⁶)C(O);

N(R⁶)C(S);

N(R⁶)C(NR⁶);

SC(O);

CH(R⁶)C(S);

SC(NR⁶);

C≡CCH₂;

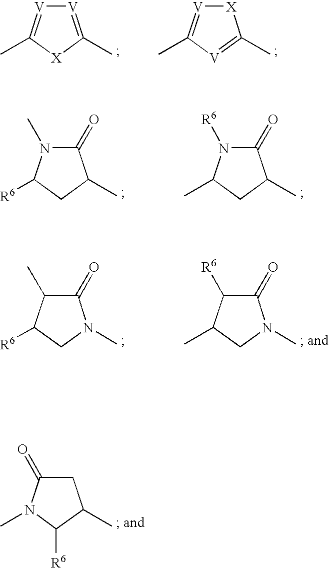

Q, when bonded to a carbon atom in group D, is as defined above and may further be selected from:

OCH₂;

N(R⁶)CH₂;

trans-(H)C═C(H);

cis-(H)C═C(H);

C≡C;

CH₂C≡C;

CF₂C≡C;

C≡CCF₂;

Each R⁶ independently is H, C₁-C₆ alkyl, C₃-C₆ cycloalkyl; 3- to 6-membered heterocycloalkyl; phenyl; benzyl; or 5- or 6-membered heteroaryl;

X is O, S, N(H), or N(C₁-C₆ alkyl);

Each V is independently C(H) or N;

D is a cyclic diradical group selected from:

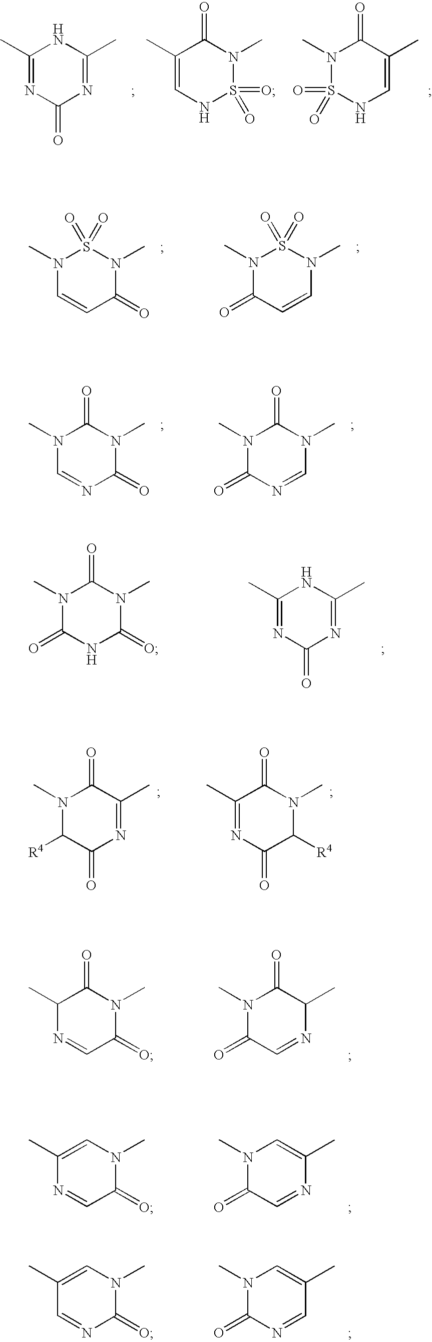

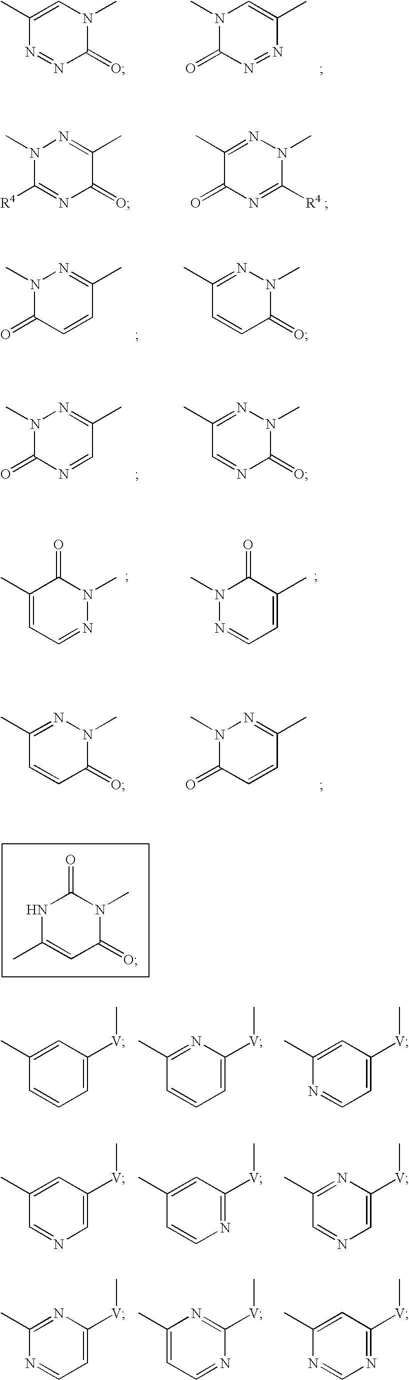

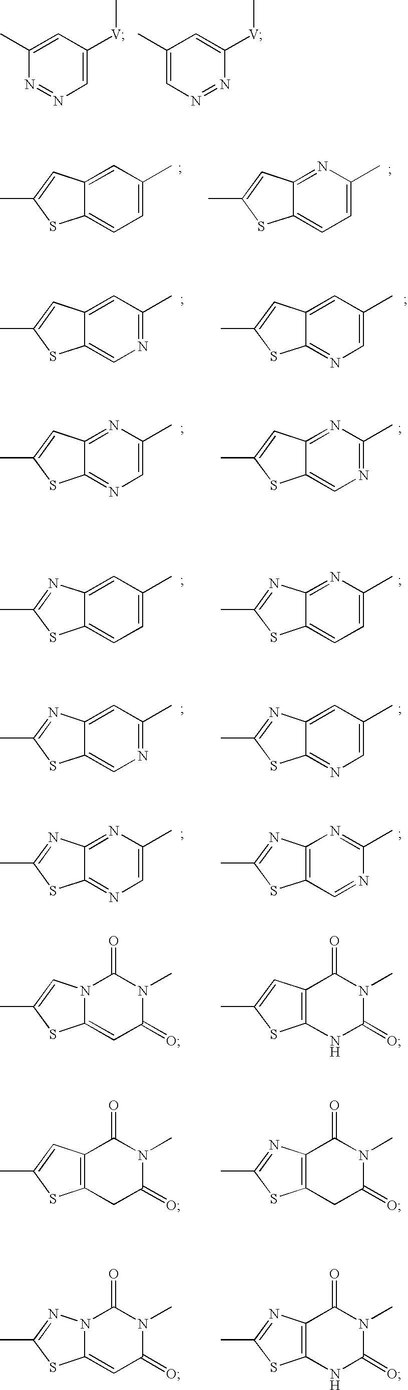

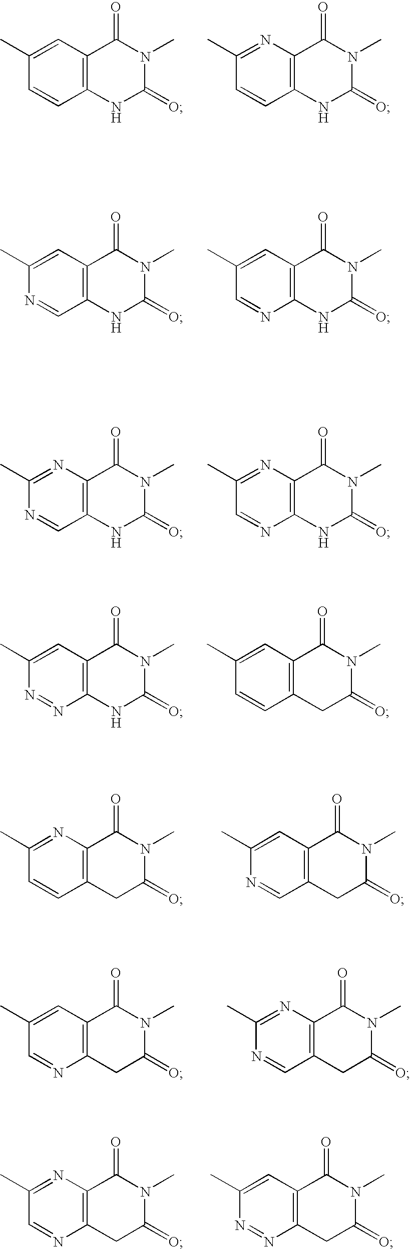

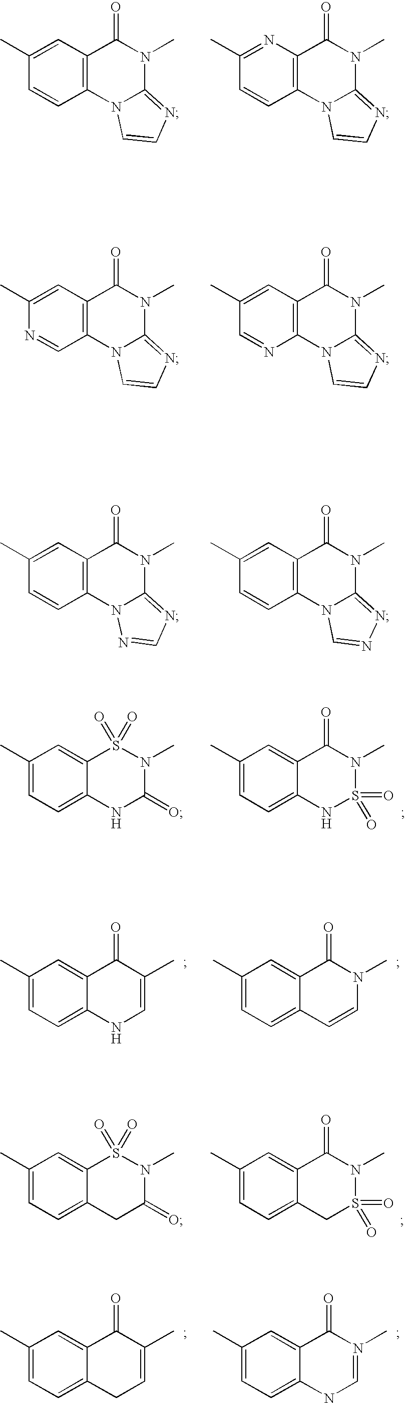

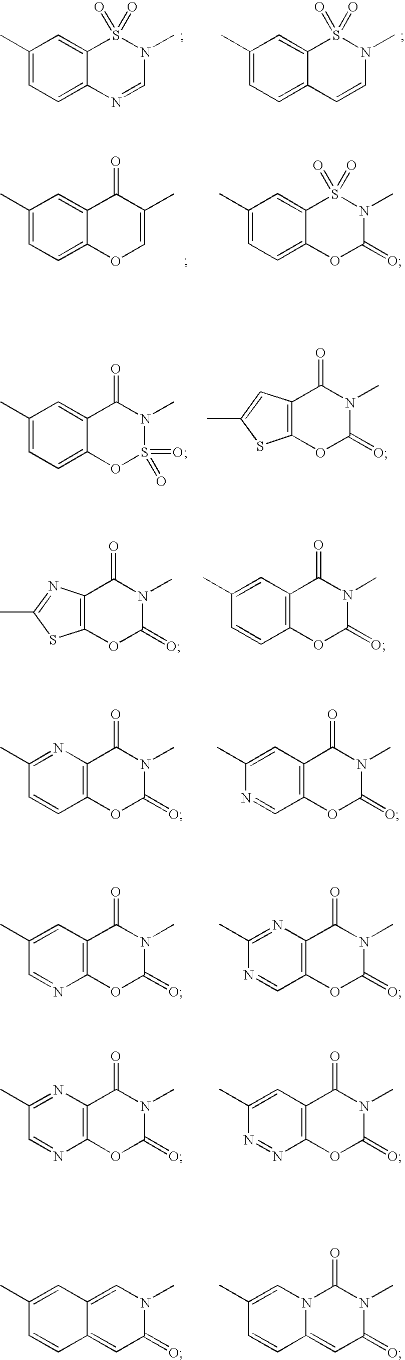

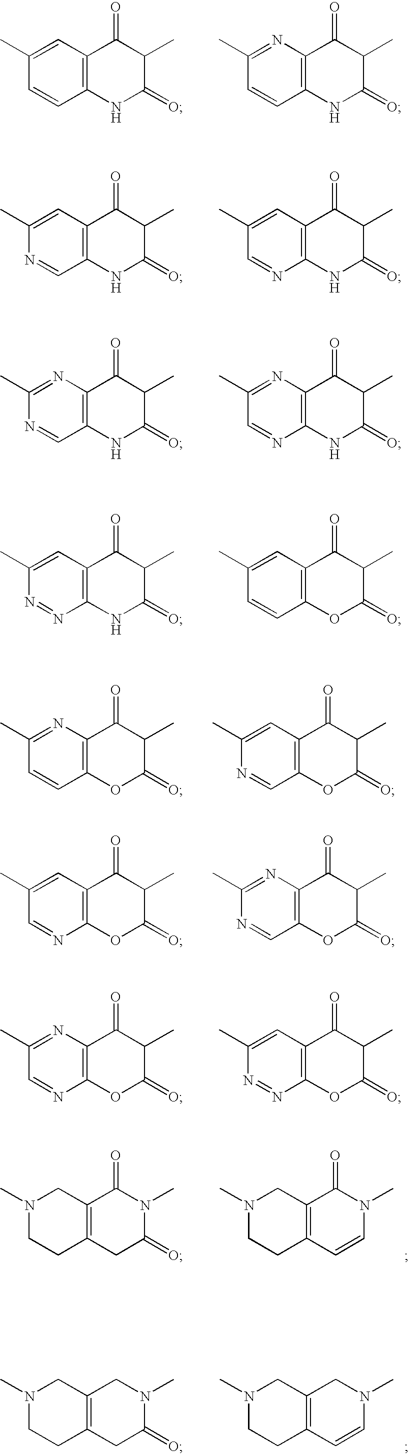

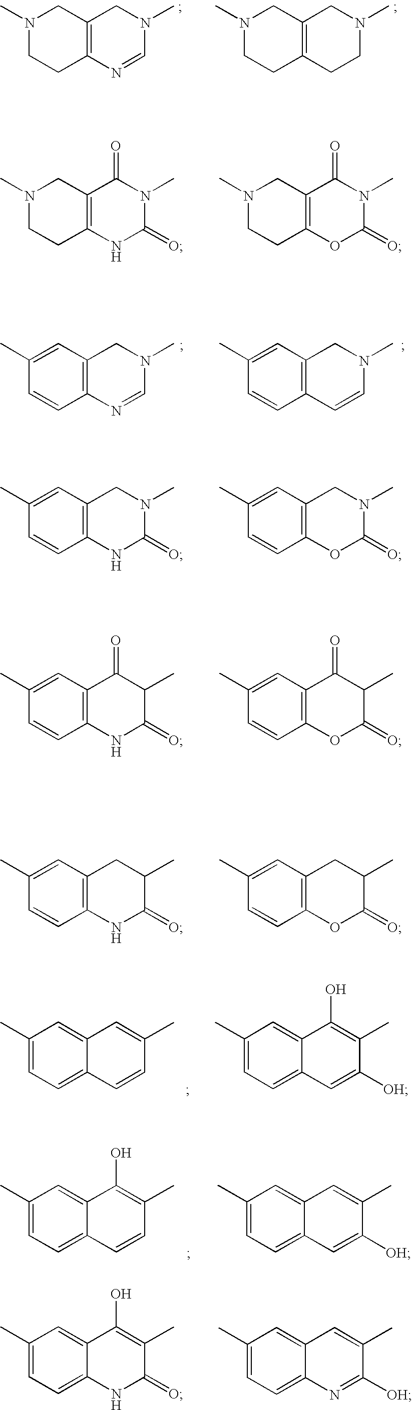

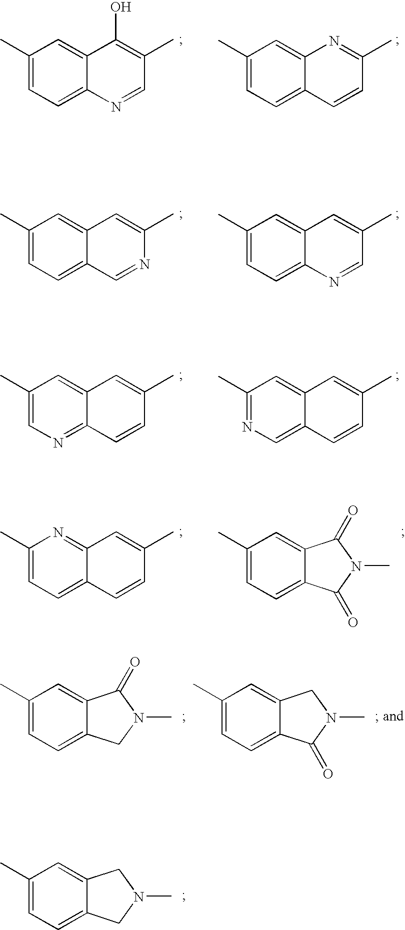

wherein the group D may be unsubstituted or substituted on a carbon atom or a nitrogen atom by replacement of a hydrogen atom with a group selected from:

CH₃;

CF₃;

C(O)H;

CN;

HO;

CH₃O;

C(F)H₂O;

C(H)F₂O; and

CF₃O;

wherein a carbon atom in the group D may further be substituted with F;

V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;

wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,

wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond; p2 wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;

wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;

wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

2. The compound according to Embodiment 1, or a pharmaceutically acceptable salt thereof, wherein Z is HO₂C.

3. The compound according to Embodiment 1, or a pharmaceutically acceptable salt thereof, wherein Z is HO(H)N(O)C.

4. The compound according to Embodiment 1, or a pharmaceutically acceptable salt thereof, wherein Z is CH₃(H)N(O)C—N(OH).

5. The compound according to Embodiment 1, or a pharmaceutically acceptable salt thereof, wherein Z is HS.

6. The compound according to Embodiment 1, or a pharmaceutically acceptable salt thereof, wherein Z is H₂N(O)₂S or CH₃(H)N(O)₂S.

7. The compound according to Embodiment 1, or a pharmaceutically acceptable salt thereof, wherein Z is selected from:

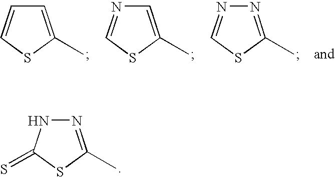

8. The compound according to Embodiment 1, or a pharmaceutically acceptable salt thereof, wherein Z is:

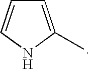

9. The compound according to Embodiment 1, or a pharmaceutically acceptable salt thereof, wherein Z is selected from:

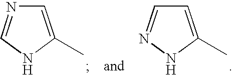

10. The compound according to Embodiment 1, or a pharmaceutically acceptable salt thereof, wherein Z is:

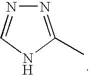

11. The compound according to Embodiment 1, or a pharmaceutically acceptable salt thereof, wherein Z is selected from:

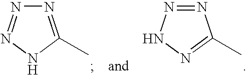

12. The compound according to Embodiment 1, or a pharmaceutically acceptable salt thereof, wherein Z is selected from:

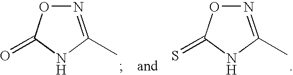

13. The compound according to Embodiment 1, or a pharmaceutically acceptable salt thereof, wherein Z is selected from:

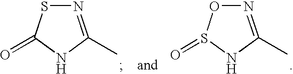

14. The compound according to any one of Embodiments 1 to 13, or a pharmaceutically acceptable salt thereof, wherein Q is OC(O).

15. The compound according to any one of Embodiments 1 to 13, or a pharmaceutically acceptable salt thereof, wherein Q is CH(R⁶)C(O).

16. The compound according to any one of Embodiments 1 to 13, or a pharmaceutically acceptable salt thereof, wherein Q is OC(NR⁶).

17. The compound according to any one of Embodiments 1 to 13, or a pharmaceutically acceptable salt thereof, wherein Q is CH(R⁶)C(NR⁶).

18. The compound according to any one of Embodiments 1 to 13, or a pharmaceutically acceptable salt thereof, wherein Q is N(R⁶)C(O).

19. The compound according to any one of Embodiments 1 to 13, or a pharmaceutically acceptable salt thereof, wherein Q is N(R⁶)C(NR⁶).

20. The compound according to any one of Embodiments 1 to 13, or a pharmaceutically acceptable salt thereof, wherein Q is N(R⁶)CH₂.

21. The compound according to any one of Embodiments 1 to 13, or a pharmaceutically acceptable salt thereof, wherein Q is SC(O).

22. The compound according to any one of Embodiments 1 to 13, or a pharmaceutically acceptable salt thereof, wherein Q is CH(R⁶)C(S).

23. The compound according to any one of Embodiments 1 to 13, or a pharmaceutically acceptable salt thereof, wherein Q is SC(NR⁶).

24. The compound according to any one of Embodiments 1 to 13, or a pharmaceutically acceptable salt thereof, wherein Q is selected from:

C≡C;

CH₂C≡C;

C≡CCH₂;

CF₂C≡C; and

C≡CCF₂.

25. The compound according to any one of Embodiments 1 to 24, or a pharmaceutically acceptable salt thereof, wherein L is OCH₂CH₂, OCH₂CH₂CH₂, or OCH₂CH₂CH₂CH₂.

26. The compound according to any one of Embodiments 1 to 25, or a pharmaceutically acceptable salt thereof, wherein at least one of R¹ is independently selected from:

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl); or

at least one of R² is independently selected from:

Phenyl-(C₁-C₈ alkylenyl)ₘ;

Substituted phenyl-(C₁-C₈ alkylenyl)ₘ;

5- or 6-membered heteroaryl-(C₁-C₈ alkylenyl)ₘ;

Substituted 5- or 6-membered heteroaryl-(C₁-C₈ alkylenyl)ₘ;

8- to 10-membered heterobiaryl-(C₁-C₈ alkylenyl)ₘ; and

Substituted 8- to 10-membered heterobiaryl-(C₁-C₈ alkylenyl)ₘ;

wherein m is an integer of 0 or 1; and

wherein each group and each substituent is independently selected.

27. The compound according to any one of Embodiments 1 to 26, or a pharmaceutically acceptable salt thereof, wherein R¹ is independently selected from:

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl); and

R² is independently selected from:

Phenyl-(C₁-C₈ alkylenyl)ₘ;

Substituted phenyl-(C₁-C₈ alkylenyl)ₘ;

5- or 6-membered heteroaryl-(C₁-C₈ alkylenyl)ₘ;

Substituted 5- or 6-membered heteroaryl-(C₁-C₈ alkylenyl)ₘ;

8- to 10-membered heterobiaryl-(C₁-C₈ alkylenyl)ₘ; and

Substituted 8- to 10-membered heterobiaryl-(C₁-C₈ alkylenyl)ₘ;

wherein m is an integer of 0 or 1; and

wherein each group and each substituent is independently selected.

28. The compound according to any one of Embodiments 1 to 27, or a pharmaceutically acceptable salt thereof, wherein R¹ is independently selected from:

Phenylenyl-(C₁-C₈ alkylenyl);

Substituted phenylenyl-(C₁-C₈ alkylenyl);

5- or 6-membered heteroarylenyl-(C₁-C₈ alkylenyl); and

Substituted 5- or 6-membered heteroarylenyl-(C₁-C₈ alkylenyl);

and R² is independently selected from:

Phenyl-(C₁-C₈ alkylenyl)ₘ;

Substituted phenyl-(C₁-C₈ alkylenyl)ₘ;

5- or 6-membered heteroaryl-(C₁-C₈ alkylenyl)ₘ; and

Substituted 5- or 6-membered heteroaryl-(C₁-C₈ alkylenyl)ₘ;

wherein m is an integer of 0 or 1; and

wherein each group and each substituent is independently selected.

29. The compound according to any one of Embodiments 1 to 25, or a pharmaceutically acceptable salt thereof, wherein R¹ is independently selected from:

C₅ or C₆ cycloalkylenyl-(C₁-C₈ alkylenyl);

Substituted C₃-C₆ cycloalkylenyl-(C₁-C₈ alkylenyl);

5- or 6-membered heterocycloalkylenyl-(C₁-C₈ alkylenyl); and

Substituted 5- or 6-membered heterocycloalkylenyl-(C₁-C₈ alkylenyl) and wherein each group and each substituent recited above is independently selected.

30. The compound according to any one of Embodiments 1 to 25, or a pharmaceutically acceptable salt thereof, wherein R¹ is substituted phenylenyl-(C₁-C₈ alkylenyl).

31. The compound according to any one of Embodiments 1 to 30, or a pharmaceutically acceptable salt thereof, wherein R⁶ is H or CH₃.

32. The compound according to any one of Embodiments 1 to 31, or a pharmaceutically acceptable salt thereof, wherein each C₁-C₈ alkylenyl is CH₂, C(CH₃)₂, C(═O), or CF₂.

33. The compound according to any one of Embodiments 1 to 32, or a pharmaceutically acceptable salt thereof, wherein each C₁-C₈ alkylenyl is CH₂.

34. The compound according to any one of Embodiments 1 to 33, or a pharmaceutically acceptable salt thereof, wherein at least one substituent is selected from the groups:

CO₂H;

CO₂CH₃;

F;

Cl;

CN;

CF₃;

HO;

CH₃O; and

CH₃.

35. A compound of Formula II

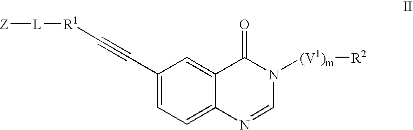

or a pharmaceutically acceptable salt thereof,

wherein:

Z is selected from:

HO₂C;

HO(H)N(O)C;

H(O)C—N(OH);

CH₃(O)C—N(OH);

CH₃(H)N(O)C—N(OH);

HS;

H₂N(O)₂S;

CH₃(H)N(O)₂S;

HO(O)P;

(HO)₂(O)P;

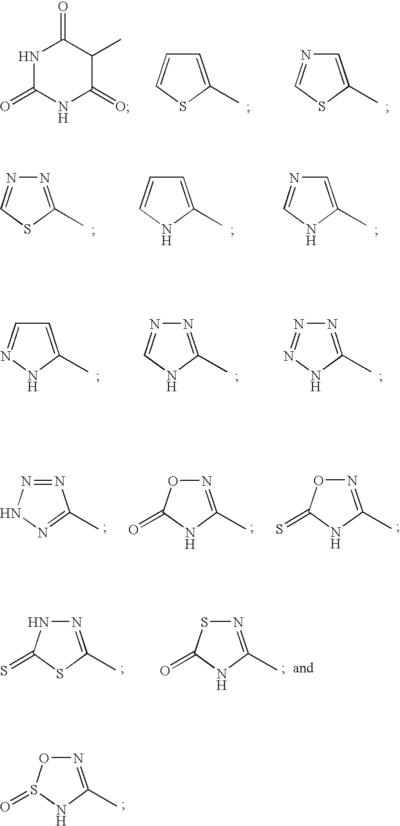

L is selected from:

C₃-C₅ alkylenyl;

Substituted C₃-C₅ alkylenyl;

3- to 5-membered heteroalkylenyl; and

Substituted 3- to 5-membered heteroalkylenyl;

Substituted L groups contain 1 or 2 substituents on a carbon atom or nitrogen atom independently selected from:

HO;

CN; and

CF₃;

wherein each substituent on a carbon atom may further be independently F, and wherein 2 substituents may be taken together with a carbon atom to which they are both bonded to form the group C═O;

R¹ is independently selected from:

C₅ or C₆ cycloalkylenyl-(C₁-C₈ alkylenyl);

Substituted C₅ or C₆ cycloalkylenyl-(C₁-C₈ alkylenyl);

5- or 6-membered heterocycloalkylenyl-(C₁-C₈ alkylenyl);

Substituted 5- or 6-membered heterocycloalkylenyl-(C₁-C₈ alkylenyl);

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl);

Phenyl;

Substituted phenyl;

Naphthyl;

Substituted naphthyl;

5- or 6-membered heteroaryl;

Substituted 5- or 6-membered heteroaryl;

8- to 10-membered heterobiaryl; and

Substituted 8- to 10-membered heterobiaryl;

R² is independently selected from:

H;

C₁-C₆ alkyl;

Phenyl-(C₁-C₈ alkylenyl);

Substituted phenyl-(C₁-C₈ alkylenyl);

Naphthyl-(C₁-C₈ alkylenyl);

Substituted naphthyl-(C₁-C₈ alkylenyl);

5- or 6-membered heteroaryl-(C₁-C₈ alkylenyl);

Substituted 5- or 6-membered heteroaryl-(C₁-C₈ alkylenyl);

8- to 10-membered heterobiaryl-(C₁-C₈ alkylenyl);

Substituted 8- to 10-membered heterobiaryl-(C₁-C₈ alkylenyl);

Phenyl-O—(C₁-C₈ alkylenyl);

Substituted phenyl-O—(C₁-C₈ alkylenyl);

Phenyl-S—(C₁-C₈ alkylenyl);

Substituted phenyl-S—(C₁-C₈ alkylenyl);

Phenyl-S(O)—(C₁-C₈ alkylenyl);

Substituted phenyl-S(O)—(C₁-C₈ alkylenyl);

Phenyl-S(O)₂—(C₁-C₈ alkylenyl); and

Substituted phenyl-S(O)₂—(C₁-C₈ alkylenyl);

Each substituted R¹ group contains from 1 to 3 substituents, and each substituted R² group contains from 1 to 4 substituents, wherein each substituent is independently on a carbon or nitrogen atom, independently selected from:

C₁-C₆ alkyl;

CN;

CF₃;

HO;

(C₁-C₆ alkyl)-O;

(C₁-C₆ alkyl)-S(O)₂;

H₂N;

(C₁-C₆ alkyl)-N(H);

(C₁-C₆ alkyl)₂-N;

(C₁-C₆ alkyl)-C(O)O—(C₁-C₈ alkylenyl)ₘ;

(C₁-C₆ alkyl)-C(O)O-(1- to 8-membered heteroalkylenyl)ₘ;

(C₁-C₆ alkyl)-C(O)N(H)—(C₁-C₈ alkylenyl)ₘ;

(C₁-C₆ alkyl)-C(O)N(H)-(1- to 8-membered heteroalkylenyl)ₘ;

H₂NS(O)₂—(C₁-C₈ alkylenyl);

(C₁-C₆ alkyl)-N(H)S(O)₂—(C₁-C₈ alkylenyl)ₘ;

(C₁-C₆ alkyl)₂-NS(O)₂—(C₁-C₈ alkylenyl)ₘ;

3- to 6-membered heterocycloalkyl-(G)ₘ;

Substituted 3- to 6-membered heterocycloalkyl-(G)ₘ;

5- or 6-membered heteroaryl-(G)ₘ;

Substituted 5- or 6-membered heteroaryl-(G)ₘ;

(C₁-C₆ alkyl)-S(O)₂—N(H)—C(O)—(C₁-C₈ alkylenyl)ₘ; and

(C₁-C₆ alkyl)-C(O)—N(H)—S(O)₂—(C₁-C₈ alkylenyl)ₘ;

wherein each substituent on a carbon atom may further be independently selected from:

Halo; and

HO₂C;

wherein 2 substituents may be taken together with a carbon atom to which they are both bonded to form the group C═O;

wherein two adjacent, substantially sp² carbon atoms may be taken together with a diradical substituent to form a cyclic diradical selected from:

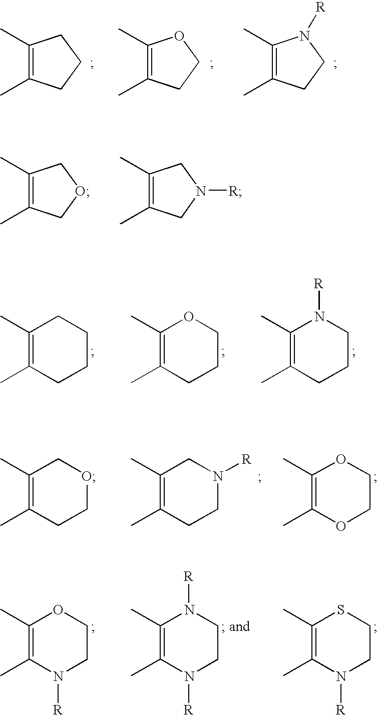

R is H or C₁-C₆ alkyl;

G is CH₂; O, S, S(O); or S(O)₂;

Each m is an integer of 0 or 1;

wherein the group D may be unsubstituted or substituted on a carbon atom or a nitrogen atom by replacement of a hydrogen atom with a group selected from:

CH₃;

CF₃;

C(O)H;

CN;

HO;

CH₃O;

C(F)H₂O;

C(H)F₂O; and

CF₃O;

wherein a carbon atom in the group D may further be substituted with F;

V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;

wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,

wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;

wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;

wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

36. The compound according to Embodiment 35, named:

4-(3-{3-[3-(3,4-Difluoro-benzyl)-4-oxo-3,4-dihydro-quinazolin-6-yl]-prop-2-ynyl}-phenyl)-butyric acid;

or a pharmaceutically acceptable salt thereof.

37. A compound of Formula III

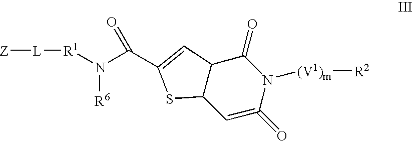

or a pharmaceutically acceptable salt thereof,

wherein:

Z is selected from:

HO₂C;

HO(H)N(O)C;

H(O)C—N(OH);

CH₃(O)C—N(OH);

CH₃(H)N(O)C—N(OH);

HS;

H₂N(O)₂S;

CH₃(H)N(O)₂S;

HO(O)P;

(HO)₂(O)P;

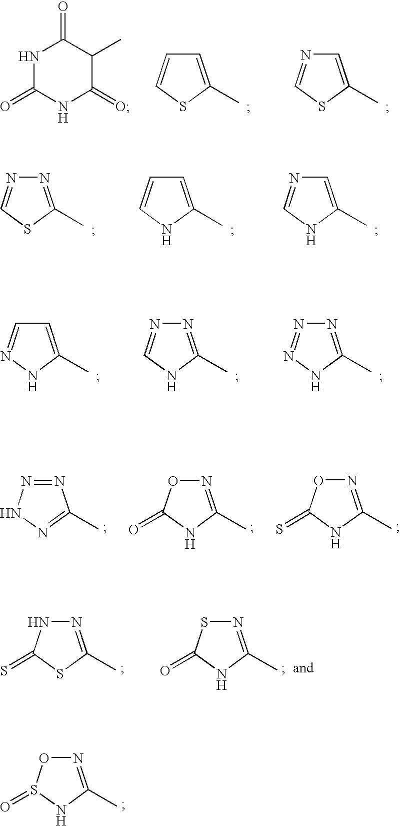

L is selected from:

C₃-C₅ alkylenyl;

Substituted C₃-C₅ alkylenyl;

3- to 5-membered heteroalkylenyl; and

Substituted 3- to 5-membered heteroalkylenyl;

Substituted L groups contain 1 or 2 substituents on a carbon atom or nitrogen atom independently selected from:

HO;

CN; and

CF₃;

wherein each substituent on a carbon atom may further be independently F, and wherein 2 substituents may be taken together with a carbon atom to which they are both bonded to form the group C═O;

R¹ is independently selected from:

C₅ or C₆ cycloalkylenyl-(C₁-C₈ alkylenyl);

Substituted C₅ or C₆ cycloalkylenyl-(C₁-C₈ alkylenyl);

5- or 6-membered heterocycloalkylenyl-(C₁-C₈ alkylenyl);

Substituted 5- or 6-membered heterocycloalkylenyl-(C₁-C₈ alkylenyl);

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl);

Phenyl;

Substituted phenyl;

Naphthyl;

Substituted naphthyl;

5- or 6-membered heteroaryl;

Substituted 5- or 6-membered heteroaryl;

8- to 10-membered heterobiaryl; and

Substituted 8- to 10-membered heterobiaryl;

R² is independently selected from:

H;

C₁-C₆ alkyl;

Phenyl-(C₁-C₈ alkylenyl);

Substituted phenyl-(C₁-C₈ alkylenyl);

Naphthyl-(C₁-C₈ alkylenyl);

Substituted naphthyl-(C₁-C₈ alkylenyl);

5- or 6-membered heteroaryl-(C₁-C₈ alkylenyl);

Substituted 5- or 6-membered heteroaryl-(C₁-C₈ alkylenyl);

8- to 10-membered heterobiaryl-(C₁-C₈ alkylenyl);

Substituted 8- to 10-membered heterobiaryl-(C₁-C₈ alkylenyl);

Phenyl-O—(C₁-C₈ alkylenyl);

Substituted phenyl-O—(C₁-C₈ alkylenyl);

Phenyl-S—(C₁-C₈ alkylenyl);

Substituted phenyl-S—(C₁-C₈ alkylenyl);

Phenyl-S(O)—(C₁-C₈ alkylenyl);

Substituted phenyl-S(O)—(C₁-C₈ alkylenyl);

Phenyl-S(O)₂—(C₁-C₈ alkylenyl); and

Substituted phenyl-S(O)₂—(C₁-C₈ alkylenyl);

Each substituted R¹ group contains from 1 to 3 substituents, and each substituted R² group contains from 1 to 4 substituents, wherein each substituent is independently on a carbon or nitrogen atom, independently selected from:

C₁-C₆ alkyl;

CN;

CF₃;

HO;

(C₁-C₆ alkyl)-O;

(C₁-C₆ alkyl)-S(O)₂;

H₂N;

(C₁-C₆ alkyl)-N(H);

(C₁-C₆ alkyl)₂-N;

(C₁-C₆ alkyl)-C(O)O—(C₁-C₈ alkylenyl)ₘ;

(C₁-C₆ alkyl)-C(O)O-(1- to 8-membered heteroalkylenyl)ₘ;

(C₁-C₆ alkyl)-C(O)N(H)—(C₁-C₈ alkylenyl)ₘ;

(C₁-C₆ alkyl)-C(O)N(H)-(1- to 8-membered heteroalkylenyl)ₘ;

H₂NS(O)₂—(C₁-C₈ alkylenyl);

(C₁-C₆ alkyl)-N(H)S(O)₂—(C₁-C₈ alkylenyl)ₘ;

(C₁-C₆ alkyl)₂-NS(O)₂—(C₁-C₈ alkylenyl)ₘ;

3- to 6-membered heterocycloalkyl-(G)ₘ;

Substituted 3- to 6-membered heterocycloalkyl-(G)ₘ;

5- or 6-membered heteroaryl-(G)ₘ;

Substituted 5- or 6-membered heteroaryl-(G)ₘ;

(C₁-C₆ alkyl)-S(O)₂—N(H)—C(O)—(C₁-C₈ alkylenyl)ₘ; and

(C₁-C₆ alkyl)-C(O)—N(H)—S(O)₂—(C₁-C₈ alkylenyl)ₘ;

wherein each substituent on a carbon atom may further be independently selected from:

Halo; and

HO₂C;

wherein 2 substituents may be taken together with a carbon atom to which they are both bonded to form the group C═O;

wherein two adjacent, substantially sp² carbon atoms may be taken together with a diradical substituent to form a cyclic diradical selected from:

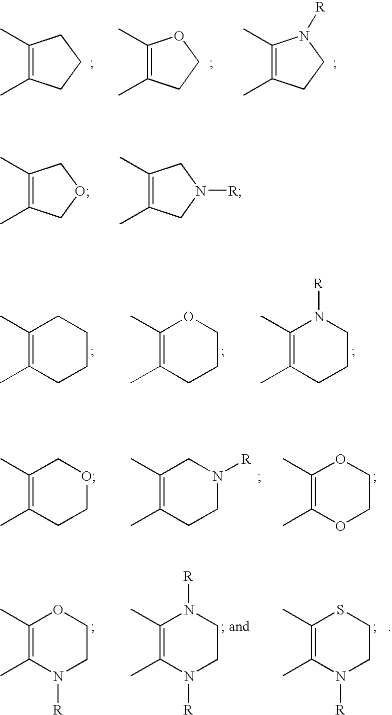

R is H or C₁-C₆ alkyl;

G is CH₂; O, S, S(O); or S(O)₂;

Each m is an integer of 0 or 1;

R⁶ independently is H, C₁-C₆ alkyl, C₃-C₆ cycloalkyl; 3- to 6-membered heterocycloalkyl; phenyl; benzyl; or 5- or 6-membered heteroaryl;

wherein the group D may be unsubstituted or substituted on a carbon atom or a nitrogen atom by replacement of a hydrogen atom with a group selected from:

CH₃;

CF₃;

C(O)H;

CN;

HO;

CH₃O;

C(F)H₂O;

C(H)F₂O; and

CF₃O;

wherein a carbon atom in the group D may further be substituted with F;

V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;

wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,

wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;

wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;

wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

38. The compound according to Embodiment 37, named:

5-(3,4-Difluoro-benzyl)-7-methyl-4,6-dioxo-3a,4,5,6-tetrahydro-thieno[3,2-c]pyridine-2-carboxylic acid [2-(3-mercapto-propoxy)-pyridin-4-ylmethyl]-amide;

or a pharmaceutically acceptable salt thereof.

39. A compound of Formula IV

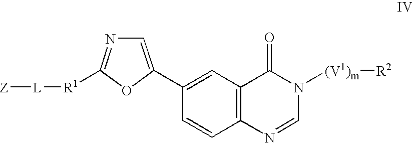

or a pharmaceutically acceptable salt thereof,

wherein:

Z is selected from:

HO₂C;

HO(H)N(O)C;

H(O)C—N(OH);

CH₃(O)C—N(OH);

CH₃(H)N(O)C—N(OH);

HS;

H₂N(O)₂S;

CH₃(H)N(O)₂S;

HO(O)P;

(HO)₂(O)P;

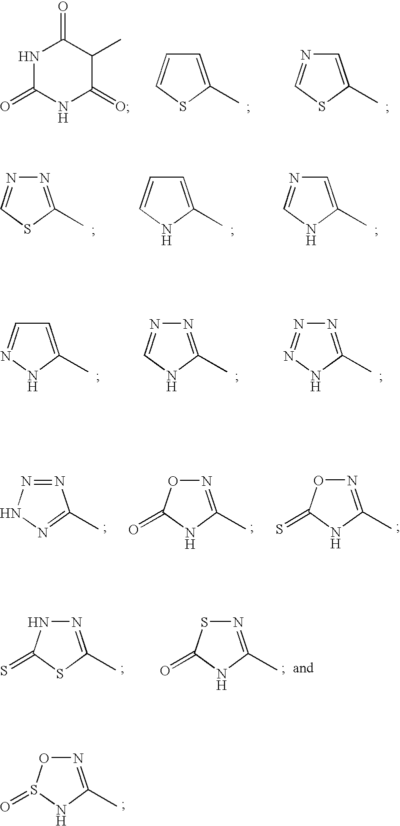

L is selected from:

C₃-C₅ alkylenyl;

Substituted C₃-C₅ alkylenyl;

3- to 5-membered heteroalkylenyl; and

Substituted 3- to 5-membered heteroalkylenyl;

Substituted L groups contain 1 or 2 substituents on a carbon atom or nitrogen atom independently selected from:

HO;

CN; and

CF₃;

wherein each substituent on a carbon atom may further be independently F, and wherein 2 substituents may be taken together with a carbon atom to which they are both bonded to form the group C═O;

R¹ is independently selected from:

C₅ or C₆ cycloalkylenyl-(C₁-C₈ alkylenyl);

Substituted C₅ or C₆ cycloalkylenyl-(C₁-C₈ alkylenyl);

5- or 6-membered heterocycloalkylenyl-(C₁-C₈ alkylenyl);

Substituted 5- or 6-membered heterocycloalkylenyl-(C₁-C₈ alkylenyl);

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl);

Phenyl;

Substituted phenyl;

Naphthyl;

Substituted naphthyl;

5- or 6-membered heteroaryl;

Substituted 5- or 6-membered heteroaryl;

8- to 10-membered heterobiaryl; and

Substituted 8- to 10-membered heterobiaryl;

R² is independently selected from:

H;

C₁-C₆ alkyl;

Phenyl-(C₁-C₈ alkylenyl);

Substituted phenyl-(C₁-C₈ alkylenyl);

Naphthyl-(C₁-C₈ alkylenyl);

Substituted naphthyl-(C₁-C₈ alkylenyl);

5- or 6-membered heteroaryl-(C₁-C₈ alkylenyl);

Substituted 5- or 6-membered heteroaryl-(C₁-C₈ alkylenyl);

8- to 10-membered heterobiaryl-(C₁-C₈ alkylenyl);

Substituted 8- to 10-membered heterobiaryl-(C₁-C₈ alkylenyl);

Phenyl-O—(C₁-C₈ alkylenyl);

Substituted phenyl-O—(C₁-C₈ alkylenyl);

Phenyl-S—(C₁-C₈ alkylenyl);

Substituted phenyl-S—(C₁-C₈ alkylenyl);

Phenyl-S(O)—(C₁-C₈ alkylenyl);

Substituted phenyl-S(O)—(C₁-C₈ alkylenyl);

Phenyl-S(O)₂—(C₁-C₈ alkylenyl); and

Substituted phenyl-S(O)₂—(C₁-C₈ alkylenyl);

Each substituted R¹ group contains from 1 to 3 substituents, and each substituted R² group contains from 1 to 4 substituents, wherein each substituent is independently on a carbon or nitrogen atom, independently selected from:

C₁-C₆ alkyl;

CN;

CF₃;

HO;

(C₁-C₆ alkyl)-O;

(C₁-C₆ alkyl)-S(O)₂;

H₂N;

(C₁-C₆ alkyl)-N(H);

(C₁-C₆ alkyl)₂-N;

(C₁-C₆ alkyl)-C(O)O—(C₁-C₈ alkylenyl)ₘ;

(C₁-C₆ alkyl)-C(O)O-(1- to 8-membered heteroalkylenyl)ₘ;

(C₁-C₆ alkyl)-C(O)N(H)—(C₁-C₈ alkylenyl)ₘ;

(C₁-C₆ alkyl)-C(O)N(H)-(1- to 8-membered heteroalkylenyl)ₘ;

H₂NS(O)₂—(C₁-C₈ alkylenyl);

(C₁-C₆ alkyl)-N(H)S(O)₂—(C₁-C₈ alkylenyl)ₘ;

(C₁-C₆ alkyl)₂-NS(O)₂—(C₁-C₈ alkylenyl)ₘ;

3- to 6-membered heterocycloalkyl-(G)ₘ;

Substituted 3- to 6-membered heterocycloalkyl-(G)ₘ;

5- or 6-membered heteroaryl-(G)ₘ;

Substituted 5- or 6-membered heteroaryl-(G)ₘ;

(C₁-C₆ alkyl)-S(O)₂—N(H)—C(O)—(C₁-C₈ alkylenyl)ₘ; and

(C₁-C₆ alkyl)-C(O)—N(H)—S(O)₂—(C₁-C₈ alkylenyl)ₘ;

wherein each substituent on a carbon atom may further be independently selected from:

Halo; and

HO₂C;

wherein 2 substituents may be taken together with a carbon atom to which they are both bonded to form the group C═O;

wherein two adjacent, substantially sp² carbon atoms may be taken together with a diradical substituent to form a cyclic diradical selected from:

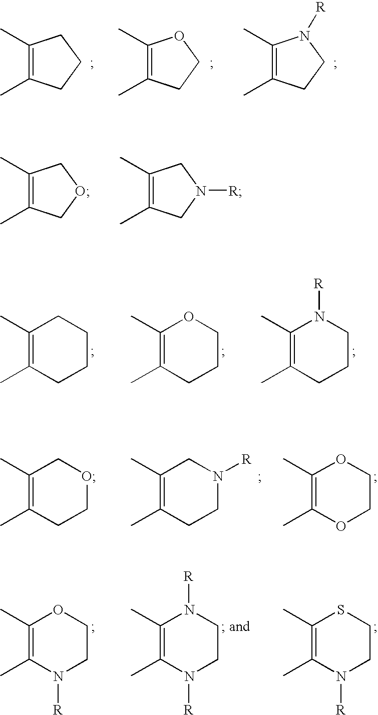

R is H or C₁-C₆ alkyl;

G is CH₂; O, S, S(O); or S(O)₂;

Each m is an integer of 0 or 1;

wherein the group D may be unsubstituted or substituted on a carbon atom or a nitrogen atom by replacement of a hydrogen atom with a group selected from:

CH₃;

CF₃;

C(O)H;

CN;

HO;

CH₃O;

C(F)H₂O;

C(H)F₂O; and

CF₃O;

wherein a carbon atom in the group D may further be substituted with F;

V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;

wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,

wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;

wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;

wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

40. The compound according to Embodiment 39, named:

4-(6-{2-[3-(2-Hydroxycarbamoyl-ethoxy)-phenyl]-oxazol-5-yl}-4-oxo-4H-quinazolin-3-ylmethyl)-benzoic acid;

or a pharmaceutically acceptable salt thereof.

41. The compound according to Embodiment 1, wherein Q is

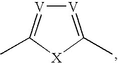

wherein V and X are as defined above.

42. The compound according to Embodiment 1, wherein Q is

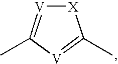

wherein V and X are as defined above.

43. The compound according to Embodiment 1, wherein Q is

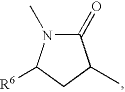

wherein R⁶ is as defined above.

44. The compound according to Embodiment 1, wherein Q is

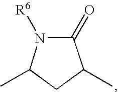

wherein R⁶ is as defined above.

45. The compound according to Embodiment 1, wherein Q is

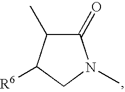

wherein R⁶ is as defined above.

46. The compound according to Embodiment 1, wherein Q is selected from:

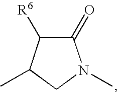

wherein R⁶ is as defined above.

47. The compound according to Embodiment 1, wherein Q is selected from:

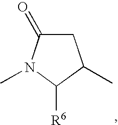

wherein R⁶ is as defined above.

48. A pharmaceutical composition, comprising a compound of Formula I according to Embodiment 1, or a pharmaceutically acceptable salt thereof, admixed with a pharmaceutically acceptable carrier, excipient, or diluent.

49. The pharmaceutical composition according to Embodiment 48, comprising a compound of Formula I according to any one of Embodiments 2 to 47, or a pharmaceutically acceptable salt thereof, admixed with a pharmaceutically acceptable carrier, excipient, or diluent.

50. A method for inhibiting an MMP-13 enzyme in an animal, comprising administering to the animal an MMP-13 inhibiting amount of a compound of Formula I according to Embodiment 1, or a pharmaceutically acceptable salt thereof.

51. The method according to Embodiment 50, wherein the compound of Formula I is according to any one of Embodiments 2 to 47, or a pharmaceutically acceptable salt thereof.

52. A method for treating a disease mediated by an MMP-13 enzyme, comprising administering to a patient suffering from such a disease a nontoxic effective amount of a compound of Formula I according to Embodiment 1, or a pharmaceutically acceptable salt thereof.

53. The method according to Embodiment 52, wherein the compound of Formula I is according to any one of Embodiments 2 to 47, or a pharmaceutically acceptable salt thereof.

54. A method for treating arthritis, comprising administering to a patient suffering from an arthritis disease a nontoxic antiarthritic effective amount of a compound of Formula I according to Embodiment 1, or a pharmaceutically acceptable salt thereof.

55. The method according to Embodiment 54, wherein the compound of Formula I is according to any one of Embodiments 2 to 47, or a pharmaceutically acceptable salt thereof.

56. A method for treating osteoarthritis, comprising administering to a patient suffering from osteoarthritis a nontoxic effective amount of a compound of Formula I according to Embodiment 1, or a pharmaceutically acceptable salt thereof.

57. The method according to Embodiment 56, wherein the compound of Formula I is according to any one of Embodiments 2 to 47, or a pharmaceutically acceptable salt thereof.

58. A method for treating rheumatoid arthritis, comprising administering to a patient suffering from rheumatoid arthritis a nontoxic effective amount of a compound of Formula I according to Embodiment 1, or a pharmaceutically acceptable salt thereof.

59. The method according to Embodiment 58, wherein the compound of Formula I is according to any one of Embodiments 2 to 47, or a pharmaceutically acceptable salt thereof.

60. A method for treating psoriatic arthritis, comprising administering to a patient suffering from psoriatic arthritis a nontoxic effective amount of a compound of Formula I according to Embodiment 1, or a pharmaceutically acceptable salt thereof.

61. The method according to Embodiment 60, wherein the compound of Formula I is according to any one of Embodiments 2 to 47, or a pharmaceutically acceptable salt thereof.

62. A method for treating a cancer, comprising administering to a patient suffering from a cancer a nontoxic anti-cancer effective amount of a compound of Formula I according to Embodiment 1, or a pharmaceutically acceptable salt thereof.

63. The method according to Embodiment 62, wherein the compound of Formula I is according to any one of Embodiments 2 to 47, or a pharmaceutically acceptable salt thereof.

64. A method for treating breast carcinoma, comprising administering to a patient suffering from breast carcinoma a nontoxic effective amount of a compound of Formula I according to Embodiment 1, or a pharmaceutically acceptable salt thereof.

65. The method according to Embodiment 64, wherein the compound of Formula I is according to any one of Embodiments 2 to 47, or a pharmaceutically acceptable salt thereof.

66. A method for treating atherosclerosis, comprising administering to a patient suffering from atherosclerosis a nontoxic effective amount of a compound of Formula I according to Embodiment 1, or a pharmaceutically acceptable salt thereof.

67. The method according to Embodiment 66, wherein the compound of Formula I is according to any one of Embodiments 2 to 47, or a pharmaceutically acceptable salt thereof.

68. A method for treating inflammation, comprising administering to a patient suffering from inflammation a nontoxic effective amount of a compound of Formula I according to Embodiment 1, or a pharmaceutically acceptable salt thereof.

69. The method according to Embodiment 68, wherein the compound of Formula I is according to any one of Embodiments 2 to 47, or a pharmaceutically acceptable salt thereof.

70. A method for treating heart failure, comprising administering to a patient suffering from heart failure a nontoxic effective amount of a compound of Formula I according to Embodiment 1, or a pharmaceutically acceptable salt thereof.

71. The method according to Embodiment 70, wherein the compound of Formula I is according to any one of Embodiments 2 to 47, or a pharmaceutically acceptable salt thereof.

72. A method for treating age-related macular degeneration, comprising administering to a patient suffering from age-related macular degeneration a nontoxic effective amount of a compound of Formula I according to Embodiment 1, or a pharmaceutically acceptable salt thereof.

73. The method according to Embodiment 72, wherein the compound of Formula I is according to any one of Embodiments 2 to 47, or a pharmaceutically acceptable salt thereof.

74. A method for treating chronic obstructive pulmonary disease, comprising administering to a patient suffering from chronic obstructive pulmonary disease a nontoxic effective amount of a compound of Formula I according to Embodiment 1, or a pharmaceutically acceptable salt thereof.

75. The method according to Embodiment 74, wherein the compound of Formula I is according to any one of Embodiments 2 to 47, or a pharmaceutically acceptable salt thereof.

76. A method for treating heart disease, comprising administering to a patient suffering from heart disease a nontoxic effective amount of a compound of Formula I according to Embodiment 1, or a pharmaceutically acceptable salt thereof.

77. The method according to Embodiment 76, wherein the compound of Formula I is according to any one of Embodiments 2 to 47, or a pharmaceutically acceptable salt thereof.

78. A method for treating multiple sclerosis, comprising administering to a patient suffering from multiple sclerosis a nontoxic effective amount of a compound of Formula I according to Embodiment 1, or a pharmaceutically acceptable salt thereof.

79. The method according to Embodiment 78, wherein the compound of Formula I is according to any one of Embodiments 2 to 47, or a pharmaceutically acceptable salt thereof.

80. A method for treating psoriasis, comprising administering to a patient suffering from psoriasis a nontoxic effective amount of a compound of Formula I according to Embodiment 1, or a pharmaceutically acceptable salt thereof.

81. The method according to Embodiment 80, wherein the compound of Formula I is according to any one of Embodiments 2 to 47, or a pharmaceutically acceptable salt thereof.

82. A method for treating asthma, comprising administering to a patient suffering from asthma a nontoxic effective amount of a compound of Formula I according to Embodiment 1, or a pharmaceutically acceptable salt thereof.

83. The method according to Embodiment 82, wherein the compound of Formula I is according to any one of Embodiments 2 to 47, or a pharmaceutically acceptable salt thereof.

84. A method for treating cardiac insufficiency, comprising administering to a patient suffering from cardiac insufficiency a nontoxic effective amount of a compound of Formula I according to Embodiment 1, or a pharmaceutically acceptable salt thereof.

85. The method according to Embodiment 84, wherein the compound of Formula I is according to any one of Embodiments 2 to 47, or a pharmaceutically acceptable salt thereof.

86. A method for treating inflammatory bowel disease, comprising administering to a patient suffering from inflammatory bowel disease a nontoxic effective amount of a compound of Formula I according to Embodiment 1, or a pharmaceutically acceptable salt thereof.

87. The method according to Embodiment 86, wherein the compound of Formula I is according to any one of Embodiments 2 to 47, or a pharmaceutically acceptable salt thereof.

88. A method for treating osteoporosis, comprising administering to a patient suffering from osteoporosis a nontoxic effective amount of a compound of Formula I according to Embodiment 1, or a pharmaceutically acceptable salt thereof.

89. The method according to Embodiment 88, wherein the compound of Formula I is according to any one of Embodiments 2 to 47, or a pharmaceutically acceptable salt thereof.

90. A method for treating periodontal diseases, comprising administering to a patient suffering from periodontal diseases a nontoxic effective amount of a compound of Formula I according to Embodiment 1, or a pharmaceutically acceptable salt thereof.

91. The method according to Embodiment 90, wherein the compound of Formula I is according to any one of Embodiments 2 to 47, or a pharmaceutically acceptable salt thereof.

92. The method according to any one of Embodiments 50 to 91, wherein the compound of Formula I according to Embodiment 1, or a pharmaceutically acceptable salt thereof, is administered as a pharmaceutical composition according to Embodiment 48 or 49.

93. The compound according to Embodiment 1, wherein V¹ is selected from the groups:

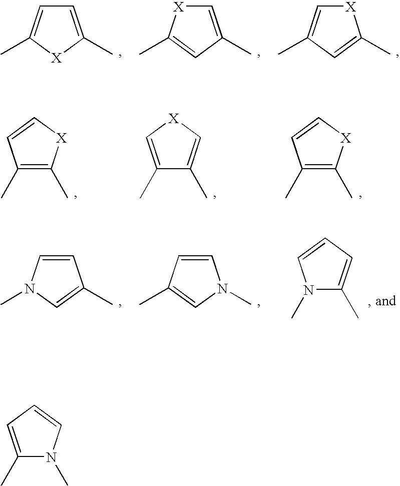

wherein X is O, S, N(H), or N(C₁-C₆ alkyl) and V¹ may optionally be unsubstituted or substituted at C(H) or N(H) with 1 substituent selected from fluoro, methyl, hydroxy, trifluoromethyl, cyano, and acetyl.

94. The compound according to Embodiment 1, or a pharmaceutically acceptable salt thereof, wherein V¹ is selected from the groups:

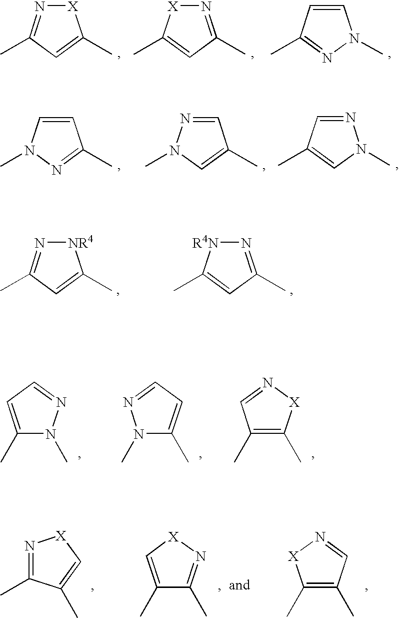

wherein X is O, S, N(H), or N(C₁-C₆ alkyl), R⁴ is H or C₁-C₆ alkyl, and V¹ may optionally be unsubstituted or substituted at C(H) or N(H) with 1 substituent selected from fluoro, methyl, hydroxy, trifluoromethyl, cyano, and acetyl.

95. The compound according to Embodiment 1, or a pharmaceutically acceptable salt thereof, wherein V¹ is selected from the groups:

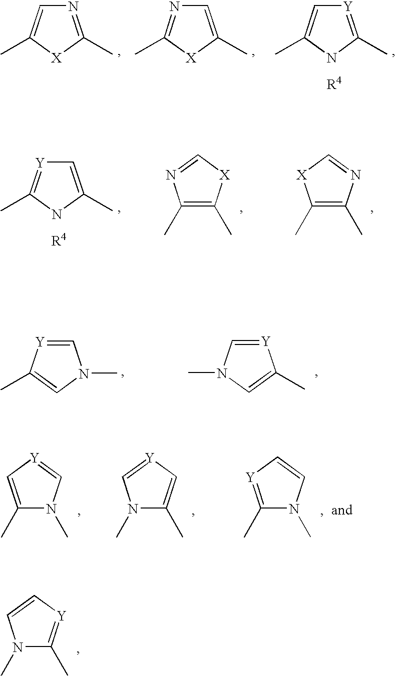

wherein X is O, S, N(H), or N(C₁-C₆ alkyl), Y is O, S, or N, and R⁴ is H or C₁-C₆ alkyl, and V¹ may optionally be unsubstituted or substituted at C(H) or N(H) with 1 substituent selected from fluoro, methyl, hydroxy, trifluoromethyl, cyano, and acetyl.

96. The compound according to Embodiment 1, or a pharmaceutically acceptable salt thereof, wherein V¹ is selected from the groups:

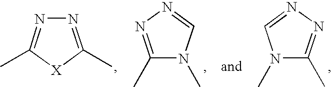

wherein X is O, S, N(H), or N(C₁-C₆ alkyl), and V¹ may optionally be unsubstituted or substituted at C(H) with 1 substituent selected from fluoro, methyl, hydroxy, trifluoromethyl, cyano, and acetyl.

97. The compound according to Embodiment 1, or a pharmaceutically acceptable salt thereof, wherein V¹ is selected from the groups:

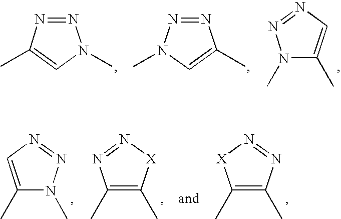

wherein X is O, S, N(H), or N(C₁-C₆ alkyl), and V¹ may optionally be unsubstituted or substituted at C(H) with 1 substituent selected from fluoro, methyl, hydroxy, trifluoromethyl, cyano, and acetyl.

98. The compound according to Embodiment 1, or a pharmaceutically acceptable salt thereof, wherein V¹ is selected from the groups:

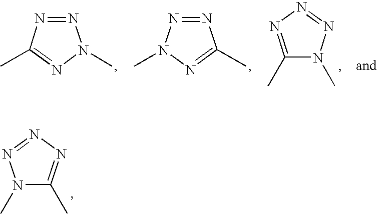

99. A compound of Formula I

Z—L—R¹—Q—D—(V¹)ₘ—R² I

or a pharmaceutically acceptable salt thereof,

wherein:

Z is selected from:

HO₂C;

HO(H)N(O)C;

H(O)C—N(OH);

CH₃(O)C—N(OH);

CH₃(H)N(O)C—N(OH);

HS;

H₂N(O)₂S;

CH₃(H)N(O)₂S;

HO(O)P;

(HO)₂(O)P;

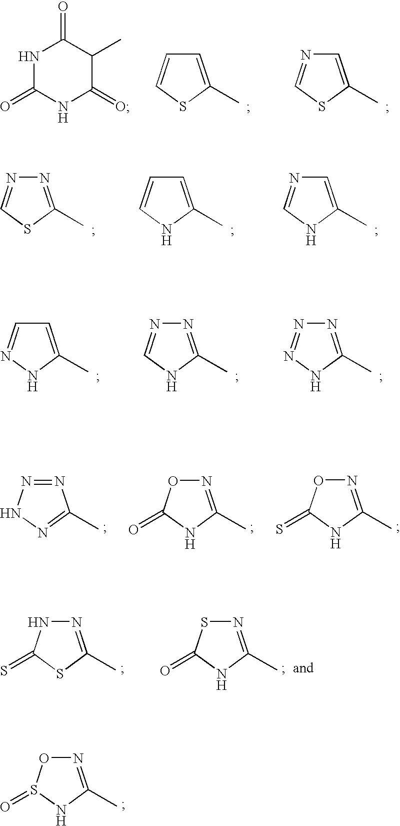

L is selected from:

C₃-C₅ alkylenyl;

Substituted C₃-C₅ alkylenyl;

3- to 5-membered heteroalkylenyl; and

Substituted 3- to 5-membered heteroalkylenyl;

Substituted L groups contain 1 or 2 substituents on a carbon atom or nitrogen atom independently selected from:

HO;

CN; and

CF₃;

wherein each substituent on a carbon atom may further be independently F, and wherein 2 substituents may be taken together with a carbon atom to which they are both bonded to form the group C═O;

R¹ is independently selected from:

C₅ or C₆ cycloalkylenyl-(C₁-C₈ alkylenyl);

Substituted C₅ or C₆ cycloalkylenyl-(C₁-C₈ alkylenyl);

5- or 6-membered heterocycloalkylenyl-(C₁-C₈ alkylenyl);

Substituted 5- or 6-membered heterocycloalkylenyl-(C₁-C₈ alkylenyl);

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl);

Phenyl;

Substituted phenyl;

Naphthyl;

Substituted naphthyl;

5- or 6-membered heteroaryl;

Substituted 5- or 6-membered heteroaryl;

8- to 10-membered heterobiaryl; and

Substituted 8- to 10-membered heterobiaryl;

R² is independently selected from:

H;

C₁-C₆ alkyl;

Phenyl-(C₁-C₈ alkylenyl);

Substituted phenyl-(C₁-C₈ alkylenyl);

Naphthyl-(C₁-C₈ alkylenyl);

Substituted naphthyl-(C₁-C₈ alkylenyl);

5- or 6-membered heteroaryl-(C₁-C₈ alkylenyl);

Substituted 5- or 6-membered heteroaryl-(C₁-C₈ alkylenyl);

8- to 10-membered heterobiaryl-(C₁-C₈ alkylenyl);

Substituted 8- to 10-membered heterobiaryl-(C₁-C₈ alkylenyl);

Phenyl-O—(C₁-C₈ alkylenyl);

Substituted phenyl-O—(C₁-C₈ alkylenyl);

Phenyl-S—(C₁-C₈ alkylenyl);

Substituted phenyl-S—(C₁-C₈ alkylenyl);

Phenyl-S(O)—(C₁-C₈ alkylenyl);

Substituted phenyl-S(O)—(C₁-C₈ alkylenyl);

Phenyl-S(O)₂—(C₁-C₈ alkylenyl); and

Substituted phenyl-S(O)₂—(C₁-C₈ alkylenyl);

Each substituted R¹ group contains from 1 to 3 substituents, and each substituted R² group contains from 1 to 4 substituents, wherein each substituent is independently on a carbon or nitrogen atom, independently selected from:

C₁-C₆ alkyl;

CN;

CF₃;

HO;

(C₁-C₆ alkyl)-O;

(C₁-C₆ alkyl)-S(O)₂;

H₂N;

(C₁-C₆ alkyl)-N(H);

(C₁-C₆ alkyl)₂-N;

(C₁-C₆ alkyl)-C(O)O—(C₁-C₈ alkylenyl)ₘ;

(C₁-C₆ alkyl)-C(O)O-(1- to 8-membered heteroalkylenyl)ₘ;

(C₁-C₆ alkyl)-C(O)N(H)—(C₁-C₈ alkylenyl)ₘ;

(C₁-C₆ alkyl)-C(O)N(H)-(1- to 8-membered heteroalkylenyl)ₘ;

H₂NS(O)₂—(C₁-C₈ alkylenyl);

(C₁-C₆ alkyl)-N(H)S(O)₂—(C₁-C₈ alkylenyl)ₘ;

(C₁-C₆ alkyl)₂-NS(O)₂—(C₁-C₈ alkylenyl)ₘ;

3- to 6-membered heterocycloalkyl-(G)ₘ;

Substituted 3- to 6-membered heterocycloalkyl-(G)ₘ;

5- or 6-membered heteroaryl-(G)ₘ;

Substituted 5- or 6-membered heteroaryl-(G)ₘ;

(C₁-C₆ alkyl)-S(O)₂—N(H)—C(O)—(C₁-C₈ alkylenyl)ₘ; and

(C₁-C₆ alkyl)-C(O)—N(H)—S(O)₂—(C₁-C₈ alkylenyl)ₘ;

wherein each substituent on a carbon atom may further be independently selected from:

Halo; and

HO₂C;

wherein 2 substituents may be taken together with a carbon atom to which they are both bonded to form the group C═O;

wherein two adjacent, substantially sp² carbon atoms may be taken together with a diradical substituent to form a cyclic diradical selected from:

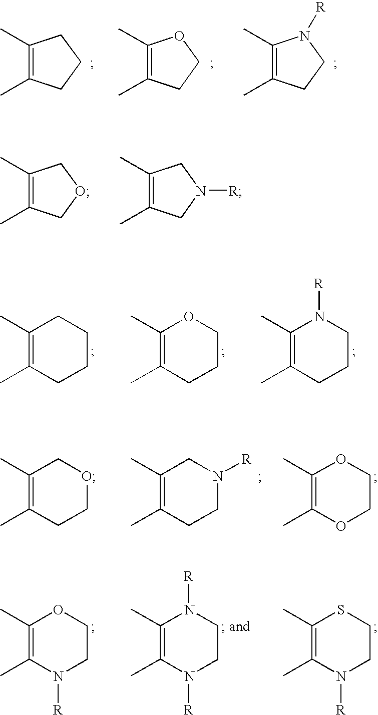

R is H or C₁-C₆ alkyl;

G is CH₂; O, S, S(O); or S(O)₂;

Each m is an integer of 0 or 1;

Q, when bonded to a nitrogen atom in group D, is selected from:

OC(O);

CH(R⁶)C(O);

OC(NR⁶);

CH(R⁶)C(NR⁶);

N(R⁶)C(O);

N(R⁶)C(S);

N(R⁶)C(NR⁶);

SC(O);

CH(R⁶)C(S);

SC(NR⁶);

C≡CCH₂;

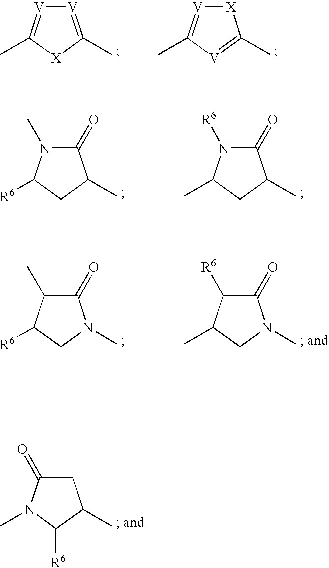

Q, when bonded to a carbon atom in group D, is as defined above and may further be selected from:

OCH₂;

N(R⁶)CH₂;

trans-(H)C═C(H);

cis-(H)C═C(H);

C≡C;

CH₂C≡C;

CF₂C≡C;

C≡CCF₂;

Each R⁶ independently is H, C₁-C₆ alkyl, C₃-C₆ cycloalkyl; 3- to 6-membered heterocycloalkyl; phenyl; benzyl; or 5- or 6-membered heteroaryl;

X is O, S, N(H), or N(C₁-C₆ alkyl);

Each V is independently C(H) or N;

D is a cyclic diradical group selected from:

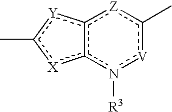

R³ is suitably substituted and is independently selected from the group consisting of hydrogen, —(C₁-C₆)alkyl, —(C₂-C₆)alkenyl, —(C₂-C₆)alkynyl, (C₅-C₁₀)aryl-(C₂-C₆)alkynyl-, (C₅-C₁₀)heteroaryl-(C₂-C₆)alkynyl-, (C₃-C₁₀)cycloalkyl-(C₂-C₆)alkynyl-, (C₃-C₁₀)heterocyclyl-(C₂-C₆)alkynyl-, —(CH₂)ₘ(C₅-C₁₀)aryl, —(CH₂)ₘ(C₅-C₁₀)heteroaryl, and —(CH₂)ₘ(C₃-C₁₀)cycloalkyl,

Y is selected from N, CR⁴, O, and S;

X is selected from NH, C(R⁴)₂, O, and S;

Z is selected from C(R⁴)₂, C═O, S═O, SO₂;

V is selected from C═O and C(R⁴)₂;

R⁴ is H or —(C₁-C₆)alkyl;

m is an integer from 0-6; and

dashed lines represent optional double bonds;

with the proviso that when Y is O or S, X is not O or S,

wherein the group D may be unsubstituted or substituted on a carbon atom or a nitrogen atom by replacement of a hydrogen atom with a group selected from:

CH₃;

CF₃;

C(O)H;

CN;

HO;

CH₃O;

C(F)H₂O;

C(H)F₂O; and

CF₃O;

wherein a carbon atom in the group D may further be substituted with F;

V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;

wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,

wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;

wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;

wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

100. A compound of Formula I

Z—L—R¹—Q—D—(V¹)ₘ—R² I

or a pharmaceutically acceptable salt thereof,

wherein:

Z is selected from:

HO₂C;

HO(H)N(O)C;

H(O)C—N(OH);

CH₃(O)C—N(OH);

CH₃(H)N(O)C—N(OH);

HS;

H₂N(O)₂S;

CH₃(H)N(O)₂S;

HO(O)P;

(HO)₂(O)P;

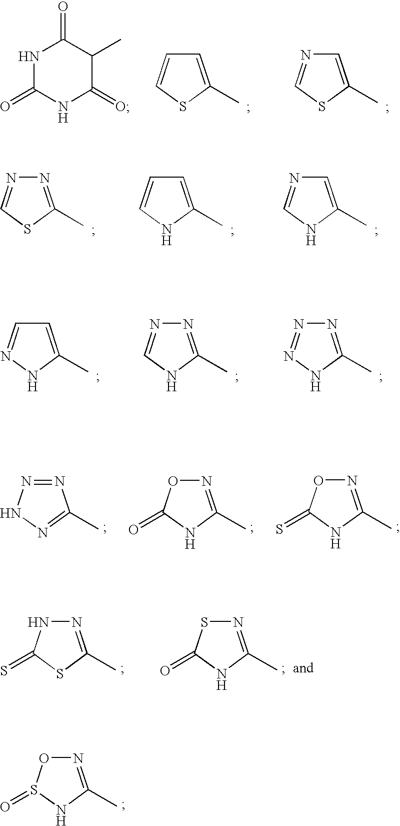

L is selected from:

C₃-C₅ alkylenyl;

Substituted C₃-C₅ alkylenyl;

3- to 5-membered heteroalkylenyl; and

Substituted 3- to 5-membered heteroalkylenyl;

Substituted L groups contain 1 or 2 substituents on a carbon atom or nitrogen atom independently selected from:

HO;

CN; and

CF₃;

wherein each substituent on a carbon atom may further be independently F, and

wherein 2 substituents may be taken together with a carbon atom to which they are both bonded to form the group C═O;

R¹ is independently selected from:

C₅ or C₆ cycloalkylenyl-(C₁-C₈ alkylenyl);

Substituted C₅ or C₆ cycloalkylenyl-(C₁-C₈ alkylenyl);

5- or 6-membered heterocycloalkylenyl-(C₁-C₈ alkylenyl);

Substituted 5- or 6-membered heterocycloalkylenyl-(C₁-C₈ alkylenyl);

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl);

Phenyl;

Substituted phenyl;

Naphthyl;

Substituted naphthyl;

5- or 6-membered heteroaryl;

Substituted 5- or 6-membered heteroaryl;

8- to 10-membered heterobiaryl; and

Substituted 8- to 10-membered heterobiaryl;

R² is independently selected from:

H;

C₁-C₆ alkyl;

Phenyl-(C₁-C₈ alkylenyl);

Substituted phenyl-(C₁-C₈ alkylenyl);

Naphthyl-(C₁-C₈ alkylenyl);

Substituted naphthyl-(C₁-C₈ alkylenyl);

5- or 6-membered heteroaryl-(C₁-C₈ alkylenyl);

Substituted 5- or 6-membered heteroaryl-(C₁-C₈ alkylenyl);

8- to 10-membered heterobiaryl-(C₁-C₈ alkylenyl);

Substituted 8- to 10-membered heterobiaryl-(C₁-C₈ alkylenyl);

Phenyl-O—(C₁-C₈ alkylenyl);

Substituted phenyl-O—(C₁-C₈ alkylenyl);

Phenyl-S—(C₁-C₈ alkylenyl);

Substituted phenyl-S—(C₁-C₈ alkylenyl);

Phenyl-S(O)—(C₁-C₈ alkylenyl);

Substituted phenyl-S(O)—(C₁-C₈ alkylenyl);

Phenyl-S(O)₂—(C₁-C₈ alkylenyl); and

Substituted phenyl-S(O)₂—(C₁-C₈ alkylenyl);

Each substituted R¹ group contains from 1 to 3 substituents, and each substituted R² group contains from 1 to 4 substituents, wherein each substituent is independently on a carbon or nitrogen atom, independently selected from:

C₁-C₆ alkyl;

CN;

CF₃;

HO;

(C₁-C₆ alkyl)-O;

(C₁-C₆ alkyl)-S(O)₂;

H₂N;

(C₁-C₆ alkyl)-N(H);

(C₁-C₆ alkyl)₂-N;

(C₁-C₆ alkyl)-C(O)O—(C₁-C₈ alkylenyl)ₘ;

(C₁-C₆ alkyl)-C(O)O—(1- to 8-membered heteroalkylenyl)ₘ;

(C₁-C₆ alkyl)-C(O)N(H)—(C₁-C₈ alkylenyl)ₘ;

(C₁-C₆ alkyl)-C(O)N(H)-(1- to 8-membered heteroalkylenyl)ₘ;

H₂NS(O)₂—(C₁-C₈ alkylenyl);

(C₁-C₆ alkyl)-N(H)S(O)₂—(C₁-C₈ alkylenyl)ₘ;

(C₁-C₆ alkyl)₂-NS(O)₂—(C₁-C₈ alkylenyl)ₘ;

3- to 6-membered heterocycloalkyl-(G)ₘ;

Substituted 3- to 6-membered heterocycloalkyl-(G)ₘ;

5- or 6-membered heteroaryl-(G)ₘ;

Substituted 5- or 6-membered heteroaryl-(G)ₘ;

(C₁-C₆ alkyl)-S(O)₂—N(H)—C(O)—(C₁-C₈ alkylenyl)ₘ; and

(C₁-C₆ alkyl)-C(O)—N(H)—S(O)₂—(C₁-C₈ alkylenyl)ₘ;

wherein each substituent on a carbon atom may further be independently selected from:

Halo; and

HO₂C;

wherein 2 substituents may be taken together with a carbon atom to which they are both bonded to form the group C═O;

wherein two adjacent, substantially sp² carbon atoms may be taken together with a diradical substituent to form a cyclic diradical selected from:

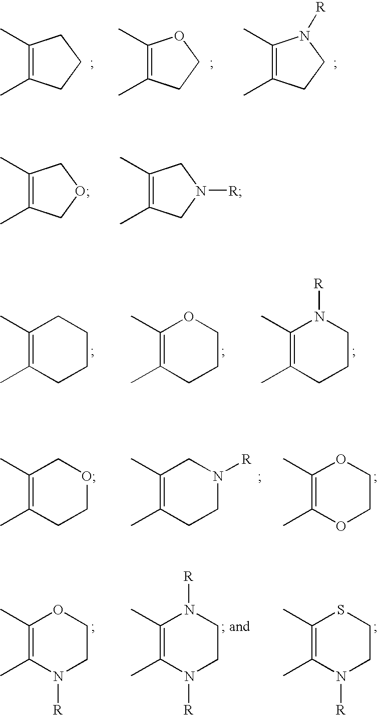

R is H or C₁-C₆ alkyl;

G is CH₂; O, S, S(O); or S(O)₂;

Each m is an integer of 0 or 1;

Q, when bonded to a nitrogen atom in group D, is selected from:

OC(O);

CH(R⁶)C(O);

OC(NR⁶);

CH(R⁶)C(NR⁶);

N(R⁶)C(O);

N(R⁶)C(S);

N(R⁶)C(NR⁶);

SC(O);

CH(R⁶)C(S);

SC(NR⁶);

C≡CCH₂;

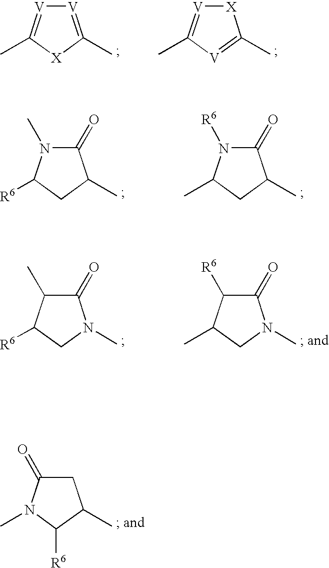

Q, when bonded to a carbon atom in group D, is as defined above and may further be selected from:

OCH₂;

N(R⁶)CH₂;

trans-(H)C═C(H);

cis-(H)C═C(H);

C≡C;

CH₂C≡C;

CF₂C≡C;

C≡CCF₂;

Each R⁶ independently is H, C₁-C₆ alkyl, C₃-C₆ cycloalkyl; 3- to 6-membered heterocycloalkyl; phenyl; benzyl; or 5- or 6-membered heteroaryl;

X is O, S, N(H), or N(C₁-C₆ alkyl);

Each V is independently C(H) or N;

D is a cyclic diradical group selected from:

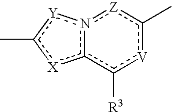

wherein R³ is suitably substituted and is independently selected from the group consisting of hydrogen, —(C₁-C₆)alkyl, —(C₂-C₆)alkenyl, —(C₂-C₆)alkynyl, (C₅-C₁₀)aryl-(C₂-C₆)alkynyl-, (C₅-C₁₀)heteroaryl-(C₂-C₆)alkynyl-, (C₃-C₁₀)cycloalkyl-(C₂-C₆)alkynyl-, (C₃-C₁₀)heterocyclyl-(C₂-C₆)alkynyl-, —(CH₂)ₘ(C₅-C₁₀)aryl, —(CH₂)ₘ(C₅-C₁₀)heteroaryl, and —(CH₂)ₘ(C₃-C₁₀)cycloalkyl,

Y is selected from N, CR⁴, O, and S;

X is selected from NH, C(R⁴)₂, O, and S;

Z is selected from C(R⁴)₂, C═O, S═O, SO₂;

V is selected from C═O and C(R⁴)₂;

m is an integer from 0-6; and

dashed lines represent optional double bonds;

with the proviso that when Y is O or S, X is not O or S,

wherein the group D may be unsubstituted or substituted on a carbon atom or a nitrogen atom by replacement of a hydrogen atom with a group selected from:

CH₃;

CF₃;

C(O)H;

CN;

HO;

CH₃O;

C(F)H₂O;

C(H)F₂O; and

CF₃O;

wherein a carbon atom in the group D may further be substituted with F;

V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;

wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,

wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;

wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;

wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

101. A compound of Formula I

Z—L—R¹—Q—D—(V¹)ₘ—R² I

or a pharmaceutically acceptable salt thereof,

wherein:

Z is selected from:

HO₂C;

HO(H)N(O)C;

H(O)C—N(OH);

CH₃(O)C—N(OH);

CH₃(H)N(O)C—N(OH);

HS;

H₂N(O)₂S;

CH₃(H)N(O)₂S;

HO(O)P;

(HO)₂(O)P;

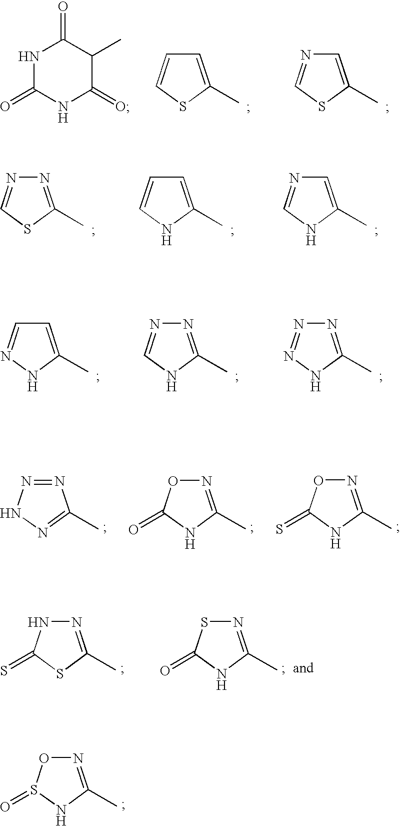

L is selected from:

C₃-C₅ alkylenyl;

Substituted C₃-C₅ alkylenyl;

3- to 5-membered heteroalkylenyl; and

Substituted 3- to 5-membered heteroalkylenyl;

Substituted L groups contain 1 or 2 substituents on a carbon atom or nitrogen atom independently selected from:

HO;

CN; and

CF₃;

wherein each substituent on a carbon atom may further be independently F, and wherein 2 substituents may be taken together with a carbon atom to which they are both bonded to form the group C═O;

R¹ is independently selected from:

C₅ or C₆ cycloalkylenyl-(C₁-C₈ alkylenyl);

Substituted C₅ or C₆ cycloalkylenyl-(C₁-C₈ alkylenyl);

5- or 6-membered heterocycloalkylenyl-(C₁-C₈ alkylenyl);

Substituted 5- or 6-membered heterocycloalkylenyl-(C₁-C₈ alkylenyl);

Phenylenyl-(C₁-C₈ alkylenyl);

Substituted phenylenyl-(C₁-C₈ alkylenyl);

5- or 6-membered heteroarylenyl-(C₁-C₈ alkylenyl);

Substituted 5- or 6-membered heteroarylenyl-(C₁-C₈ alkylenyl);

Phenyl;

Substituted phenyl;

Naphthyl;

Substituted naphthyl;

5- or 6-membered heteroaryl;

Substituted 5- or 6-membered heteroaryl;

8- to 10-membered heterobiaryl; and

Substituted 8- to 10-membered heterobiaryl;

R² is independently selected from:

H;

C₁-C₆ alkyl;

Phenyl-(C₁-C₈ alkylenyl);

Substituted phenyl-(C₁-C₈ alkylenyl);

Naphthyl-(C₁-C₈ alkylenyl);

Substituted naphthyl-(C₁-C₈ alkylenyl);

5- or 6-membered heteroaryl-(C₁-C₈ alkylenyl);

Substituted 5- or 6-membered heteroaryl-(C₁-C₈ alkylenyl);

8- to 10-membered heterobiaryl-(C₁-C₈ alkylenyl);

Substituted 8- to 10-membered heterobiaryl-(C₁-C₈ alkylenyl);

Phenyl-O—(C₁-C₈ alkylenyl);

Substituted phenyl-O—(C₁-C₈ alkylenyl);

Phenyl-S—(C₁-C₈ alkylenyl);

Substituted phenyl-S—(C₁-C₈ alkylenyl);

Phenyl-S(O)—(C₁-C₈ alkylenyl);

Substituted phenyl-S(O)—(C₁-C₈ alkylenyl);

Phenyl-S(O)₂—(C₁-C₈ alkylenyl); and

Substituted phenyl-S(O)₂—(C₁-C₈ alkylenyl);

Each substituted R¹ group contains from 1 to 3 substituents, and each substituted R² group contains from 1 to 4 substituents, wherein each substituent is independently on a carbon or nitrogen atom, independently selected from:

C₁-C₆ alkyl;

CN;

CF₃;

HO;

(C₁-C₆ alkyl)-O;

(C₁-C₆ alkyl)-S(O)₂;

H₂N;

(C₁-C₆ alkyl)-N(H);

(C₁-C₆ alkyl)₂-N;

(C₁-C₆ alkyl)-C(O)O—(C₁-C₈ alkylenyl)ₘ;

(C₁-C₆ alkyl)-C(O)O-(1- to 8-membered heteroalkylenyl)ₘ;

(C₁-C₆ alkyl)-C(O)N(H)—(C₁-C₈ alkylenyl)ₘ;

(C₁-C₆ alkyl)-C(O)N(H)-(1- to 8-membered heteroalkylenyl)ₘ;

H₂NS(O)₂—(C₁-C₈ alkylenyl);

(C₁-C₆ alkyl)-N(H)S(O)₂—(C₁-C₈ alkylenyl)ₘ;

(C₁-C₆ alkyl)₂-NS(O)₂—(C₁-C₈ alkylenyl)ₘ;

3- to 6-membered heterocycloalkyl-(G)ₘ;

Substituted 3- to 6-membered heterocycloalkyl-(G)ₘ;

5- or 6-membered heteroaryl-(G)ₘ;

Substituted 5- or 6-membered heteroaryl-(G)ₘ;

(C₁-C₆ alkyl)-S(O)₂—N(H)—C(O)—(C₁-C₈ alkylenyl)ₘ; and

(C₁-C₆ alkyl)-C(O)—N(H)—S(O)₂—(C₁-C₈ alkylenyl)ₘ;

wherein each substituent on a carbon atom may further be independently selected from:

Halo; and

HO₂C;

wherein 2 substituents may be taken together with a carbon atom to which they are both bonded to form the group C═O;

wherein two adjacent, substantially sp² carbon atoms may be taken together with a diradical substituent to form a cyclic diradical selected from:

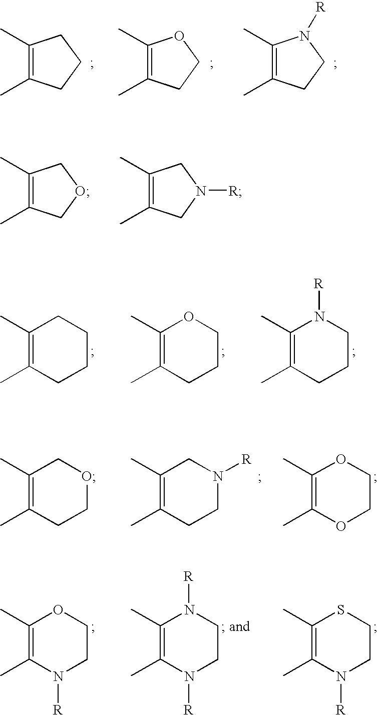

R is H or C₁-C₆ alkyl;

G is CH₂; O, S, S(O); or S(O)₂;

Each m is an integer of 0 or 1;

Q, when bonded to a nitrogen atom in group D, is selected from:

OC(O);

CH(R⁶)C(O);

OC(NR⁶);

CH(R⁶)C(NR⁶);

N(R⁶)C(O);

N(R⁶)C(S);

N(R⁶)C(NR⁶);

SC(O);

CH(R⁶)C(S);

SC(NR⁶);

C≡CCH₂;

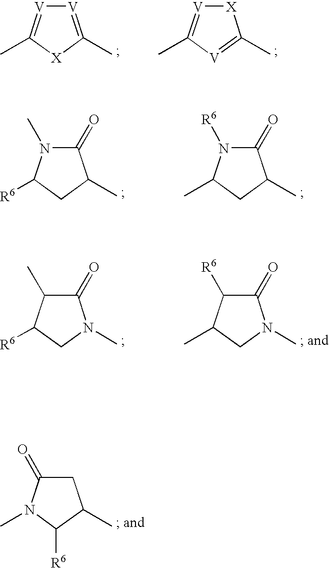

Q, when bonded to a carbon atom in group D, is as defined above and may further be selected from:

OCH₂;

N(R⁶)CH₂;

trans-(H)C═C(H);

cis-(H)C═C(H);

C≡C;

CH₂C≡C;

CF₂C≡C;

C≡CCF₂;

Each R⁶ independently is H, C₁-C₆ alkyl, C₃-C₆ cycloalkyl; 3- to 6-membered heterocycloalkyl; phenyl; benzyl; or 5- or 6-membered heteroaryl;

X is O, S, N(H), or N(C₁-C₆ alkyl);

Each V is independently C(H) or N;

D is a cyclic diradical group selected from:

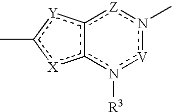

wherein R³ is suitably substituted and is independently selected from the group consisting of hydrogen, —(C₁-C₆)alkyl, —(C₂-C₆)alkenyl, —(C₂-C₆)alkynyl, (C₅-C₁₀)aryl-(C₂-C₆)alkynyl-, (C₅-C₁₀)heteroaryl-(C₂-C₆)alkynyl-, (C₃-C₁₀)cycloalkyl-(C₂-C₆)alkynyl-, (C₃-C₁₀)heterocyclyl-(C₂-C₆)alkynyl-, —(CH₂)ₘ(C₅-C₁₀)aryl, —(CH₂)ₘ(C₅-C₁₀)heteroaryl, and —(CH₂)ₘ(C₃-C₁₀)cycloalkyl,

Y is selected from N, CR⁴, O, and S;

X is selected from NH, C(R⁴)₂, O, and S;

Z is selected from C(R⁴)₂, C═O, S═O, SO₂;

V is selected from C═O and C(R⁴)₂;

m is an integer from 0-6; and

dashed lines represent optional double bonds;

with the proviso that when Y is O or S, X is not O or S,

wherein the group D may be unsubstituted or substituted on a carbon atom or a nitrogen atom by replacement of a hydrogen atom with a group selected from:

CH₃;

CF₃;

C(O)H;

CN;

HO;

CH₃O;

C(F)H₂O;

C(H)F₂O; and

CF₃O;

wherein a carbon atom in the group D may further be substituted with F;

V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;

wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,

wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;

wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;

wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

### DETAILED DESCRIPTION OF THE INVENTION

This invention provides compounds defined by Formula I

Z—L—R¹—Q—D—(V¹)ₘ—R² I

or a pharmaceutically acceptable salt thereof,

wherein Z, L, R¹, Q, D, V¹, m, and R² are as defined above.

The invention also provides pharmaceutical compositions comprising a compound of Formula I, or a pharmaceutically acceptable salt thereof, as defined above, together with a pharmaceutically acceptable carrier, diluent, or excipient.

The invention also provides methods of inhibiting an MMP-13 enzyme in an animal, comprising administering to the animal a compound of Formula I, or a pharmaceutically acceptable salt thereof.

The invention also provides methods of treating a disease mediated by an MMP-13 enzyme in a patient, comprising administering to the patient a compound of Formula I, or a pharmaceutically acceptable salt thereof, either alone or in a pharmaceutical composition.

The invention also provides methods of treating diseases such as heart disease, multiple sclerosis, osteo- and rheumatoid arthritis, arthritis other than osteo- or rheumatoid arthritis, cardiac insufficiency, inflammatory bowel disease, heart failure, age-related macular degeneration, chronic obstructive pulmonary disease, asthma, periodontal diseases, psoriasis, atherosclerosis, and osteoporosis in a patient, comprising administering to the patient a compound of Formula I, or a pharmaceutically acceptable salt thereof, either alone or in a pharmaceutical composition.

The invention also provides combinations, comprising a compound of Formula I, or a pharmaceutically acceptable salt thereof, together with another pharmaceutically active component as described.

As seen above, the groups of Formula I include “C₁-C₆ alkyl” groups. C₁-C₆ alkyl groups are straight and branched carbon chains having from 1 to 6 carbon atoms. Examples of C₁-C₆ alkyl groups include methyl, ethyl, 1-propyl, 2-propyl, 1-butyl, 2-butyl, 2,2-dimethylethyl, 1-pentyl, 2-pentyl, 2,2-dimethylpropyl, and 1-hexyl.

The phrase “substituted C₁-C₆ alkyl” means a C₁-C₆ alkyl group as defined above that is substituted with from 1 to 4 substituents independently selected from the list above. Illustrative examples of substituted C₁-C₆ alkyl groups include CH₂OH, CF₂OH, CH₂C(CH₃)₂CO₂CH₃, CF₃, C(O)CF₃, C(O)—CH₃, (CH₂)₄—S—CH₃, CH(CO₂H)CH₂CH₂C(O)NMe₂, (CH₂)₅NH—C(O)—NH₂, CH₂—CH₂—C(H)-(4-fluorophenyl), CH(OCH₃)CH₂CH₃, CH₂SO₂NH₂, and CH(CH₃)CH₂CH₂OC(O)CH₃.

The term “C₂-C₆ alkenyl” means a straight or branched, unsubstituted hydrocarbon group having from 2 to 6 carbon atoms and 1 or 2 carbon-carbon double bonds, and include allenyl groups. Typical examples of C₂-C₆ alkenyl groups include ethenyl, 1-propen-1-yl, 1-propen-2-yl, 2-propen-1-yl, 1-buten-3-yl, 2-penten-2-yl, and 1-hexen-6-yl.

The phrase “substituted C₂-C₆ alkenyl” means a C₂-C₆ alkenyl as defined above, which is substituted with from 1 to 4 substituents independently selected from the list above. Illustrative examples of substituted C₂-C₆ alkenyl groups include C(H)═C(H)CH₂OH, CH═CF₂, CH₂C(H)═C(H)—(CH₂)₂CF₂OH, CH₂C(═CH₂)CO₂CH₃, C(H)═C(H)—CF₃, CH₂—CH₂—C(H)═C(H)—C(O)—CH₃, C(H)═C(CH₃)—S—CH₃, C(H)═C(H)—C(H)═C(CH₃)—CO₂Me, and C(H)═C═C(H)OC(O)CH₃.

The term “C₂-C₆ alkynyl” means a straight or branched, unsubstituted hydrocarbon group having from 2 to 6 carbon atoms and 1 or 2 carbon-carbon triple bonds. Typical examples of C₂-C₆ alkynyl groups include ethenyl, 1-propyn-1-yl, 1-propyn-3-yl, 1-butyn-3-yl, 2-pentyn-1-yl, and 1-hexyn-6-yl.

The phrase “substituted C₂-C₆ alkynyl” means a C₂-C₆ alkynyl as defined above, which is substituted with from 1 to 4 substituents independently selected from the list above. Illustrative examples of substituted C₂-C₆ alkynyl groups include C≡CCH₂OH, C≡CF, CH₂C≡C—(CH₂)₂CF₂OH, C≡C—CH₂CO₂CH₃, CH₂C≡C—CF₃, CH₂—CH₂—C≡C—C(O)—CH₃, C≡C—S—CH₃, and C≡C—C(O)OC(O)CH₃.

The term “C₃-C₆ cycloalkyl” means an unsubstituted cyclic hydrocarbon group having from 3 to 6 carbon atoms. C₃-C₆ cycloalkyl may optionally contain one carbon-carbon double bond. The group C₃-C₆ cycloalkyl includes cyclopropyl, cyclobutyl, cyclopentyl, cyclopenten-1-yl, cyclopenten-4-yl, and cyclohexyl.

The phrase “substituted C₃-C₆ cycloalkyl” means a C₃-C₆ cycloalkyl as defined above, which is substituted with from 1 to 4 substituents independently selected from the list above. Illustrative examples of substituted C₃-C₆ cycloalkyl groups include 1-hydroxy-cyclopropyl, cyclobutanon-3-yl, 3-(3-phenyl-ureido)-cyclopent-1-yl, and 4-carboxy-cyclohexyl.

The phrase “3- to 6-membered heterocycloalkyl” means an unsubstituted saturated cyclic group having carbon atoms and 1 or 2 heteroatoms independently selected from 2 O, 1 S, 1 S(O), 1 S(O)₂, 1 N, 2 N(H), and 2 N(C₁-C₆ alkyl), wherein when two O atoms or one O atom and one S atom are present, the two O atoms or one O atom and one S atom are not bonded to each other. Optionally, a 3- to 6-membered heterocycloalkyl may contain one carbon-carbon or carbon-nitrogen double bond. Illustrative examples of 3- to 6-membered heterocycloalkyl includes aziridin-1-yl, 1-oxa-cyclobutan-2-yl, tetrahyrdofuran-3-yl, morpholin-4-yl, 2-thiacyclohex-1-yl, 2-oxo-2-thiacyclohe-1-yl, 2,2-dioxo-2-thiacyclohex-1-yl, and 4-methyl-piperazin-2-yl.

The phrase “substituted 3- to 6-membered heterocycloalkyl” means a 3- to 6-membered heterocycloalkyl as defined above, which is substituted with from 1 to 4 substituents independently selected from the list above. Illustrative examples of substituted 3- to 6-membered heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, and 1-cyclopropyl-4-methyl-piperazin-2-yl.

The term “C₁-C₈ alkylenyl” means a saturated hydrocarbon diradical that is straight or branched and has from 1 to 8 carbon atoms. C₁-C₈ alkylenyl having from 2 to 8 carbon atoms may optionally independently contain one carbon-carbon double bond. Illustrative examples of C₁-C₈ alkylenyl include CH₂, CH₂CH₂, C(CH₃)H, C(H)(CH₃)CH₂CH₂, and CH₂C(H)═C(H)CH₂CH₂CH₂CH₂CH₂.

The term “C₃-C₅ alkylenyl” means a saturated hydrocarbon diradical that is straight or branched and has from 3 to 5 carbon atoms. C₃-C₅ alkylenyl may optionally independently contain one carbon-carbon double bond. Illustrative examples of C₃-C₅ alkylenyl include CH₂CH₂CH₂, CH₂CH₂C(CH₃)H, C(H)(CH₃)CH₂CH₂, and CH₂C(H)═C(H)CH₂CH₂.

The phrase “Substituted C₃-C₅ alkylenyl” means a C₃-C₅ alkylenyl, as defined above, substituted with 1 or 2 substituents, as defined above. Illustrative examples of substituted C₃-C₅ alkylenyl include CH₂CH₂CF₂, C(H)OHCH₂ CH₂CH₂, CH₂CH₂C(CH₃)CN, C(H)(CF₃)CH₂CH₂, and C(O)C(H)═C(H)CH₂CH₂.

The term “1- to 8-membered heteroalkylenyl” means a saturated diradical chain that is straight or branched and contains from 1 to 7 carbon atoms and 1 heteroatom selected from O, S, N(H), and N(C₁-C₆ alkyl). 2- to 8-membered heteroalkylenyl, having from 2 to 8 chain atoms, may optionally independently contain one carbon-carbon double bond. Illustrative examples of 1- to 8-membered heteroalkylenyl include OCH₂, CH₂CH₂O, C(CH₃)HS, and CH₂C(H)═C(H)CH₂N(H)CH₂CH₂CH₂.

The term “3- to 5-membered heteroalkylenyl” means a saturated diradical chain that is straight or branched and contains from 2 to 4 carbon atoms and 1 heteroatom selected from O, S, N(H), and N(C₁-C₆ alkyl). 3- to 5-membered heteroalkylenyl may optionally independently contain one carbon-carbon double bond or one carbon-nitrogen double bond. Illustrative examples of 3- to 5-membered heteroalkylenyl include CH₂CH₂OCH₂, CH₂CH₂O, C(CH₃)(H)SCH₂CH₂, and CH₂C(H)═C(H)CH₂N(H).

The phrase “Substituted 3- to 5-membered heteroalkylenyl” means a 3- to 5-membered heteroalkylenyl, as defined above, that is substituted with 1 or 2 substituents as defined above. Illustrative examples of substituted 3- to 5-membered heteroalkylenyl include CH₂CH₂OC(O), CF₂CH₂O, C(CH₃)(CN)SCH₂CH₂, and CH₂C(H)═C(H)CH₂N(OH).

The phrase “C₃-C₆ cycloalkyl-(C₁-C₈ alkylenyl)” means a C₃-C₆ cycloalkyl, as defined above, bonded through a C₁-C₈ alkylenyl, as defined above. Illustrative examples of C₃-C₆ cycloalkyl-(C₁-C₈ alkylenyl include cyclopropylmethyl, 1-cyclopentyl-hex-2-yl, and 2-cyclobutyl-but-2-yl.

The phrase “Substituted C₃-C₆ cycloalkyl-(C₁-C₈ alkylenyl)” means a C₃-C₆ cycloalkyl-(C₁-C₈ alkylenyl), as defined above, substituted on C₃-C₆ cycloalkyl and/or C₁-C₈ alkylenyl with from 1 to 4 substituents, as defined above. Illustrative examples of substituted C₃-C₆ cycloalkyl-(C₁-C₈ alkylenyl include cyclopropylcarbonyl and 1-(1-aminomethyl-cyclopentyl)-hex-2-yl.

The phrase “C₅ or C₆ cycloalkyl-(C₁-C₈ alkylenyl)” means a cyclopentyl or cyclohexyl bonded through a C₁-C₈ alkylenyl, as defined above, wherein the cycloalkyl optionally contains 1 carbon-carbon double bond.

The phrase “Substituted C₅ or C₆ cycloalkyl-(C₁-C₈ alkylenyl)” means a substituted cyclopentyl or cyclohexyl, wherein the substituents are as defined above, bonded through a C₁-C₈ alkylenyl, as defined above, wherein the cycloalkyl optionally contains 1 carbon-carbon double bond.

The phrase “C₈-C₁₀ bicycloalkyl-(C₁-C₈ alkylenyl)” means a cyclopentyl or cyclohexyl fused to another cyclopentyl or cyclohexyl to give a 5,5-, 5,6-, or 6,6-fused bicyclic carbocyclic group, which is bonded through a C₁-C₈ alkylenyl, as defined above, wherein the bicycloalkyl optionally contains 1 carbon-carbon double bond.

The phrase “Substituted C₈-C₁₀ bicycloalkyl-(C₁-C₈ alkylenyl)” means a C₈-C₁₀ bicycloalkyl, as defined above, substituted with from 1 to 4 substituents, as defined above, bonded through a C₁-C₈ alkylenyl, as defined above.

The phrase “5- or 6-membered heterocycloalkyl-(C₁-C₈ alkylenyl)” means a 5- or 6-membered ring containing carbon atoms and 1 or 2 heteroatoms selected from 1 O, 1 S, 1 N, 2 N(H), and 2 N(C₁-C₆ alkyl), bonded through a C₁-C₈ alkylenyl, as defined above.

The phrase “Substituted 5- or 6-membered heterocycloalkyl-(C₁-C₆ alkylenyl)” means a 5- or 6-membered heterocycloalkyl, as defined above, substituted with from 1 to 4 substituents, as defined above, bonded through a C₁-C₈ alkylenyl, as defined above.

The phrase “8- to 10-membered heterobicycloalkyl-(C₁-C₈ alkylenyl)” means a 5- or 6-membered ring fused to another 5- or 6-membered ring to give a 5,5-, 5,6-, or 6,6-fused bicyclic group containing carbon atoms and from 1 to 4 heteroatoms independently selected from 2 O, 1 S, 1 S(O), 1 S(O)₂, 1 N, 4 N(H), and 4 N(C₁-C₆ alkyl), bonded through a C₁-C₈ alkylenyl, as defined above, wherein the bicycloalkyl optionally contains 1 carbon-carbon double bond or 1 carbon-nitrogen double bond.

The phrase “Substituted 8- to 10-membered heterobicycloalkyl-(C₁-C₆ alkylenyl)” means an 8- to 10-membered heterobicycloalkyl, as defined above, substituted with from 1 to 4 substituents, as defined above, bonded through a C₁-C₈ alkylenyl, as defined above.

The phrase “3- to 6-membered heterocycloalkyl-(C₁-C₈ alkylenyl)” means a 3- to 6-membered heterocycloalkyl, as defined above, bonded through a C₁-C₈ alkylenyl, as defined above.

The phrase “Substituted 3- to 6-membered heterocycloalkyl-(C₁-C₈ alkylenyl)” means a substituted 3- to 6-membered heterocycloalkyl, as defined above, bonded through a C₁-C₈ alkylenyl, as defined above.

The phrase “Phenyl-(C₁-C₈ alkylenyl)” means a phenyl group bonded through a C₁-C₈ alkylenyl diradical, wherein C₁-C₈ alkylenyl is as defined above. Illustrative examples of phenyl-(C₁-C₈ alkylenyl) include benzyl, 2-phenylethyl, 1-phenyl-prop-1-yl, and 3-phenyl-heptyl.

The phrase “Substituted phenyl-(C₁-C₈ alkylenyl)” means a phenyl-(C₁-C₈ alkylenyl) as defined above, which is substituted on phenyl and/or C₁-C₈ alkylenyl with from 1 to 4 substituents independently selected from the list above. Illustrative examples of substituted phenyl-(C₁-C₈ alkylenyl) include 4-fluoro-phenylmethyl, 2-(4-carboxy-phenyl)-ethyl, 1-(2,4-dimethoxy-phenyl)-2-oxo-propyl, and 1-phenyl-5,5-difluoro-oct-3-yl.

The term “naphthyl” includes 1-naphthyl and 2-napthyl.

The phrase “Naphthyl-(C₁-C₈ alkylenyl)” means a naphthyl group as defined above bonded through a C₁-C₈ alkylenyl diradical, wherein C₁-C₈ alkylenyl is as defined above. Illustrative examples of naphthyl-(C₁-C₈ alkylenyl) include naphth-1-ylmethyl, 2-(naphth-1-yl)ethyl, and 3-(naphth-2-yl)-1-heptyl.

The phrase “Substituted naphthyl-(C₁-C₈ alkylenyl)” means a naphthyl-(C₁-C₈ alkylenyl) as defined above, which is substituted on naphthyl and/or C₁-C₈ alkylenyl with from 1 to 4 substituents independently selected from the list above. Illustrative examples of substituted phenyl-(C₁-C₈ alkylenyl) include 4-fluoro-(naphth-1-yl)methyl, 2-(4-carboxy-(naphth-1-yl))-ethyl, 1-(2,4-dimethoxy-(naphth-1-yl))-2-oxo-propyl, and 1-(naphth-2-yl)-5,5-difluorohept-2-yl.

The phrase “5- or 6-membered heteroaryl” means a 5-membered, monocyclic heteroaryl having carbon atoms and from 1 to 4 heteroatoms independently selected from 1 O, 1 S, 1 N(H), 1 N(C₁-C₆ alkyl), and 4 N, or a 6-membered, monocyclic heteroaryl having carbon atoms and 1 or 2 heteroatoms selected from 2 N, and wherein:

(i) The phrase “5-membered, monocyclic heteroaryl” means a 5-membered, monocyclic, aromatic ring group as defined above having carbon atoms and from 1 to 4 heteroatoms selected from 1 O, 1 S, 1 N(H), 1 N(C₁-C₆ alkyl), and 4 N. Illustrative examples of a 5-membered, monocyclic heteroaryl include thiophen-2-yl, furan-2-yl, pyrrol-3-yl, pyrrol-1-yl, imidazol-4-yl, isoxazol-3-yl, oxazol-2-yl, thiazol-4-yl, tetrazol-1-yl, 1,2,4-oxadiazol-3-yl, 1,2,4-triazol-1-yl, and pyrazol-3-yl; and

(ii) The phrase “6-membered, monocyclic heteroaryl” means a 6-membered, monocyclic, aromatic ring group as defined above having carbon atoms and 1 or 2 N. Illustrative examples of a 6-membered, monocyclic heteroaryl include pyridin-2-yl, pyridin-4-yl, pyrimidin-2-yl, pyridazin-4-yl, and pyrazin-2-yl.

The phrase “8- to 10-membered heterobiaryl” means an 8-membered, 5,5-fused bicyclic heteroaryl, a 9-membered, 6,5-fused bicyclic heteroaryl, or a 10-membered, 6,6-fused bicyclic heteroaryl, having carbon atoms and from 1 to 4 heteroatoms independently selected from 1 O, 1 S, 1 N(H), 1 N(C₁-C₆ alkyl), and 4 N, wherein at least one of the 2 fused rings is aromatic, and wherein when the O and S atoms both are present, the O and S atoms are not bonded to each other, which are as defined below:

(iii) The phrase “8-membered, 5,5-fused bicyclic heteroaryl” means a an 8-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of an 8-membered, fused-bicyclic heteroaryl include

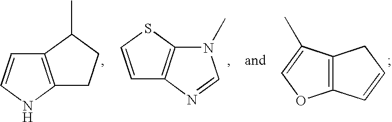

(iv) The phrase “9-membered, 6,5-fused bicyclic heteroaryl” means a 9-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 9-membered, fused-bicyclic heteroaryl include indol-2-yl, indol-6-yl, iso-indol-2-yl, benzimidazol-2-yl, benzimidazol-1-yl, benztriazol-1-yl, benztriazol-5-yl, benzoxazol-2-yl, benzothiophen-5-yl, and benzofuran-3-yl; and

(v) The phrase “10-membered, 6,5-fused bicyclic heteroaryl” means a 10-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 10-membered, fused-bicyclic heteroaryl include quinolin-2-yl, isoquinolin-7-yl, and benzopyrimidin-2-yl.

The phrases “substituted 5- or 6-membered heteroaryl” and “substituted 8-to 10-membered heterobiaryl” means a 5- or 6-membered heteroaryl, as defined above, or an 8- to 10-membered heterobiaryl, as defined above, respectively, which is substituted on a carbon (CH) atom, and/or nitrogen [N(H)] atom in the case of 5-, 8- to 10-membered heterobiaryl, with from 1 to 4 substituents independently selected from the list above.

Illustrative examples of substituted 5-membered, monocyclic heteroaryl groups include 2-hydroxy-oxoazol-4-yl, 5-chloro-thiophen-2-yl, 1-methylimidazol-5-yl, 1-propyl-pyrrol-2-yl, 1-acetyl-pyrazol-4-yl, 1-methyl-1,2,4-triazol-3-yl, and 2-hexyl-tetrazol-5-yl.

Illustrative examples of substituted 6-membered, monocyclic heteroaryl groups include 4-acetyl-pyridin-2-yl, 3-fluoro-pyridin-4-yl, 5-carboxy-pyrimidin-2-yl, 6-tertiary butyl-pyridazin-4-yl, and 5-hdyroxymethyl-pyrazin-2-yl.

Illustrative examples of substituted 8-membered, 5,5-fused bicyclic heteroaryl include:

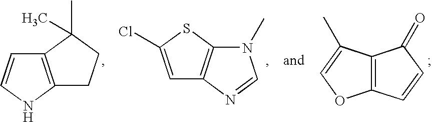

Illustrative examples of substituted 9-membered, 5,6-fused bicyclic heteroaryl include 3-(2-aminomethyl)-indol-2-yl, 2-carboxy-indol-6-yl, 1-(methanesulfonyl)-iso-indol-2-yl, 5-trifluorometyl-6,7-difluoro-4-hydroxymethyl-benzimidazol-2-yl, 4-(3-methylureido)-2-cyano-benzimidazol-1-yl, 1-methylbenzimidazol-6-yl, 1-acetylbenztriazol-7-yl, 1-methanesulfonyl-indol-3-yl, 1-cyano-6-aza-indol-5-yl, and 1-(2,6-dichlorophenylmethyl)-benzpyrazol-3-yl.

Illustrative examples of substituted 10-membered, 6,6-fused bicyclic heteroaryl include 5,7-dichloro-quinolin-2-yl, isoquinolin-7-yl-1-carboxylic acid ethyl ester, and 3-bromo-benzopyrimidin-2-yl.

The phrase “5- or 6-membered heteroaryl-(C₁-C₈ alkylenyl)” means a 5- or 6-membered heteroaryl, as defined above, bonded through a C₁-C₈ alkylenyl, as defined above.

The phrase “Substituted 5- or 6-membered heteroaryl-(C₁-C₈ alkylenyl)” means a 5- or 6-membered heteroaryl-(C₁-C₈ alkylenyl), as defined above, which is substituted on 5- or 6-membered heteroaryl and/or C₁-C₈ alkylenyl with from 1 to 4 substituents independently selected from the list above.

Illustrative examples of substituted 5-membered heteroaryl-(C₁-C₈ alkylenyl) groups include 2-hydroxy-oxoazol-4-ylmethyl, 4-(5-chloro-thiophen-2-yl)-hex-1-yl, and 2-tetrazol-5-yloctyl.

Illustrative examples of substituted 6-membered heteroaryl-(C₁-C₈ alkylenyl) groups include 4-acetyl-pyridin-2-ylmethyl, 7-(3-fluoro-pyridin-4-yl)-hept-2-yl, and 2-(5-hdyroxymethyl-pyrazin-2-yl)-1,1-difluoro-2-hydroxy-prop-2-yl.

The phrase “8- to 10-membered heterobiaryl-(C₁-C₈ alkylenyl)” means an 8- to 10-membered heterobiaryl, as defined above, bonded through a C₁-C₈ alkylenyl, as defined above.

The phrase “Substituted 8- to 10-membered heterobiaryl-(C₁-C₈ alkylenyl)” means an 8- to 10-membered heterobiaryl-(C₁-C₈ alkylenyl), as defined above, which is substituted on 8- to 10-membered heterobiaryl and/or C₁-C₈ alkylenyl with from 1 to 4 substituents independently selected from the list above.

Illustrative examples of substituted 8-membered heterobiaryl-(C₁-C₈ alkylenyl) include:

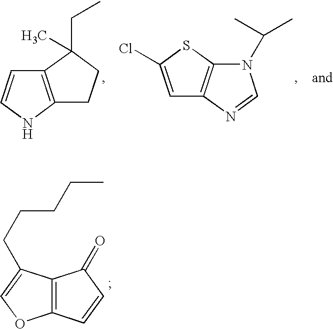

Illustrative examples of substituted 9-membered heterobiaryl-(C₁-C₈ alkylenyl) include 3-(2-aminomethyl)-indol-2-ylmethyl, and 1-(1-(2,6-dichlorophenylmethyl)-benzpyrazol-3-yl)-prop-3-yl.

Illustrative examples of substituted 10-membered heterobiaryl-(C₁-C₈ alkylenyl) include 5,7-dichloro-quinolin-2-ylmethyl, and 5-(3-bromo-benzopyrimidin-2-yl)-oct-2-yl.

The phrase “C₅ or C₆ cycloalkylenyl” means a 5-membered or 6-membered monocyclic diradical ring containing carbon atoms. Illustrative examples of C₅ or C₆ cycloalkylenyl include:

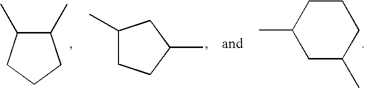

The phrase “C₅ or C₆ cycloalkylenyl-(C₁-C₈ alkylenyl)” means a C₅ or C₆ cycloalkylenyl, as defined above, bonded via one of its two radicals through a C₁-C₈ alkylenyl, as defined above. Illustrative examples of C₅ or C₆ cycloalkylenyl-(C₁-C₈ alkylenyl) include:

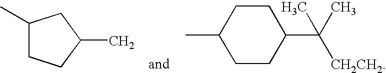

The phrase “Substituted C₅ or C₆ cycloalkylenyl-(C₁-C₈ alkylenyl)” means a C₅ or C₆ cycloalkylenyl-(C₁-C₈ alkylenyl), as defined above, that is substituted with 1 to 3 substituents as defined above. Illustrative examples of substituted C₅ or C₆ cycloalkylenyl-(C₁-C₈ alkylenyl) include:

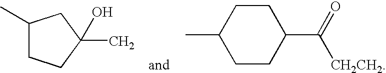

The phrase “5- or 6-membered heterocycloalkylenyl” means a 5-membered or 6-membered monocyclic diradical containing carbon atoms and from 1 to 3 heteroatoms independently selected from 1 O, 1 S, 1 S(O), 1S(O)₂, 1N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond. Illustrative examples of 5- or 6-membered heterocycloalkylenyl include:

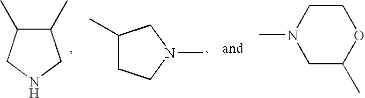

The phrase “5- or 6-membered heterocycloalkylenyl-(C₁-C₈ alkylenyl)” means a 5- or 6-membered heterocycloalkylenyl, as defined above, bonded via one of its two radicals through a C₁-C₈ alkylenyl, as defined above. Illustrative examples of 5- or 6-membered heterocycloalkylenyl-(C₁-C₈ alkylenyl) include:

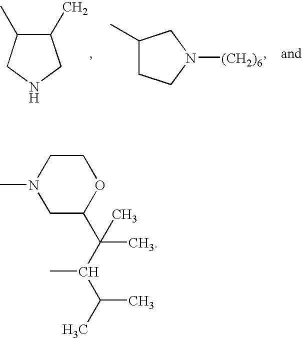

The phrase “Substituted 5- or 6-membered heterocycloalkylenyl-(C₁-C₈ alkylenyl)” means a 5- or 6-membered heterocycloalkylenyl-(C₁-C₈ alkylenyl), as defined above, substituted with from 1 to 3 substituents as defined above. Illustrative examples of substituted 5- or 6-membered heterocycloalkylenyl-(C₁-C₈ alkylenyl) include:

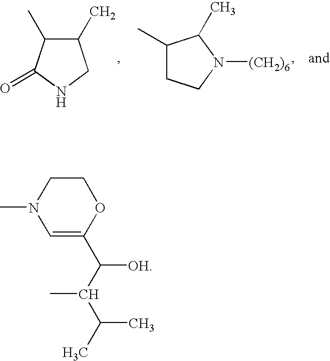

The term “Phenylenyl” means a diradical group derived from benzene by removing any two hydrogen atoms.

The phrase “Phenylenyl-(C₁-C₈ alkylenyl)” means a phenylenyl group, as defined above, bonded via one of its two radicals through a C₁-C₈ alkylenyl, as defined above. Illustrative examples of phenylenyl-(C₁-C₈ alkylenyl) include:

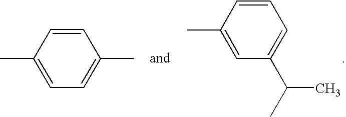

The phrase “Substituted phenylenyl-(C₁-C₈ alkylenyl)” means a phenylenyl-(C₁-C₈ alkylenyl) group, as defined above, substituted with from 1 to 3 substituents, as defined above. Illustrative examples of substituted phenylenyl-(C₁-C₈ alkylenyl) include:

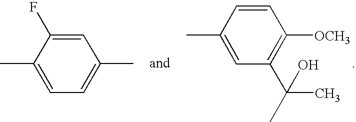

The phrase “5- or 6-membered heteroarylenyl” means a 5-membered monocyclic aromatic ring diradical containing carbon atoms and from 1 to 4 heteroatoms independently selected from 1 O, 1 S, 1 N(H), 1 N(C₁-C₆ alkyl), and 4 N or a 6-membered monocyclic aromatic ring diradical containing carbon atoms and 1 or 2 heteroatoms selected from 2 N, wherein the 1 O atom and the 1 S atom may not both be present in a ring. Illustrative examples of 5- or 6-membered heteroarylenyl include:

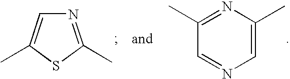

The phrase “5- or 6-membered heteroarylenyl-(C₁-C₈ alkylenyl)” means a 5- or 6-membered heteroarylenyl, as defined above, bonded via one of its two radicals through a C₁-C₈ alkylenyl, as defined above. Illustrative examples of 5- or 6-membered heteroarylenyl-(C₁-C₈ alkylenyl) include:

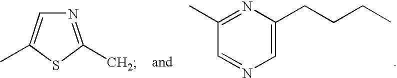

The phrase Substituted 5- or 6-membered heteroarylenyl-(C₁-C₈ alkylenyl)” means a 5- or 6-membered heteroarylenyl-(C₁-C₈ alkylenyl), as defined above, substituted with from 1 to 3 substituents, as defined above. Illustrative examples of substituted 5- or 6-membered heteroarylenyl-(C₁-C₈ alkylenyl) include:

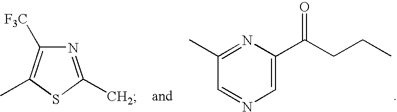

The phrase “(C₁-C₆ alkyl)-O” means a C₁-C₆ alkyl group, as defined above, bonded through an oxygen atom.

The phrase “(C₁-C₆ alkyl)-S” means a C₁-C₆ alkyl group, as defined above, bonded through an sulfur atom.

The phrase “(C₁-C₆ alkyl)-S(O)₂” means a C₁-C₆ alkyl group, as defined above, bonded through a sulfur atom, which sulfur atom is substituted with two oxygen atoms.

The phrase “(C₁-C₆ alkyl)-N(H)” means a C₁-C₆ alkyl group, as defined above, bonded through a nitrogen atom, which is bonded to a hydrogen atom.

The phrase “(C₁-C₆ alkyl)₂-N” means two independently selected C₁-C₆ alkyl groups, as defined above, including cyclic groups wherein the two C₁-C₆ alkyl groups are taken together with the nitrogen atom to which they are both bonded to form a 5- or 6-membered heterocycloalkyl, bonded through a nitrogen atom.

The phrase “(C₁-C₆ alkyl)-OC(O)” means a C₁-C₆ alkyl, as defined above, bonded through an oxygen atom-carbonyl carbon atom.

The phase “(C₁-C₆ alkyl)-C(O)O—(C₁-C₈ alkylenyl)ₘ”, wherein m is an integer of 0 or 1, means when, m is 0, a C₁-C₆ alkyl group, as defined above, bonded through a carbonyl carbon atom-oxygen atom, and, when m is 1, a C₁-C₆ alkyl group, as defined above, bonded through a carbonyl carbon atom-oxygen atom-(C₁-C₈ alkylenyl), wherein C₁-C₈ alkylenyl is as defined above.

The phase “(C₁-C₆ alkyl)-C(O)O-(1- to 8-membered heteroalkylenyl)ₘ”, wherein m is an integer of 0 or 1, means, when m is 0, a C₁-C₆ alkyl group, as defined above, bonded through a carbonyl carbon atom-oxygen atom, and, when m is 1, a C₁-C₆ alkyl group, as defined above, bonded through a carbonyl carbon atom-oxygen atom-(1- to 8-membered heteroalkylenyl), wherein 1- to 8-membered heteroalkylenyl is as defined above.

The phase “(C₁-C₆ alkyl)-C(O)N(H)—(C₁-C₈ alkylenyl)ₘ”, wherein m is an integer of 0 or 1, means, when m is 0, a C₁-C₆ alkyl group, as defined above, bonded through a carbonyl carbon atom-nitrogen atom, which is bonded to a hydrogen atom, and, when m is 1, a C₁-C₆ alkyl group, as defined above, bonded through a carbonyl carbon atom-nitrogen atom-(C₁-C₈ alkylenyl), wherein C₁-C₈ alkylenyl is as defined above and the nitrogen atom is bonded to a hydrogen atom.

The phase “(C₁-C₆ alkyl)-C(O)N(H)-(1- to 8-membered heteroalkylenyl)ₘ”, wherein m is an integer of 0 or 1, means when, m is 0, a C₁-C₆ alkyl group, as defined above, bonded through a carbonyl carbon atom-nitrogen atom, which is bonded to a hydrogen atom, and, when m is 1, a C₁-C₆ alkyl group, as defined above, bonded through a carbonyl carbon atom-nitrogen atom-(1- to 8-membered heteroalkylenyl), wherein 1- to 8-membered heteroalkylenyl is as defined above and the nitrogen atom is bonded to a hydrogen atom.

The phrase “H₂NS(O)₂—(C₁-C₈ alkylenyl)” means an amino bonded through a sulfur atom-(C₁-C₈ alkylenyl), wherein the C₁-C₈ alkylenyl is as defined above and the sulfur atom is bonded to two oxygen atoms.

The phrase “(C₁-C₆ alkyl)-N(H)S(O)₂—(C₁-C₈ alkylenyl)ₘ”, wherein m is an integer of 0 or 1, means, when m is 0, a C₁-C₆ alkyl, as defined above, bonded through a nitrogen atom-sulfur atom, and, when m is 1, a C₁-C₆ alkyl, as defined above, bonded through a nitrogen atom-sulfur atom-(C₁-C₈ alkylenyl), wherein the nitrogen atom is bonded to a hydrogen atom, the sulfur atom is bonded to two oxygen atoms, and C₁-C₈ alkylenyl is as defined above.

The phrase “(C₁-C₆ alkyl)₂-NS(O)₂—(C₁-C₈ alkylenyl)ₘ”, wherein m is an integer of 0 or 1, means, when m is 0, two C₁-C₆ alkyl groups, as defined above, including cyclic groups wherein the two C₁-C₆ alkyl groups are taken together with the nitrogen atom to which they are both bonded to form a 5- or 6-membered heterocycloalkyl, each bonded through a nitrogen atom-sulfur atom, and, when m is 1, two C₁-C₆ alkyl groups, as defined above, each bonded through a nitrogen atom-sulfur atom-(C₁-C₈ alkylenyl), wherein the nitrogen atom is bonded to a hydrogen atom, the sulfur atom is bonded to two oxygen atoms, and C₁-C₈ alkylenyl is as defined above.

The phrase “3- to 6-membered heterocycloalkyl-(G)ₘ”, wherein m is an integer of 0 or 1, means, when m is 0, a 3- to 6-membered heterocycloalkyl, as defined above, and, when m is 1, a 3- to 6-membered heterocycloalkyl, as defined above, bonded through a group G, as defined above.

The phrase “Substituted 3- to 6-membered heterocycloalkyl-(G)ₘ”, wherein m is an integer of 0 or 1, means, when m is 0, a substituted 3- to 6-membered heterocycloalkyl, as defined above, and, when m is 1, a substituted 3- to 6-membered heterocycloalkyl, as defined above, bonded through a group G, as defined above.

The phrase “5- or 6-membered heteroaryl-(G)ₘ”, wherein m is an integer of 0 or 1, means, when m is 0, a 5- or 6-membered heteroaryl, as defined above, and, when m is 1, a 5- or 6-membered heteroaryl, as defined above, bonded through a group G, as defined above.

The phrase “Substituted 5- or 6-membered heteroaryl-(G)ₘ”, wherein m is an integer of 0 or 1, means, when m is 0, a substituted 5- or 6-membered heteroaryl, as defined above, and, when m is 1, a substituted 5- or 6-membered heteroaryl, as defined above, bonded through a group G, as defined above.

The phrase “5-membered heteroarylenyl” means a 5-membered, monocyclic, aromatic ring diradical group having carbon atoms and from 1 to 4 heteroatoms selected from 1 O, 1 S, 1 N(H), 1 N(C₁-C₆ alkyl), and 4 N. Optionally, heteroarylenyl may be unsubstituted or substituted on a carbon atom (CH) or nitrogen atom [N(H)] with 1 substituent selected from fluoro, methyl, hydroxy, trifluoromethyl, cyano, and acetyl Illustrative examples of a 5-membered heteroarylenyl include thiophen-2,5-diyl, furan-2,3-di-yl, pyrrol-1,3-di-yl, imidazol-1,4-diyl, tetrazol-2,5-diyl, tetrazol-1,5-dicyl, oxadiazol-3,5-diyl, thiazol-2,4-diyl, and pyrazol-1,3-diyl.

The term “Phenyl-O—(C₁-C₈ alkylenyl)” means a phenyl bonded through an oxygen atom, which is bonded through a C₁-C₈ alkylenyl, wherein C₁-C₈ alkylenyl is as defined above. Illustrative examples of phenyl-O—(C₁-C₈ alkylenyl) include phenoxymethyl and 2-phenoxyethyl.

The term “Substituted phenyl-O—(C₁-C₈ alkylenyl)” means a phenyl-O—(C₁-C₈ alkylenyl) group, as defined above, that is substituted with from 1 to 4 substituents as defined above for R². Illustrative examples of substituted phenyl-O—(C₁-C₈ alkylenyl) include 4-fluorophenoxymethyl and 2-phenoxy-methylcarbonyl.

The term “Phenyl-S—(C₁-C₈ alkylenyl)” means a phenyl bonded through an sulfur atom, which is bonded through a C₁-C₈ alkylenyl, wherein C₁-C₈ alkylenyl is as defined above. Illustrative examples of phenyl-S—(C₁-C₈ alkylenyl) include thiophenoxymethyl and 2-thiophenoxyethyl.

The term “Substituted phenyl-S—(C₁-C₈ alkylenyl)” means a phenyl-S—(C₁-C₈ alkylenyl) group, as defined above, that is substituted with from 1 to 4 substituents as defined above for R². Illustrative examples of substituted phenyl-S—(C₁-C₈ alkylenyl) include 4-fluorothiophenoxymethyl and 2-thiophenoxy-methylcarbonyl.

The term “Phenyl-S(O)—(C₁-C₈ alkylenyl)” means a phenyl bonded through an sulfur atom, which is bonded through a C₁-C₈ alkylenyl, wherein C₁-C₈ alkylenyl is as defined above and the sulfur atom is also bonded to an oxygen atom. Illustrative examples of phenyl-S(O)—(C₁-C₈ alkylenyl) include phenyl-S(═O)—CH₂ and phenyl-S(═O)—CH₂CH₂.

The term “Substituted phenyl-S(O)—(C₁-C₈ alkylenyl)” means a phenyl-S(O)—(C₁-C₈ alkylenyl) group, as defined above, that is substituted with from 1 to 4 substituents as defined above for R². Illustrative examples of substituted phenyl-S(O)—(C₁-C₈ alkylenyl) include (4-Fluoro-phenyl)-S(═O)—CH₂ and phenyl-S(═O)—CH₂C(═O).

The term “Phenyl-S(O)₂—(C₁-C₈ alkylenyl)” means a phenyl bonded through an sulfur atom, which is bonded through a C₁-C₈ alkylenyl, wherein C₁-C₈ alkylenyl is as defined above and the sulfur atom is also bonded to two oxygen atoms. Illustrative examples of phenyl-S(O)₂—(C₁-C₈ alkylenyl) include phenyl-S(═O)₂—CH₂ and phenyl-S(═O)₂—CH₂CH₂.

The term “Substituted phenyl-S(O)₂—(C₁-C₈ alkylenyl)” means a phenyl-S(O)₂—(C₁-C₈ alkylenyl) group, as defined above, that is substituted with from 1 to 4 substituents as defined above for R². Illustrative examples of substituted phenyl-S(O)₂—(C₁-C₈ alkylenyl) include (4-Fluoro-phenyl)-S(═O)₂—CH₂ and phenyl-S(═O)₂—CH₂C(═O).

The term “(C₁-C₆ alkyl)-S(O)₂—N(H)—C(O)—(C₁-C₈ alkylenyl)ₘ”, wherein m is an integer of 0 or 1, means, when m is 0, a C₁-C₆ alkyl group, as defined above, bonded through a sulfur atom, which is bonded through a nitrogen atom, which is bonded through a carbon atom, wherein the sulfur atom is bonded to two oxygen atoms, the nitrogen atom is bonded to a hydrogen atom, and the carbon atom is doubly bonded to an oxygen atom to form a carbonyl group; and when m is 1, the term means a C₁-C₆ alkyl group, as defined above, bonded through a sulfur atom, which is bonded through a nitrogen atom, which is bonded through a carbon atom, which is bonded through a C₁-C₈ alkylenyl group, as defined above, wherein the sulfur atom is bonded to two oxygen atoms, the nitrogen atom is bonded to a hydrogen atom, and the carbon atom is doubly bonded to an oxygen atom to form a carbonyl group. Illustrative examples of (C₁-C₆ alkyl)-S(═O)₂—N(H)—C(O)—(C₁-C₈ alkylenyl)ₘ include CH₃—S(O)₂—N(H)—C(═O) and CH₃—S(O)₂—N(H)—C(═O)—CH₂.

The term “(C₁-C₆ alkyl)-C(O)—N(H)—S(O)₂—(C₁-C₈ alkylenyl)ₘ”, wherein m is an integer of 0 or 1, means, when m is 0, a C₁-C₆ alkyl group, as defined above, bonded through a carbon atom, which is bonded through a nitrogen atom, which is bonded through a sulfur atom, wherein the sulfur atom is bonded to two oxygen atoms, the nitrogen atom is bonded to a hydrogen atom, and the carbon atom is doubly bonded to an oxygen atom to form a carbonyl group; and when m is 1, the term means a C₁-C₆ alkyl group, as defined above, bonded through a carbon atom, which is bonded through a nitrogen atom, which is bonded through a sulfur atom, which is bonded through a C₁-C₈ alkylenyl group, as defined above, wherein the sulfur atom is bonded to two oxygen atoms, the nitrogen atom is bonded to a hydrogen atom, and the carbon atom is doubly bonded to an oxygen atom to form a carbonyl group. Illustrative examples of (C₁-C₆ alkyl)-C(O)—N(H)—S(O)₂—(C₁-C₈ alkylenyl)ₘ include CH₃—C(═O)—N(H)—S(═O)₂ and CH₃—C(═O)—N(H)—S(═O)₂—CH₂.

Preferred substituents for substituted phenyl, substituted naphthyl (i.e., substituted 1-naphthyl or substituted 2-naphthyl), and preferred substituents at carbon atoms for substituted 5-membered, monocyclic heteroaryl, substituted 6-membered, monocyclic heteroaryl, and substituted 9- or 10-membered, fused-bicyclic heteroaryl are C₁-C₄ alkyl, halo, OH, O—C₁-C₄ alkyl, 1,2-methylenedioxy, CN, NO₂, N₃, NH₂, N(H)CH₃, N(CH₃)₂, C(O)CH₃, OC(O)—C₁-C₄ alkyl, C(O)—H, CO₂H, CO₂—(C₁-C₄ alkyl), C(O)—N(H)OH, C(O)NH₂, C(O)NHMe, C(O)N(Me)₂, NHC(O)CH₃, N(H)C(O)NH₂, SH, S—C₁-C₄ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃.

Especially preferred substituents are 1,2-methylenedioxy, methoxy, ethoxy, —O—C(O)CH₃, carboxy, carbomethoxy, and carboethoxy.

The term “1,2-methylenedioxy” means the diradical group —O—CH₂—O—, wherein the substituent 1,2-methylenedioxy is bonded to adjacent carbon atoms of the group which is substituted to form a 5-membered ring. Illustrative examples of groups substituted by 1,2-methylenedioxy include 1,3-benzoxazol-5-yl of formula B

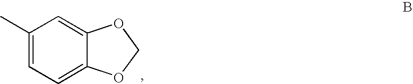

which is a phenyl group substituted by 1,2-methylenedioxy.

A fused-bicyclic group is a group wherein two ring systems share two, and only two, atoms.

It should be appreciated that the groups heteroaryl or heterocycloalkyl may not contain two ring atoms bonded to each other which atoms are oxygen and/or sulfur atoms.

The term “oxo” means ═O. Oxo is attached at a carbon atom unless otherwise noted. Oxo, together with the carbon atom to which it is attached forms a carbonyl group (i.e., C═O).

The term “heteroatom” includes O, S, S(O), S(O)₂, N, N(H), and N(C₁-C₆ alkyl).

The term “halo” includes fluoro, chloro, bromo, and iodo.

The term “amino” means NH₂.

The phrase “two adjacent, substantially sp² carbon atoms” means carbon atoms that comprise a carbon-carbon double bond that is capable of being substituted on each carbon atom, wherein the carbon-carbon double bond is contained in an aromatic or nonaromatic, cyclic or acyclic, or carbocyclic or heterocyclic group.

The term “(C₆-C₁₀)aryl”, as used herein, unless otherwise indicated, includes an organic radical derived from an aromatic hydrocarbon by removal of one or more hydrogens, such as phenyl, naphthyl indanyl or tetrahydronaphthyl; optionally substituted by 1 to 3 suitable substituents such as fluoro, chloro, cyano, nitro, trifluoromethyl, (C₁-C₆)alkoxy, (C₆-C₁₀)aryloxy, (C₃-C₈)cycloalkyloxy, trifluoromethoxy, difluoromethoxy, (C═O), O—(C═O), (C═O)—O, or (C₁-C₆)alkyl. The term “aryl” also encompasses fused aryl groups, including but not limited to pentalene, indene, naphthalene, azulene, and fluorene.

The term “(C₅-C₁₀)heteroaryl”, as used herein, unless otherwise indicated, includes an organic radical derived from an aromatic heterocyclic compound by removal of one or more hydrogens, such as benzimidazolyl, benzofuranyl, benzofurazanyl, 2H-1-benzopyranyl, benzothiadiazine, benzothiazinyl, benzothiazolyl, benzothiophenyl, benzoxazolyl, chromanyl, cinnolinyl, furazanyl, furopyridinyl, furyl, imidazolyl, indazolyl, indolinyl, indolizinyl, indolyl, 3H-indolyl, isoindolyl, isoquinolinyl, isothiazolyl, isoxazolyl, naphthyridinyl, oxadiazolyl, oxazolyl, phthalazinyl, pteridinyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, and triazolyl, wherein said heteroaryl is optionally substituted on any of the ring carbon atoms capable of forming an additional bond by one or two suitable substituents such as F, Cl, Br, CN, OH, (C₁-C₄)alkyl, (C₁-C₄)perfluoroalkyl, (C₁-C₄)perfluoroalkoxy, (C₁-C₄)alkoxy, (C═O), O—(C═O), (C═O)—O, and (C₃-C₈)cycloalkyloxy. The heteroaryl may also be optionally interrupted by (C═O) and (C═O)—O. The foregoing groups, as derived from the compounds listed above, can be C-attached or N-attached where such is possible. For instance, a group derived from pyrrole can be pyrrol-1-yl (N-attached) or pyrrol-3-yl (C-attached). The term “heteroaryl”, therefore includes aromatic heterocycles having one or more heteroatoms, such as N, O, or S. In addition, “heteroaryl” also refers to fused heteroaryl ring systems, including without limitation, benzofuran, isobenzofuran, benzothiofuran, isobenzothiofuran, indole, indolenine, 2-isobenzazole, 1,5-pyrindine, pyrano[3,4-b]-pyrrole, isoindazole, indoxazine, benzoxazole, anthranil, benzopyran, coumarin, chromone, isocoumarin, 2,3-benzopyrone, quinoline, isoquinoline, cinnoline, quinazoline, naphthyridine, pyrido[3,4-b]-pyridine, pyrido[3,2-b]-pyridine, pyrido[4,3-b]pyridine, and benzoxazine.

The term “(C₃-C₁₀)cycloalkyl”, as used herein, unless otherwise indicated, includes a mono or bicyclic carbocyclic ring (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, etc.); optionally containing 1-2 double bonds and optionally substituted by 1 to 3 suitable substituents as defined below such as fluoro, chloro, trifluoromethyl, (C₁-C₄)alkoxy, (C₆-C₁₀)aryloxy, trifluoromethoxy, difluoromethoxy (C═O), O—(C═O), (C═O)—O, or (C₁-C₄)alkyl, more preferably fluoro, chloro, methyl, ethyl and methoxy. The term “cycloalkyl” also includes bridged cycloalkyl groups, including, without limitation, norbornyl and adamantanyl, as well as spiro cycloalkyl groups, i.e., multi-ring systems joined by a single atom, such as:

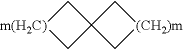

The term “(C₃-C₁₀)heterocyclyl”, as used herein, unless otherwise indicated, includes an organic radical derived from a non-aromatic heterocyclic compound by removal of one or more hydrogens, such as 3-azabicyclo[3.1.0]hexanyl, 3-azabicyclo[4.1.0]-heptanyl, azetidinyl, dihydrofuranyl, dihydropyranyl, dihydrothienyl, dioxanyl, 1,3-dioxolanyl, 1,4-dithianyl, hexahydroazepinyl, hexahydropyrimidine, imidazolidinyl, imidazolinyl, isoxazolidinyl, morpholinyl, oxazolidinyl, piperazinyl, piperidinyl, 2H-pyranyl, 4H-pyranyl, pyrazolidinyl, pyrazolinyl, pyrrolidinyl, 2-pyrrolinyl, 3-pyrrolinyl, quinolizinyl, tetrahydrofuranyl, tetrahydropyranyl, 1,2,3,6-tetrahydropyridinyl, tetrahydrothienyl, tetrahydrothiopyranyl, thiomorpholinyl, thioxanyl, and trithianyl. The foregoing groups, as derived from the compounds listed above, can be C-attached or N-attached where such is possible. For example, a group derived from piperidine can be piperidin-1-yl (N-attached) or piperidin-4-yl (C-attached). The foregoing groups, as derived from the compounds listed above, can be optionally substituted where such is possible by a suitable substituent, such as oxo F, Cl, Br, CN, OH, (C₁-C₄)alkyl, (C₁-C₄)perfluoroalkyl, (C₁-C₄)perfluoroalkoxy, (C₁-C₄)alkoxy, (C═O), O—(C═O), (C═O)—O, and (C₃-C₈)cycloalkyloxy. The term “heterocyclyl”, therefore includes heterocycles having one or more heteroatoms, such as N, O, or S. In addition, a “heterocyclyl” group may be optionally interrupted by one or more (C═O) or O—(C═O).

The phrase “a suitable substituent” is intended to mean a chemically and pharmaceutically acceptable functional group i.e., a moiety that does not negate the inhibitory activity of the inventive compounds. Such suitable substituents may be routinely selected by those skilled in the art. Illustrative examples of suitable substituents include, but are not limited to halo groups, perfluoroalkyl groups, perfluoroalkoxy groups, alkyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, carboxy groups, amino groups, alkyl- and dialkylamino groups, carbamoyl groups, alkylcarbonyl groups, alkoxycarbonyl groups, alkylaminocarbonyl groups dialkylamino carbonyl groups, arylcarbonyl groups, aryloxycarbonyl groups, alkylsulfonyl groups, an arylsulfonyl groups and the like.

The compounds of the invention possess a fused bicyclic ring structure of the formula:

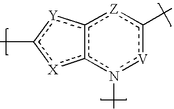

wherein dashed lines within the rings of the fused system represent optional double bonds. The position of a double bond within the ring system will depend, at least in part, on the nature of the atom at any given position in the ring system. For example, it will be understood that if Z is C═O, then neither bond to which Z is attached in the ring system depicted above can be a double bond.

The phrase “tertiary organic amine” means a trisubstituted nitrogen group wherein the 3 substituents are independently selected from C₁-C₁₂ alkyl, C₃-C₁₂ cycloalkyl, benzyl, or wherein two of the substituents are taken together with the nitrogen atom to which they are bonded to form a 5- or 6-membered, monocyclic heterocycle containing one nitrogen atom and carbon atoms, and the third substituent is selected from C₁-C₁₂ alkyl and benzyl, or wherein the three substituents are taken together with the nitrogen atom to which they are bonded to form a 7- to 12-membered bicyclic heterocycle containing 1 or 2 nitrogen atoms and carbon atoms, and optionally a C═N double bond when 2 nitrogen atoms are present. Illustrative examples of tertiary organic amine include triethylamine, diisopropylethylamine, benzyl diethylamino, dicyclohexylmethyl-amine, 1,8-diazabicycle[5.4.0]undec-7-ene (DBU), 1,4-diazabicyclo[2.2.2]octane (TED), and 1,5-diazabicycle[4.3.0]non-5-ene.

The phrase “pharmaceutical composition” means a composition suitable for administration in medical or veterinary use.

The term “admixed” and the phrase “in admixture” are synonymous and mean in a state of being in a homogeneous or heterogeneous mixture. Preferred is a homogeneous mixture.

The term “patient” means a mammal. Preferred patients are humans, cats, dogs, cows, horses, pigs, and sheep.

The term “animal” means a mammal, as defined above. Preferred animals include humans, cats, dogs, horses, pigs, sheep, cows, monkeys, rats, mice, guinea pigs, and rabbits.

The term “mammal” includes humans, companion animals such as cats and dogs, primates such as monkeys and chimpanzees, and livestock animals such as horses, cows, pigs, and sheep.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

The phrase “anticancer effective amount” means an amount of invention compound, or a pharmaceutically acceptable salt thereof, or a tautomer thereof, sufficient to inhibit, halt, or cause regression of the cancer being treated in a particular patient or patient population. For example in humans or other mammals, an anticancer effective amount can be determined experimentally in a laboratory or clinical setting, or may be the amount required by the guidelines of the United States Food and Drug Administration, or equivalent foreign agency, for the particular cancer and patient being treated.

The phrase “anti-arthritic effective amount” means an amount of invention compound, or a pharmaceutically acceptable salt thereof, or a tautomer thereof, sufficient to inhibit, halt, or cause regression of the arthritis being treated in a particular patient or patient population. For example in humans or other mammals, an anti-arthritic effective amount can be determined experimentally in a laboratory or clinical setting, or may be the amount required by the guidelines of the United States Food and Drug Administration, or equivalent foreign agency, for the particular arthritis and patient being treated.

The phrase “MMP-13 inhibiting amount” means an amount of invention compound, or a pharmaceutically acceptable salt thereof, or a tautomer thereof, sufficient to inhibit an enzyme matrix metalloproteinase-13, including a truncated form thereof, including a catalytic domain thereof, in a particular animal or animal population. For example in a human or other mammal, an MMP-13 inhibiting amount can be determined experimentally in a laboratory or clinical setting, or may be the amount required by the guidelines of the United States Food and Drug Administration, or equivalent foreign agency, for the particular MMP-13 enzyme and patient being treated.

It should be appreciated that determination of proper dosage forms, dosage amounts, and routes of administration, is within the level of ordinary skill in the pharmaceutical and medical arts, and is described below.

The phrases “effective amount” and “therapeutically effective amount” are synonymous and mean an amount of a compound of the present invention, a pharmaceutically acceptable salt thereof, or a solvate thereof, sufficient to effect an improvement of the condition being treated when administered to a patient suffering from a disease that is mediated by MMP-13 and optionally from 0 to 12 additional MMP enzymes.

The term “tautomer” means a form of invention compound existing in a state of equilibrium with an isomeric form of the invention compound, wherein the invention compound is able to react according to either form by virtue of the ability of the forms to interconvert by isomerization in situ, including in a reaction mixture, in an in vitro biological assay, or in vivo.

The term “(E)” means entgegen, and designates that the conformation about the double bond to which the term refers is the conformation having the two higher ranking substituent groups, as determined according to the Cahn-Ingold-Prelog ranking system, on opposite sides of the double bond. An (E) double bond is illustrated below by the compound of Formula (W)

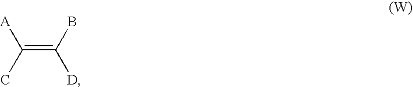

wherein the two higher-ranking substituents are groups A and D.

The term “(Z)” means zusammen, and designates that the conformation about the double bond to which the term refers is the conformation having the two higher ranking substituent groups, as determined according to the Cahn-Ingold-Prelog ranking system, on the same side of the double bond. A (Z) double bond is illustrated below by the compound of Formula (X)

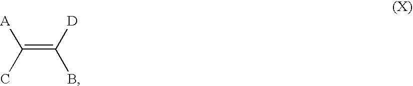

wherein the two higher-ranking substituents are groups A and D.

It should be appreciated that the S1′ site of MMP-13 was previously thought to be a grossly linear channel which contained an opening at the top that allowed an amino acid side chain from a substrate molecule to enter during binding, and was closed at the bottom. Applicants has discovered that the S1′ site is actually composed of an S1′ channel angularly connected to a newly discovered pocket which applicant calls the S1″ site. The S1″ site is open to solvent at the bottom, which can expose a functional group of Applicants' invention compounds to solvent. For illustrative purposes, the S1′ site of the MMP-13 enzyme can now be thought of as being like a sock with a hole in the toes, wherein the S1′ channel is the region from approximately the opening to the ankle, and the S1″ site is the foot region below the ankle, which foot region is angularly connected to the ankle region. However, the invention compounds do not necessarily bind in the S1′ site of MMP-13.

More particularly, the S1′ channel is a specific part of the S1′ site and is formed largely by Leu218, Val219, His222 and by residues from Leu239 to Tyr244. The S1″ binding site which has been newly discovered is defined by residues from Tyr246 to Pro255. The S1″ site contains at least two hydrogen bond donors and aromatic groups which interact with an invention compound.

Without wishing to be bound by any particular theory, the inventors believe that the S1″ site could be a recognition site for triple helix collagen, the natural substrate for MMP-13. It is possible that the conformation of the S1″ site is modified only when an appropriate compound binds to MMP-13, thereby interfering with the collagen recognition process. This newly discovered pattern of binding offers the possibility of greater selectivity than what is achievable with the binding pattern of known selective inhibitors of MMP-13, wherein the known binding pattern requires ligation of the catalytic zinc atom at the active site and occupation the S1′ channel, but not the S1″ site. Alternatively, inhibition of the MMP may result from a suitable electronic interaction (e.g., hydrogen bonding) between an invention compound and one or more of the histidine residues that ligate the catalytic zinc of MMP-13.

The compounds of Formula I, or pharmaceutically acceptable salts thereof, or tautomers thereof, include compounds which are invention compounds. An allosteric inhibitor of MMP-13 is any compound of Formula I that binds allosterically into the S1′ site of the MMP-13 enzyme, including the S1′ channel, and a newly discovered S1″ site, and ligates, coordinates, or binds the catalytic zinc of the MMP-13 with a group Z, wherein Z is as defined above.

The term “Thr245” means threonine 245 of an MMP-13 enzyme.

The term “Thr247” means threonine 247 of an MMP-13 enzyme.

The term “Met253” means methionine 253 of an MMP-13 enzyme.

The term “His251” means histidine 251 of an MMP-13 enzyme.

It should be appreciated that the matrix metalloproteinases include, but are not limited to, the following enzymes:

MMP-1, also known as interstitial collagenase, collagenase-1, or fibroblast-type collagenase;

MMP-2, also known as gelatinase A or 72 kDa Type IV collagenase;

MMP-3, also known as stromelysin or stromelysin-1;

MMP-7, also known as matrilysin or PUMP-1;

MMP-8, also known as collagenase-2, neutrophil collagenase or polymorphonuclear-type (“PMN-type”) collagenase;

MMP-9, also known as gelatinase B or 92 kDa Type IV collagenase;

MMP-10, also known as stromelysin-2;

MMP-11, also known as stromelysin-3;

MMP-12, also known as macrophage metalloelastase;

MMP-13, also known as collagenase-3;

MMP-14, also known as membrane-type (“MT”) 1-MMP or MT1-MMP;

MMP-15, also known as MT2-MMP;

MMP-16, also known as MT3-MMP;

MMP-17, also known as MT4-MMP;

MMP-18, also known as collagenase-4;

MMP-19, also known as RASI-1 and RASI-6;

MMP-20, also known as enamelysin;

MMP-23, also referred to as “MMP-21” in reproductive tissues;

MMP-24, also known as MT5-MMP;

MMP-25, also known as MT6-MMP and leukolysin;

MMP-26, also known as matrilysin-2 and endometase;

MMP-27; and

MMP-28, also known as epilysin.

For the purposes of this invention, the term “arthritis”, which is synonymous with the phrase “arthritic condition”, includes osteoarthritis, rheumatoid arthritis, degenerative joint disease, spondyloarthropathies, gouty arthritis, systemic lupus erythematosus, juvenile arthritis, and psoriatic arthritis. An inhibitor of MMP-13 having an anti-arthritic effect is a compound as defined above that inhibits the progress, prevents further progress, or reverses progression, in part or in whole, of any one or more symptoms of any one of the arthritic diseases and disorders listed above.

The term “IC₅₀” means the concentration of a compound, usually expressed as micromolar or nanomolar, required to inhibit an enzyme's catalytic activity by 50%.

The term “ED₄₀” means the concentration of a compound, usually expressed as micromolar or nanomolar, required to treat a disease in about 40% of a patient group.

The term “ED₃₀” means the concentration of a compound, usually expressed as micromolar or nanomolar, required to treat a disease in 30% of a patient group.

The phrase “pharmaceutical composition” means a composition suitable for administration in medical or veterinary use.

The term “admixed” and the phrase “in admixture” are synonymous and mean in a state of being in a homogeneous or heterogeneous mixture. Preferred is a homogeneous mixture.

As used herein, the phrase “cartilage damage” means a disorder of hyaline cartilage, including articular cartilage, and subchondral bone characterized by hypertrophy of tissues in and around the involved joints, which may or may not be accompanied by deterioration of hyaline cartilage surface.

The phrase “treating”, which is related to the terms “treat” and “treated”, means administration of an invention combination as defined above that inhibits the progress, prevents further progress, or reverses progression, in part or in whole, of any one or more symptoms of any one of the diseases and disorders listed above.

The phrase “invention compound” means a compound of Formula I, or a pharmaceutically acceptable salt thereof, as fully defined above.

The term “nontoxic” means the efficacious dose is 10 times or greater than the dose at which a toxic effect is observed in 10% or more of a patient population.

The term “celecoxib” means the compound named 4-(5-(4-methylphenyl)-3-(trifluoromethyl)-1H-pyrazol-1-yl)-benzenesulfonamide. Celecoxib is a selective cyclooxygenase-2 (“COX-2”) inhibitor currently approved by the FDA for the treatment of osteoarthritis, rheumatoid arthritis, and Polyposis-familial adenomatus. Celecoxib is marketed under the tradename “Celebrex”. Celecoxib is currently in clinical trials for the treatment of bladder cancer, chemopreventative-lung cancer, and post-operative pain, and is registered for the treatment of dysmenorrhea. Celecoxib has the structure drawn below:

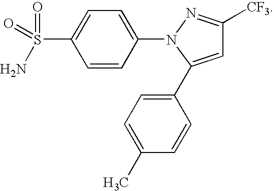

The term “valdecoxib” means the compound named 4-(5-methyl-3-phenyl-4-isoxazolyl)-benzenesulfonamide. Valdecoxib is a selective COX-2 inhibitor that has been approved by the FDA for treating osteoarthritis, rheumatoid arthritis, dysmenorrhea, and general pain, and is marketed under the tradename “Bextra”. Valdecoxib is in clinical trials for the treatment of migraine. Valdecoxib has the structure drawn below:

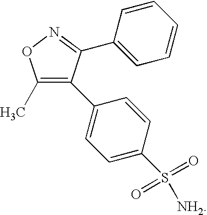

It should be appreciated that COX-2 is also known as prostaglandin synthase-2, prostaglandin PGH₂ synthase, and prostaglandin-H₂ synthase-2.

A selective inhibitor of COX-2 means compounds that inhibit COX-2 selectively versus COX-1 such that a ratio of IC₅₀ for a compound with COX-1 divided by a ratio of IC₅₀ for the compound with COX-2 is greater than, or equal to, 5, where the ratios are determined in one or more assays. All that is required to determine whether a compound is a selective COX-2 inhibitor is to assay a compound in one of a number of well know assays in the art.

The term “NSAID” is an acronym for the phrase “nonsteroidal anti-inflammatory drug”, which means any compound which inhibits cyclooxygenase-1 (“COX-1”)and cyclooxygenase-2. Most NSAIDs fall within one of the following five structural classes: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxicam. Other useful NSAIDs include aspirin, acetominophen, indomethacin, and phenylbutazone. Selective inhibitors of cyclooxygenase-2 as described above may be considered to be NSAIDs also.

The term “drugs”, which is synonymous with the phrases “active components”, “active compounds”, and “active ingredients”, includes celecoxib, or a pharmaceutically acceptable salt thereof, valdecoxib, or a pharmaceutically acceptable salt thereof, and an inhibitor of MMP-13, and may further include one or two of the other therapeutic agents described above.

An invention compound that is an allosteric inhibitor of MMP-13 may be readily identified by one of ordinary skill in the pharmaceutical or medical arts by assaying a test compound for inhibition of MMP-13 as described below in Biological Methods 1 or 2, and for allosteric inhibition of MMP-13 by assaying the test invention compound for inhibition of MMP-13 in the presence of an inhibitor to the catalytic zinc of MMP-13 as described below in Biological Methods 3 or 4.

Further, an invention compound having an anti-inflammatory, an analgesic, anti-arthritic, or a cartilage damage inhibiting effect, or any combination of these effects, may be readily identified by one of ordinary skill in the pharmaceutical or medical arts by assaying the invention compound in any number of well known assays for measuring determining the invention compound's effects on cartilage damage, arthritis, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of an invention compound or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content. Further, in vivo assays to assay cartilage damage may be performed as follows: an amount of an invention compound or control vehicle may be administered with a cartilage damaging agent to an animal, and the effects of the invention compound being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content.

Several methods of identifying an invention compound with cartilage damage inhibiting properties are described below. The amount to be administered in an assay is dependent upon the particular assay employed, but in any event is not higher than the well known maximum amount of a compound that the particular assay can effectively accommodate.

Similarly, invention compounds having pain-alleviating properties may be identified using any one of a number of in vivo animal models of pain.

Still similarly, invention compounds having anti-inflammatory properties may be identified using any one of a number of in vivo animal models of inflammation. For example, for an example of inflammation models, see U.S. Pat. No. 6,329,429, which is incorporated herein by reference.

Still similarly, invention compounds having anti-arthritic properties may be identified using any one of a number of in vivo animal models of arthritis. For example, for an example of arthritis models, see also U.S. Pat. No. 6,329,429.

Other mammalian diseases and disorders which are treatable by administration of an invention combination alone, or contained in a pharmaceutical composition as defined below, include: fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

Other aspects of the present invention are compounds of Formula I, or a pharmaceutically acceptable salt thereof, that are ≧10, ≧20, ≧50, ≧100, or ≧1000 times more potent versus MMP-13 than versus at least two of any other MMP enzyme or TACE.

Still other aspects of the present invention are compounds of Formula I, or a pharmaceutically acceptable salt thereof, that are selective inhibitors of MMP-13 versus 2, 3, 4, 5, 6, or 7 other MMP enzymes, or versus TACE and 1, 2, 3, 4, 5, 6, or 7 other MMP enzymes.

It should be appreciated that selectivity of a compound of Formula I, or a pharmaceutically acceptable salt thereof, is a multidimensional characteristic that includes the number of other MMP enzymes and TACE over which selectivity for MMP-13 inhibition is present and the degree of selectivity of inhibition of MMP-13 over another particular MMP or TACE, as measured by, for example, the IC₅₀ in micromolar concentration of the compound for the inhibition of the other MMP enzyme or TACE divided by the IC₅₀ in micromolar concentration of the compound for the inhibition of MMP-13.

As discussed above, one aspect of the present invention is novel compounds that are selective inhibitors of the enzyme MMP-13. A selective inhibitor of MMP-13, as used in the present invention, is a compound that is ≧5× more potent in vitro versus MMP-13 than versus at least one other matrix metalloproteinase enzyme such as, for example, MMP-1, MMP-2, MMP-3, MMP-7, MMP-8, MMP-9, or MMP-14, or versus tumor necrosis factor alpha convertase (“TACE”). A preferred aspect of the present invention is novel compounds that are selective inhibitors of MMP-13 versus MMP-1.

The invention provides a compound of Formula I, or a pharmaceutically acceptable salt thereof, which has an IC₅₀ with any MMP enzyme that is less than or equal to 50 micromolar. Preferred are compounds of Formula I, or a pharmaceutically acceptable salt thereof, which have an IC₅₀ with a human full-length MMP-13 (“hMMP-13FL”) or a human MMP-13 catalytic domain (“hMMP-13CD”) that is less than or equal to 50 micromolar. More preferred are compounds of Formula I, or a pharmaceutically acceptable salt thereof, which have an IC₅₀ with a human full-length MMP-13 (“hMMP-13FL”) or a human MMP-13 catalytic domain (“hMMP-13CD”) that is less than or equal to 10 micromolar.

Examples of biological methods useful for determining IC₅₀s for the invention compounds with an MMP are described below in Biological Methods 1 to 4. Any compound of Formula I, or a pharmaceutically acceptable salt thereof, or any form thereof as defined above, that does not have an IC₅₀ with any MMP enzyme that is less than, or equal to, 10 micromolar is excluded from this invention.

Some of the invention compounds are capable of further forming nontoxic pharmaceutically acceptable salts, including, but not limited to, acid addition and/or base salts. The acid addition salts are formed from basic invention compounds, whereas the base addition salts are formed from acidic invention compounds. All of these forms are within the scope of the compounds useful in the invention.

Pharmaceutically acceptable acid addition salts of the basic invention compounds include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a basic invention compound is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the invention compounds and their respective acid addition salt forms are equivalent for purposes of the present invention.

A nontoxic pharmaceutically acceptable base addition salt of an acidic invention compound may be prepared by contacting the free acid form of the compound with a metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic invention compound may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the invention compounds differ from their respective salt forms somewhat in certain physical properties such as solubility, crystal structure, hygroscopicity, and the like, but otherwise the salts are equivalent to their respective free acid for purposes of the present invention.

Certain invention compounds can exist in unsolvated forms as well as solvated forms, including hydrated forms. In general, the solvated forms, including hydrated forms, are equivalent to unsolvated forms and are encompassed within the scope of the present invention.

Certain of the invention compounds possess one or more chiral centers, and each center may exist in the R or S configuration. An invention compound includes any diastereomeric, enantiomeric, or epimeric form of the compound, as well as mixtures thereof.

Additionally, certain invention compounds may exist as geometric isomers such as the entgegen (E) and zusammen (Z) isomers of 1,2-disubstituted alkenyl groups or cis and trans isomers of disubstituted cyclic groups. An invention compound includes any cis, trans, syn, anti, entgegen (E), or zusammen (Z) isomer of the compound, as well as mixtures thereof.

Certain invention compounds can exist as two or more tautomeric forms. Tautomeric forms of the invention compounds may interchange, for example, via enolization/de-enolization, 1,2-hydride, 1,3-hydride, or 1,4-hydride shifts, and the like. An invention compound includes any tautomeric form of the compound, as well as mixtures thereof.

Some compounds of the present invention have alkenyl groups, which may exist as entgegen or zusammen conformations, in which case all geometric forms thereof, both entgegen and zusammen, cis and trans, and mixtures thereof, are within the scope of the present invention.

Some compounds of the present invention have cycloalkyl groups, which may be substituted at more than one carbon atom, in which case all geometric forms thereof, both cis and trans, and mixtures thereof, are within the scope of the present invention.

The invention compounds also include isotopically-labelled compounds, which are identical to those recited above, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts of said compounds which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by carrying out the procedures incorporated by reference above or disclosed in the Schemes and/or in the Examples and Preparations below, by substituting a readily available isotopically labelled reagent for a non-isotopically labelled reagent.

All of the above-describe forms of an invention compound are included by the phrase “invention compound”, a “compound of Formula I”, a “compound of Formula I, or a pharmaceutically acceptable salt thereof”, or any named species thereof, unless specifically excluded therefrom.

One of ordinary skill in the art will appreciate that the compounds of the invention are useful in treating a diverse array of diseases. One of ordinary skill in the art will also appreciate that when using the compounds of the invention in the treatment of a specific disease that the compounds of the invention may be combined with various existing therapeutic agents used for that disease.

For the treatment of rheumatoid arthritis, the compounds of the invention may be combined with agents such as TNF-α inhibitors such as anti-TNF monoclonal antibodies and TNF receptor immunoglobulin molecules (such as Enbrel®), low dose methotrexate, lefunimide, hydroxychloroquine, d-penicillamine, auranofin or parenteral or oral gold.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as etoricoxib and rofecoxib, analgesics and intraarticular therapies such as corticosteroids and hyaluronic acids such as hyalgan and synvisc.

This invention also relates to a method of or a pharmaceutical composition for treating inflammatory processes and diseases comprising administering a compound of this invention to a mammal, including a human, cat, livestock or dog, wherein said inflammatory processes and diseases are defined as above and said inhibitory compound is used in combination with one or more other therapeutically active agents under the following conditions:

A.) where a joint has become seriously inflamed as well as infected at the same time by bacteria, fungi, protozoa and/or virus, said inhibitory compound is administered in combination with one or more antibiotic, antifungal, antiprotozoal and/or antiviral therapeutic agents;

B.) where a multi-fold treatment of pain and inflammation is desired, said inhibitory compound is administered in combination with inhibitors of other mediators of inflammation, comprising one or more members independently selected from the group consisting essentially of:

(1) NSAIDs;

(2) H₁-receptor antagonists;

(3) kinin-B₁- and B₂-receptor antagonists;

(4) prostaglandin inhibitors selected from the group consisting of PGD-, PGF- PGI₂- and PGE-receptor antagonists;

(5) thromboxane A₂ (TXA₂-) inhibitors;

(6) 5-, 12- and 15-lipoxygenase inhibitors;

(7) leukotriene LTC₄-, LTD₄/LTE₄- and LTB₄-inhibitors;

(8) PAF-receptor antagonists;

(9) gold in the form of an aurothio group together with one or more hydrophilic groups;

(10) immunosuppressive agents selected from the group consisting of cyclosporine, azathioprine and methotrexate;

(11) anti-inflammatory glucocorticoids;

(12) penicillamine;

(13) hydroxychloroquine;

(14) anti-gout agents including colchicine; xanthine oxidase inhibitors including allopurinol; and uricosuric agents selected from probenecid, sulfinpyrazone and benzbromarone;

C. where older mammals are being treated for disease conditions, syndromes and symptoms found in geriatric mammals, said inhibitory compound is administered in combination with one or more members independently selected from the group consisting essentially of:

(1) cognitive therapeutics to counteract memory loss and impairment;

(2) anti-hypertensives and other cardiovascular drugs intended to offset the consequences of atherosclerosis, hypertension, myocardial ischemia, angina, congestive heart failure and myocardial infarction, selected from the group consisting of:

a. diuretics;

b. vasodilators;

c. β-adrenergic receptor antagonists;

d. angiotensin-II converting enzyme inhibitors (ACE-inhibitors), alone or optionally together with neutral endopeptidase inhibitors;

e. angiotensin II receptor antagonists;

f. renin inhibitors;

g. calcium channel blockers;

h. sympatholytic agents;

i. α₂-adrenergic agonists;

j. α-adrenergic receptor antagonists; and

k. HMG-CoA-reductase inhibitors (anti-hypercholesterolemics);

(3) antineoplastic agents selected from:

a. antimitotic drugs selected from:

i. vinca alkaloids selected from:

[1] vinblastine and

[2] vincristine;

(4) growth hormone secretagogues;

(5) strong analgesics;

(6) local and systemic anesthetics; and

(7) H₂-receptor antagonists, proton pump inhibitors and other gastroprotective agents.

The active ingredient of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, ILra, H₁-receptor antagonists; kinin-B₁- and B₂-receptor antagonists; prostaglandin inhibitors such as PGD-, PGF- PGI₂- and PGE-receptor antagonists; thromboxane A₂ (TXA2-) inhibitors; 5- and 12-lipoxygenase inhibitors; leukotriene LTC₄-, LTD₄/LTE₄- and LTB₄-inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The compounds of the present invention may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The compounds of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The compounds of the present invention may also be administered in combination with one or more antibiotic, antifungal, antiprotozoal, antiviral or similar therapeutic agents.

The compounds of the present invention may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The compounds of the present invention may also be used in combination with osteoporosis agents such as roloxifene, lasofoxifene, droloxifene or fosomax and immunosuppressant agents such as FK-506 and rapamycin.

The present invention also relates to the formulation of a compound of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

The invention method is useful in human and veterinary medicines for treating mammals suffering from one or more of the above-listed diseases and disorders.

All that is required to practice a method of this invention is to administer a compound of Formula I, or a pharmaceutically acceptable salt thereof, in an amount that is therapeutically effective for preventing, inhibiting, or reversing the condition being treated. The invention compound can be administered directly or in a pharmaceutical composition as described below.

A therapeutically effective amount, or, simply, effective amount, of an invention compound will generally be from about 1 to about 300 mg/kg of subject body weight of the compound of Formula I, or a pharmaceutically acceptable salt thereof. Typical doses will be from about 10 to about 5000 mg/day for an adult subject of normal weight for each component of the combination. In a clinical setting, regulatory agencies such as, for example, the Food and Drug Administration (“FDA”) in the U.S. may require a particular therapeutically effective amount.

In determining what constitutes a nontoxic effective amount or a therapeutically effective amount of an invention compound for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including the Food and Drug Administration guidelines, or guidelines from an equivalent agency, published clinical studies, the subject's (e.g., mammal's) age, sex, weight and general condition, as well as the type and extent of the disease, disorder or condition being treated, and the use of other medications, if any, by the subject. As such, the administered dose may fall within the ranges or concentrations recited above, or may vary outside them, ie, either below or above those ranges, depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the invention compound that are less than optimum for a particular subject. Thereafter, the dosage can be increased by small increments until the optimum effect under the circumstance is reached. For convenience, the total daily dosage may be divided and administered in portions during the day, if desired.

Pharmaceutical compositions, described briefly here and more fully below, of an invention combination may be produced by formulating the invention combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Alternatively, the invention compounds may be formulated separately.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat any of the above-listed diseases and disorders.

The percentage of the active ingredients of a compound of Formula I, or a pharmaceutically acceptable salt thereof, in the foregoing compositions can be varied within wide limits, but for practical purposes it is preferably present in a total concentration of at least 10% in a solid composition and at least 2% in a primary liquid composition. The most satisfactory compositions are those in which a much higher proportion of the active ingredients are present, for example, up to about 95%.

Preferred routes of administration of an invention compound are oral or parenteral. However, another route of administration may be preferred depending upon the condition being treated. For exampled, topical administration or administration by injection may be preferred for treating conditions localized to the skin or a joint. Administration by transdermal patch may be preferred where, for example, it is desirable to effect sustained dosing.

It should be appreciated that the different routes of administration may require different dosages. For example, a useful intravenous (“IV”) dose is between 5 and 50 mg, and a useful oral dosage is between 20 and 800 mg, of a compound of Formula I, or a pharmaceutically acceptable salt thereof. The dosage is within the dosing range used in treatment of the above-listed diseases, or as would be determined by the needs of the patient as described by the physician.

The invention compounds may be administered in any form. Preferably, administration is in unit dosage form. A unit dosage form of the invention compound to be used in this invention may also comprise other compounds useful in the therapy of diseases described above. A further description of pharmaceutical formulations useful for administering the invention compounds and invention combinations is provided below.

The active components of the invention combinations, may be formulated together or separately and may be administered together or separately. The particular formulation and administration regimens used may be tailored to the particular patient and condition being treated by a practitioner of ordinary skill in the medical or pharmaceutical arts.

The advantages of using an invention compound in a method of the instant invention include the nontoxic nature of the compounds at and substantially above therapeutically effective doses, their ease of preparation, the fact that the compounds are well-tolerated, and the ease of topical, IV, or oral administration of the drugs.

Another important advantage is that the present invention compounds more effectively target a particular disease that is responsive to inhibition of MMP-13 with fewer undesirable side effects than similar compounds that inhibit MMP-13 that are not invention compounds. This is so because the instant invention compounds of Formula I, or a pharmaceutically acceptable salt thereof, bind at a different location from where natural substrate binds to MMP-13, and also reach up the S′ binding pocket of the MMP-13 to ligate, coordinate, or bind to the catalytic zinc cation. The binding requirements of an allosteric MMP-13 binding site are unique to MMP-13, and account for the specificity of the invention compounds for inhibiting MMP-13 over any other MMP enzyme. This binding mode has not been reported in the art. Indeed, prior art inhibitors of MMP-13 bind to MMP-13 in a manner similar to the binding of substrate, including binding to the catalytic zinc cations of other MMP enzymes as well as to the catalytic zinc cation of MMP-13, and are consequently significantly less selective inhibitors of MMP-13 enzyme.

The invention compounds which are invention compounds, and pharmaceutically acceptable salts thereof, are thus therapeutically superior to other inhibitors of MMP-13, or even tumor necrosis factor-alpha converting enzyme (“TACE”), because of fewer undesirable side effects from inhibition of the other MMP enzymes or TACE. For example, virtually all prior art MMP inhibitors tested clinically to date have exhibited an undesirable side effect known as muscoloskeletal syndrome (“MSS”). MSS is associated with administering an inhibitor of multiple MMP enzymes or an inhibitor of a particular MMP enzyme such as MMP-1. MSS will be significantly reduced in type and severity by administering the invention compound instead of any prior art MMP-13 inhibitor, or a pharmaceutically acceptable salt thereof. The invention compounds are superior to similar compounds that interact with the catalytic zinc cation of the MMP-13 enzyme as discussed above, even if similar compounds show some selectivity for the MMP-13.

It is expected that nearly all, if not all, compounds of Formula I, or pharmaceutically acceptable salts thereof, are invention compounds.

This advantage of the instant compounds will also significantly increase the likelihood that agencies which regulate new drug approvals, such as the United States Food and Drug Administration, will approve the instant compounds versus a competing similar compound that does not allosterically bind to MMP-13 as discussed above even in the unlikely event that the two compounds behaved similarly in clinical trials. These regulatory agencies are increasingly aware that clinical trials, which test drug in limited population groups, do not always uncover safety problems with a drug, and thus all other things being equal, the agencies will favor the drug with the lowest odds of producing undesirable side effects.

Another important advantage is that the disease modifying properties of the invention compounds provide patients suffering from cartilage damage, arthritis, preferably osteoarthritis, inflammation and/or pain with both relief of symptoms and prevention or inhibition of the underlying disease pathology such as cartilage degradation. There is no currently approved drug for disease modification of cartilage damage, including in osteoarthritis.

Any invention compound is readily available, either commercially, or by synthetic methodology, well known to those skilled in the art of organic chemistry. For specific syntheses, see the examples below and the preparations of invention compound outlined in the Schemes below.

Intermediates for the synthesis of a compound of Formula I, or a pharmaceutically acceptable salt thereof, may be prepared by one of ordinary skill in the art of organic chemistry by adapting various synthetic procedures incorporated by reference above or that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods, 1989, by Wiley-Interscience; the text Advanced Organic Chemistry, 4^{th} edition, by Jerry March, Wiley-Interscience, New York, 1992; or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the invention compounds may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A.G., Switzerland, or Lancaster Synthesis Ltd, United Kingdom.

Syntheses of some invention compounds may utilize starting materials, intermediates, or reaction products that contain a reactive functional group. During chemical reactions, a reactive functional group may be protected from reacting by a protecting group that renders the reactive functional group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of a compound of Formula I, or a pharmaceutically acceptable salt thereof, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: N.Y., 1991, which is hereby incorporated by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

General syntheses of the compounds of Formula I are outlined below in more than 100 schemes. The schemes, and description which accompanies the schemes, are for illustration purposes only and should not be construed as limiting the preparation of the compounds of Formula I in any way. In the schemes, Z, L, R¹, R², and Q are as defined above or may be protected and then later deprotected as described above to give a compound of Formula I.

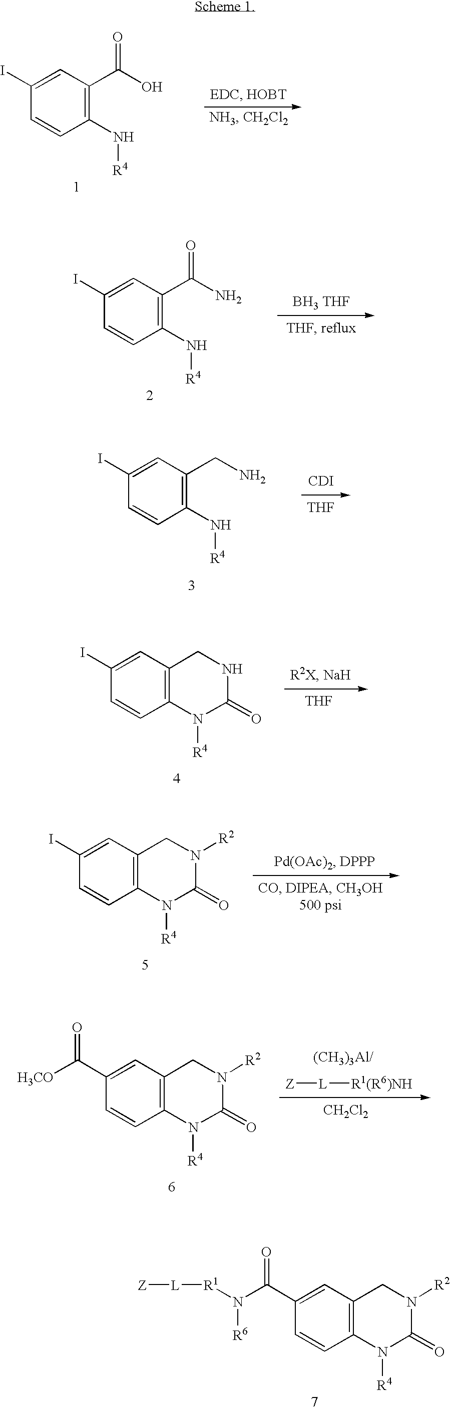

wherein R⁴ is H or C₁-C₆ alkyl and R², R⁶, Z, L, and R¹ are as defined above.

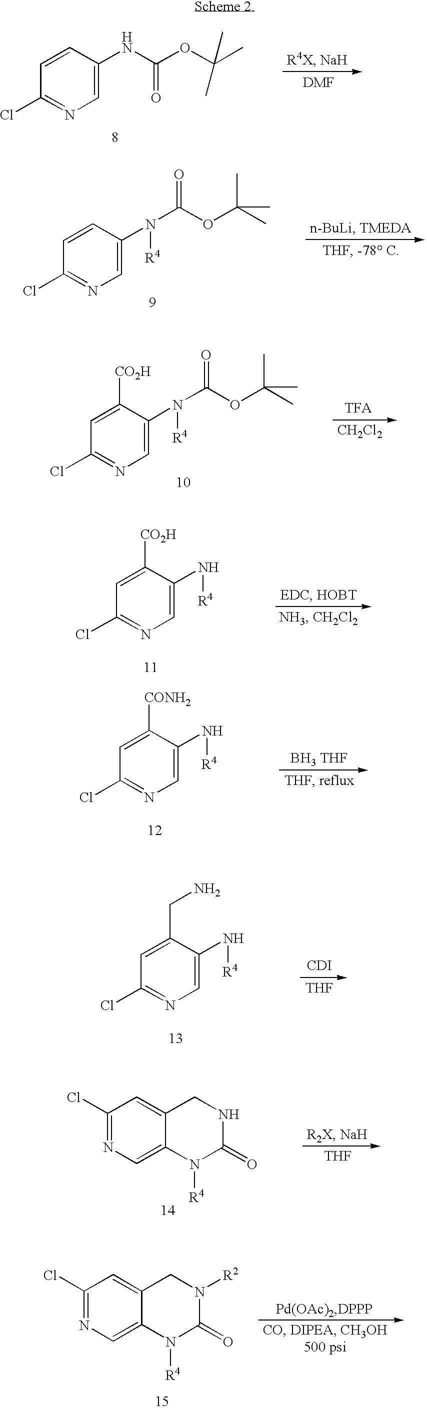

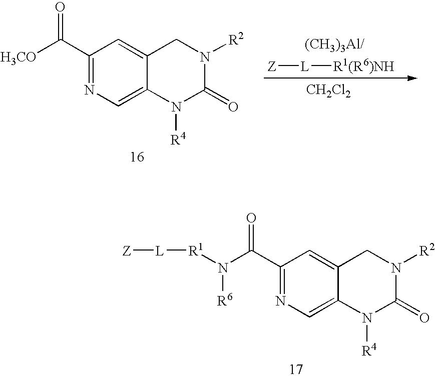

wherein R⁴ is H or C₁-C₆ alkyl and R², R⁶, Z, L, and R¹ are as defined above.

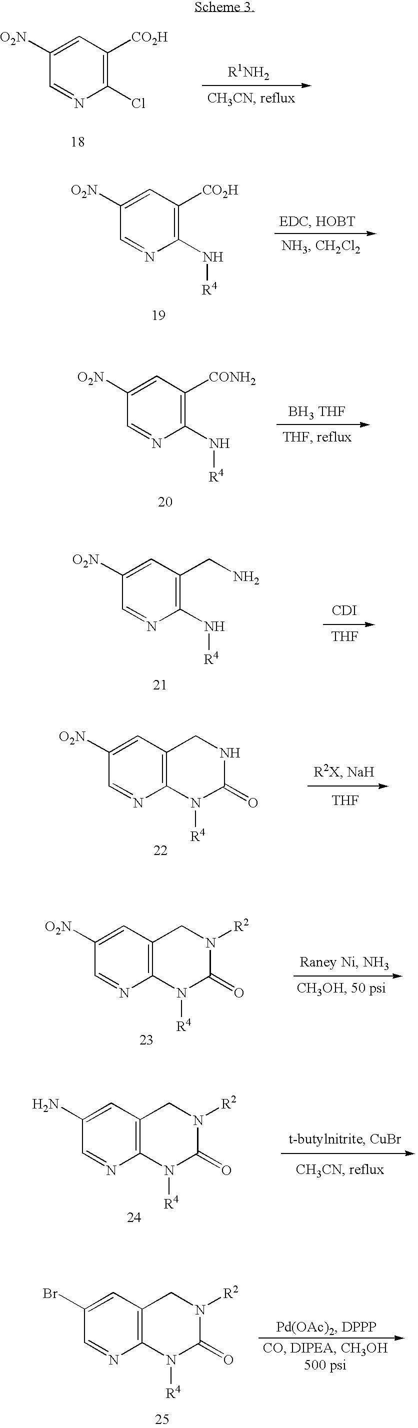

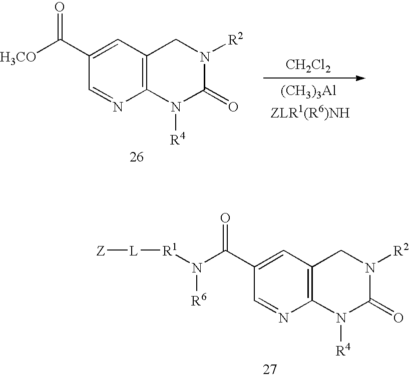

wherein R⁴ is H or C₁-C₆ alkyl and R², R⁶, Z, L, and R¹ are as defined above.

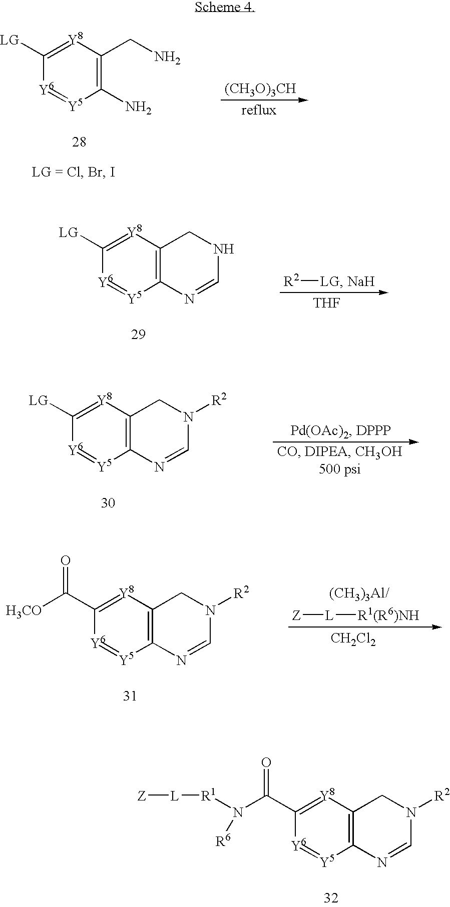

wherein Y⁵, Y⁶, and Y⁸ are each independently C(H) or N, and R², R⁶, Z, L, and R¹ are as defined above.

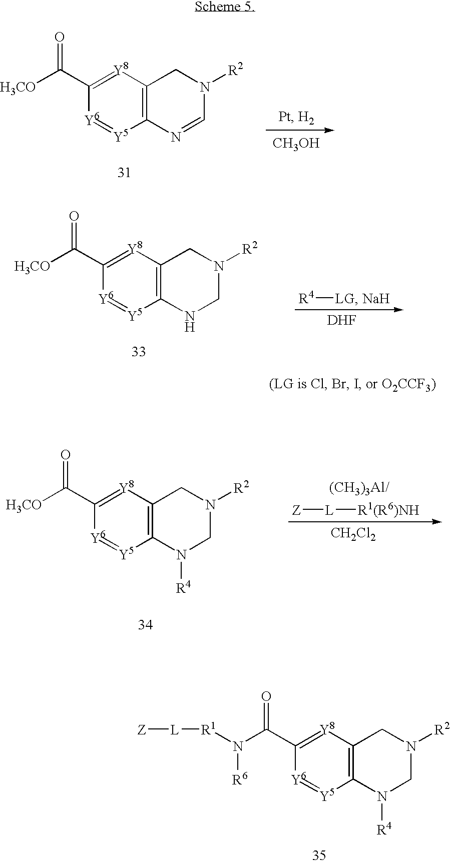

wherein R⁴ is H or C₁-C₆ alkyl, Y⁵, Y⁶, and Y⁸ are each independently C(H) or N, and R², R⁶, Z, L, and R¹ are as defined above.

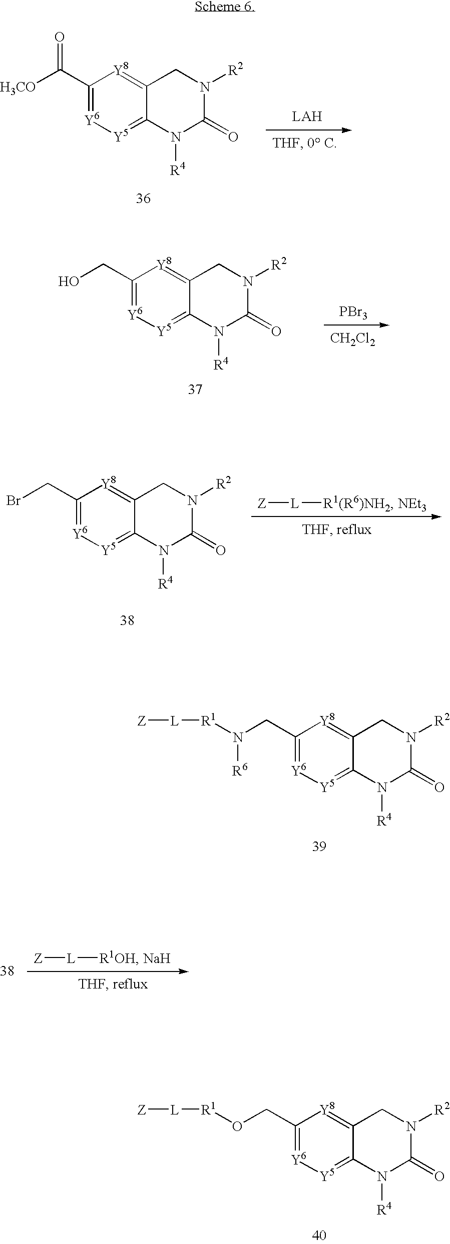

wherein R⁴ is H or C₁-C₆ alkyl, Y⁵, Y⁶, and Y⁸ are each independently C(H) or N, and R², R⁶, Z, L, and R¹ are as defined above.

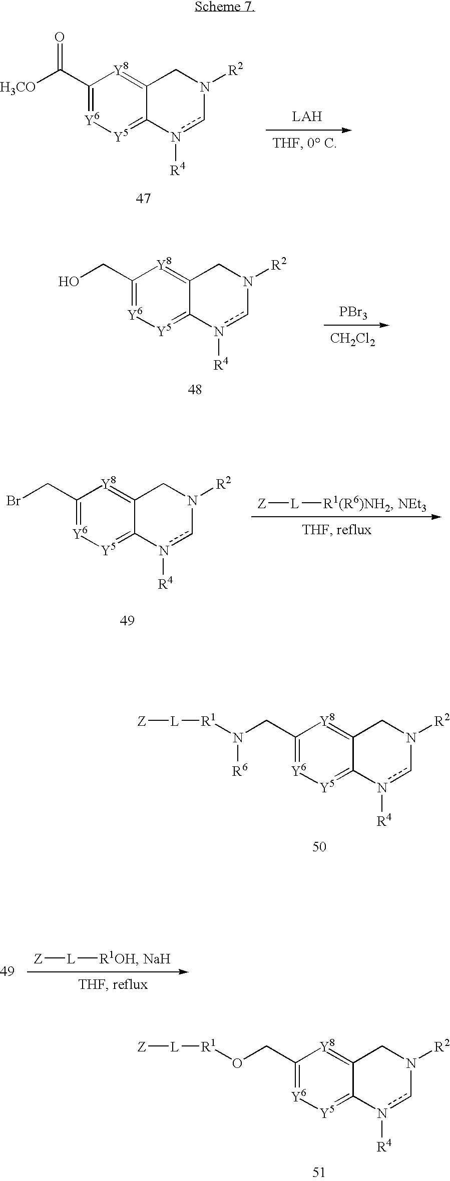

wherein - - - means a bond that is present or absent; and when the bond is present, the group R⁴ is absent.

wherein R⁴ is H or C₁-C₆ alkyl, Y⁵, Y⁶, and Y⁸ are each independently C(H) or N, and R², R⁶, Z, L, and R¹ are as defined above.

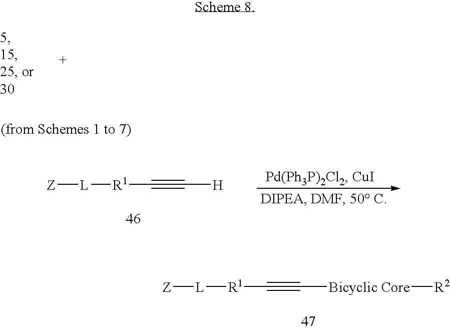

wherein Bicyclic core means group D from Schemes 1 to 7

wherein R², Z, L, and R¹ are as defined above.

Another typical synthesis of the invention compounds of Formula I is shown in Scheme 9 below. The first step in Scheme 9 comprises reacting a substituted urea or thiourea (1) with a substituted or unsubstituted malonic acid or ester (2) in the presence of acetic anhydride (with malonic acids) or alkali alkoxide (with malonic esters), respectively, to give a pyrimidinetrione (3). Reaction of the pyrimidinetrione (3) with phosphorus oxychloride at reflux for 1 to 2 hours gives the chloropyrimidinedione (4). Reaction of the chloropyrimidinedione (4) with excess sodium hydrosulfide in dimethylformamide at 40° C. to 45° C., followed by reaction with bromoacetaldehyde dimethylacetal at 40° C. to 50° C. gives a thio substituted pyrimidinedione (5). Cyclization of the thio-substituted pyrimidinedione (5) in the presence of catalytic para-toluenesulfonic acid in refluxing xylenes with azeotropic removal of methanol gives a thiazolopyrimidine of Formula I (6). Esters of structure (7) are prepared by deprotonation of (6) with lithium hexamethyldisilazane at −70° C. to −80° C. and reaction with chloroformates. Amides or thioamides are obtained by reaction of the lithiothiazolopyrimidines with isocyanates and isothiocyanates, respectively.

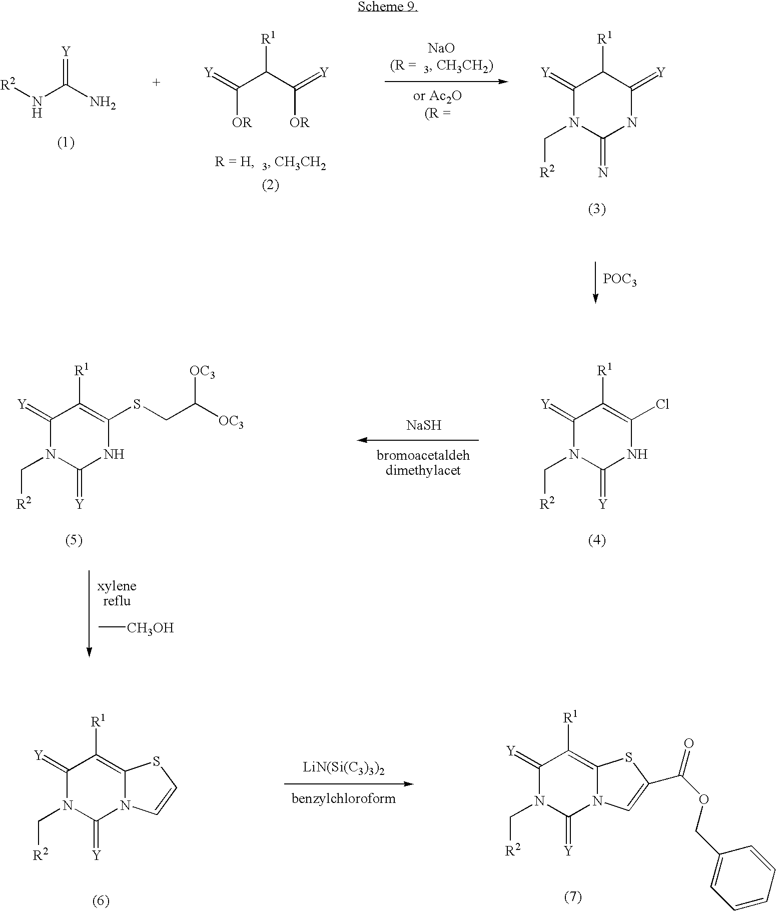

wherein in Scheme 9, R²CH₂ is a subgroup of R² as defined above for Formula I, Y is O or S, and R¹ means Z—L—R¹, wherein Z, L, and R¹ are as defined above for Formula I.

An alternative method for making invention compounds of Formula I is illustrated below in Scheme 10. 6-Chloropyrimidine-2,4-dione (1) is reacted with sodium hydrogen sulfide and bromoacetaldehyde dimethyl acetal, and then treated with 1-(trimethylsilyl)iodide (TMSI), to afford a key intermediate, namely 5,7-dioxo-6,7-dihydro-5H-thiazolo[3,2-c]pyrimidine (2). This unsubstituted thiazolopyrimidine readily reacts with alkylating agents such as alkylhalides, arylalkyl halides, and heteroarylalkyl halides, (where L is a good leaving group such as halo) generally in the presence of a base such as triethylamine or cesium carbonate, to effect alkylation at the 6-position to give 6-alkyl, 6-arylalkyl, and 6-heteroarylalkyl thiazolopyrimidines of Formula I (3). These compounds are especially useful as intermediates to 2-substituted thiazolopyrimidines. For example, the compounds (3) are readily converted to invention esters and amides by the general method described above in Scheme 9.

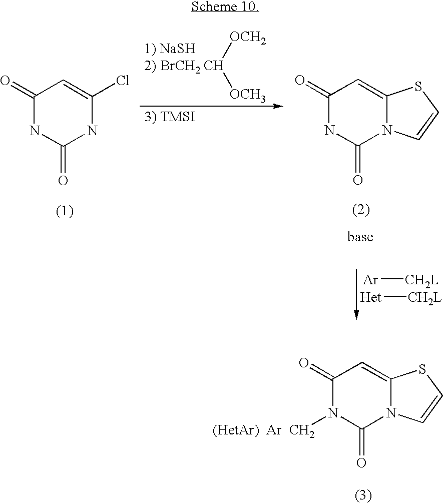

wherein HetArCH₂ and ArCH₂ mean R² of Formula I wherein HetAr is an unsubstituted or substituted 5-membered, 6-membered heteroaryl or an 8- to 10-membered heterobiaryl and Ar is an unsubstituted or substituted phenyl or naphthyl.

A method of preparing an intermediate that may be used to prepare a variety of compounds of Formula I is shown below in Scheme 11.

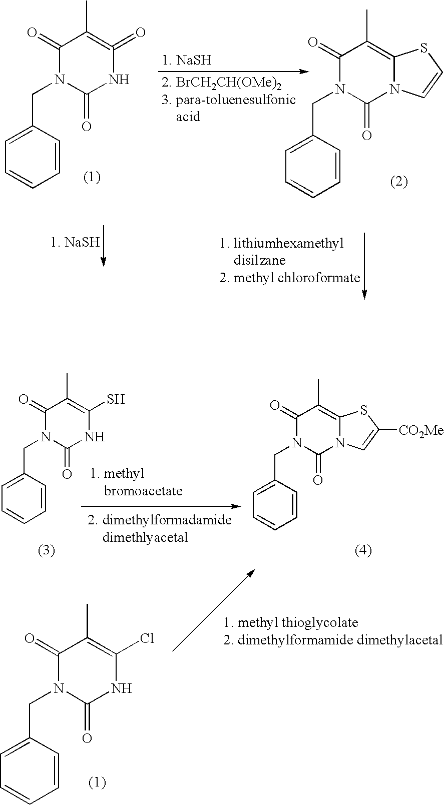

In Scheme 11, a compound of formula (1) is allowed to react with NaSH to give an intermediate thiol derivative, which is allowed to react with 2-bromoacetaldehyde dimethyl acetal, followed by acid-catalyzed ring closure of the resulting thioether, to give a compound of formula (2). Deprotonation of the compound of formula (2) with lithium hexamethyldisilazane (“LiHMDS”), and quenching of the resulting anion with methylchloroformate gives a compound of formula (4).

Alternately in Scheme 11, the intermediate thiol derivative described above may be allowed to react with methyl bromoacetate, and the resulting thioether allowed to condense and cyclize with dimethylformamide dimethylacetal to give a compound of formula (4).

Still alternatively in Scheme 11, the compound of formula (1) may be allowed to react with methyl thioglycolate to give the same intermediate as that obtained by reaction of the thiol derivative described above with methyl bromoacetate. This intermediate so formed may again be allowed to react with dimethylformamide as described above to give a compound of formula (4).

Another typical synthesis of the invention compounds of Formula I is shown in Scheme 12 below. The first step in Scheme 12 comprises reacting a diacid with a chlorinating reagent such as thionyl chloride or oxalyl chloride in a nonprotic solvent such as dichloromethane to give the diacid chloride. This acid chloride can then be reacted with an amine, NHR⁴R⁵, in excess or with an organic base such as triethylamine, to give a bis-amide of Formula I. Alternately, the acid chloride can be reacted with an alcohol, R⁴OH, in a nonprotic solvent such as dichloromethane along with an organic or inorganic base such as triethylamine or potassium carbonate to give a bis-ester of Formula I. The bis-ester can in some circumstances be reacted with an amine, NHR⁴R⁵, at elevated temperatures to give a bis-amide of Formula I. The diacid can also be reacted with an alkyl halide in a nonprotic solvent containing an organic or inorganic base to give a bis-ester of Formula I. A third sequence involves the reaction of the diacid with hydroxybenzotriazole, HOBt, and dicyclohexylcarbodiimide, DCC, and an amine, NHR⁴R⁵, in a solvent such as dimethylformamide, DMF, or dichloromethane to give a bis-amide of Formula I.

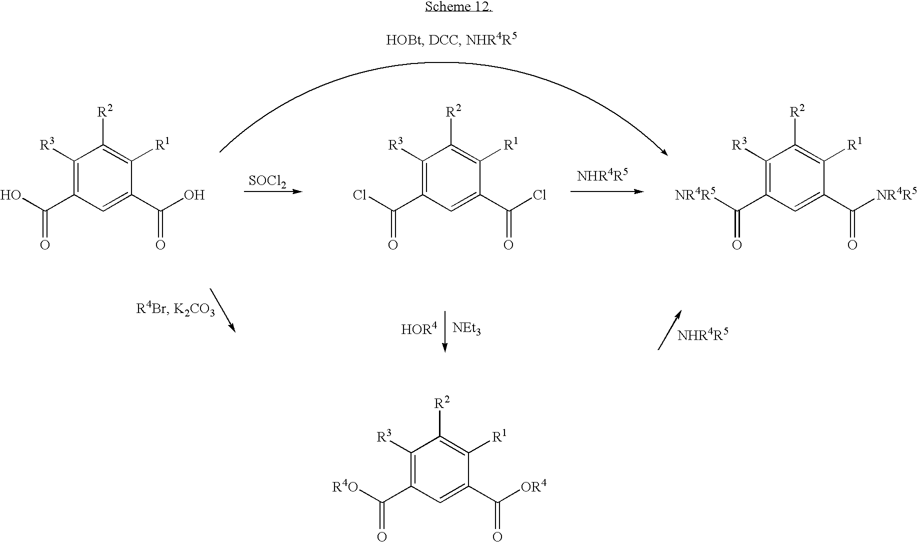

wherein in Scheme 12, R¹, R², and R³ is H, methyl, or methoxy, one R⁴O—C(O) or R⁴R⁵NC(O) means Z—L—R¹—Q, and the other R⁴O—C(O) or R⁴R⁵NC(O) means R², wherein Z, L, R¹, and R² are as defined above for Formula I.

Another typical synthesis of the invention compounds of Formula I is shown in Scheme 13 below. The first step in Scheme 13 comprises reacting a chlorouracil analog with 2-mercapto acetate ester. The reaction generally is carried out in a solvent such as an alkanol, for example ethanol, and in the presence of a base such as sodium carbonate. The reaction is usually substantially complete after about 2 to 6 hours when carried out at an elevated temperature of about 40° C. to about 80° C. The product, an alkylthio substituted tetrahydro pyrimidine, can be isolated and purified if desired, or can be used directly in the next step. The next step is a cyclization reaction (Vilsmeier reaction). The alkylthio substituted tetrahydro pyrimidine is reacted with POCl₃ in a polar solvent such as dimethylformamide or dimethylsulfoxide to effect cyclization to the corresponding tetrahydro-thieno[2,3-d]pyrimidine-2,4-dione. The thienopyrimidinone can be further modified by standard procedures, for example alkylation at the 1-position by reaction with an alkylating agent R⁴L, where L is a leaving group such as chloro or bromo. Ester groups can be hydrolyzed by reaction with a base such as sodium hydroxide, and carboxylic groups can be esterified by standard procedures such as reaction with an alcohol R³OH in the presence of an acid such as hydrochloric acid, or in the presence of a coupling reagent such as DCC (dicyclohexylcarbodiimide) and CMC (1-cyclohexyl-3-(2-morpholinoethyl)carbodiimide metho-p-toluenesulfonate. Carboxylic acid groups can be converted to amides by standard methods, for example by first reaction with oxalyl chloride to form an acid chloride, and then reaction of the acid chloride with an amine of the formula HNR⁴R⁵.

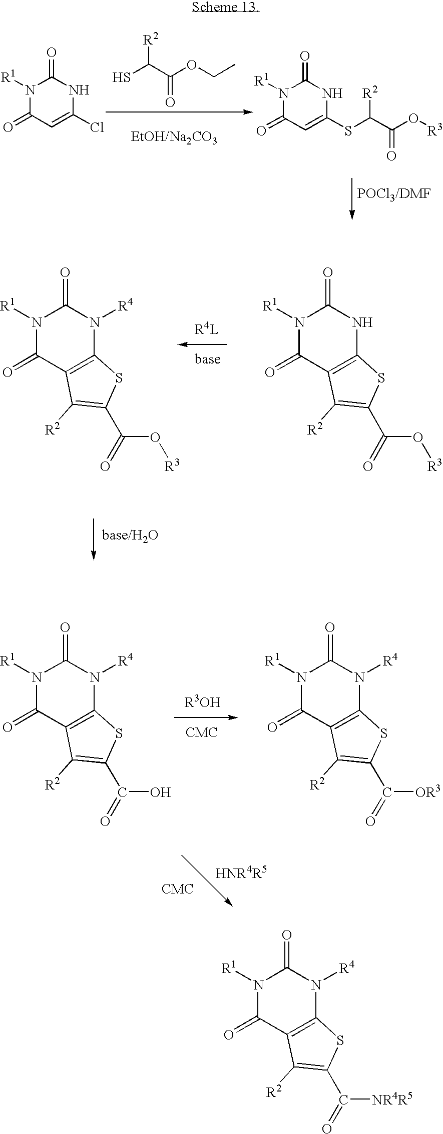

wherein in Scheme 13, R⁴ is H or C₁-C₆ alkyl, R² is H, F, or C₁-C₆ alkyl, C(O)OR³ and C(O)NR⁴R⁵ are Q—R¹—L—Z groups of Formula I, and R¹ means R² of Formula I.

Scheme 14 illustrates the synthesis of compounds of Formula 1 starting from a benzyl alkanoylacetate, which reacts with a cyanoacetic acid ester; in the presence of powdered sulfur and a base such as morpholine to give an amino substituted heterocycle. This condensation typically is carried out by combining the reactants in a solvent such as methanol or ethanol, and generally is complete within about 2 to 10 hours when carried out at an elevated temperature of about 40° C. to 60° C. The 5-benzyloxycarbonyl-2-amino-substituted heterocycle (e.g., thiophene is next reacted with an isocyanate (R¹NCO) to effect cyclization to form the pyrimidinone ring. This cyclization reaction is carried out by mixing the reactants in a solvent such as dioxane in the presence of a strong base such as sodium hydride. The cyclization is generally complete within about 8 to 24 hours when carried out at a temperature of about 24° C. to 60° C. The product, a compound of Formula I, can be alkylated or arylated by reaction with an alkyl or aryl halide (R⁴L, where L is a leaving group such as chloro or bromo). The invention compound can be further modified by standard methods, for instance by hydrolyzing the ester; forming group R³ to give the corresponding acid (where R³=H), and then re-esterifying or amidating by reaction with an amine in the presence of a coupling agent such as DCC or CMC.

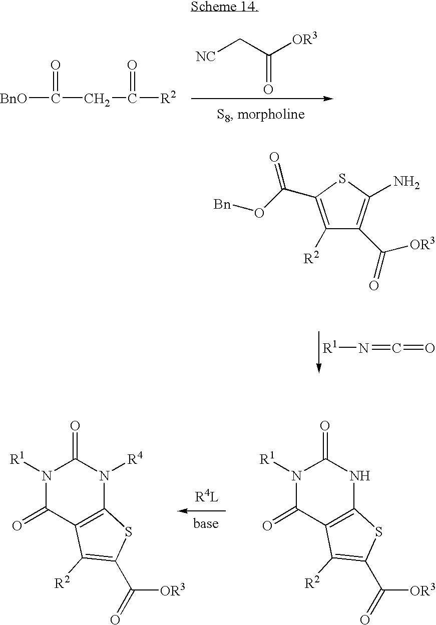

wherein in Scheme 14, R⁴ is H or C₁-C₆ alkyl, R² is H, F, or C₁-C₆ alkyl, C(O)OR³ is a Q—R¹—L—Z group of Formula I, and R¹ means R² of Formula I.

Scheme 15 illustrates reaction of a 4-alkoxycarbonyl-5-amino thiazole with an isocyanate in the presence of a strong base such as sodium hydride to form the 6-member pyrimidinone ring (X is S). The unsubstituted ring nitrogen can be alkylated or arylated by standard reactions, for example by reactions with a alkylating agent R⁴L, where L is a leaving group such as halo.

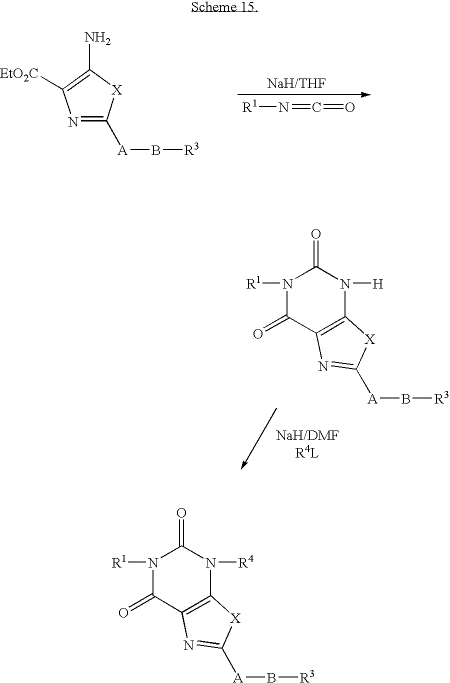

wherein in Scheme 15, X is O, S, N(H), or N(CH₃), R⁴ is H or C₁-C₆ alkyl, A—B—R³ is a Q—R¹—L—Z group of Formula I, and R¹ means R² of Formula I.

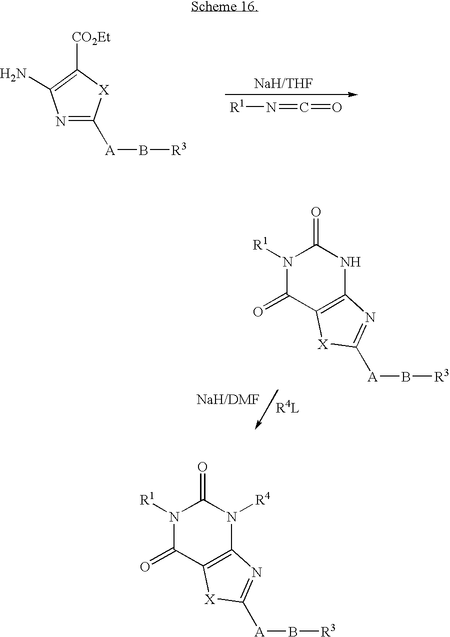

wherein in Scheme 16, X is O, S, N(H), or N(CH₃), R⁴ is H or C₁-C₆ alkyl, A—B—R³ is a Q—R¹—L—Z group of Formula I, and R¹ means R² of Formula I.

The corresponding sulfoxide and sulfone analogs can be prepared in the same fashion or by oxidizing the product of Scheme 16 when X is S with an appropriate stochiometric amount of a suitable oxidant such as meta-chloroperbenzoic acid (“mCPBA”).

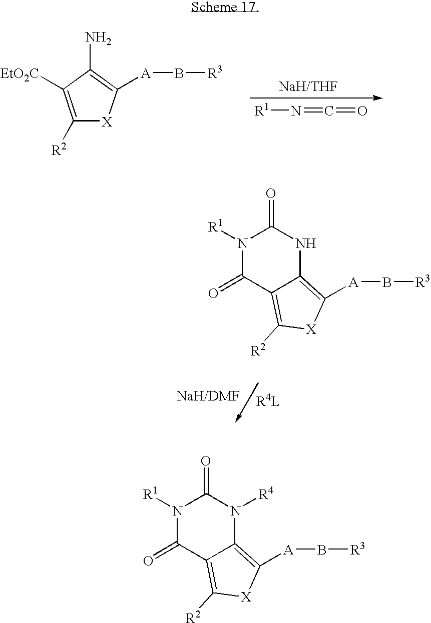

wherein in Scheme 17, X is O, S, N(H), or N(CH₃), R⁴ is H or C₁-C₆ alkyl, A—B—R³ is a Q—R¹—L—Z group of Formula I, R² is H, F, or CH₃, and R¹ means R² of Formula I.

The corresponding sulfoxide and sulfone analogs can be prepared in the same fashion or by oxidizing the product of Scheme 17 when X is S with an appropriate stochiometric amount of a suitable oxidant such as meta-chloroperbenzoic acid (“mCPBA”).

The corresponding ester and amide analogs can be prepared in the same fashion.

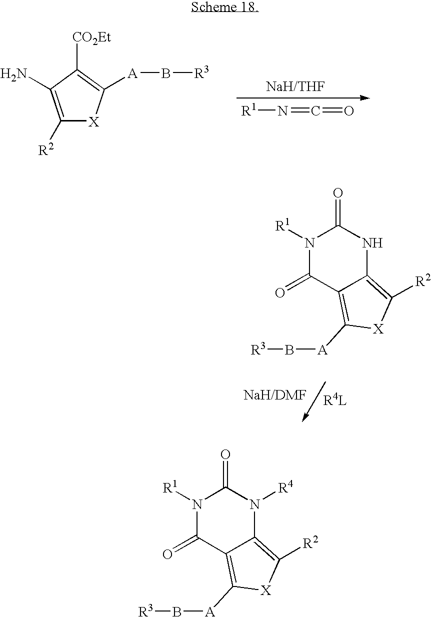

wherein in Scheme 18, X is O, S, N(H), or N(CH₃), R⁴ is H or C₁-C₆ alkyl, A—B—R³ is a Q—R¹—L—Z group of Formula I, and R¹ means R² of Formula I.

The corresponding sulfoxide and sulfone analogs can be prepared in the same fashion or by oxidizing the product of Scheme 18 when X is S with an appropriate stochiometric amount of a suitable oxidant such as meta-chloroperbenzoic acid (“mCPBA”).

The corresponding ester and amide analogs can be prepared in the same fashion.

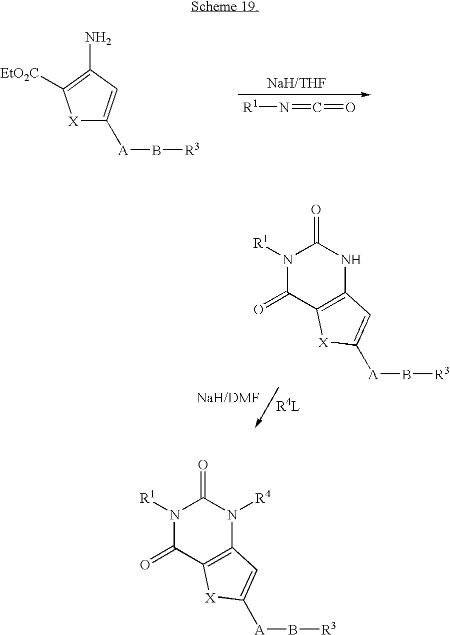

wherein in Scheme 19, X is O, S, N(H), or N(CH₃), R⁴ is H or C₁-C₆ alkyl, A—B—R³ is a Q—R¹—L—Z group of Formula I, and R¹ means R² of Formula I.

The corresponding sulfoxide and sulfone analogs can be prepared in the same fashion or by oxidizing the product of Scheme 19 when X is S with an appropriate stochiometric amount of a suitable oxidant such as meta-chloroperbenzoic acid (“mCPBA”).

The corresponding ester and amide analogs can be prepared in the same fashion.

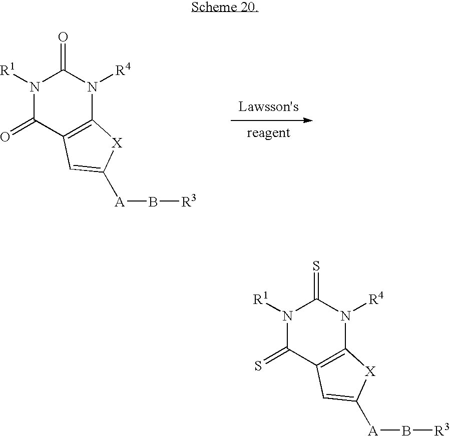

wherein in Scheme 20, X is O, S, N(H), or N(CH₃), R⁴ is H or C₁-C₆ alkyl, A—B—R³ is a Q—R¹—L—Z group of Formula I, and R¹ means R² of Formula I.

The corresponding sulfoxide and sulfone analogs can be prepared in the same fashion or by oxidizing the product of Scheme 20 when X is S with an appropriate stochiometric amount of a suitable oxidant such as meta-chloroperbenzoic acid (“mCPBA”).

The corresponding ester and amide analogs can be prepared in the same fashion.

The alkynes can be prepared in a conventional manner as illustrated in Scheme 21. In Scheme 21, an aryl iodide (or, optionally, an aryl bromide, aryl chloride, or aryl trifluoromethanesulfonate) is coupled to a terminal alkyne in the presence of a palladium catalyst, cuprous (I) iodide, and a base such as a tertiary amine base.

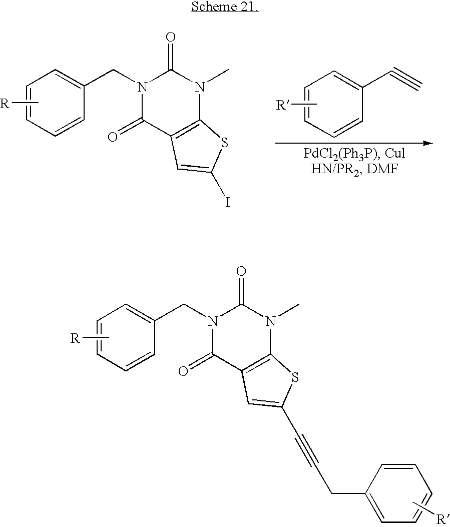

wherein R independently is hydrogen or from 1 to 3 substituents as defined above for substituted phenyl and C≡C—CH₂C₆H₄—R′ is Q—R¹—L—Z or Formula I, wherein Q is C≡C, R¹ is CH₂C₆H₄—, and L and Z are as defined above for Formula I.

Another general process for the synthesis of the compounds of Formula I is described in the following Schemes 22a and 22b.

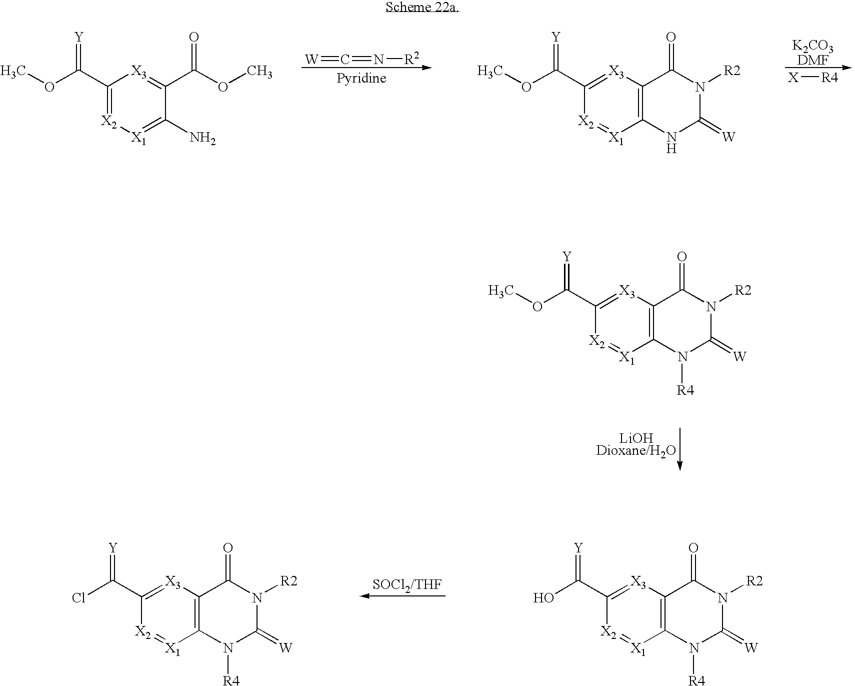

wherein X₁, X₂, and X₃ are each independently C(H) or N, Y is O or S, R⁴ is H or C₁-C₆ alkyl, and R² is as defined above for Formula I.

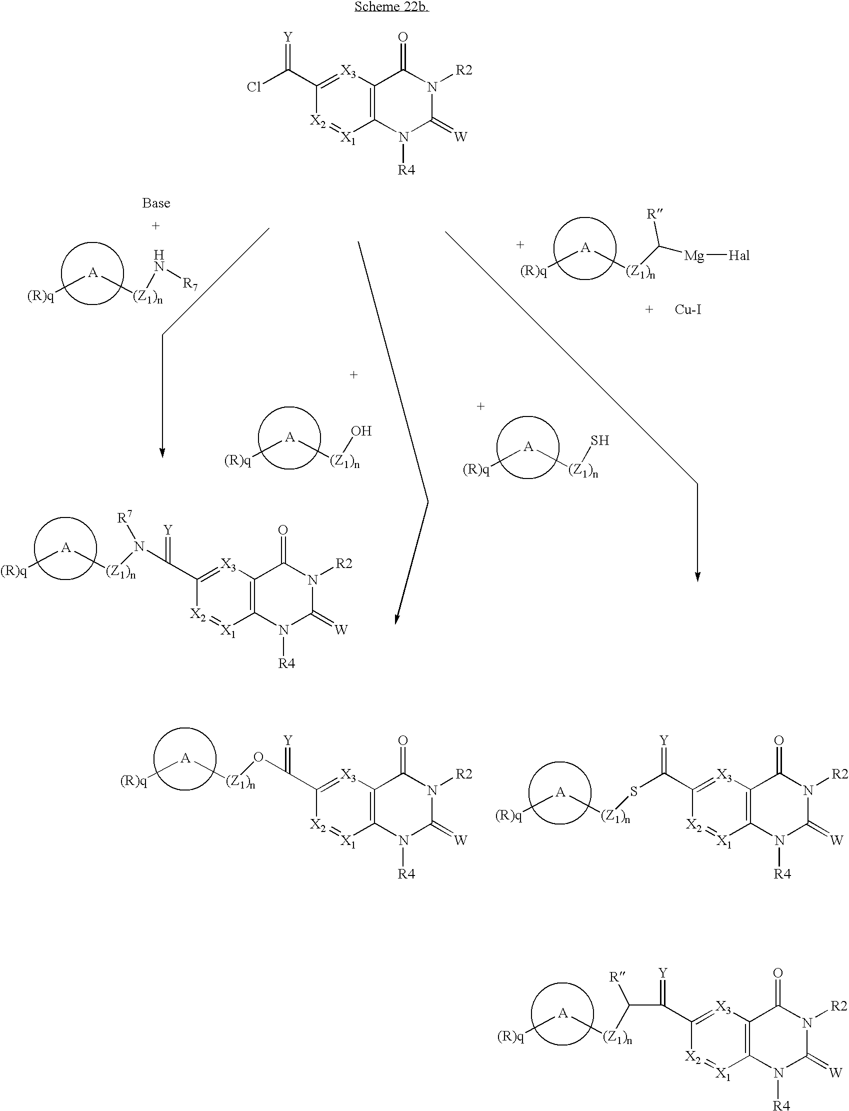

in which R₇ is hydrogen, (C₁-C₆)alkyl, aryl(C₁-C₆)alkyl, cycloalkyl, aryl or heteroaryl, R″ is (C₁-C₆)alkyl, aryl, aryl(C₁-C₆)alkyl, aromatic or non-aromatic heterocycle or cycloalkyl, R is a substituent as defined above, q is an integer of from 0 to 4, X₁, X₂, and X₃ are C(H) or N, C(Y)C(H)(R″)(Z₁)ₙA is Q—R¹—L—Z as defined above for Formula I, R⁴ is H or C₁-C₆ alkyl, and R² is as defined above for Formula I.

The compounds of the present invention may be obtained firstly by the method represented in Schemes 23a and 23b below.

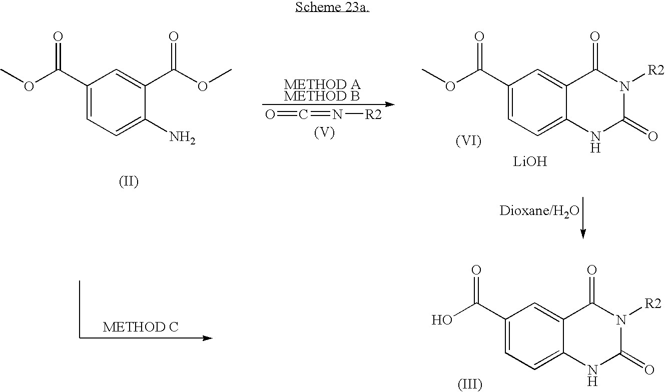

wherein R² is as defined above for Formula I.

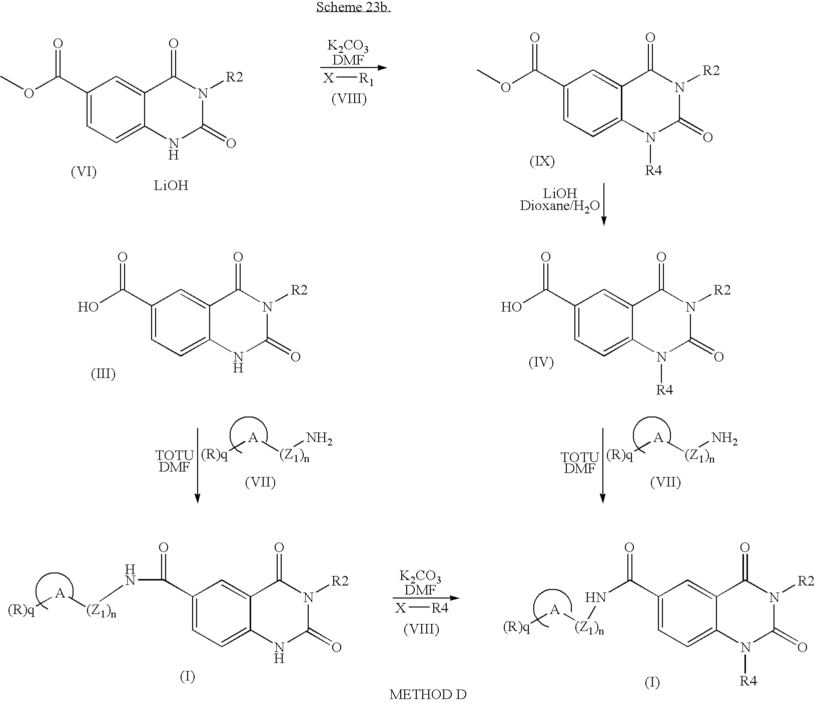

wherein DMF is dimethylformamide, TOTU is a carboxylic acid activator named O-[(ethoxycarbonyl)-cyanomethylenamino]-N,N,N′,N′-tetramethyluronium tetrafluoroborate, R² is as defined above for Formula I, R⁴ is H or C₁-C₆ alkyl, R is a substituent as defined above for Formula I, q is an integer of from 0 to 4, and C(O)N(H)Z₁)ₙA is a group Q—R¹—L—Z as defined above for Formula I.

The intermediate compound of formula (II) which constitutes the starting material for the synthetic process illustrated by Schemes 22a and 22b above may be prepared in accordance with Scheme 24 below:

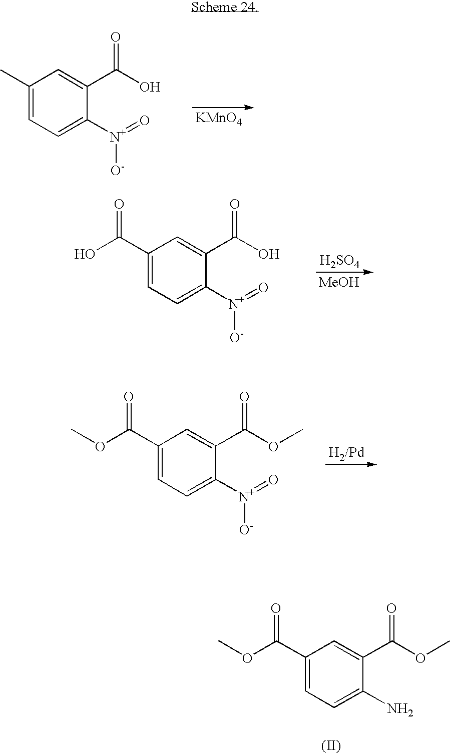

The intermediate compound of formula (II) which constitutes the starting material in the process to synthesize the compounds of general Formula I according to the invention as illustrated in Schemes 22a and 22b above may also be prepared according to the process illustrated in Scheme 25 below.

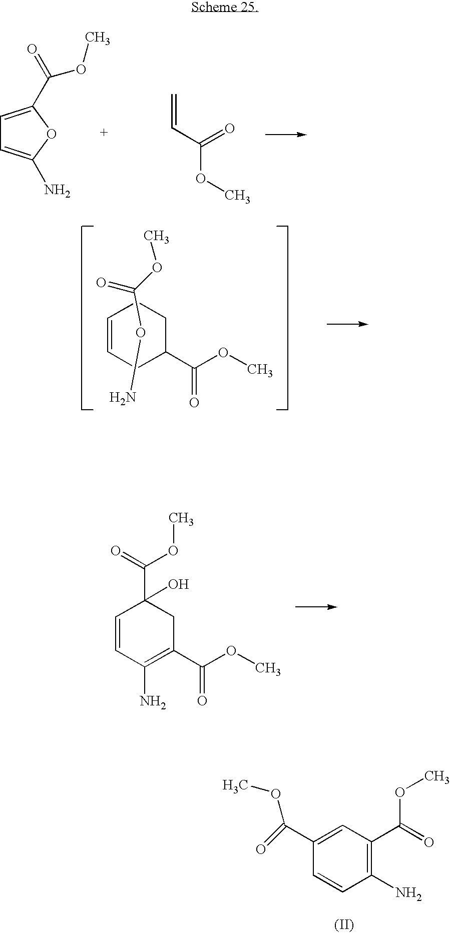

The compound of general formula (III) may be prepared, in accordance with the process described in Schemes 22a and 22b above, from the compound of formula (II), according to the synthetic Scheme 26 (Method A) below:

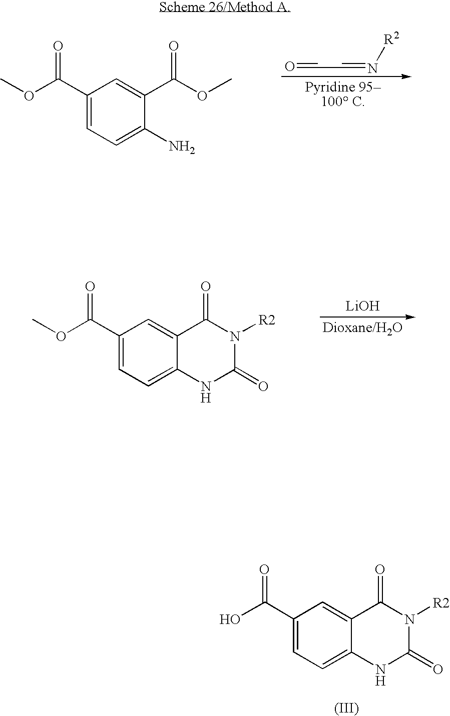

wherein R² is as defined above for Formula I.

According to another aspect, the intermediate compound of formula (III) may be prepared, in accordance with the synthetic process illustrated in Scheme 26 above, according to Method B, as illustrated in Synthetic Scheme 26/Method B below:

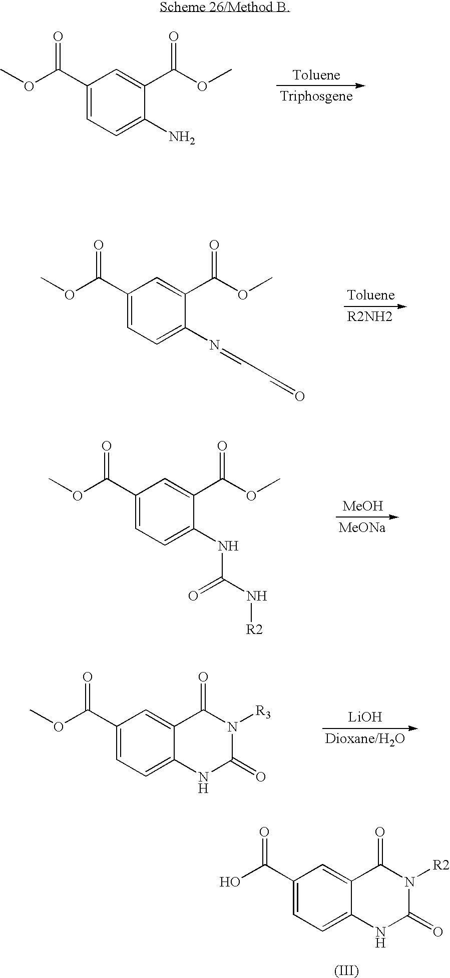

wherein R² is as defined above for Formula I.

According to yet another aspect, an intermediate compound of general formula (III), may be obtained, in accordance with the synthetic process illustrated in Schemes 22a and 22b above, according to Scheme 26/Method C illustrated below:

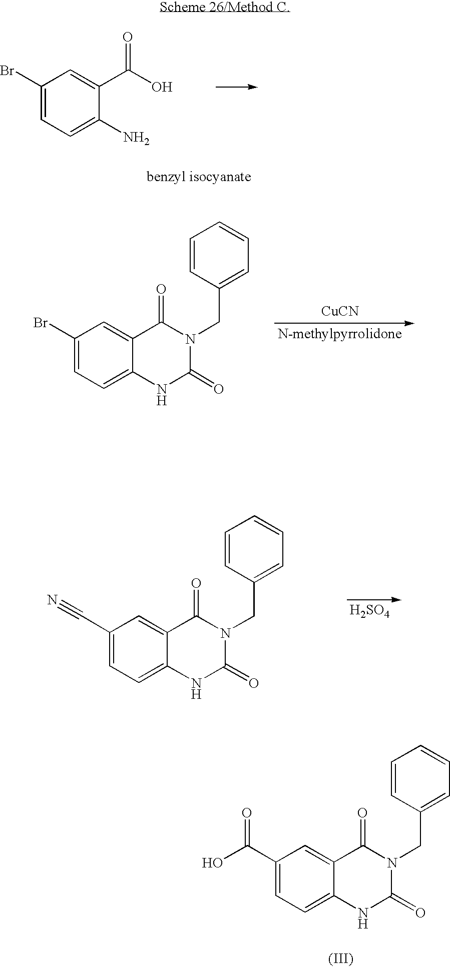

Another process for preparing a compound of Formula I is illustrated in Scheme 27 below.

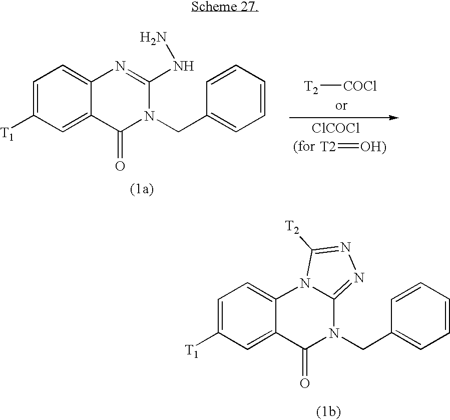

In Scheme 27 above, T₁ represents an exchangeable group, for example bromine, or a protecting group such as, for example, COOEt, and T₂ represents a group selected from aryl, alkyl and cycloalkyl. In this process, the 2-hydrazino 3,4-dihydroquinazolin-4-one (1a) is coupled, in a first stage, with an alkylcarboxylic, cycloalkylcarboxylic or arylcarboxylic acid chloride (or optionally a phosgene derivative) to give the corresponding hydrazide intermediate, which is cyclized, in a second stage, into a 1-alkyl, cycloalkyl or aryl (or optionally hydroxyl) triazolo[4,3-a]quinazolin-5-one (1b), by heating in a suitable solvent. It is clearly understood that the acid chloride reagent T₂—COCl may be replaced with a reactive derivative of the acid, such as an ortho ester.

The process for synthesizing intermediate compounds (1a) is disclosed in the PCT patent application published under No. WO 00/66584.

Another process is illustrated in Scheme 28 below.

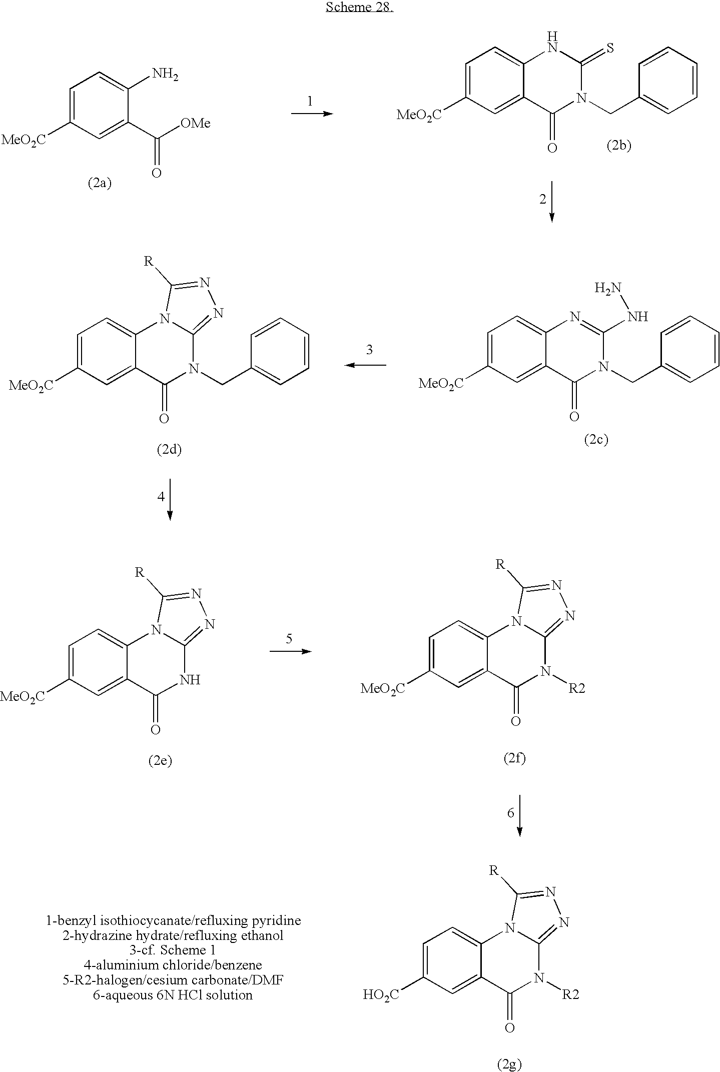

wherein R is H, OH, CH₃, and R² is as defined above for Formula I.

In Scheme 28, methyl 4-aminoisophthalate (2a) is treated with benzyl isothiocyanate, in a solvent such as pyridine or acetic acid, to give 3-benzyl-6-(methoxycarbonyl)-2-thioxo-1,2,3,4-tetrahydroquinazolin-4-one (2b). This compound is heated, in a refluxing alcohol, in the presence of hydrazine hydrate to give the corresponding hydrazine which is in turn cyclized by reaction with a carboxylic acid derivative RCOOH (such as an acid chloride or an ortho ester). The 4-benzyl-7-(methoxycarbonyl)-4,5-dihydrotriazolo[4,3-a]quinazolin-5-one (2d) obtained is N4-debenzylated using aluminum chloride in benzene, and the intermediate secondary lactam is then substituted with a halide, in the presence of a base such as cesium carbonate, in a solvent such as dimethylformamide. The N-substituted analogue obtained (2f) is then hydrolyzed, preferably in acidic medium, to give the corresponding acid (2g) which may be subsequently subjected to a coupling reaction of peptide type.

The order of the steps in the above process may be modified for the synthesis of certain compounds. For example, when R² is para-cyanobenzyl, step 5 will be carried out last since the para-cyanobenzyl group would not withstand the conditions of step 6.

Another preparation process is illustrated in Schemes 29a and 29b below.

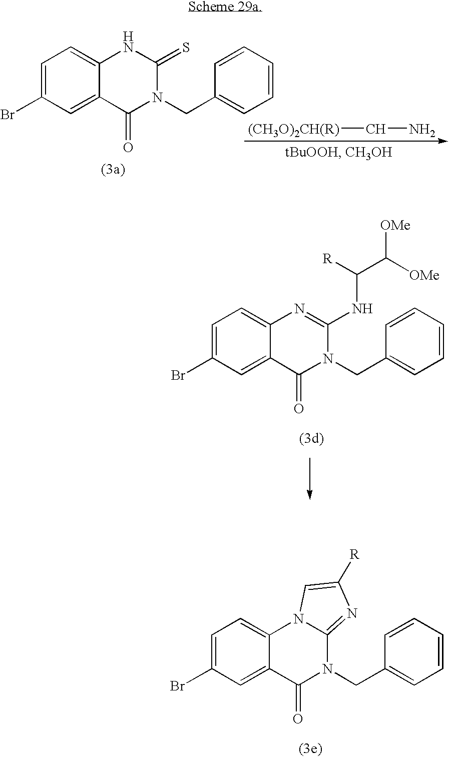

wherein R is H, OH, CH₃.

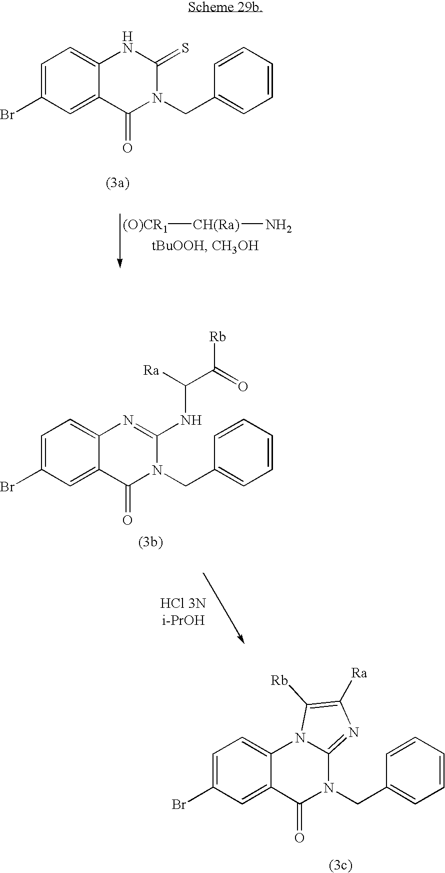

wherein Ra and Rb are independently H, CH₃, F, or OH.

In Schemes 29a and 29b, 3-benzyl-6-bromo-2-thioxo-1,2,3,4-tetrahydroquinazolin-4-one (3a) is treated, depending on the case, with a 2-amino acetal or a 2-amino ketone, in an alcoholic solvent such as methanol or ethanol, in the presence of a peroxide for initiating the oxidation of the starting thiol and converting it, depending on the circumstance, into a better exchangeable group. The intermediate amino ketone (3d) or amino acetal (3b) obtained is readily cyclized in the presence of acid, in an alcoholic solvent such as isopropanol, into 4-benzyl-6-bromo-4,5-dihydroimidazolo[1,2-a]quinazolin-5-one (3c) or (3e) depending on the desired degree of substitution. The bromine atom in position 7 may then be subsequently exchanged with a potential carboxylic function. See, for example, Scheme 31 below.

Another process is illustrated in Scheme 30 below:

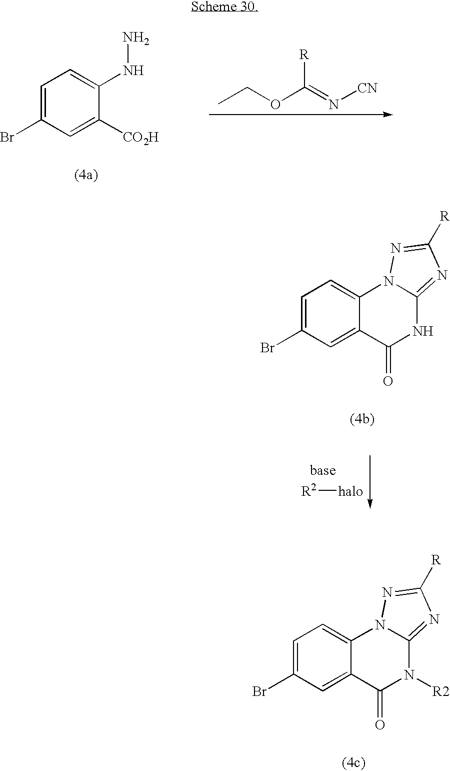

wherein R is H, F, CH₃, OH, and R² is as defined above for Formula I.

In Scheme 30, 5-bromo-2-hydrazinobenzoic acid (4a) is treated with an alkyl N-cyanoimidate to give the 4-benzyl-6-bromo-4,5-dihydrotriazolo[2,3-a]quinazolin-5-one (4b) in a single step. This compound is substituted with a halide, in the presence of a base such as cesium carbonate, in a solvent such as dimethylformamide, to give the N4-substituted analogue (4c). The bromine atom in position 7 may then be subsequently exchanged with a potential carboxylic function. See, for example, Scheme 31 below.

Another process for synthesizing intermediate compounds in the manufacture of the compounds of Formula I according to the invention is illustrated in Scheme 31 below.

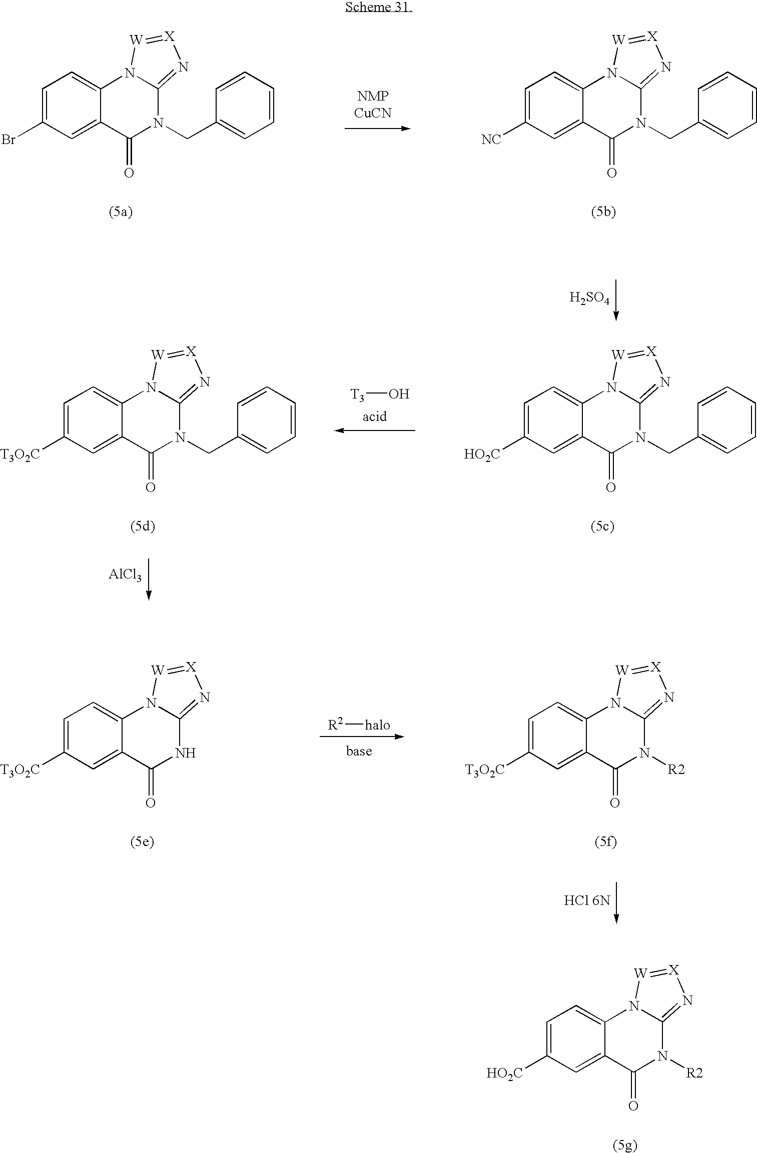

wherein R² is as defined above for Formula I, W is N or C(H), X is N or C(H), and T₃ is C₁-C₆ alkyl.

In Scheme 31 above, 4-benzyl-7-bromo-4,5-dihydrotriazolo[4,3-a]quinazolin-5-one, triazolo[2,3-a]quinazolin-5-one or imidazo[4,3-a]quinazolin-5-one (5a) is converted into the corresponding 7-cyano derivative (5b) by an exchange reaction with copper cyanide, in a solvent such as N-methylpyrrolidinone. The nitrile function is hydrolyzed in acidic medium, for example in the presence of sulphuric acid, and the carboxylic acid (5c) obtained is then esterified to (5d) with an alcohol in acidic medium. This intermediate is then N4-debenzylated with aluminum chloride, in a solvent such as benzene, and substituted with a halide to give compound (5f), in the presence of a base such as sodium hydride or cesium carbonate, in a solvent such as dimethylformamide or N-methylpyrrolidinone. The ester (5f) is finally hydrolyzed, in acidic medium, to a corresponding carboxylic acid (5g).

Another process allows the synthesis of intermediate compounds that are useful for manufacturing compounds of Formula I as illustrated in Scheme 32 below.

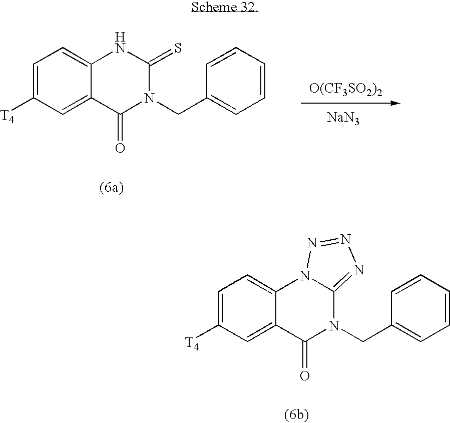

wherein in Scheme 32, T₄ is Z—L—R¹—Q as defined above for Formula I.

Another typical synthesis of the invention compounds of Formula I is shown in Scheme 33 below. The first step in Scheme 33 comprises reacting a substituted anthranilate of formula (A) with N-chlorosulfonyl isocyanate (CSI) followed by an appropriate Lewis acid such as aluminum trichloride in the manner described by Girared Y et al., (J. Chem. Soc. Perkins I, 1979:1043-1047). The resulting 1,2,4-benzothiadiazone carboxylate (B) can then be alkylated in the 3-position to give the compound (C) (for example by reaction with a common alkylating agent such as an alkyl halide, generally in the presence of a base such as triethylamine or pyridine). Simple hydrolysis of the ester under standard conditions (eg, alkaline conditions) affords the carboxylic acid (D). This acid can then be further reacted with alcohols or amines to provide the desired ester or carboxylic amide (E) using standard coupling conditions known to those skilled in the art (such as 1,3-dicyclohexylcarbodiimide (DCC) activation, in situ acid halide formation, 1,1-carbonyldiimidazole (CDI) activation, etc.). The invention compounds can be isolated and purified by standard methods such as crystallization (from solvents such as alcohols, alkyl esters, haloalkanes, alkanes) and chromatography over solid supports such as silica gel (eluting with solvents such as dichloromethane, ethyl acetate, methanol). Optically active compounds can be isolated by standard methods, for example fractional crystallization, chiral synthesis, and classical resolution.

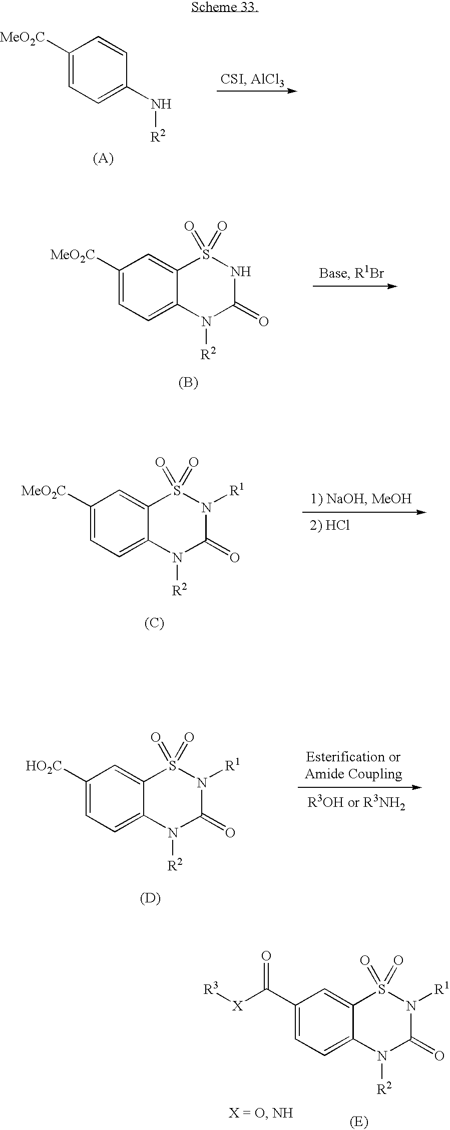

wherein in Scheme 33, R² is H or C₁-C₆ alkyl, R¹ is R² in Formula I, and C(O)—X—R³ is a Q—R¹—L—Z group of Formula I.

An alternative synthesis of the benzothiadiazines of the invention is given in Scheme 34 below. In this case, a substituted anthranilate of formula (A) is reacted with excess chlorosulfonic acid to give the sulfonyl chloride (F). This sulfonyl chloride is readily converted to the corresponding sulfonamide (G) by reaction with saturated ammonium hydroxide or liquid ammonia. Reaction of this sulfonamide with urea (or a similar C═O synthon such as phosgene or triphosgene) affords the desired 1,1,3-trioxo-1,2,3,4-tetrahydro-1l⁶-benzo[1,2,4]thiadiazine ring system (B) which can be further elaborated to the compounds of the present invention as demonstrated in Scheme 33.

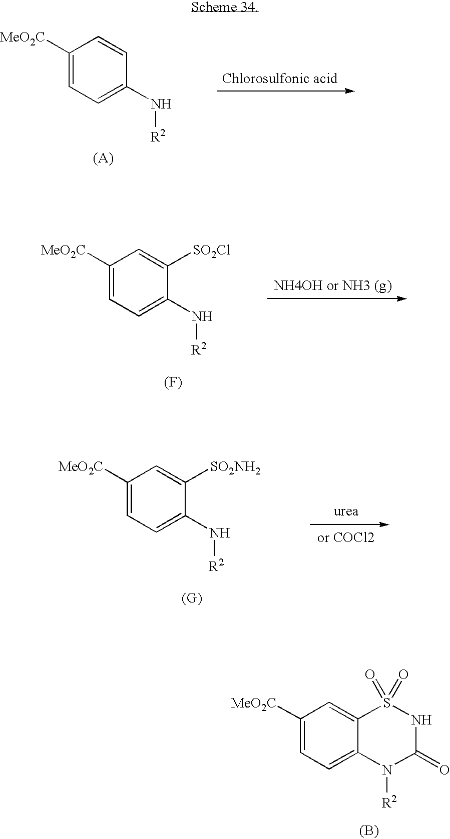

wherein in Scheme 34, R² is H or C₁-C₆ alkyl.

It should be appreciated that sulfoxides and sulfones of Formula I may be prepared by oxidation of the corresponding sulfides with one or two equivalents of an oxidizing agent such as peracetic acid or meta-chloroperbenzoic acid.

Other compounds of Formula I may be prepared according to the synthetic route outlined in Schemes 35 to 39, wherein S, T, and U are CH, N, or substituted C. In Scheme 35, commercially available 3-cyanobenzoic acid 1 undergoes a 3+2 cycloaddition reaction with azides selected from sodium azide, tributyltin azide, or trimethylsilyl azide in a suitable solvent such as toluene or p-dioxane and in the presence of triethylamine hydrochloride or ammonium chloride to form the corresponding tetrazole derivative. The carboxylic acid functionality is reacted with HCl in methanol at room temperature or under reflux conditions to give the ester intermediate 2. Compound 2 is allowed to react with a variety of alkyl halides or mesylates of commercially available alcohols in the presence of a base such as triethylamine, cesium carbonate, or sodium carbonate in a suitable solvent such as acetonitrile or dimethylformamide.

The resulting 1- and 2-substituted regioisomers are separated analytically pure using purification methods known in the art such as silica gel chromatography or recrystallization from solvents such as hexane/ethyl acetate or petroleum ether/diethyl ether. The ester functionality of intermediate 3 is converted to the corresponding acid 5 in the presence of a base such as sodium or lithium hydroxide in a protic solvent such as ethanol, methanol, or water. Acidification of the carboxylate salt using an acid such as hydrochloric acid, acetic acid, or trifluoroacetic acid yields the acid intermediate 5. The acid is converted to the acid chloride with oxalyl chloride or allowed to react with a coupling agent such as DCC or EDC in the presence of HOBT in a suitable solvent such as dichloromethane, tetrahydrofuran, or dimethylformamide. These reactive intermediates are coupled with a variety of primary and secondary amine nucleophiles including benzylamine, isopropylamine, and 3-picolylmethylamine to name a few.

Other compounds of Formula I may be prepared as shown in Scheme 36. In Scheme 36, the 3-bromo-4-methoxybenzonitrile 7 is converted to the tetrazole and alkylated to compounds 9 and 10 using reaction conditions described for intermediates 2,3, and 4 in Scheme 35. Intermediate 9 is carbonylated in the presence of a suitable coupling reagent such as a palladium catalyst, including bis(triphenylphosphinyl)chloride, palladium acetate, or palladium tetrakis triphenylphosphine, in the presence of a base such as a tertiary organic amine, including triethylamine or diisopropylethylamine, in a protic solvent such as methanol and under an atmosphere of carbon monoxide whose pressure and temperature may require as high as 500 psi and 100° C. Compound 11 can then be converted to a variety of amides 13 utilizing the experimental conditions previously described in Scheme 35.

Compound 7 in Scheme 36 can be replaced with commercially available pyridine based nitriles 14 as shown in Scheme 37. These compounds are converted to the corresponding tetrazole amides 20 utilizing the reaction conditions described in Scheme 36 for compound 14.

Alternatively in Scheme 38, the acid chloride prepared in Scheme 35 can be converted to the corresponding primary alcohol 22 in the presence of a suitable reducing agent such as lithium aluminum hydride or sodium borohydride in an aprotic solvent such a dichloromethane or tetrahydrofuran at temperatures ranging between 0° C. and 60° C. The alcohol 22 is converted to the corresponding bromide 23 using phosphorous tribromide in a halogenated solvent including dichloromethane, carbon tetrachloride, or chloroform. Intermediate 23 can be coupled with a variety of primary and secondary amines in the presence of a tertiary amine including diisopropylethylamine or triethylamine in suitable solvent such as dichloromethane or tetrahydrofuran at temperatures ranging from as low as room temperature to as high as reflux to give tetrazole amine 24. Coupling alkyl halide 23 with a variety of alcohols including benzyl alcohol or phenol and in the presence of a base such as sodium hydride or cesium carbonate in an appropriate solvent such as dimethylformamide or tetrahydrofuran yields the corresponding tetrazole ether derivative 25.

The synthesis of alkyne derivatives is presented in Scheme 39. The iodo substituted intermediate 28 is coupled with an appropriately substituted alkyne such as 3-phenyl-1-propyne or (1,1-difluoro-prop-2-ynyl)-benzene in the presence of copper(I) iodide and a tertiary organic base including diisopropylethylamine or triethylamine. The reaction is catalyzed by a palladium catalyst such as tetrakis (triphenylphosphine) palladium(0) or bis(triphenylphosphine)palladium(II) dichloride to yield the corresponding alkyne derivatives 30.

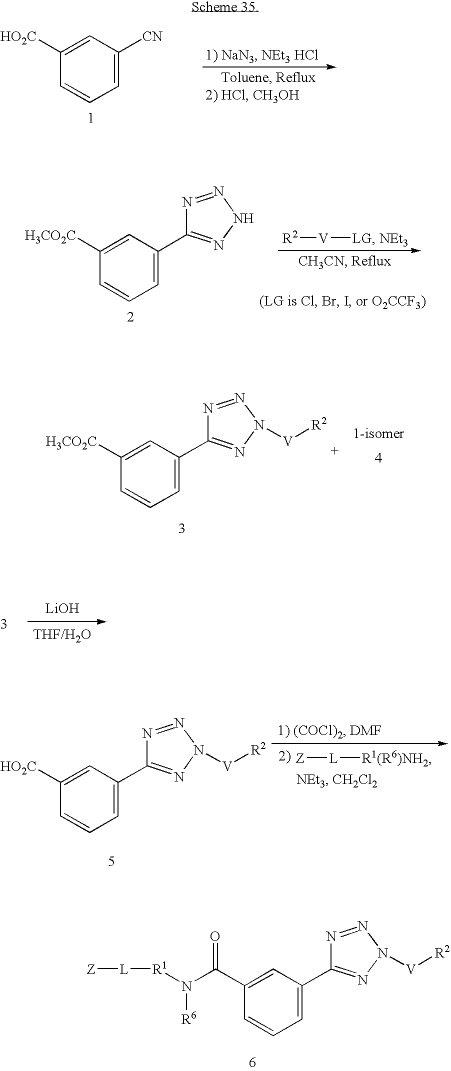

wherein Z, L, R¹, R⁶, V, and R² are as defined above for Formula I.

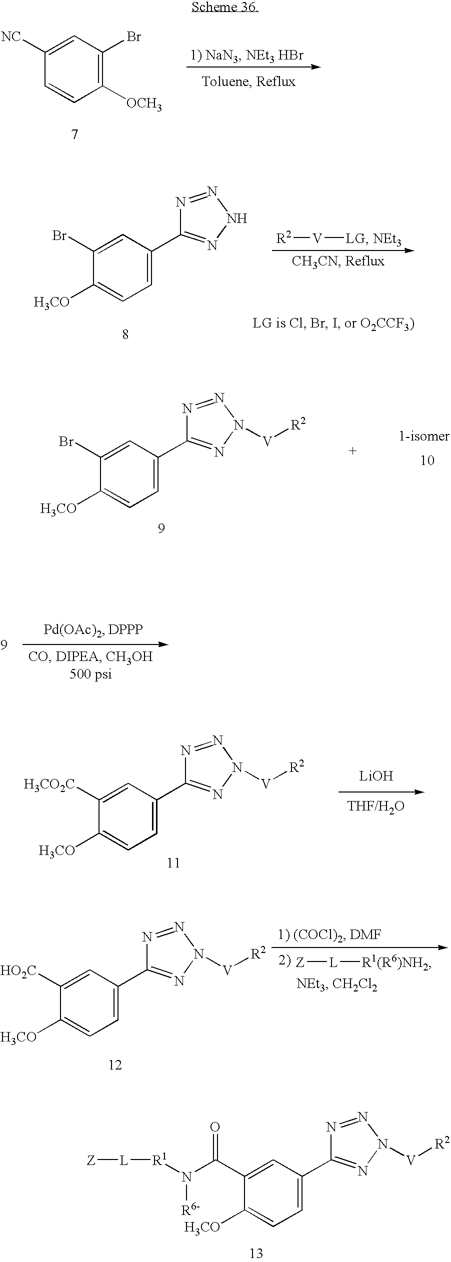

wherein Z, L, R¹, R⁶, V and R² are as defined above for Formula I.

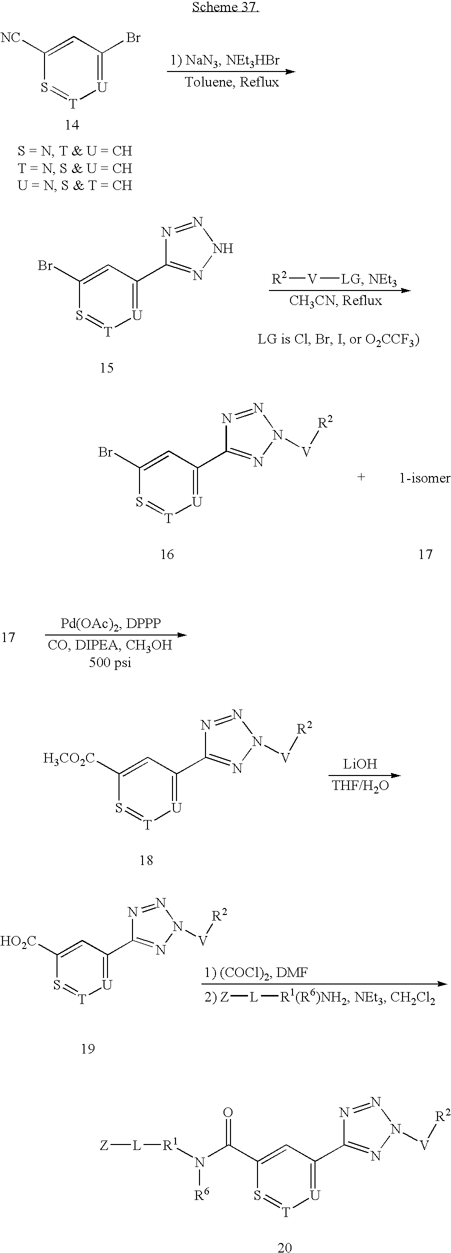

wherein S, T, and U are C(H) or N, and Z, L, R¹, R⁶, V, and R² are as defined above for Formula I.

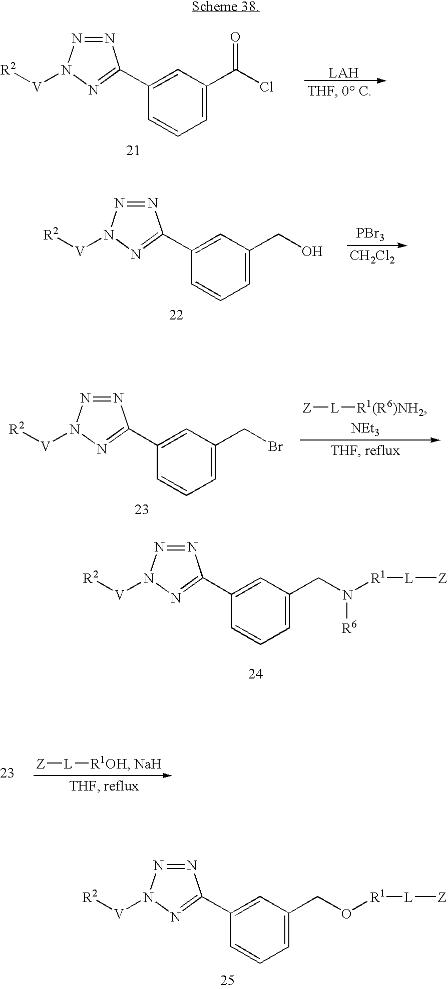

wherein R⁶, R², V, R¹, L, and Z are as defined above for Formula I.

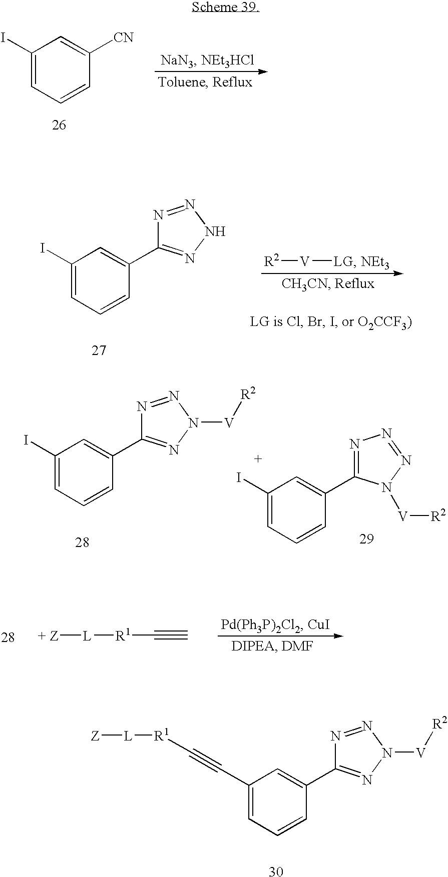

wherein Z, L, R¹, V, and R² are as defined above for Formula I.

Another synthesis of the compounds of Formula I is outlined below in Scheme 40.

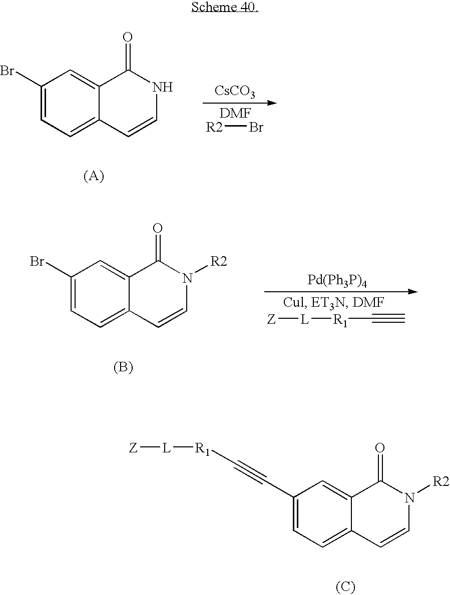

wherein R², Z, L, and R¹ are as defined above for Formula I.

In Scheme 40, a suspension of 7-bromo-1-hydroxyisoquinoline (A) can be alkylated in an aprotic solvent such as dimethylformamide when treated with a common alkylating agent such as an alkyl halide or benzyl halide, generally in the presence of a base such as cesium carbonate, potassium carbonate, or triethylamine.

The alkylated isoquinoline (B) can be further reacted with a variety of alkynes using standard coupling conditions known to those skilled in the art, for example, using a catalyst such as Pd(PPh₃)₄ or PdCl₂((PPh₃)₂, with or without an accompanying ligand, and in the presence of a base, such as triethylamine or diisopropylamine, to give compounds of this invention (C). Where appropriate, cleavage of t-butyl protecting groups is carried out under standard conditions, for example, moderately acidic hydrolysis, to afford the carboxylic acid.

The invention compounds can be isolated and purified by standard methods such as crystallization from solvents such as alkanes, alkyl esters, and ethyl acetate, and chromatography over solid supports such as silica gel, eluting with solvents such as dichloromethane, acetonitrile, tetrahydrofuran, hexanes, ethyl acetate.

The synthesis is further illustrated in Scheme 41 below.

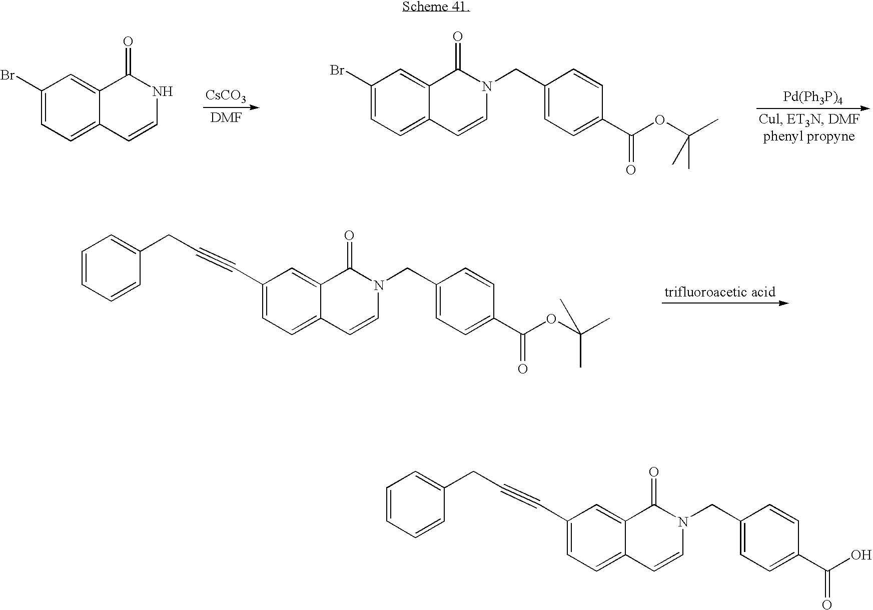

Another synthesis of the compounds of Formula I is outlined below in Scheme 42. For illustration purposes, Scheme 42 describes the preparation of a compound of Formula I wherein Q is a carbon-carbon triple bond. In principle, any compound of Formula I wherein Q is a carbon-carbon triple bond may be prepared according to the procedure outlined in Scheme 42.

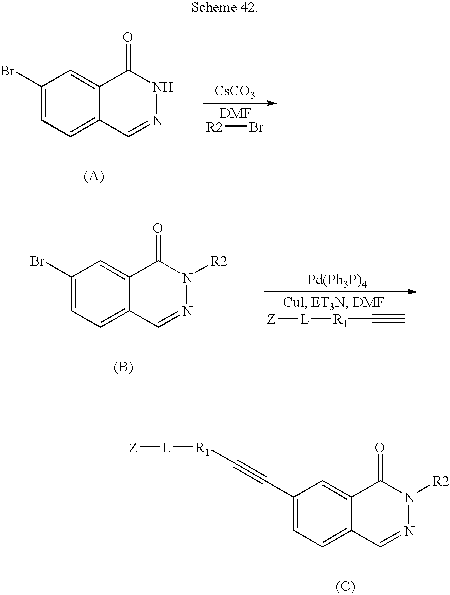

In Scheme 42, a suspension of 7-bromo-1-hydroxy-3-azaisoquinoline (A) can be alkylated in an aprotic solvent such as dimethylformamide when treated with a common alkylating agent such as an alkyl halide or benzyl halide, generally in the presence of a base such as cesium carbonate, potassium carbonate, or triethylamine.

The alkylated isoquinoline (B) can be further reacted with a variety of alkynes using standard coupling conditions known to those skilled in the art, for example, using a catalyst such as Pd(PPh₃)₄ or PdCl₂((PPh₃)₂, with or without an accompanying ligand, and in the presence of a base, such as triethylamine or diisopropylamine, to give compounds of this invention (C). Where appropriate, cleavage of t-butyl protecting groups is carried out under standard conditions, for example, moderately acidic hydrolysis, to afford the carboxylic acid.

The invention compounds can be isolated and purified by standard methods such as crystallization from solvents such as alkanes, alkyl esters, and ethyl acetate, and chromatography over solid supports such as silica gel, eluting with solvents such as dichloromethane, acetonitrile, tetrahydrofuran, hexanes, ethyl acetate.

The synthesis is further illustrated in Scheme 43a below.

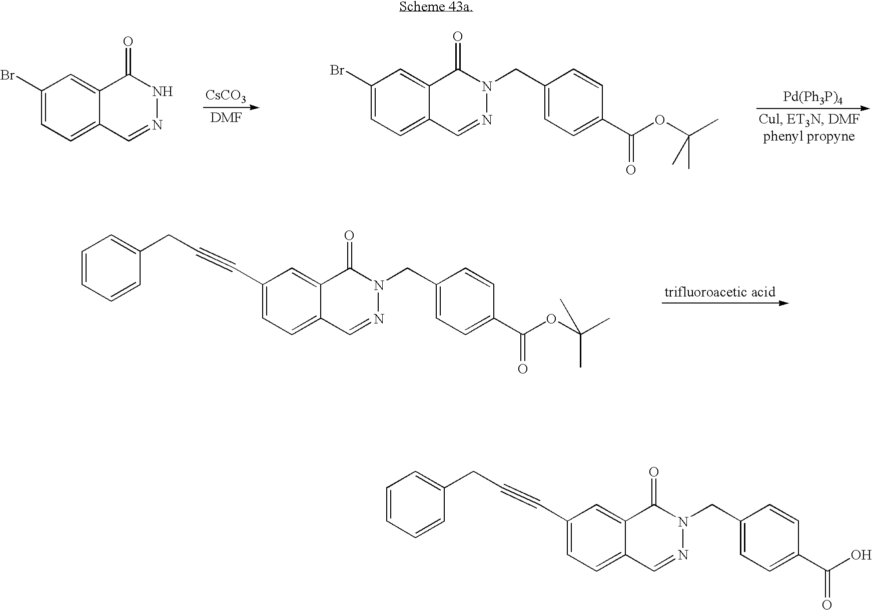

Compounds of Formula I that are phthalazinone derivatives may also be prepared according to the procedures referenced below for FIG. 1.

In FIG. 1, a bromo-substituted phthalazinone of formula (D) and a carboxylic acid ester of formula (E) may be prepared according to the procedure described in Chem. Pharm. Bull., 1985;33(7):2809-2820. Compounds of formulas (D) and (E) may be used to prepare compounds of Formula I wherein Q is a carbon-carbon triple bond, amide, or ester linker.

FIG. 1.

[image]

wherein Z, L, R¹, Q, and R² are as defined above for Formula I.

Compounds of Formula I that are naphthyridine derivatives may also be prepared according to the procedures referenced below for FIG. 2.

In FIG. 2, bromo-substituted naphthyridines or carboxylic acid ester naphthyridines of formulas (F), (G), and (H), wherein R is Br or, for example, EtOC(O), respectively, may be prepared according to the procedure described in Chem. Pharm. Bull., 1985;33(2):626-633. Compounds of formulas (F), (G), and (H) may be used to prepare compounds of Formula I wherein Q is a carbon-carbon triple bond, amide, or ester linker.

[image]

wherein Z, L, R¹, Q, and R² are as defined above for Formula I.

Compounds of Formula I wherein Q is a heterocyclic linker may be prepared according to the procedures outlined below in Scheme 43b. These are compounds of Formula I as drawn below, wherein V and X are as defined above and W₁, W₂, W₃, and W₄ are independently C(H) or N.

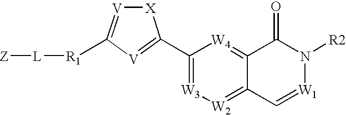

In Scheme 43b, bromo-substituted compounds of formula (I) may be converted, for example, to carboxylic acid esters of formula (J) using conventional carbonylation methods. Alkylation of the compounds of formula (J), followed by hydrolysis will afford the carboxylic acid intermediate of formula (K), which can then be used to make several of the heterocyclic linkers in compounds of Formula I.

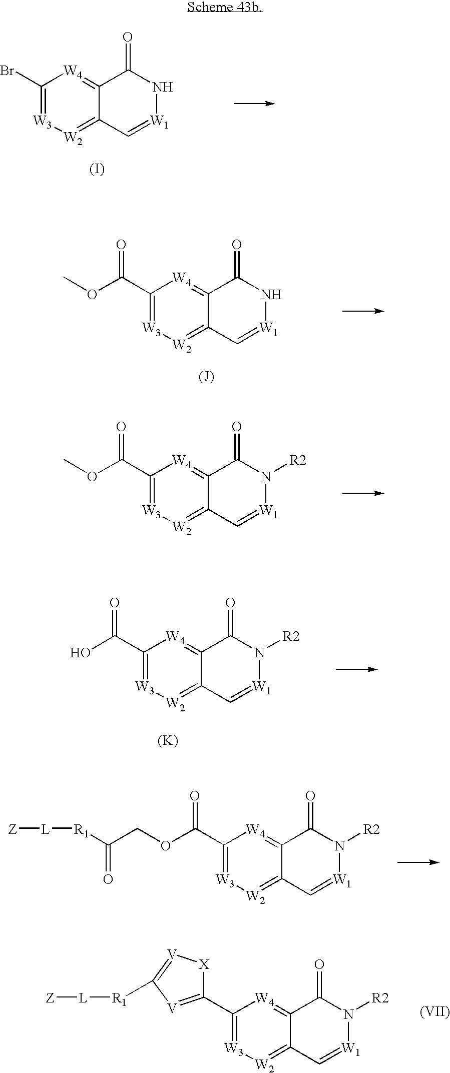

wherein Z, L, R¹, and R² are as defined above for Formula I and W₁, W₂, W₃, and W₄ are C(H) or N.

3-Isoquinolone derivatives of Formula I can be prepared according to the procedure described in Heterocycles, 1978;9(9):1197-1206.

3-Isoquinolone derivatives of Formula I wherein Q is a heterocyclic linker may be prepared according to the procedures outlined below in Scheme 44. These are compounds of Formula I as drawn below, wherein V and X are as defined above.

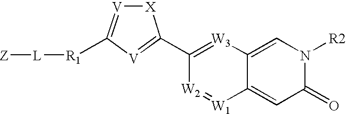

In Scheme 44, bromo-substituted compounds of formula (A) may be converted, for example, to carboxylic acid esters of formula (B) using conventional carbonylation methods. Alkylation of the compounds of formula (B), followed by hydrolysis will afford the carboxylic acid intermediate of formula (C), which can then be used to make several of the heterocyclic linkers, in compounds of Formula I.

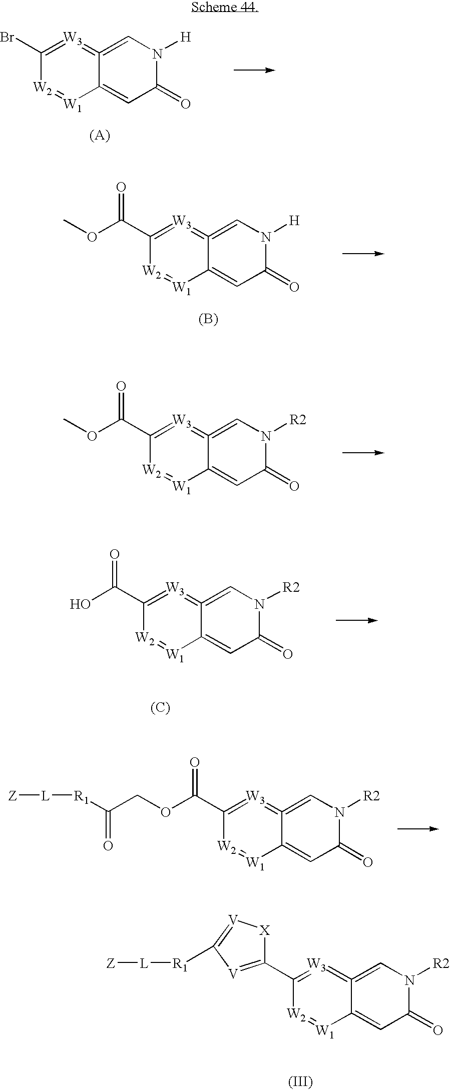

wherein Z, L, R¹, and R² are as defined above for Formula I and W₁, W₂, and W₃ are independently C(H) or N.

A general synthesis of the compounds of Formula I wherein Q is amide or carbon-carbon triple bond is shown below in Schemes 45 to 52.

In Scheme 45, 4-iodoaniline is treated with, for example, malonyl chloride methyl ester [Y² is C(O)] or chlorosulfinyl-acetic acid methyl ester [Y² is S(O)] in ethyl acetate with triethylamine to give the corresponding amide or sulfamide. Base hydrolysis of the ester with aqueous sodium hydroxide will give the acid (2). The acid (2) can be converted to the acid chloride using oxalyl chloride with catalytic dimethylformamide the treated with aluminum chloride to give the bicycle (3). Alternatively the acid (2) when heated with polyphosphoric acid gives the bicycle (3) directly.

Benzaldehyde or a suitably substituted benzaldehyde is mixed with (3) in methanol. HCL gas is added and the corresponding benzylidene (4) is isolated. The amide nitrogen is alkylated by stirring (4) in dimethylformamide and a suitable base such as sodium carbonate then addition of an alkyl halide such as iodomethane to give (5).

In addition intermediate (5) is treated with a strong base such as n-butyl lithium then carbon dioxide followed by aqueous acid to give the carboxylic acid (6). The acid (6) is coupled with a benzylamine or a suitably substituted benzylamine using a coupling reagent such as 3-(dimethylamino)propyl)ethyl carbodiimide hydrochloride (“EDAC”) and 1-hydroxybenzotriazole hydrate (“HOBT”) in dimethylformamide to give (8) [Y² is C(O)], [Y² is S(O)].

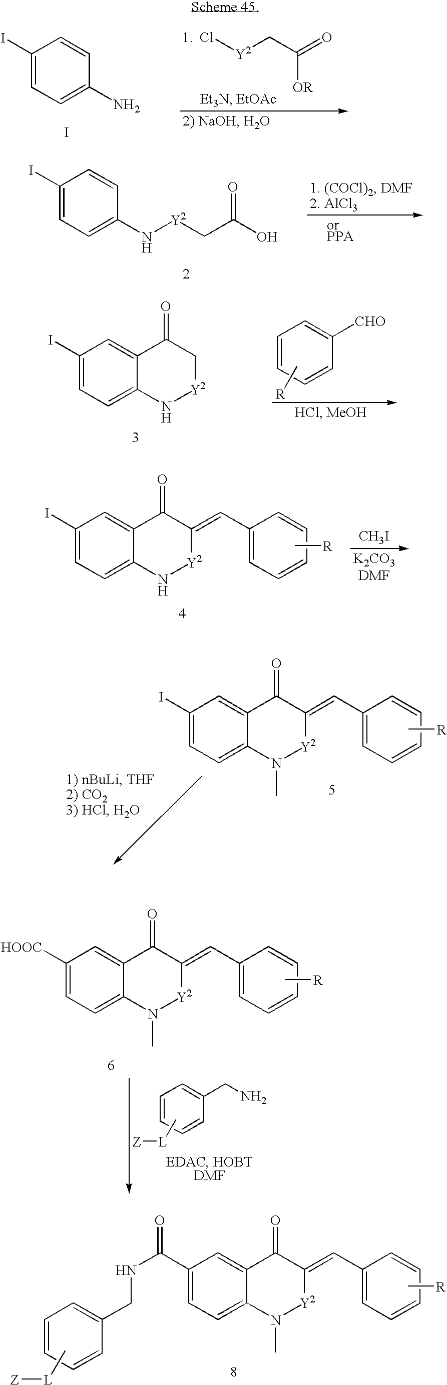

Y²=CO, S, SO, SO₂, P(OR)₂

R═H, Cl, F, Br, alkyl, O-alkyl

wherein Z and L are as defined above for Formula I.

Further, in Scheme 46 below, the iodine (5) reacts with benzylacetylene or a suitable substituted benzylacetylene in the presence of a soluble palladium catalyst such as bis-triphenylphosphine palladium dichloride and cuprous iodide in tetrahydrofuran to give (7) [Y² is C(O)] or [Y² is S(O)].

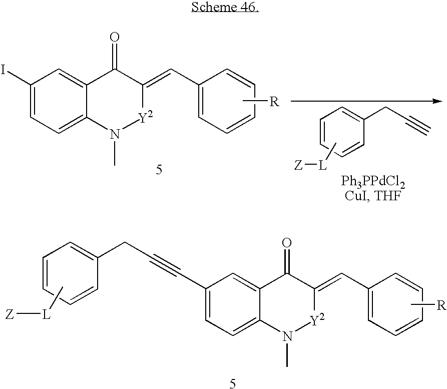

wherein Z and L are as defined above for Formula I and R and Y² are as defined above for Scheme 45.

Further, in Scheme 47 below, hydrogenation of the benzylidene double bond in compound (8) under hydrogen in the presence of palladium on carbon in ethanol will produce the compound (9) [Y² is C(O)], or [Y² is S(O)].

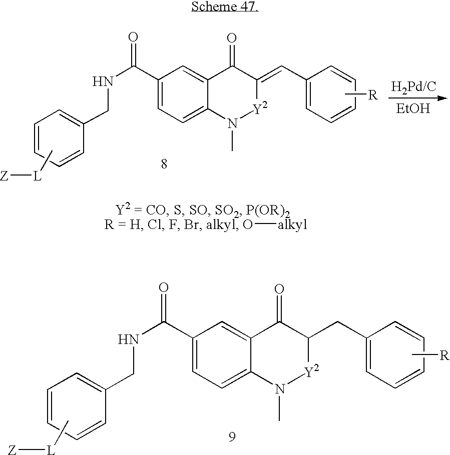

wherein Z and L are as defined above for Formula I.

A general synthesis of the compounds of Formula I wherein Q is amide or carbon-carbon triple bond is shown below in Schemes 48 to 50.

In Scheme 48, a compound of formula (10), iodophenol, for example, is treated with a base such as sodium hydride in tetrahydrofuran, followed by for example 3-bromoproionic acid methyl ester [Y¹ is C(O)]. The resulting phenoxypropionic acid methyl ester was hydrolyzed with sodium hydroxide in water to give acid (11).

The acid (11) is converted to the acid chloride with oxalyl chloride and catalytic dimethylformamide. Treating this with aluminum chloride in toluene produces the intermolecular cyclization to (12). Alternatively the acid (11) heated with polyphosphoric acid likewise will give (12) directly.

To a suspension of (12) and benzaldehyde or a substituted benzaldehyde in methanol is bubbled HCl gas. The resulting solution is heated at just below reflux to produce the benzylidene (13).

In addition intermediate (13) is treated with a strong base such as n-butyl lithium then carbon dioxide followed by aqueous acid to give the carboxylic acid (14). The acid (14) is coupled with a benzylamine or a suitably substituted benzylamine using a coupling reagent such as 3-(dimethylamino)propyl)ethyl carbodiimide hydrochloride and 1-hydroxybenzotriazole hydrate in dimethylformamide to give (16) [Y¹ is C(O) and Y³ is O].

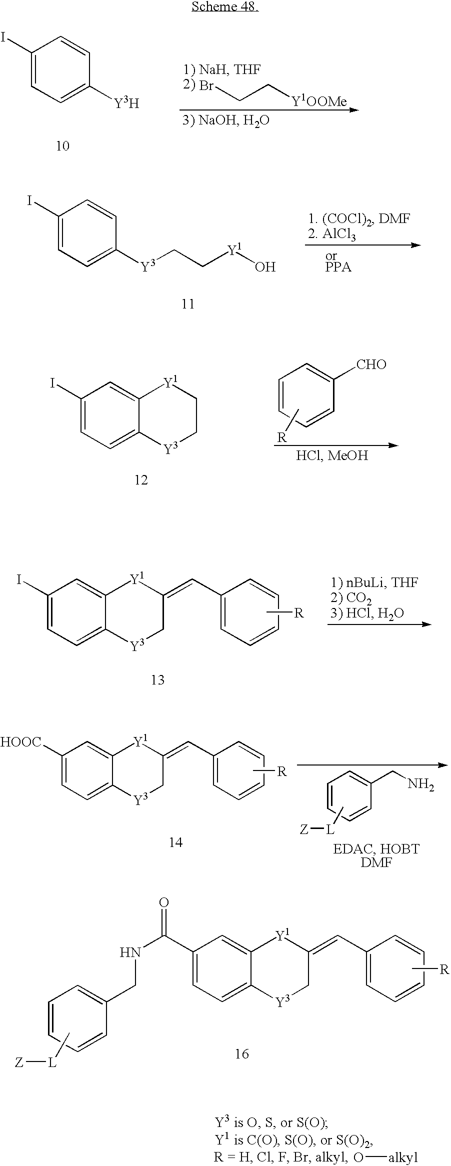

wherein Z and L are as defined above for Formula I.

Further, in Scheme 49, the iodine (13) reacts with benzylacetylene or a suitable substituted benzylacetylene in the presence of a soluble palladium catalyst such as bis-triphenylphosphine palladium dichloride and cuprous iodide in tetrahydrofuran to give (15) [Y¹ is C(O) and Y³ is O].

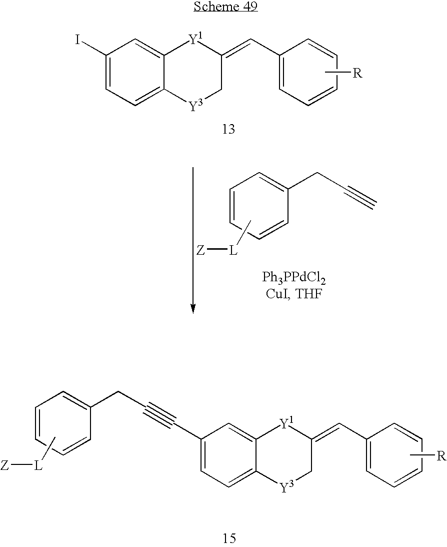

wherein Z and L are as defined above for Formula I and R, Y¹ and Y³ are as defined above for Scheme 48.

Further, in Scheme 50, hydrogenation of the benzylidene double bond in (16) under hydrogen in the presence of palladium on carbon in ethanol will produce the compound (17) [Y¹ is C(O) and Y³ is O].

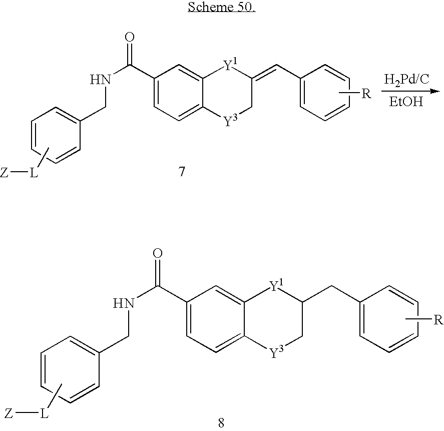

wherein Z and L are as defined above for Formula I and R, Y¹, and Y³ are as defined above for Scheme 48.

Alternatively, preparation of a compound of Formula I is outlined below in Scheme 51.

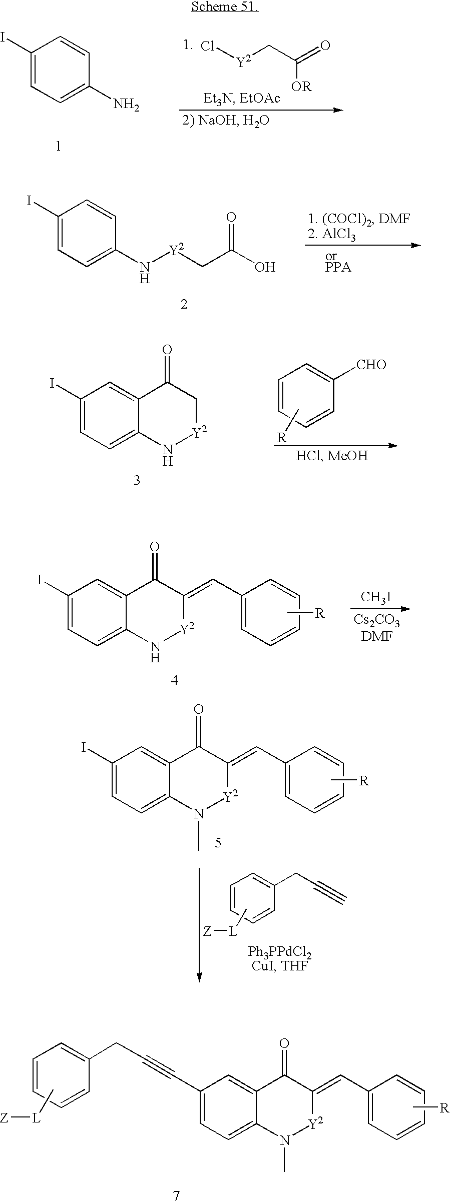

wherein Z and L are as defined above for Formula I, Y² is CH₂, C(O), or N(H), and R is as defined above for Scheme 48.

Alternatively, preparation of a compound of Formula I may be prepared as outlined below in Scheme 52.

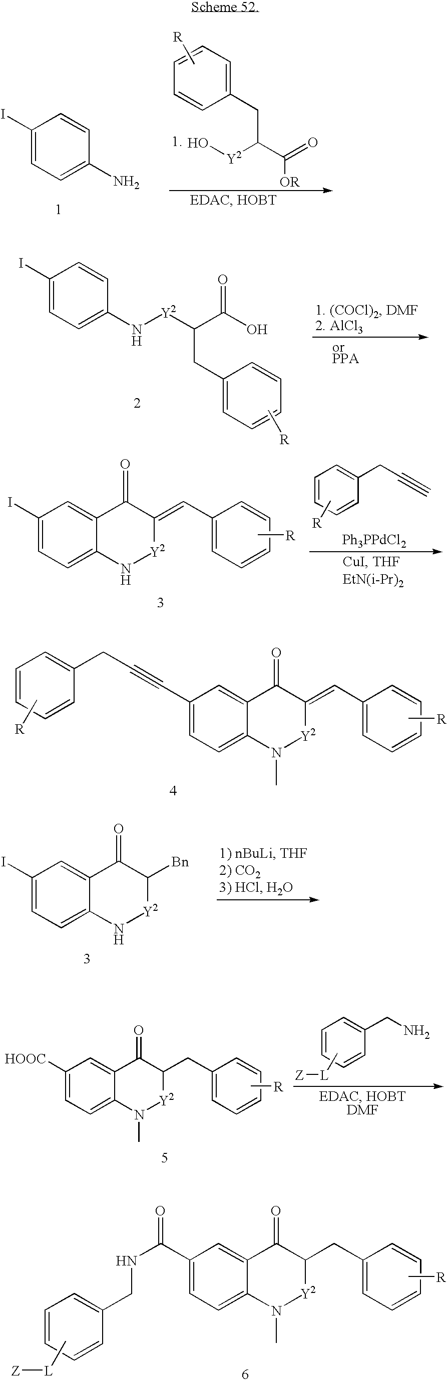

wherein Z and L are as defined above for Formula I, Y² is CH₂, C(O), or N(H), and R is as defined above for Scheme 48.

Another synthesis of the compounds of Formula I is outlined below in Scheme 53.

In Scheme 53, commercially available compound (1) is allowed to react with formamidine, for example, formamidine acetate, at elevated temperatures to give cyclic compound (2). Compound (2) is alkylated at N-3 to give compound (3), and the benzyl protecting group is removed to give compound (4). Compound (4) may be used to prepare 6-(3-substituted prop-2-ynyl) compounds of Formula I such as compound (5) or, alternatively, carbonylated derivatives of Formula I such as compound (6).

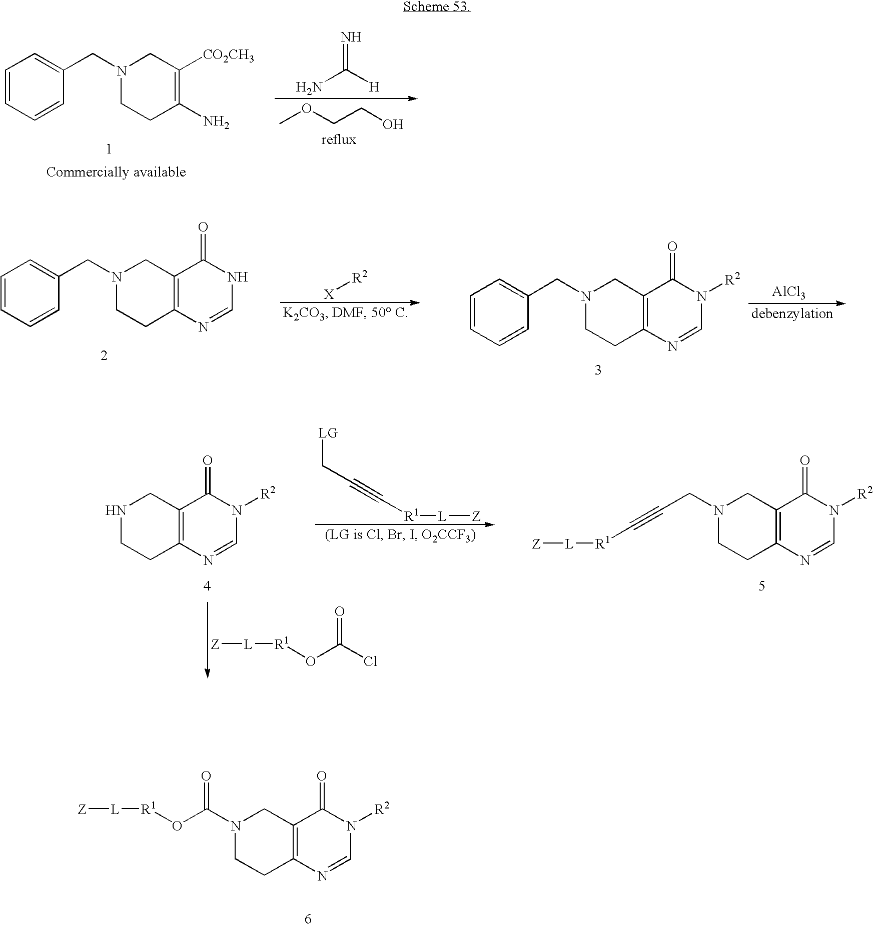

wherein Z, L, R¹, and R² are as defined above for Formula I.

Another synthesis of a compound of Formula I is outlined below in Scheme 54.

In Scheme 54, commercially available compound (1) is optionally allowed to react with formaldehyde under reducing conditions to give cyclic compound (2). Compound (2) is cyclized to give compound (3). Compound (3) is alkylated at N-3 to give compound (4), and the benzyl protecting group is removed to give compound (5). Compound (5) may be used to prepare 6-(3-substituted prop-2-ynyl) compounds of Formula I such as compound (6) or, alternatively, carbonylated derivatives of Formula I such as compound (7).

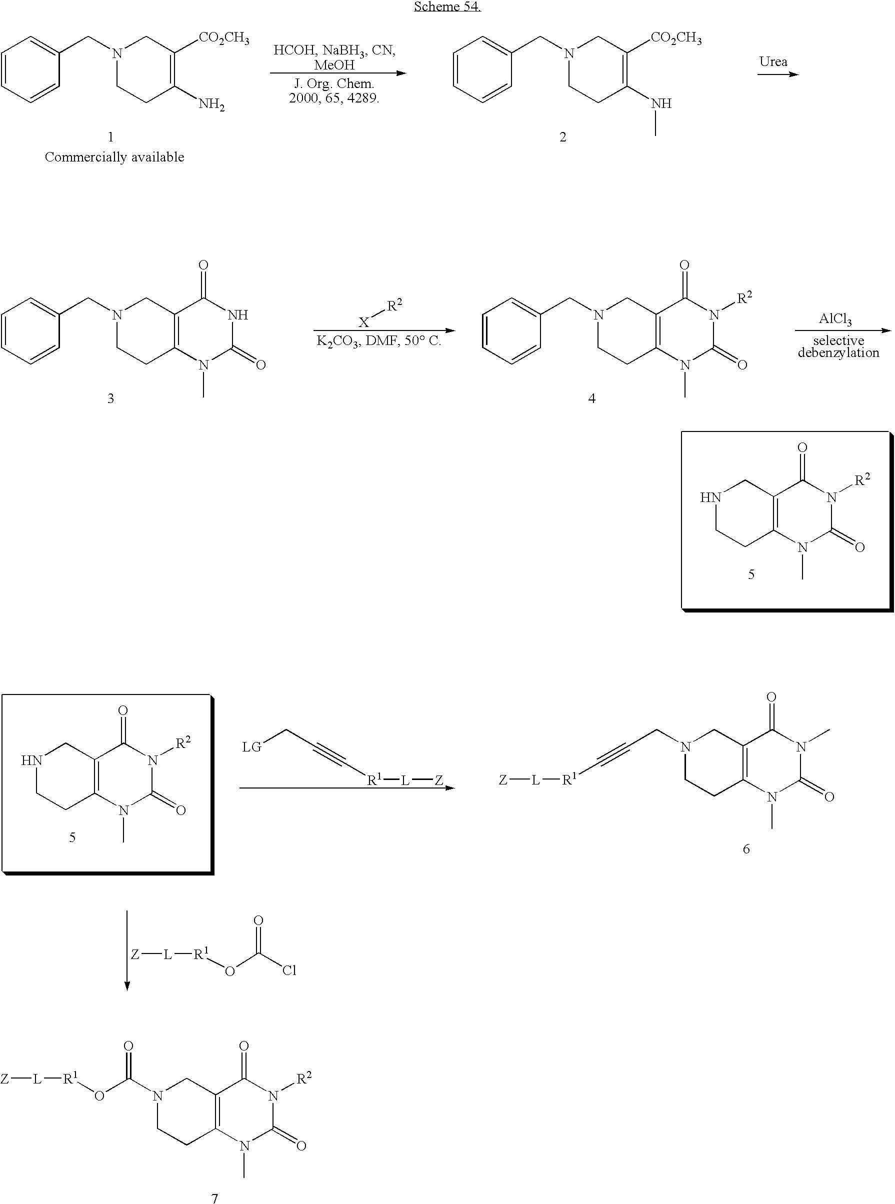

wherein Z, L, R¹, and R² are as defined above for Formula I and LG is Cl, Br, I, or CF₃CO₂.

Another synthesis of a compound of Formula I is outlined below in Scheme 55.

In Scheme 55, commercially available compound (1) is optionally allowed to react with formaldehyde under reducing conditions to give cyclic compound (2). Compound (2) is cyclized to give compound (3). Compound (3) is alkylated at N-3 to give compound (4), and the benzyl protecting group is removed to give compound (5). Compound (5) may be used to prepare 6-(3-substituted prop-2-ynyl) compounds of Formula I such as compound (6) or, alternatively, carbonylated derivatives of Formula I such as compound (7).

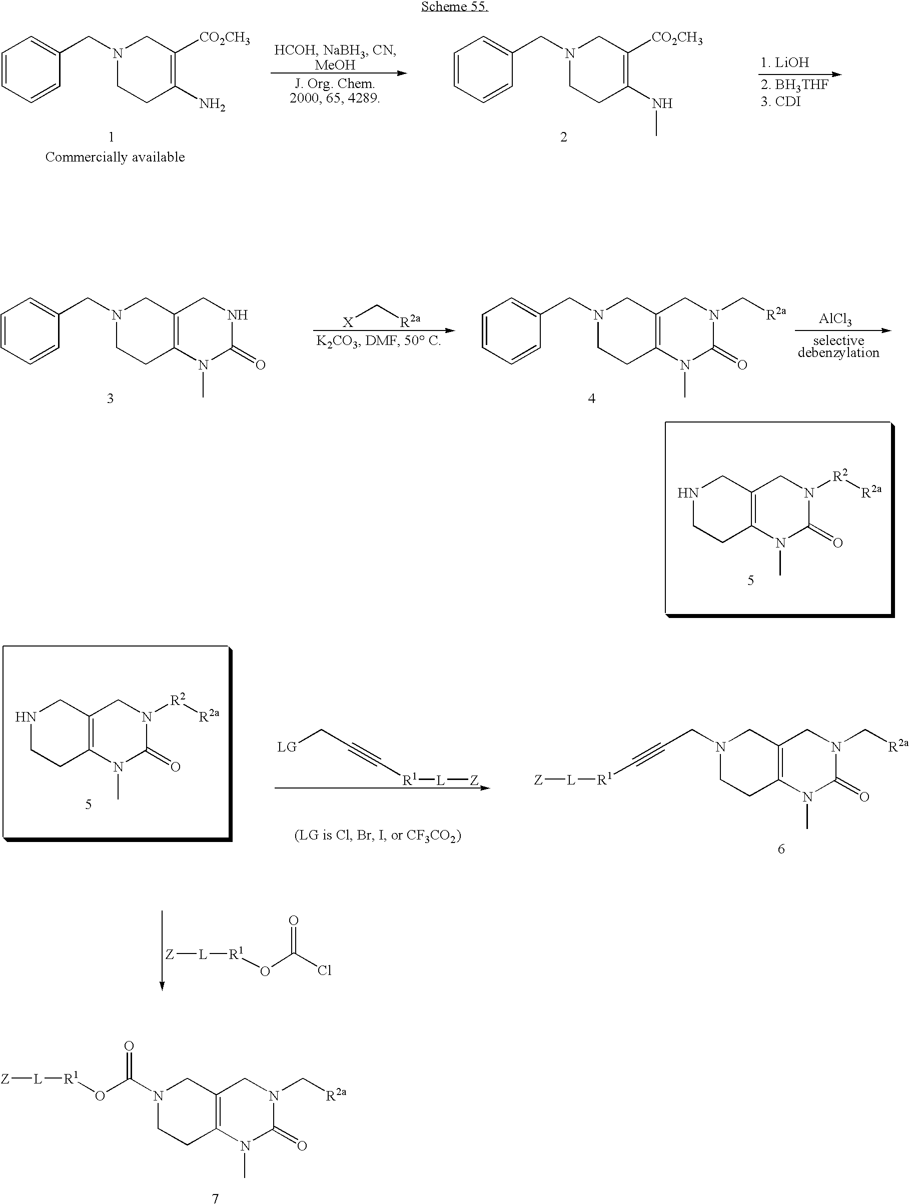

wherein Z, L, and R¹ are as defined above for Formula I and CH₂R^{2a} is a subset of the group R².

Another synthesis of a compound of Formula I is outlined below in Scheme 56.

In Scheme 56, commercially available compound (1) is hydrolyzed to give compound (2). Compound (2) is cyclized to give compound (3). Compound (3) is alkylated at N-3 to give compound (4), and the benzyl protecting group is removed to give compound (5). Compound (5) may be used to prepare 6-(3-substituted prop-2-ynyl) compounds of Formula I such as compound (6) or, alternatively, carbonylated derivatives of Formula I such as compound (7).

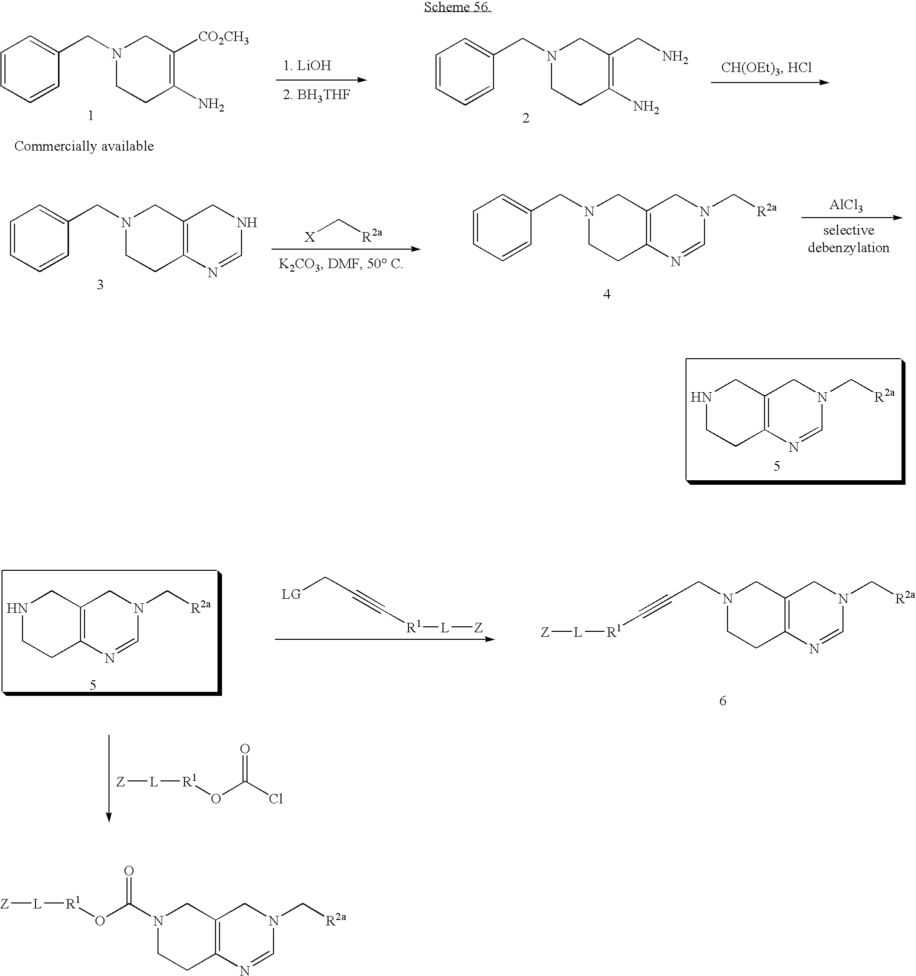

wherein Z, L, and R¹ are as defined above for Formula I and CH₂R^{2a} is a subset of the group R².

Other syntheses of the compounds of Formula I are outlined below in Schemes 57 to 62.

The pyrano[2,3-d]pyrimidine and thiopyrano[2,3-d]pyrimidine MMP-13 inhibitors are synthesized as shown in Scheme 57. In Scheme 57, condensation of malonic acid and a substituted urea in the presence of acetic anhydride or condensation of a malonic acid diester with a substituted urea in the presence of alkali alkoxide gives pyrimidinetrione 1. Reaction of 1 with paraformaldehyde followed by cycloaddition with ethyl acrylate gives ester 2. Alkylation of the available pyrimidine nitrogen of 2 with an alkyl halide in the presence of cesium carbonate followed by hydrolysis of the ester and coupling to an amine using a carbodiimide gives pyrano[2,3-d]pyrimidine 4. Alternatively, hydrolysis of the ester of 2 and coupling to an amine using a carbodiimide gives pyrano[2,3-d]pyrimidine 3.

Thiopyrano[2,3-d]pyrimidines are prepared as shown in Scheme 57. In Scheme 57, pyrimidinetrione 1 is reacted with phosphorus oxychloride and DMF followed by reaction of the chloroaldehyde with sodium hydrosulfide to give 6. Michael reaction of 6 with ethyl acrylate and treatment with base followed by dehydration, ester hydrolysis and carbodiimide coupling gives 7. Product 7 may be oxidized with hydrogen peroxide, peracetic acid, or meta-chloroperoxybenzoic acid to give sulfoxide or sulfone 8 as desired by employing one or two equivalents of oxidizing agent respectively. Alkylation of the available pyrimidine nitrogen of 7 with an alkyl halide in the presence of cesium carbonate gives 5 (n=0), while alkylation of the available pyrimidine nitrogen of 8 with an alkyl halide in the presence of cesium carbonate gives sulfoxide 5 (n=1) or sulfone 5 (n=2).

The thiopyrano[2,3-d]pyrimidines represented by 12 and 13 are prepared as shown in Scheme 58. In Scheme 58, sequential reaction of ethyl cyanoacetate with ethyl chloromethacrylate in the presence of base followed by reaction with sodium hydrosulfide gives thiopyran 9. Reaction of 9 with an isocyanate and ring closure in the presence of sodium ethoxide gives 10. Alkylation of 10 followed by ester hydrolysis and coupling with an amine gives 12, which may be oxidized with hydrogen peroxide, peracetic acid, or meta-chloroperoxybenzoic acid to give sulfoxide (n=1) or sulfone (n=2) 13.

Pyrido[2,3-d]pyrimidines 16 and 17 are prepared as shown in Scheme 59. In Scheme 59, pyrimidinetrione 1 is reacted with phosphorus oxychloride and DMF followed by reaction of the chloroaldehyde 14 with N-methyl beta-alanine ethyl ester and treatment with a base to give 15. One subjects 15 to ester hydrolysis and carbodiimide coupling with an amine gives 16. Alkylation of the available pyrimidine nitrogen of 16 with an alkyl halide in the presence of cesium carbonate gives pyrido[2,3-d]pyrimidine 17.

Scheme 60 shows that decahydro-quinazoline derivative 19 is obtained by reacting amino diester 18 with an aldehyde under reductive amination conditions followed by reaction with an isocyanate and cyclization under basic conditions to give a decahydro-quinazoline ester. The ester is hydrolyzed and then coupled with an amine to give 19.

Scheme 61 shows the synthesis of pteridine MMP 13 inhibitors. Thus reaction of malonate with a urea in the presence of alkali alkoxide followed by treatment with phosphorus oxychloride gives a chlorouracil. The chlorouracil is reacted with sodium azide and then is alkylated to give 6-azidouracil 20. Photochemical reaction of 20 with an aminopyruvic amide gives 21.

The pyrimido[5,4-b][1,4]thiazine MMP-13 inhibitors are obtained as shown in Scheme 62. In Scheme 62, aminochlorouracil 22 is reacted with 2-mercaptomalonic acid and is then cyclized in the presence of acetic anhydride to give 26. Acid 26 is coupled with an amine to give 27. One may oxidize the sulfur of 27 with hydrogen peroxide, peracetic acid, or meta-chloroperoxybenzoic acid to obtain a sulfoxide (n=1) or a sulfone (n=2). Scheme 6 also shows the synthesis of a related pyrimido[5,4-b][1,4]thiazine system 25. Reaction of 22 with methyl thioglycolate gives 23. Reaction of 23 with dimethylformamide dimethylacetal gives 24. Ester hydrolysis of 24 and carbodiimide coupling with an amine gives 25. One may oxidize the sulfur of 25 with hydrogen peroxide, peracetic acid, or meta-chloroperoxybenzoic acid to obtain a sulfoxide (n=1) or a sulfone (n=2).

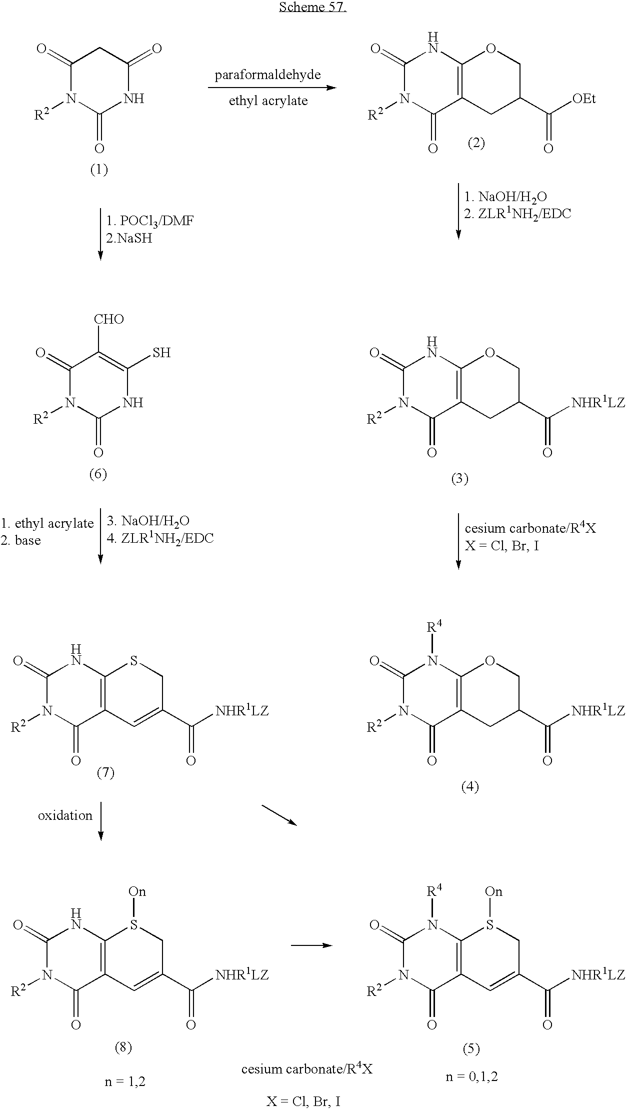

wherein Z, L, R¹, and R² are as defined above for Formula I and R⁴ is C₁-C₆ alkyl.

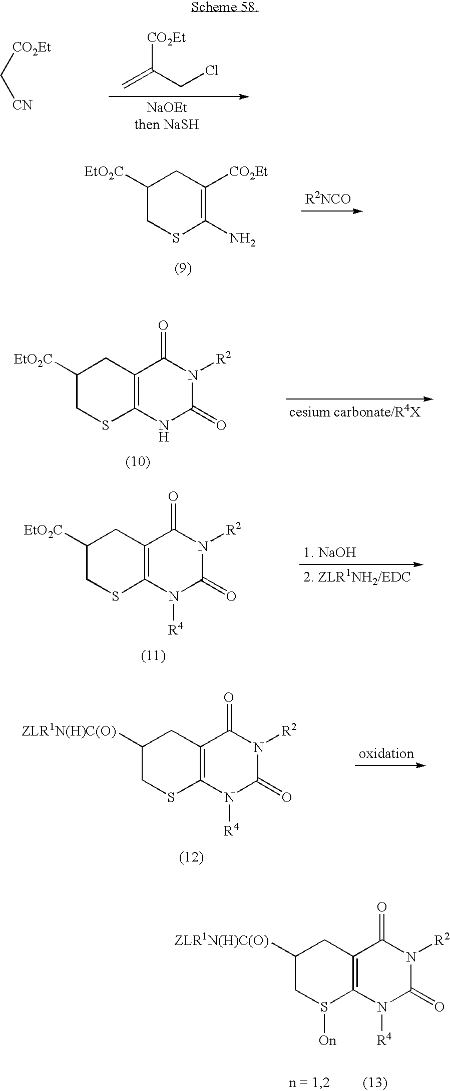

wherein Z, L, R¹, and R² are as defined above for Formula I and R⁴ is C₁-C₆ alkyl.

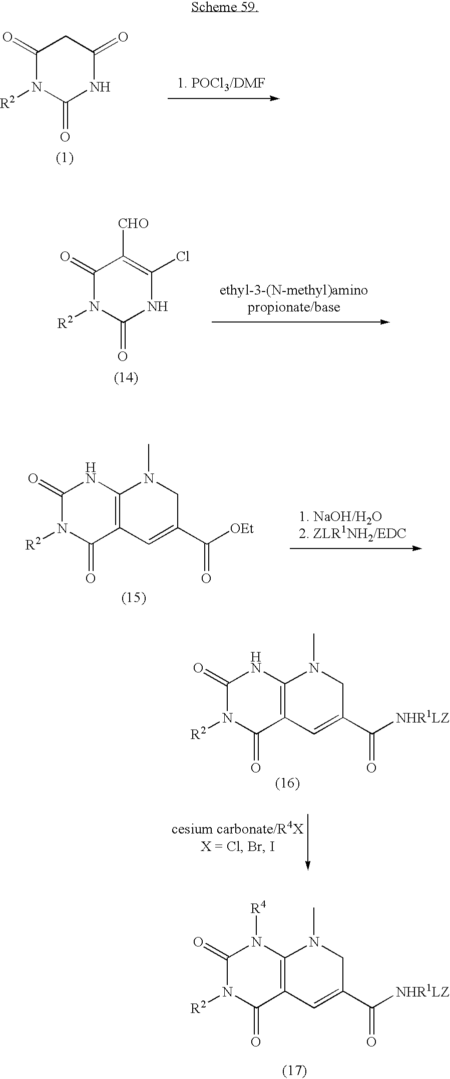

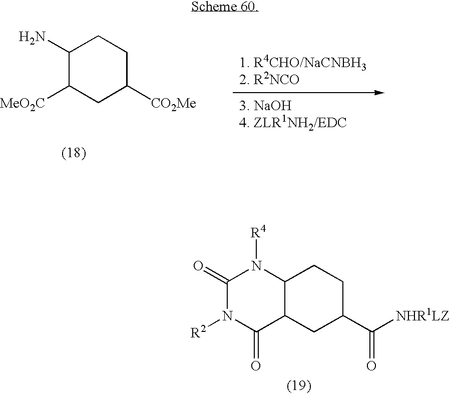

wherein Z, L, R¹, R² are as defined above for Formula I and R⁴ is C₁-C₆ alkyl.

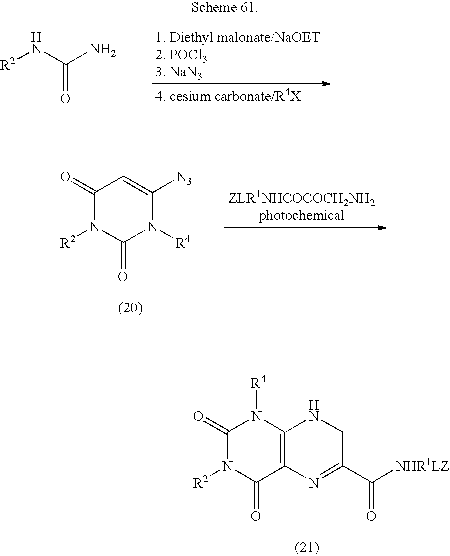

wherein Z, L, R¹, R² are as defined above for Formula I and R⁴ is C₁-C₆ alkyl.

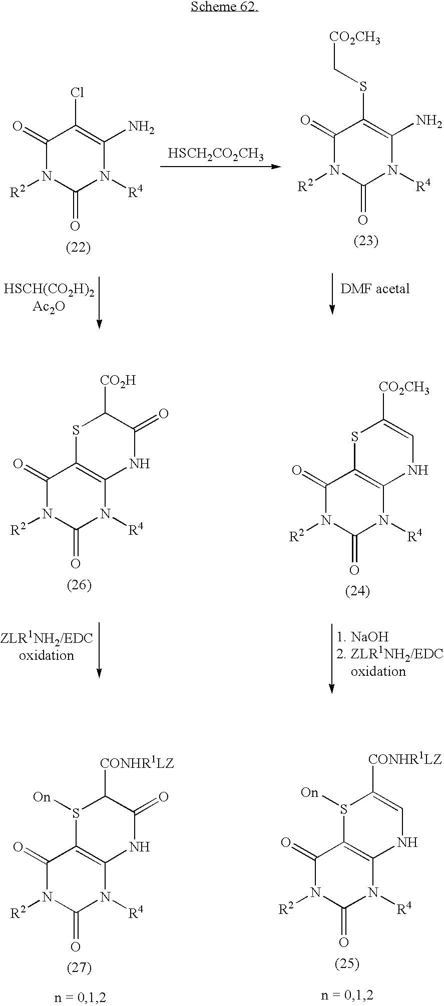

wherein Z, L, R¹, R² are as defined above for Formula I and R⁴ is C₁-C₆ alkyl.

Other syntheses of the compounds of Formula I are outlined below in Scheme 63.

The synthesis of the pyrimido[6,1-c][1,4]oxazine, pyrimido[6,1-c][1,4]thiazine, and pyrazino[1,2-c]pyrimidine systems is shown in Scheme 63. In Scheme 63, the 6-chloromethyl uracil, 29, is reacted with methylglycolate, methylthioglycolate, or glycine methyl ester to give 30 (X=O, S, NH). Reaction of 30 with dimethylformamide dimethylacetal gives 31 (X=O, S, NH). Subjecting 31 to ester hydrolysis and carbodiimide coupling with an amine gives 32 (X=O, S, NH). One may oxidize the sulfur of 32 with hydrogen peroxide, peracetic acid, or meta-chloroperoxybenzoic acid to obtain a sulfoxide (32, X=SO) or a sulfone (32, X=SO₂).

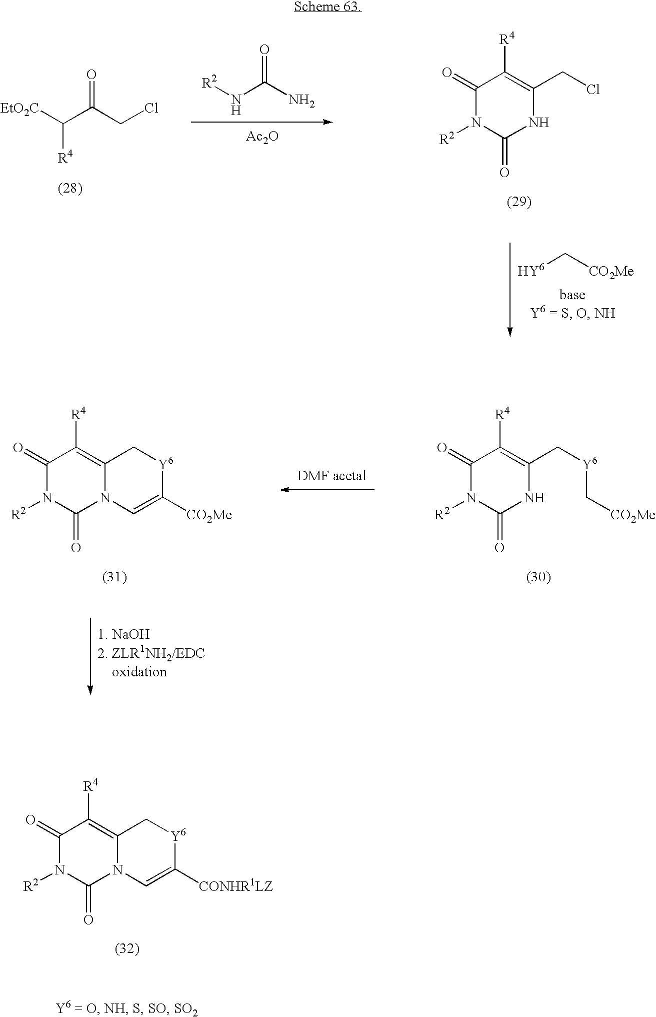

wherein Z, L, R¹, and R² are as defined above for Formula I and R⁴ is C₁-C₄ alkyl.

Other syntheses of the compounds of Formula I are outlined below in Schemes 64a, 64b, 65a, 65b, 66a, 66b, 67a, 67b, 68a, 68b, 69a, and 69b.

In Scheme 64a, a compound of formula (1) may be prepared according to the procedure described in J. Am. Chem. Soc. 1951;73:2082-2085. The compound of formula (1) is converted to (2) with chloromethyl methyl ether (“MOMCl”) in the presence of sodium ethoxide (“NaOEt”) and 18-crown-6. Compound (2) is brominated with N-bromosuccinimide (“NBS”) in the presence of 2,2′-azobisisobutyronitrile (“AIBN”) to give (3). Compound (3) is coupled with an aryl cuprate of formula (Ar²)₂CuLi, wherein Ar² means a phenyl, naphthyl, or any heteroaryl group, unsubstituted or substituted, as defined above, with tetrahydrofuran (“THF”) to give (4), wherein CH₂Ar² is a group according to R² of Formula I as described above. Compound (4) is coupled with an alkyne of formula Ar¹CH₂C≡CH, wherein Ar¹CH₂C≡C is a group according to R¹ of Formula I, in the presence of a transition metal catalyst such as bistriphenylphosphine palladium(II) chloride with diethylisopropylamine (“Et₂NiPr”) and dimethylformamide (“DMF”) to give (5). Compound (5) is deprotected with hydrogen chloride, isopropanol (“iPrOH”) and THF to give (6).

Alternatively, a compound of formula (4), prepared as outlined in Scheme 64a below, may be converted to an amide of formula (9) as outlined below in Scheme 64b. In Scheme 64b, the compound of formula (4) is carbonylated with a palladium(II) catalyst such as palladium(II) chloride 1,1′-bis(diphenylphosphino)ferrocene [“PdCl₂(dppf)”] with triethylamine (“Et₃N”) to give (7), wherein CH₂Ar² is a group according to R² of Formula I. Compound (7) is coupled with an amine of formula R¹NH₂, wherein R¹ is as defined above, with aluminum chloride and THF to give (8). Compound (8) is deprotected as described above to give (9).

Alternatively, a compound of Formula I wherein Q is OC(O) may be prepared following the procedure of Scheme 64b by substituting an alcohol of formula R¹OH for the amine of formula R¹NH₂, wherein R¹ is as defined above.

In Scheme 65a, a compound of formula (1) may be prepared according to the procedure described in J. Med. Chem., 1990;33(1):171-178. Compound (1) is protected with a MOM group as described previously to give (2). Compound (2) is reduced with lithium aluminum hydride (“LAH”) with THF to give (3). Compound (3) is coupled with para-toluenesulfonyl chloride (“TsCl”) with triethylamine, 4-dimethylaminopyridine (“DMAP”), and dichloromethane (“CH₂Cl₂”) to give (4). Compound (4) is coupled with an aryl cuprate of formula (Ar²)₂CuLi, wherein Ar² means a phenyl, naphthyl, or any heteroaryl group, unsubstituted or substituted, as defined above, to give (5), wherein CH₂Ar² is a group according to R² of Formula I, as described above. Compound (5) is coupled with an alkyne of formula Ar¹CH₂C≡CH, wherein Ar¹CH₂C≡C is a group according to R¹ of Formula I, as described above to give (6). Compound (6) is deprotected as described above to give (7).

Alternatively, a compound of formula (5) from Scheme 65a may be converted to an amide of formula (10) as outlined below in Scheme 65b. In Scheme 65b, the conversion follows the method described previously for Scheme 64b.

Alternatively, a compound of Formula I wherein Q is OC(O) may be prepared following the procedure of Scheme 65b by substituting an alcohol of formula R¹OH for the amine of formula R¹NH₂, wherein R¹ is as defined above.

In Scheme 66a, a compound of formula (1) may be prepared according to the procedure described in J. Organomet. Chem., 1977;128(1):95-98. A compound of formula (1) is alkylated with a compound of formula BrCH₂Ar², wherein Ar² means a phenyl, naphthyl, or any heteroaryl group, unsubstituted or substituted, as defined above, with normal-butyl lithium (“n-BuLi”) and TBF to give (2), wherein CH₂Ar² is a group according to R² of Formula I, as described above. Compound (2) is coupled with an alkyne of formula Ar¹CH₂C≡CH, wherein Ar¹CH₂C≡C is a group according to R¹ of Formula I, as described above to give (4).

Alternatively, a compound of formula (2) from Scheme 66a may be converted to an amide of formula (6) as outlined below in Scheme 66b. In Scheme 66b, the conversion follows the method described previously for Scheme 64b.

Alternatively, a compound of Formula I wherein Q is OC(O) may be prepared following the procedure of Scheme 66b by substituting an alcohol of formula R¹OH for the amine of formula R¹NH₂, wherein R¹ is as defined above.

In Scheme 67a, a compound of formula (1) may be prepared according to the procedure described in Journal of Heterocyclic Chemistry, 1999;36(4):895-899. The compound of formula (1) is bis-MOM protected to give (2) as described above for Scheme 1a. Compound (2) is reduced with LAH and THF as described previously to give (3). Compound (3) is converted to the tosylate (4) with TsCl, triethylamine, DMAP, and dichloromethane as described previously. Compound (4) is coupled with a cuprate of formula (Ar²)₂CuLi, wherein Ar² means a phenyl, naphthyl, or any heteroaryl group, unsubstituted or substituted, as defined above, to give (5), wherein CH₂Ar² is a group according to R² of Formula I, as described above. Compound (5) is coupled with an alkyne of formula Ar¹CH₂C≡CH, wherein Ar¹CH₂C≡C is a group according to R¹ of Formula I, as described above to give (6). Compound (6) is deprotected as described above to give (7).

Alternatively, a compound of formula (6) from Scheme 67a may be converted to an amide of formula (10) as outlined below in Scheme 67b. In Scheme 67b, the conversion follows the method described previously for Scheme 64b.

Alternatively, a compound of Formula I wherein Q is OC(O) may be prepared following the procedure of Scheme 67b by substituting an alcohol of formula R¹OH for the amine of formula R¹NH₂, wherein R¹ is as defined above.

In Scheme 68a, a compound of formula (1) may be prepared according to the procedure described in J. Med. Chem., 1978;21(3):268-272. The compound of formula (1) is MOM protected to give (2) as described above for Scheme 64a. Compound (2) is reduced with LAH and THF as described previously to give (3). Compound (3) is converted to the tosylate (4) with TsCl, triethylamine, DMAP, and dichloromethane as described previously. Compound (4) is coupled with a cuprate of formula (Ar²)₂CuLi, wherein Ar² means a phenyl, naphthyl, or any heteroaryl group, unsubstituted or substituted, as defined above, to give (5), wherein CH₂Ar² is a group according to R² of Formula I, as described above. Compound (5) is coupled with an alkyne of formula Ar¹CH₂C≡CH, wherein Ar¹CH₂C≡C is a group according to R¹ of Formula I, as described above to give (6). Compound (6) is deprotected as described above to give (7).

Alternatively, a compound of formula (6) from Scheme 68a may be converted to an amide of formula (10) as outlined below in Scheme 68b. In Scheme 68b, the conversion follows the method described previously for Scheme 64b.

Alternatively, a compound of Formula I wherein Q is OC(O) may be prepared following the procedure of Scheme 68b by substituting an alcohol of formula R¹OH for the amine of formula R¹NH₂, wherein R¹ is as defined above.

In Scheme 69a, a compound of formula (1) may be prepared according to the procedure described in J. Chem. Soc., Perkin Trans., 1981;1(5):1520-1530. The compound of formula (1) is MOM protected to give (2) as described above for Scheme 64a. Compound (2) is reduced with LAH and THF as described previously to give (3). Compound (3) is converted to the tosylate (4) with TsCl, triethylamine, DMAP, and dichloromethane as described previously. Compound (4) is coupled with a cuprate of formula (Ar²)₂CuLi, wherein Ar² means a phenyl, naphthyl, or any heteroaryl group, unsubstituted or substituted, as defined above, to give (5), wherein CH₂Ar² is a group according to R² of Formula I, as described above. Compound (5) is coupled with an alkyne of formula Ar¹CH₂C≡CH, wherein Ar¹CH₂C≡C is a group according to R¹ of Formula I, as described above to give (6). Compound (6) is deprotected as described above to give (7).

Alternatively, a compound of formula (5) from Scheme 69a may be converted to an amide of formula (10) as outlined below in Scheme 69b. In Scheme 69b, the conversion follows the method described previously for Scheme 64b.

Alternatively, a compound of Formula I wherein Q is OC(O) may be prepared following the procedure of Scheme 69b by substituting an alcohol of formula R¹OH for the amine of formula R¹NH₂, wherein R¹ is as defined above.

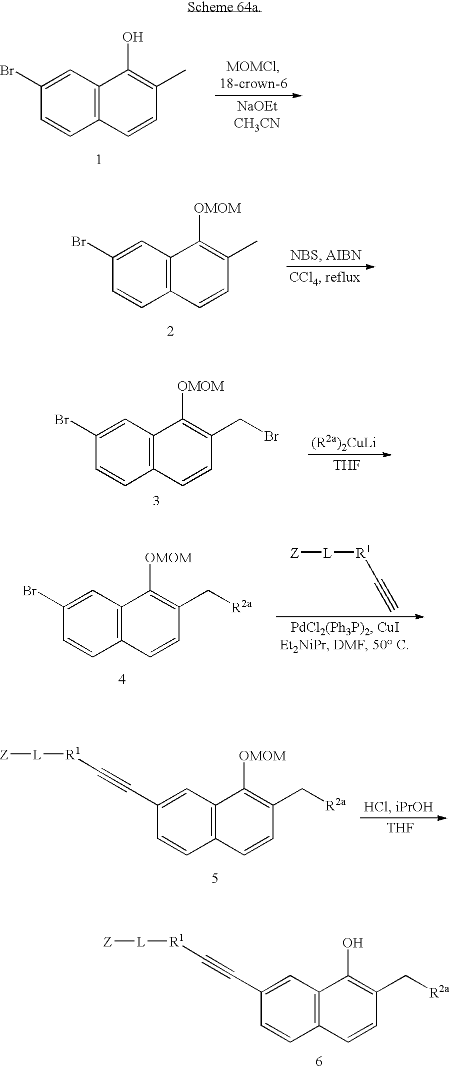

wherein Z, L, and R¹ are as defined above for Formula I and R^{2a}CH₂ is a subset of the group R².

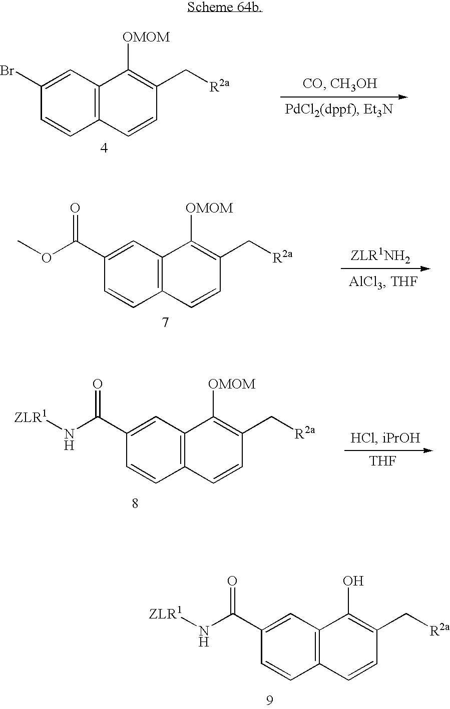

wherein Z, L, and R¹are as defined above for Formula I and R^{2a}CH₂ is a subset of the group R².

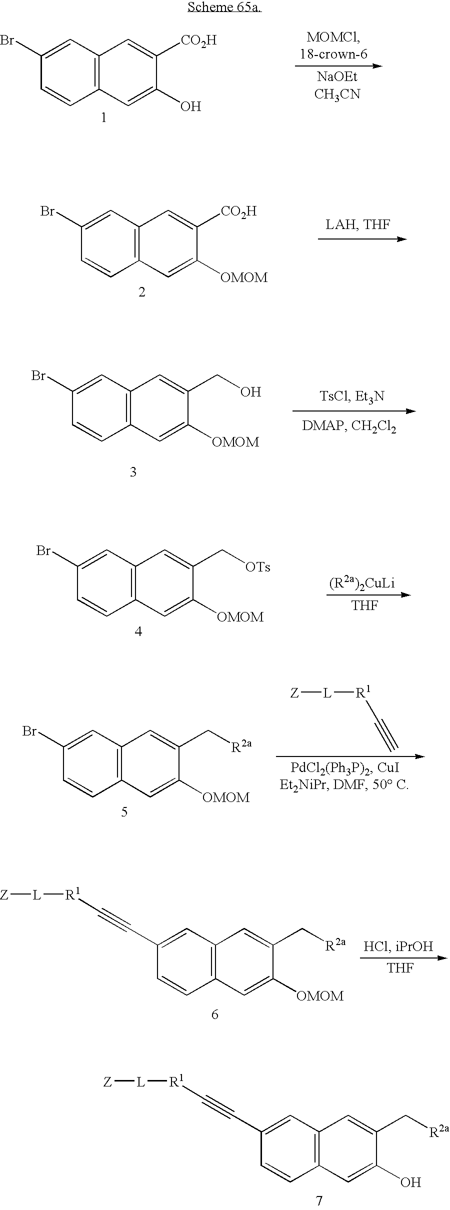

wherein Z, L, and R¹ are as defined above for Formula I and R^{2a}CH₂ is a subset of the group R².

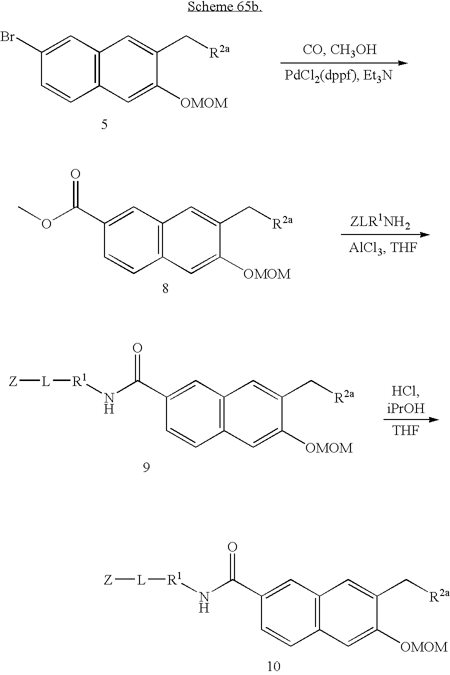

wherein Z, L, and R¹ are as defined above for Formula I and R^{2a}CH₂ is a subset of the group R².

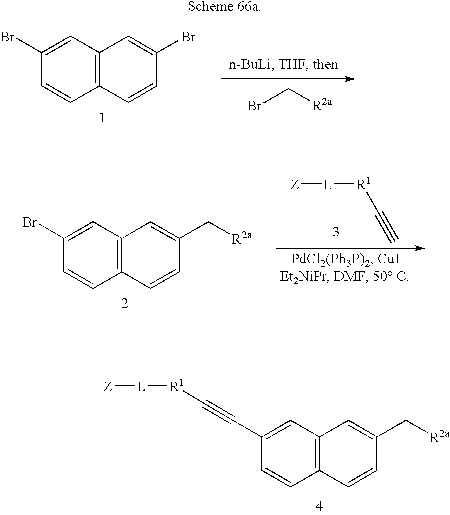

wherein Z, L, and R¹ are as defined above for Formula I and R^{2a}CH₂ is a subset of the group R².

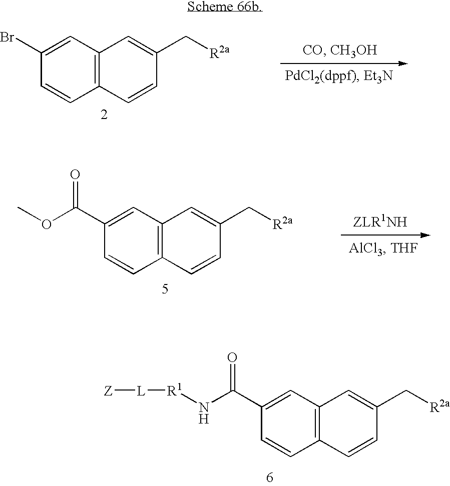

wherein Z, L, and R¹ are as defined above for Formula I and R^{2a}CH₂ is a subset of the group R².

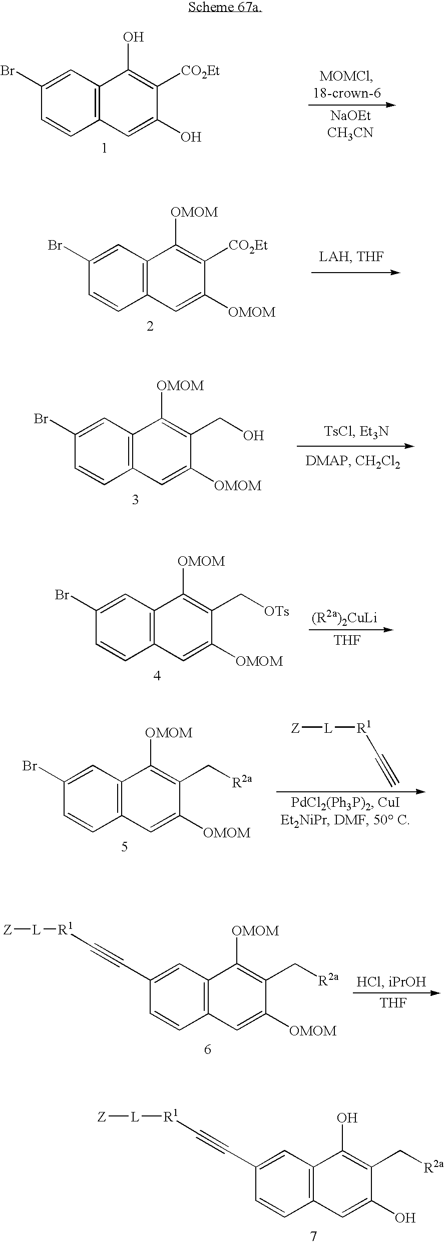

wherein Z, L, and R¹ are as defined above for Formula I and R^{2a}CH₂ is a subset of the group R².

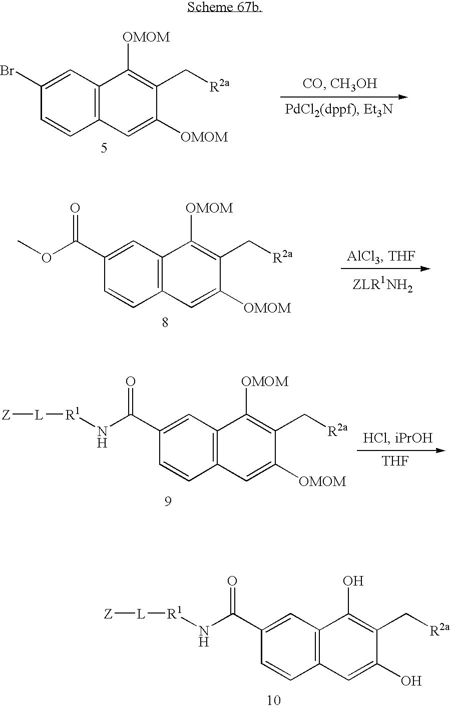

wherein Z, L, and R¹ are as defined above for Formula I and R^{2a}CH₂ is a subset of the group R².

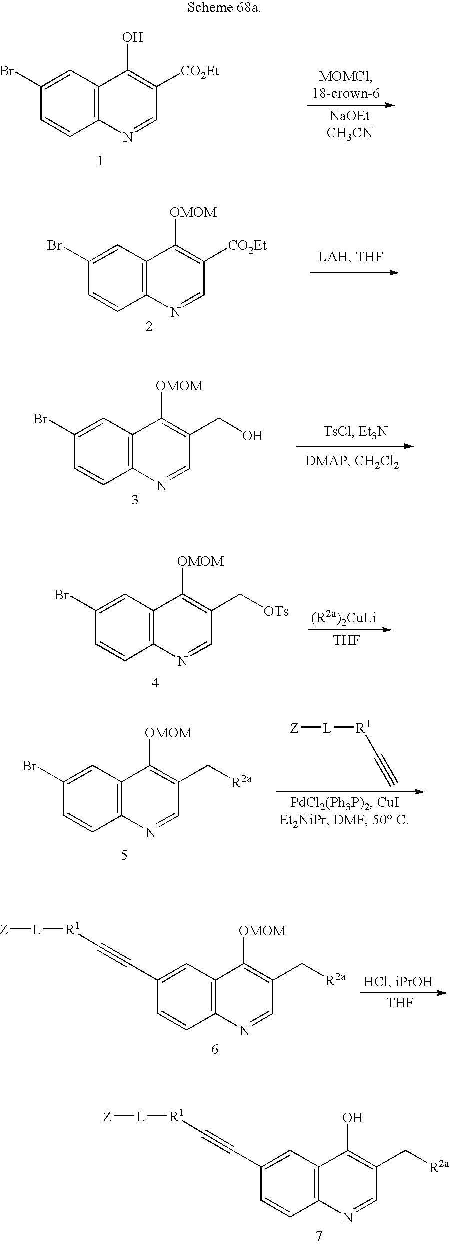

wherein Z, L, and R¹ are as defined above for Formula I and R^{2a}CH₂ is a subset of the group R².

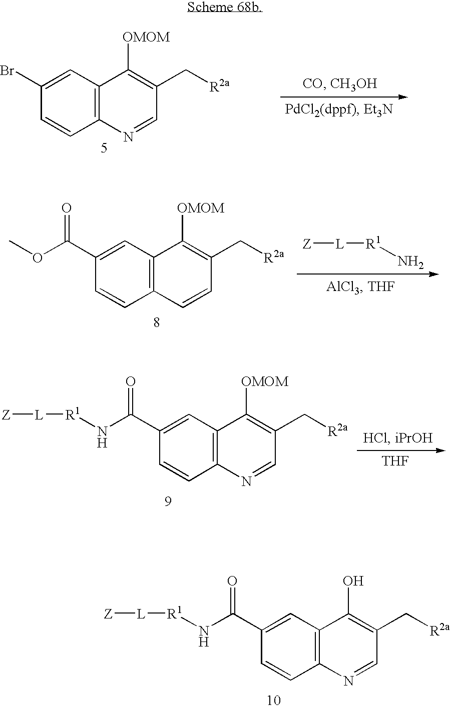

wherein Z, L, and R¹ as defined above for Formula I and R^{2a}CH₂ is a subset of the group R².

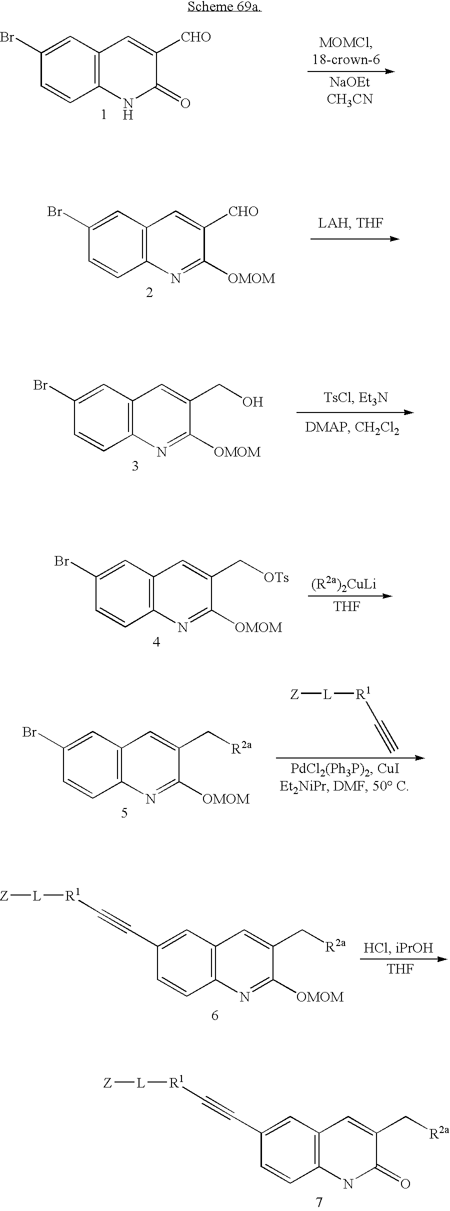

wherein Z, L, and R¹ are as defined above for Formula I and R^{2a}CH₂ is a subset of the group R².

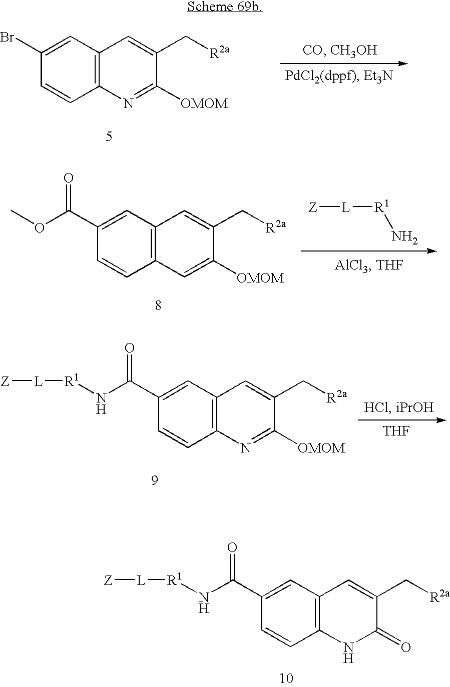

wherein Z, L, and R¹ are as defined above for Formula I and R^{2a}CH₂ is a subset of the group R².

Another preparation of a compound of Formula I is outlined below in Schemes 70 to 73.

For example, a synthesis of the compounds of Formula I wherein Q is S, S(O), or S(O)₂ is outlined below in Scheme 70. In Scheme 70, a urea of formula (1), wherein R² is as defined above, is condensed with a malonate of formula (2) in the presence of a suitable base and solvent such as sodium ethoxide (“NaOEt”) in ethanol (“EtOH”) to give the pyrimidinetrione of formula (3). Compound (3) is chlorinated with, for example, phosphorous oxychloride (“POCl₃”) to give compound (4). Compound (4) is reacted with a sulfurating reagent such as sodium hydrogen sulfide (“NaSH”) in a polar solvent such as dimethylformamide (“DMF”) followed by acidification to give compound (5). Compound (5) is alkylated, arylated, or heteroarylated with a compound (6), wherein Z, L, and R¹ are as defined above and X is a suitable leaving group such as Cl, Br, I, para-toluenesulfonate, trifluoromethanesulfonate, and the like in a polar aprotic solvent such as DMF to give compound (7), which is a compound of Formula I, wherein Q is S. Compound (8) is optionally oxidized with a suitable stoichiometry of a suitable oxidizing agent such as meta-chloroperbenzoic acid (“mCPBA”) to give compound (8), which is a compound of Formula I wherein Q is S(O) or S(O)₂.

Compounds of Formula I wherein Q is O or N(R⁵) may be prepared as outlined below in Schemes 71 and 72, respectively. In Schemes 71 and 72, an alcohol or amine, respectively, of formula (1), wherein R² is as defined above, may be prepared in a manner similar to that described above in Scheme 70 for the preparation of a compound of Formula I wherein Q is S, by replacing NaSH in Scheme 1 with NaOH or N(R⁵)H₂, respectively. Compounds (1) in Schemes 71 and 72 are alkylated, arylated, or heteroarylated with a compound (2), wherein R¹ is as defined above and X is a suitable leaving group such as Cl, Br, I, para-toluenesulfonate, trifluoromethanesulfonate, and the like in a polar aprotic solvent such as DMF with a suitable base such as diisopropylethylamine [“(iPr)₂Net”] to give compound (3), which are compounds of Formula I, wherein Q is O or N(R⁵), respectively, wherein R³, R⁴, and R⁵ are as defined above.

Another synthesis of a compound of Formula I is outlined below in Scheme 73. In Scheme 73, compound (1), which is a compound of Formula I wherein R⁴ is hydrogen, such as the compounds of formulas (7) or (8) from Scheme 70, the compound of formula (3) from Scheme 71 wherein R⁴ is H, or the compound of formula (3) from Scheme 72 wherein R⁴ is H, is alkylated with a suitable alkylating agent such as a C₁-C₆ alkyl halide (e.g., methyl iodide) in the presence of a suitable base such as potassium carbonate and suitable polar solvent such as DMF to give a compound of formula (2), which is a compound of Formula I wherein R⁴ is C₁-C₆ alkyl.

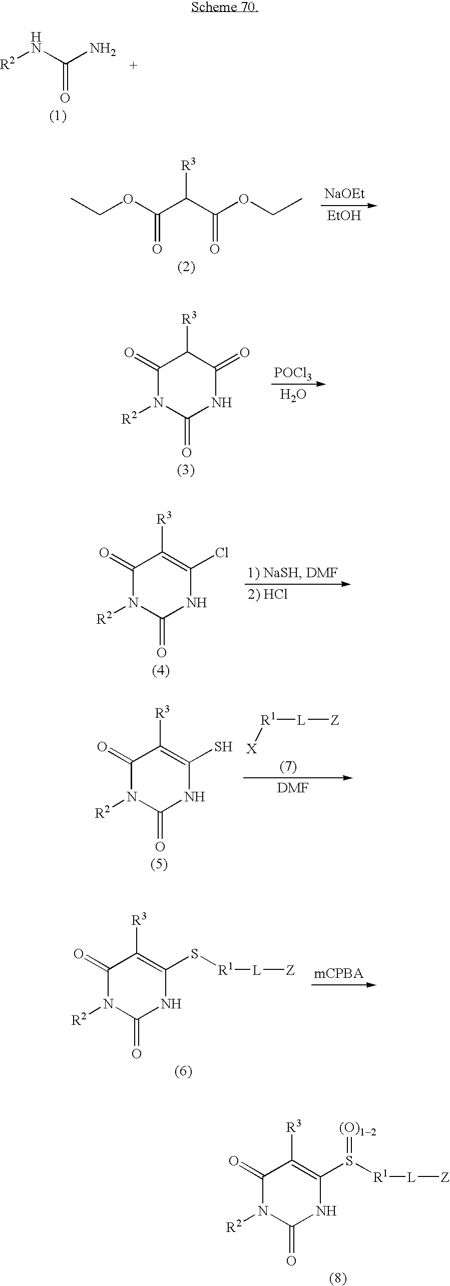

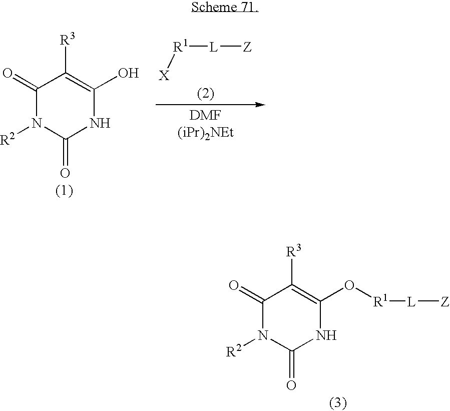

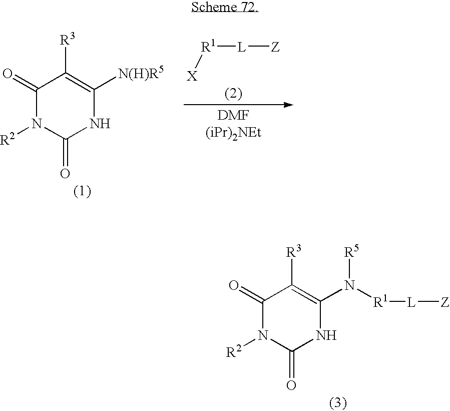

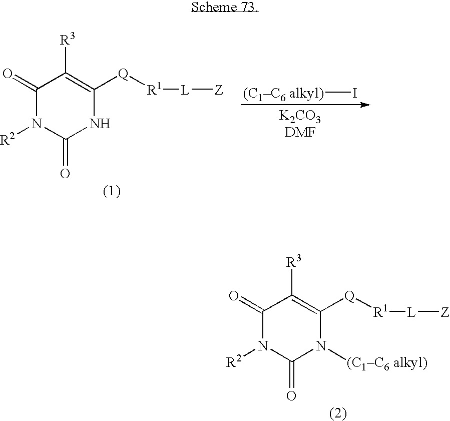

Additional methods of preparing compounds of Formula I are shown below in Schemes 74 to 78. In Scheme 74, the bromo-substituted phthalimide of formula (1) is treated with an appropriately substituted alkylating agent of formula R²—X, wherein R² is as defined above for Formula I and X is a suitable leaving group such as bromo, chloro, iodo, para-toluenesulfonate, acetate, trifluoroacetate, and the like, in the presence of a base, such as cesium carbonate, and in an aprotic solvent such as dimethylformamide (“DMF”). Compound (2) can be carbonylated to give ester (3) in the presence of a palladium(II) catalyst such as palladium(II) chloride 1,1′-bis(diphenylphosphino)ferrocene [“PdCl₂(dppf)”] and methanol under 500 psi of carbon monoxide. The carboxylic acid ester (3) can be converted to a variety of amide derivatives (4) in the presence of trimethyl aluminum and an appropriately substituted amine of formula Z—L—R¹ (R⁶)NH, wherein Z, L, R¹, and R⁶ are as defined above for Formula I, in tetrahydrofuran (“THF”). Alternatively, compound (2) can undergo palladium-catalyzed cross coupling with appropriately substituted alkynes of formula Z—L—R¹C≡CH, wherein Z, L, and R¹ are as defined above for Formula I, in the presence of a base such as diisopropylethylamine and catalyzed by bis(triphenylphosphine) palladium(II)dichloride in DMF to give the corresponding alkyne derivative (5).

In Scheme 75, a nitrile of formula (6) is reduced with hydrogen gas at 500 pounds-per-square-inch (“psi”) pressure in the presence of a catalyst such as Raney nickel in methanol and ammonia, for example, to give the aminomethyl (7). Compound (7) may be alkylated with a compound of formula R²—X as described above in Scheme 74 to give compound (8). Compound (8) may be carbonylated in a manner similar to that described above for Scheme 74 to give an intermediate which cyclizes in situ to a compound (9). Compound (9) may be converted to compound (10) by demethylation with borontribromide in dichloromethane followed by acylation of the intermediate phenol derivative with trifluoromethanesulfonic acid anhydride (“TFMSAA”). Compound (10) may be converted to compound (11) in a manner similar to that described above for Scheme 74. Alternatively, compound (11) may be converted to compound (12) by carbonylation in a manner similar to that described above for Scheme 74. Compound (12) may be converted to amide (13) in a manner similar to that described above for Scheme 74.

In Scheme 76, compound (14) may be coupled with a compound of formula R²—X, wherein R² is as defined above for Formula I and X is as defined above for Scheme 74, in a manner similar to that described above for Scheme 74 to give (15). Compound (15) may be converted to ester (16), and ester (16) coupled with a compound of formula Z—L—R¹(R⁶)NH, wherein Z, L, R¹, and R⁶ are as defined above for Formula I, in a manner similar to that described above for Scheme 74. Alternatively, compound (15) may be coupled with an alkyne of formula Z—L—R¹—C≡CH, wherein Z, L, and R¹ are as defined above for Formula I, in a manner similar to that described above for Scheme 74 to give the alkyne (18).

In Scheme 77, compound (19) may be coupled with a compound of formula R²—X, wherein R² is as defined above for Formula I and X is as defined above for Scheme 74, in a manner similar to that described above for Scheme 74 to give (20). Compound (20) may be converted to compound (21) with TFMSAA as described above for Scheme 75. Compound (21) may be coupled with an alkyne of formula Z—L—R¹—C≡CH, wherein Z, L, R¹ are as defined above for Formula I, in a manner similar to that described above for Scheme 75, to give the alkyne (22). Alternatively, compound (21) may be carbonylated to give ester (23), and ester (23) coupled with an amine of formula Z—L—R¹(R⁶)NH, wherein Z, L, R¹, and R⁶ are as defined above for Formula I, in a manner similar to that described above for Scheme 75, to give amide (24).

The chemistry of Scheme 78 is similar to the chemistry of Scheme 77.

Not shown in Schemes 74 to 78 is an alternative preparation of compounds of Formula I wherein Q is OC(O). The esters of formulas (3), (12), (16), (23), and (29), respectively, may be hydrolyzed to the corresponding carboxylic acids, which may be esterified with alcohols of formula Z—L—R¹OH, wherein Z, L, and R¹ are as defined above for Formula I. Other alternative preparations are described below.

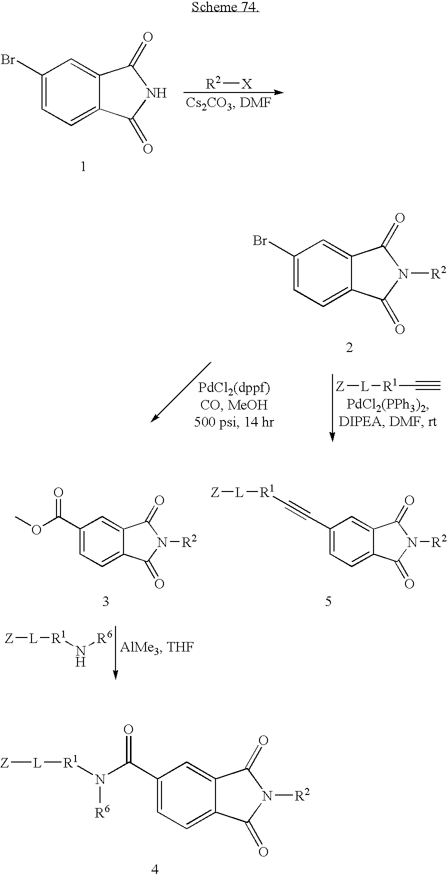

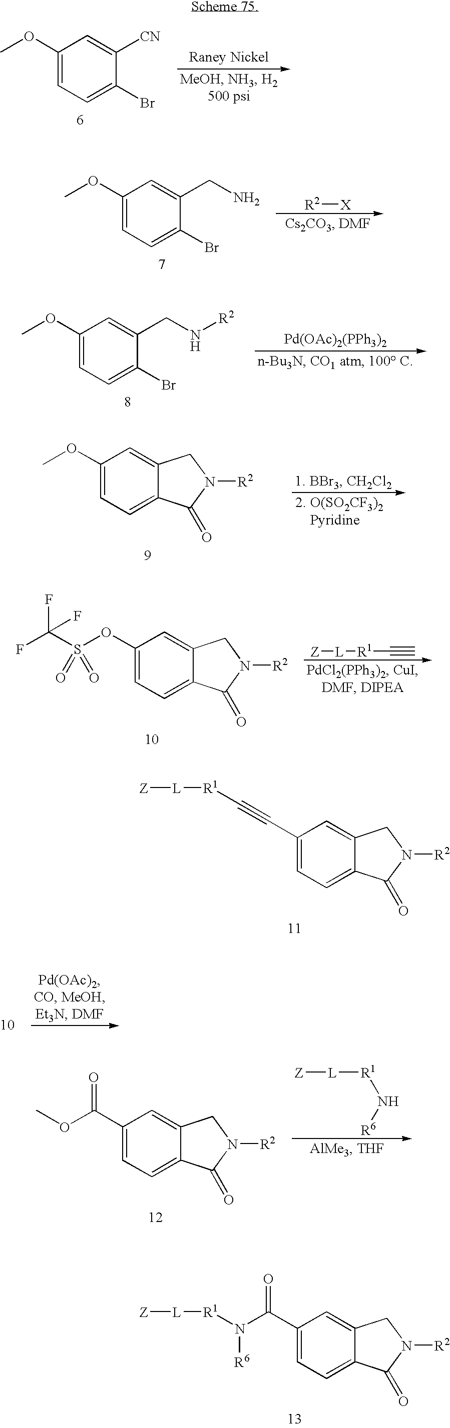

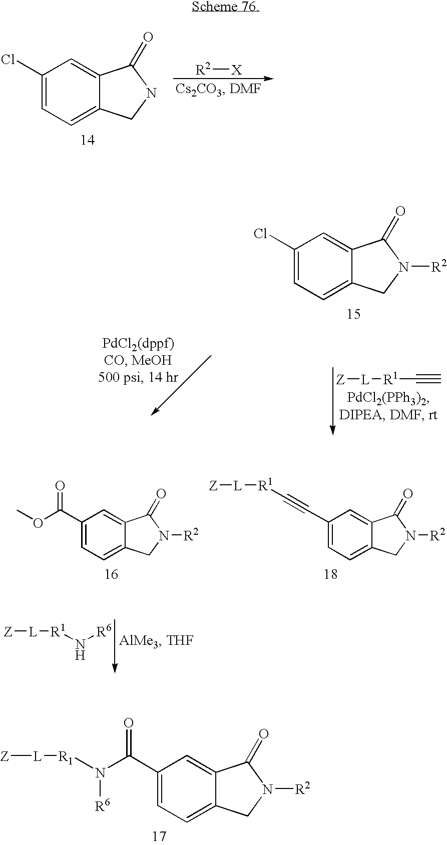

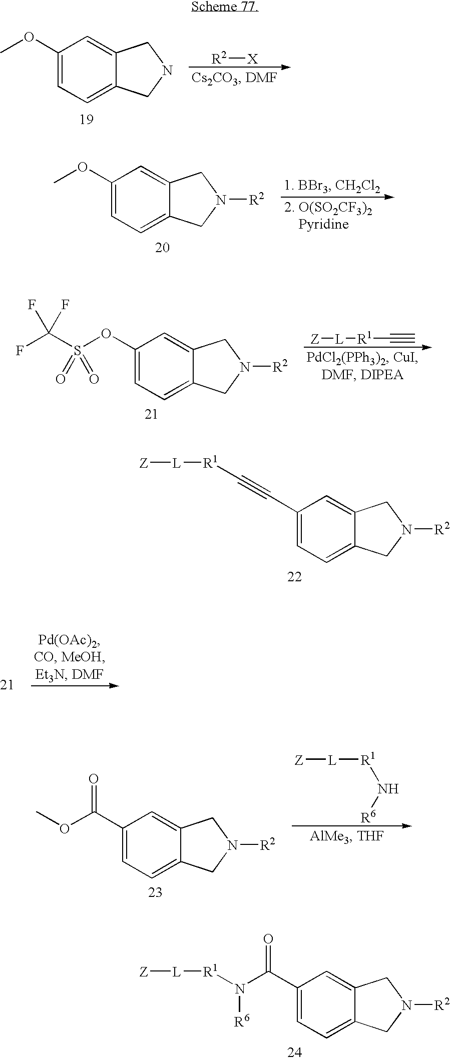

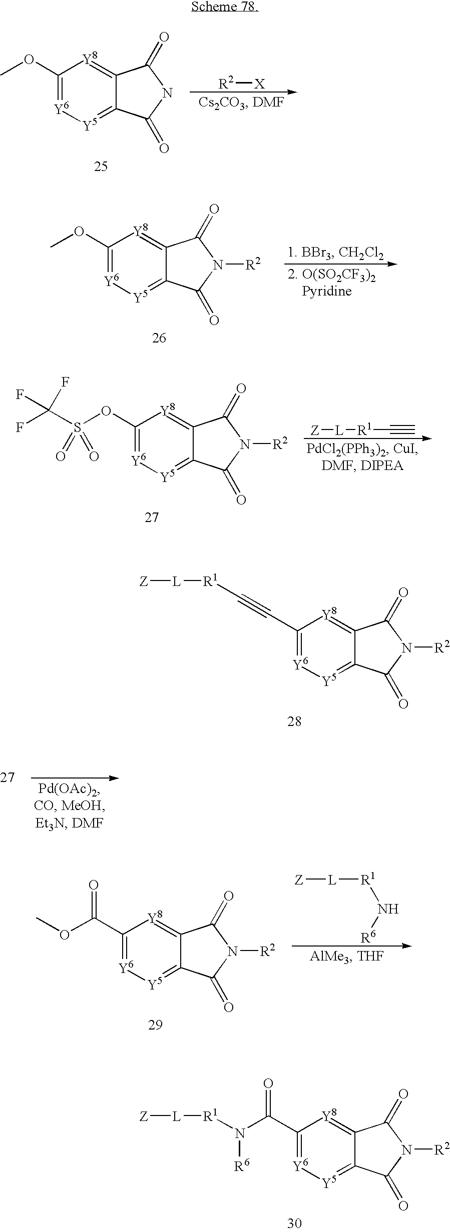

The following reaction Schemes 79 to 81 illustrate the preparation of the compounds of invention Embodiment 99.

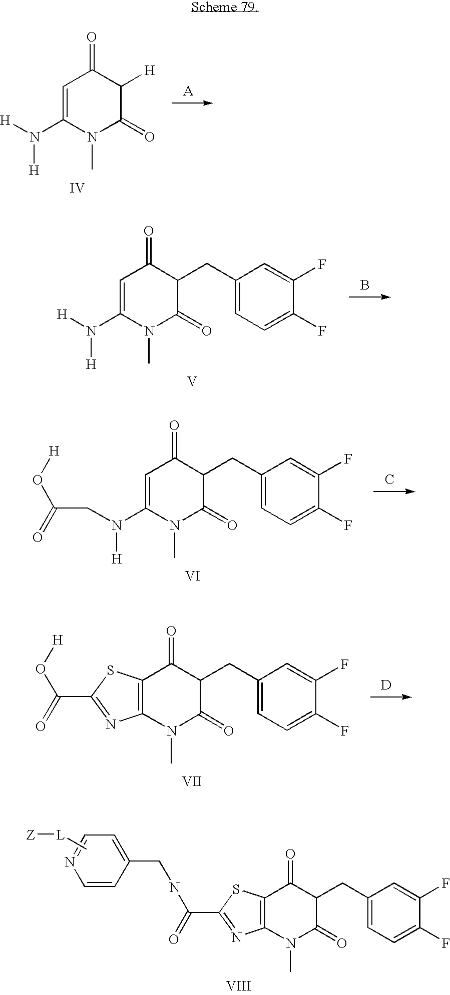

Scheme 79 refers to the preparation of compounds of Embodiment 99 in a four step process from compounds of formula IV. Referring to scheme 79, compound IV is treated with a suitable strong base, such as NaH, in a suitable protic solvent, e.g., THF or alcohol (e.g., ethanol), and preferably THF, followed by the addition of 3,4-difluorobenzyl bromide or another substituted benzyl bromide to yield compound V. This reaction is conducted at a temperature from about 20° C. to about 90° C., preferably about 50° C. to about 65° C. for a time period between about 15 minutes to about 16 hours.

Reaction of compound V with glycolic acid in the presence of sodium cyanoborohydride or another suitable reducing agent provides the N-alkyl analog VI. This reaction is carried out at a temperature from about 20° C. to about 90° C., preferably about 50° C. to about 65° C. for a time period between about 15 minutes to about 16 hours.

Compound VI is treated with thionyl chloride in pyridine at room temperature to provide the 6-(3,4-Difluoro-benzyl)-4-methyl-5,7-dioxo-4,5,6,7-tetrahydro-thiazolo[5,4-b]pyridine-2-carboxylic acid, compound VII, for a time period between about 15 minutes to about 16 hours.

Finally, coupling of compound VII with 4-aminomethyl-2-(or 3-)-(Z—L)-pyridine, e.g., using a suitable coupling agent, such as EDAC HCl provides Z—L-{6-(3,4-Difluoro-benzyl)-4-methyl-5,7-dioxo-4,5,6,7-tetrahydro-thiazolo[5,4-b]pyridine-2-carboxylic acid (pyridin-4-ylmethyl)-amide}, VIII. This reaction is carried out at a temperature from about 20° C. to about 90° C., preferably about 50° C. to about 65° C. for a time period between about 15 minutes to about 16 hours.

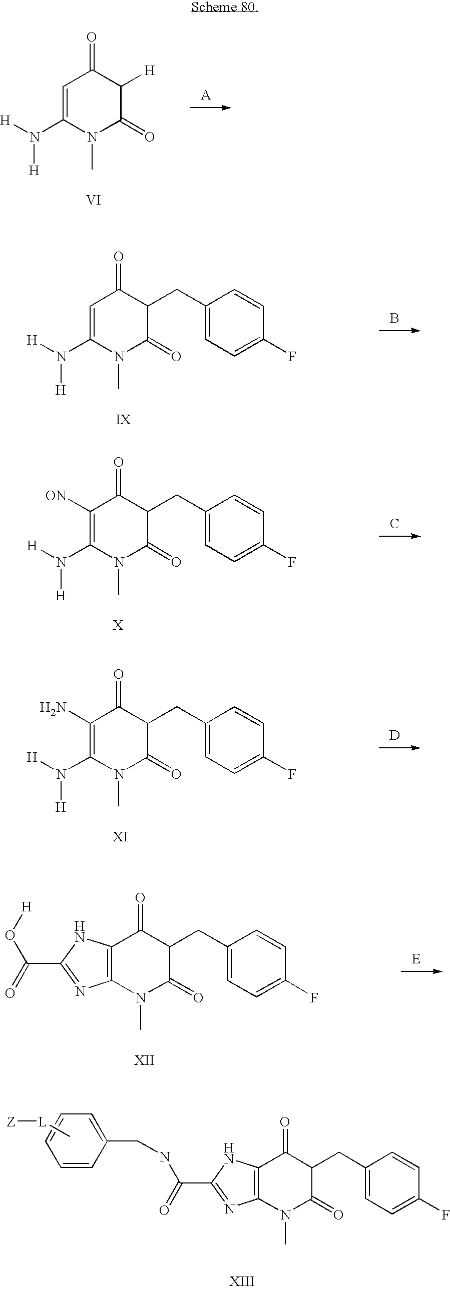

Referring to Scheme 80, which provides an alternate synthesis of compounds of Embodiment 99, compound IV is first treated with a suitable strong base, such as NaH, in a suitable protic solvent, such as THF or an alcohol, preferably THF, followed by 4-difluorobenzyl bromide or another substituted benzyl bromide to give compound IX. This reaction is carried out at a temperature from about 20° C. to about 90° C., preferably about 50° C. to about 65° C. for a time period between about 15 minutes to about 16 hours.

Reaction of IX with sodium nitrite in a weak acid, such as acetic acid, provides X which is immediately reduced to XI with 5% Pd/C in the presence of hydrogen.

Treatment of XI with oxalyl chloride in a suitable protic solvent, such as THF, followed by aqueous workup provides the acid XII.

Acid XII undergoes an EDAC HCl mediated coupling with 3-(or 4-)-(Z—L)-benzyl bromide to provide Z—L-{6-(4-fluoro-benzyl)-4-methyl-5,7-dioxo-4,5,6,7-tetrahydro-3H-imidazo[4,5-b]pyridine-2-carboxylic acid (pyridin-4-ylmethyl)-amide} XIII.

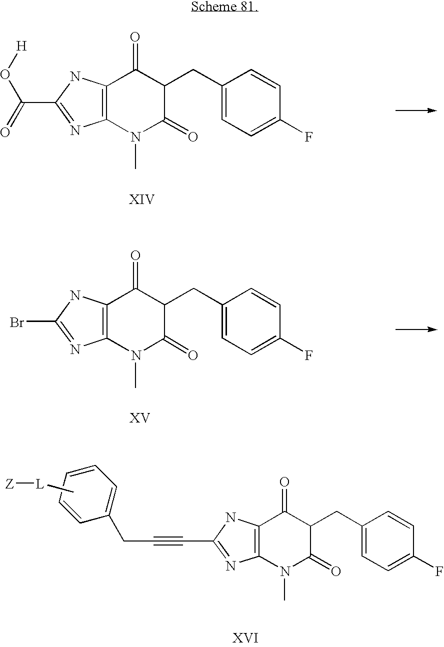

Finally, Scheme 81 provides another alternative synthesis of compounds of Embodiment 99. First, the carboxylic acid silver salt of compound XV is treated with bromine under Hunsdieker conditions to provide the iodide intermediate XVI.

Intermediate XVI in DMF is treated with di-isopropyl ethylamine, bis-triphenyl phosphine palladium dichloride (catalytic), copper (I) iodide (catalytic) and 3-[3-(or 4-)-(Z—L)-phenyl]-1-propyne. Heating the mixture at 80° C. for 6 hours provides Z—L-{6-(4-Fluoro-benzyl)-4-methyl-2-(3-phenyl-prop-1-ynyl)-1,4-dihydroimidazo[4,5b]pyridine-5,7-dione} XVII after chromatographic purification.

Compounds of formula IV and XV are commercially available or can be made by methods well known to those skilled in the art.

The following reaction Schemes 82 and 83 illustrate the preparation of the compounds of Embodiment 100.

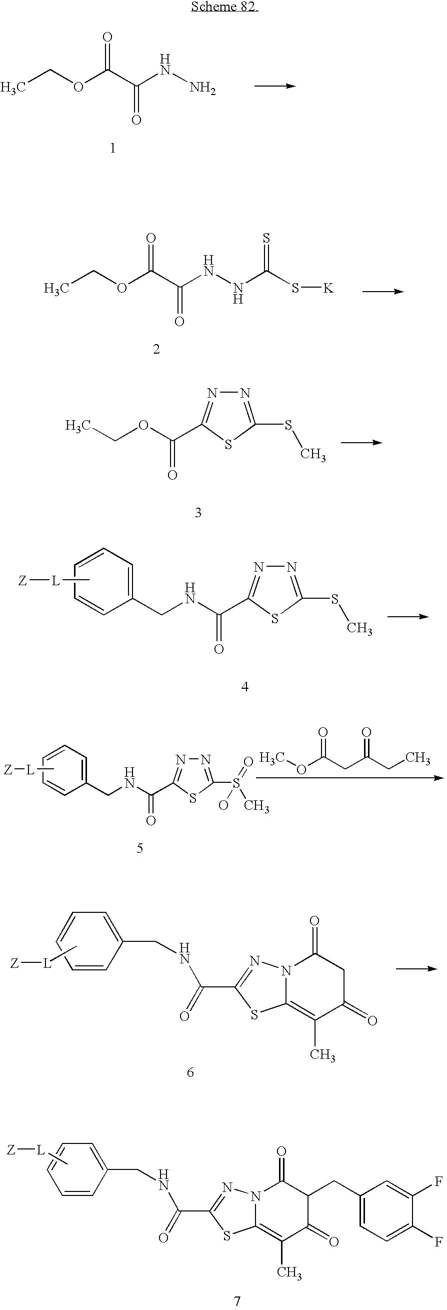

Scheme 82 shows a synthetic method that may be used to prepare compounds of Embodiment 100. First, hydrazino-oxo-acetic acid ethyl ester 1 (prepared by the method of Berdinskii, I. S.; Maslivets, A. N. Hydrazides of disubstituted glycolic acids. Zh. Org. Khim. (1982), 18(9), 1839-43) is treated with carbon disulfide (excess) and KOH (1.0 equiv.) in ethanol at 0° C. to give 2. Treatment of 2 with cold (excess) sulfuric acid for 20 minutes followed by heating to 55° C. for 1 hour gives 5-mercapto-[1,3,4]-thiadiazole-2-carboxylic acid ethyl ester which is then treated with ethyl iodide and potassium carbonate in ethanol/water at room temperature to give 3. Heating compound 4 in the presence of 3-(or 4-)-(Z—L)-benzylamine gives the amide, which is oxidized to the sulfone 5 with m-chloroperbenzoic acid (2.5 equiv) in chloroform or methylene chloride at room temperature. Treatment of 5 with 3-oxo-pentanoic acid methyl ester and excess sodium hydride in refluxing tert-butanol for 48 hours gives 6 after flash silica purification.

Treatment of 6 with sodium hydride followed by 3,4-di-fluoro benzyl bromide (or other substituted benzyl bromides) in THF gives Z—L-{6-(3,4-Difluoro-benzyl)-8-methyl-5,7-dioxo-6,7-dihydro-5H-[1,3,4]thiadiazolo[3,2-a]pyridine-2-carboxylic acid benzylamide} 7 after purification by silica gel chromatography.

With respect to Scheme 82, reference is made to Nuesslein, et al., 1,3,4-Thiadiazole-2-carboxylic acid derivatives for use in fungicides and nematicides, Ger. Offen. (1980), 51 pp.; Thiel, et al., 1,3,4-Thiadiazoles by reaction of dithiocarboxylic esters with carbonic hydrazides, J. Prakt. Chem. (1990), 332(1), 55-64; and Toyooka, et al., Synthesis of 2-Substituted 5-(1-Oxido-4-pyridyl) and 5-(1-Oxidp-2-pyridyl)-1,3,4-thiadiazole Derivatives by Substitution of 2-Methylsulfonyl Group with Various Nucleophiles, Chem. Pharm. Bull. (1987), 35(3), 1030-1035; which are incorporated herein by reference in their entirety.

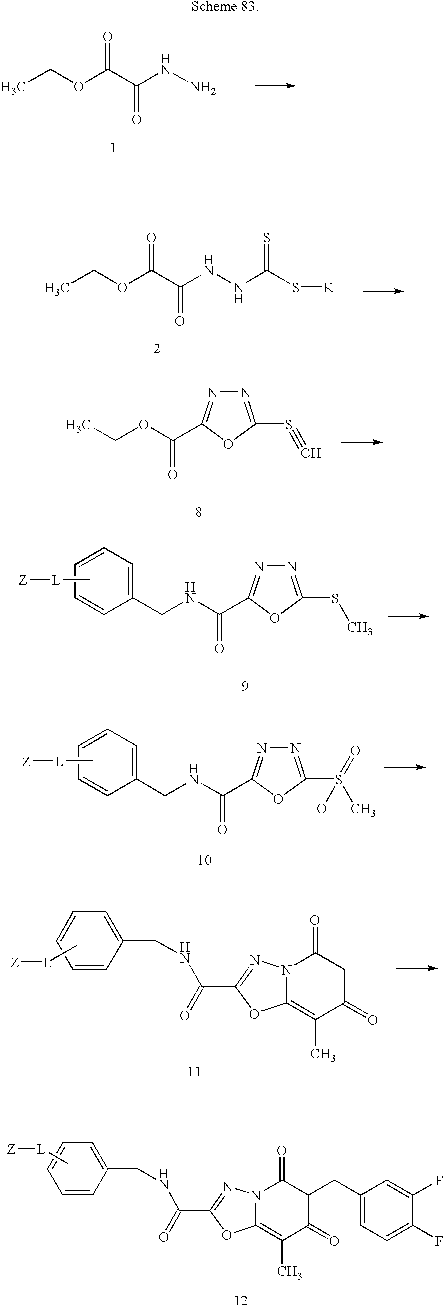

Scheme 83 represents an alternative method of synthesizing compounds of Embodiment 100. Briefly, hydrazino-oxo-acetic acid ethyl ester 1 is treated with carbon disulfide and KOH in ethanol to give 2.

Heating 2 in pyridine gives 5-methylsulfanyl-[1,3,4]oxadiazole-2-carboxylic acid ethyl ester which is treated with methyl iodide to give 8.

Heating compound 8 in the presence of 3-(or 4-)-(Z—L)-benzylamine gives the amide which is oxidized to the sulfone 10 with m-chloroperbenzoic acid. Treatment of 10 with 3-oxo-pentanoic acid methyl ester and excess sodium hydride in refluxing tert-butanol gives 11 after flash silica purification. Treatment of 11 with sodium hydride followed by 3,4-di-fluoro benzyl bromide (or other substituted benzyl bromides) in THF gives Z—L-{6-(3,4-Difluoro-benzyl)-8-methyl-5,7-dioxo-6,7-dihydro-5H-[1,3,4]oxadiazolo[3,2-a] pyridine-2-carboxylic acid benzylamide} 12.

With respect to Scheme 83, reference is made to Baron, et al., 2-Substituted-1,3,4-oxa- and thia-diazoline-5-thiones, J. Org. Chem (1959) 23, 1021-1023; and Partyka, et al., 1,3,4-Oxadiazole amides, U.S. (1977), 8 pp.; which are incorporated herein by reference in their entirety.

The following reaction Schemes 84 to 87 illustrate the preparation of the compounds of Embodiment 101.

Scheme 84 represents a method of synthesizing compounds of Embodiment 101. Briefly, compound IV, which is prepared as described in Pyrimidines, Part XXXI [1]. Synthesis, Reactions and Properties of 6-Acyl-1,3-dimethylpyrrolo[2,3-d]pyrimidine-2,4-diones. Salih, Z. S. J. Het. Chem., 25, 14413 (1988); and Uber 2,4,6-trioxo-hexahydropteridine und die homologen 7-methyl-derivate. Pfleider, W. Chem Ber. 90, 2604, (1957), each of which are incorporated herein by reference, is reacted with POCl₃ (1.1 equiv.) and DMF 1.1 equiv.) in methylene chloride from 0° C. to reflux for 5 hours to give the formylated product V.

Reaction of V with potassium carbonate (1.5 equiv.) in ethanol at reflux for 18 hours provides 3-(3,4-difluoro-benzyl)-1-methyl-2,4-dioxo-2,3,4,7-tetrahydro-1H-pyrrolo[2,3-d]pyrimidine-6-carboxylic acid ethyl ester, compound VI. This material is treated with sodium hydroxide (1.2 equiv) in methanol/water (1:1) mixture at room temperature for 18 hours to give the acid.

EDAC HCl (1.3 equiv) coupling of VII in DMF with 3-(or 4-)-(Z—L)-benzylamine (1.3 equiv) and HOBT (1.3 equiv) at room temperature for 18 hours provided Z—L-{3-(3,4-Difluorobenzyl)-1-methyl-2,4-dioxo-2,3,4,7-tetrahydro-1H-pyrrolo[2,3-d]pyrimidine-6-carboxylic acid benzylamide} VIII.

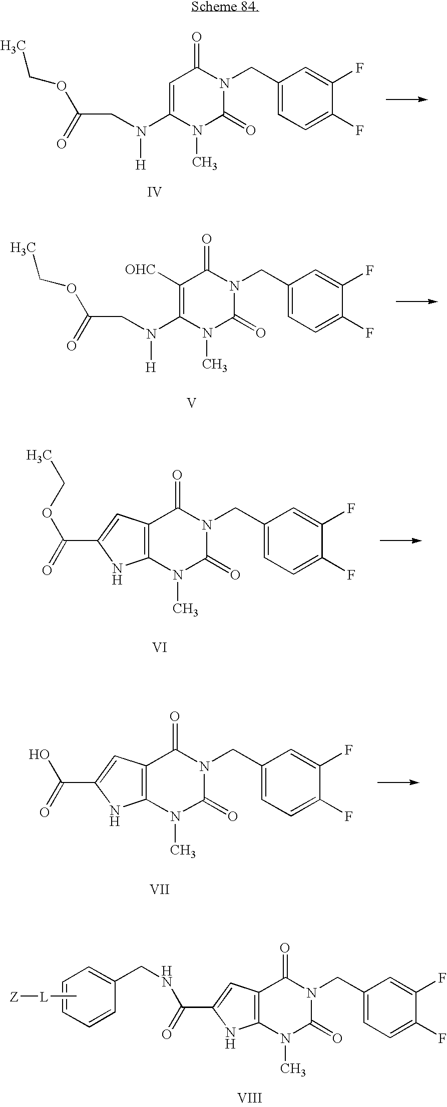

Scheme 85 represents an alternative method of synthesizing compounds of formula I-III. Briefly, compound IX, which is prepared as described in Kazimierczuk, et al., Intermediates in the synthesis of purines and pteridines: N-methylated 6-chlorouracils, Acta Biochim. Pol. (1970), 17(4), 325-9; Pfleider, et al., Liebigs Ann. Chem, 612, 158, (1958); and Hirota, et al., Pyrimidines. 65. Synthesis of 6-substituted thieno[2,3-d]pyrimidine-2,4(1H,3H)-diones, J. Heterocycl. Chem. (1990), 27(3), 717-21; each of which are incorporated herein by reference, is reacted with 3,4-di-fluorobenzyl bromide (1.1 equiv.) in DMF at room temperature for 18 hours in the presence of cesium carbonate 1.5 equiv.) to give product X.

Reaction of X (1.0 equiv.) with thiogylcolic acid ethyl ester (1.0 equiv.) in room temperature ethanol in the presence of triethyl amine (1.0 equiv.) for 1.5 hour provided [1-(3,4-Difluoro-benzyl)-3-methyl-2,6-dioxo-1,2,3,6-tetrahydro-pyrimidin-4-ylsulfanyl]-acetic acid ethyl ester, compound XI. This material is treated with POCl₃ (1.2 equiv.) and DMF (1.1 equiv.) in methylene chloride at 0° C. to reflux under Vilsmeier-Haack conditions to provide formylated product XII.

Heating this material in the presence of sodium carbonate (1.5 equiv.) in ethanol at reflux for 18 hours provides the cyclized product 3-(3,4-difluoro-benzyl)-1-methyl-2,4-dioxo-1,2,3,4-tetrahydro-thieno[2,3-d]pyrimidine-6-carboxylic acid ethyl ester XIII. Saponification of XIII with sodium hydroxide sodium hydroxide (1.2 equiv) in methanol/water (1:1) mixture at room temperature for 18 hours gives XIV. EDAC HCl (1.3 equiv.) coupling of XIV in DMF with 3-(or 4-)-(Z—L)-benzylamine (1.3 equiv.), and HOBT (1.3 equiv.) for 18 hours at room temperature provided Z—L-{3-(3,4-difluoro-benzyl)-1-methyl-2,4-dioxo-1,2,3,4-tetrahydro-thieno[2,3-d]pyrimidine-6-carboxylic acid benzylamide} XV.

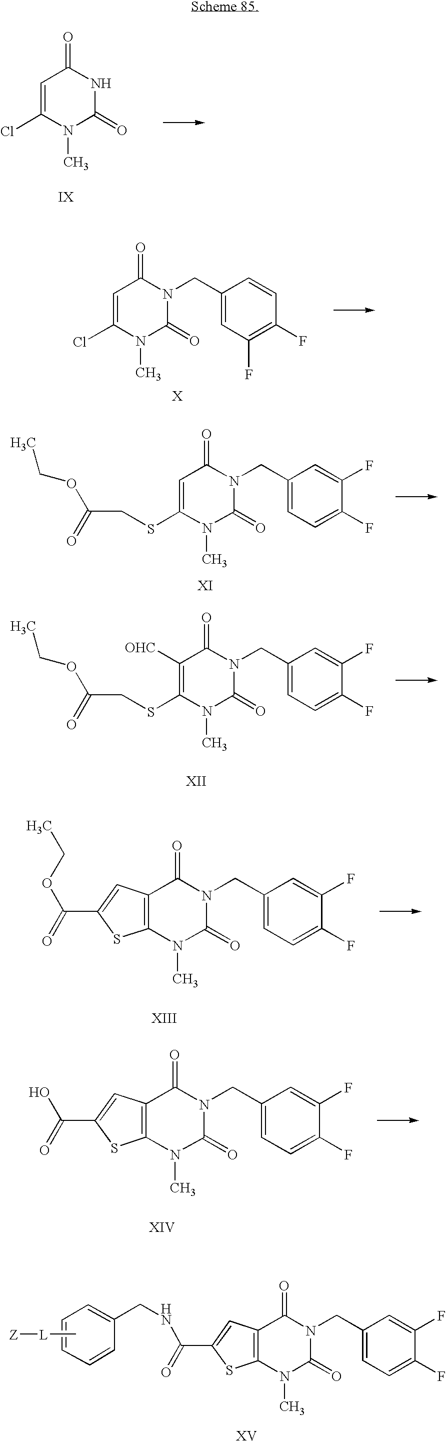

Scheme 86 represents yet another alternative synthesis of compounds of Embodiment 101. The carboxylic acid silver salt of V is treated with bromine under Hunsdiecker conditions (Hunsdiecker, H. C., Chem. Ber. (1942) 75, 291, which is incorporated herein by reference) to provide the bromide intermediate XVI.

Intermediate XVI in DMF was treated with di-isopropyl ethylamine, bis-triphenyl phosphine palladium dichloride (catalytic), copper (I) iodide (catalytic) and 3-[3-(or 4-)-(Z—L)-phenyl]-1-propyne. Heating the mixture at 80° C. for 6 hours provides Z—L-{3-(3,4-Difluoro-benzyl)-1-methyl-6-(3-phenyl-prop-1-ynyl)-1,7-dihydro-pyrrolo[2,3-d]pyrimidine-2,4-dione} XV after chromatographic purification (SiO₂, EtOAc/hexane eluent).

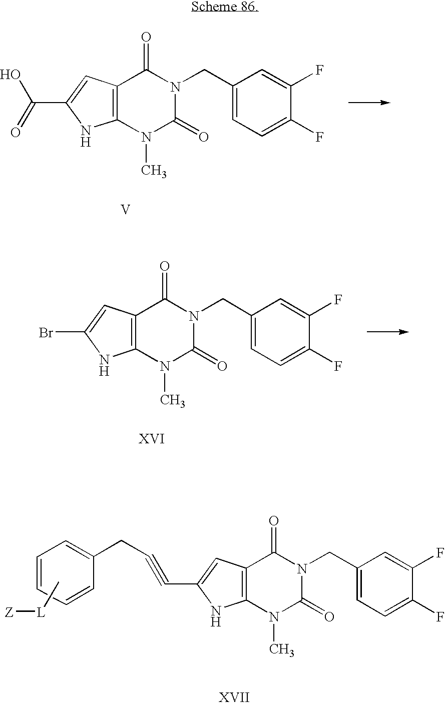

Scheme 87 represents still another alternative synthesis of compounds of Embodiment 101. Briefly, the carboxylic acid silver salt of XVI is treated with bromine under Hunsdiecker conditions to provide the bromide intermediate XVII.

Intermediate XVII in DMF was treated with di-isopropyl ethylamine, bis-triphenyl phosphine palladium dichloride (catalytic), copper (I) iodide (catalytic) and 3-[3-(or 4-)-(Z—L)-phenyl]-1-propyne. Heating the mixture at 80° C. for 6 hours provides Z—L-{3-(3,4-Difluoro-benzyl)-1-methyl-6-(3-phenyl-prop-1-ynyl)-1H-thieno[2,3-d]pyrimidine-2,4-dione} XVIII after chromatographic purification (SiO₂, EtOAc/hexane eluent).

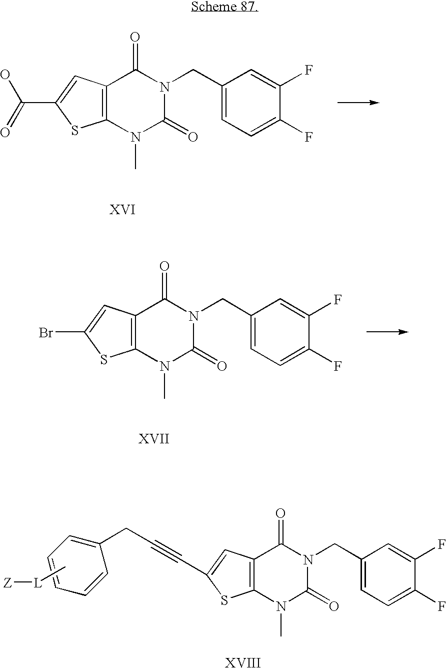

Compounds of Formula I may also be prepared utilizing the reaction conditions presented in Schemes 88 and 89. In Scheme 88, the nitrogen atom of the oxazine or thiazine heterocycle (1) can be alkylated with an appropriately substituted alkylating agent such as iodomethane in the presence of cesium carbonate in dimethylformamide (“DMF”) or tetrahydrofuran (“THF”). The halide (2) can be converted to the corresponding ester (3) in methanol/tetrahydrofuran under an atmosphere of carbon monoxide and catalyzed by bis(triphenylphosphine)Pd(II) dichloride, requiring reaction temperatures as high as 100° C. The oxazine or thiazine intermediate (3) undergo a condensation reaction with appropriately substituted aldehydes in refluxing acetic anhydride containing a suitable base such as triethylamine. The ester intermediate (4) may be coupled with a variety of amines using trimethylaluminum in dichloromethane to yield amide intermediate (5). The exocyclic double bond of (5) may be reduced to compound (6) in the presence of 10% palladium on carbon in methanolic tetrahydrofuran under an atmosphere of hydrogen.

In Scheme 89, similar reaction conditions could be used to convert the quinolinone starting material (7) to the corresponding amide (12). As described in Scheme 88, alkylation followed by carbonylation and coupling the resulting ester (9) with appropriately substituted amines should yield the amide intermediate (10). Deprotonation of (10) at −78° C. using lithium diisopropylamide (“LDA”) in THF followed by aldehyde condensation provides the exocyclic olefin (11). Hydrogenation of the exocyclic double bond of (11) using conditions previously described in Scheme 88 allows isolation of quinolinone derivative (12).

The synthesis of compounds of Formula I is also presented in Schemes 90a, 90b, and 90c. In Scheme 90a, the 2-chloro atom of compound (13) can be selectively displaced with alcohol or thiol (14) in the presence of a base such as triethylamine and a suitable solvent like acetone at reaction temperatures ranging between 0° C. and 25° C. Reduction of the nitro functionality with tin/HCl initiates intramolecular cyclization to compound (15) on warming.

Similar reaction conditions can be used to prepare compounds of Formula I as shown in Scheme 90b Replacement of compound (13) with (16) in Scheme 90a and using the reaction conditions previously described for (13) should allow the isolation of either the oxazine or thiazine derivatives (17).

However, as shown in Scheme 90b the synthesis of oxazines and thiazines of Formula I where Y⁵ is N, require separate starting materials. For the thiazine (21), the thiol (19) is coupled to the 3-position of (18) using a base such as sodium hydride in refluxing THF. Based catalyzed hydrolysis of (20) to the corresponding thiol followed by coupling with chloroacetic acid and cyclization in a suitable solvent such as THF to give thiazine (21).

In Scheme 90c, the oxazine (24) can be prepared from compound (22) using a similar strategy previously used for (21). Base catalyzed addition of (22) to chloroacetic acid followed by cyclization affords intermediate (22). Compound (22) can undergo halogenation using bromine in an aprotic solvent such as DMF to give compound (24).

The synthesis of naphthyridinone derivatives is presented in Schemes 91a, 91b, and 91c. In Scheme 91a, ortho-directed metallation of (25) in the presence of n-butyl lithium and tetramethylethylenediamine (“TMEDA”) in THF at −78° C. followed by electrophilic quench with N-formylmorpholine yields the aldehyde (26) derivative. Condensation of the aldehyde utilizing the Wittig reaction can allow isolation of the acrylate (27). Deprotection of the amine using trifluoroacetic acid (“TFA”) followed by base catalyzed cyclization should provide heterocycle (28). Hydrogenation of the lactam double bond in the presence of palladium on carbon yields the tetrahydronaphthyridinone (29).

In Scheme 91b, pyridine derivative (30) can be converted to the dihydronaphthyridinone (31) in one pot using palladium catalyzed coupling of methylacrylate with (30) in acetonitrile followed by base catalyzed cyclization to (31). Hydrogenation to the tetrahydro derivative (32) followed by nitration and subsequent reduction of the nitro functionality with Raney nickel in methanolic ammonia. Compound (33) can be diazotized and converted to the iodo derivative (34) in the presence of t-butylnitrite and copper(I)iodide in refluxing acetonitrile.

Similar reaction conditions are used as shown in Scheme 91c, methylacrylate coupling with compound (35) followed by base catalyzed cyclization and hydrogenation of the lactam double bond yields naphthyridinone (37).

Compounds of Formula I wherein Q is a triple bond can be prepared as shown in Schemes 92a and 92b. In Scheme 92a, halogen containing analogs (38) can be converted directly to the corresponding alkynes in one step using appropriately substituted acetylenes in a base such as diisopropylethylamine with copper(I)iodide and bis(triphenylphosphine)palladium(II)dichloride as the catalyst in DMF with the reaction temperature ranging between 40° C. and 110° C.

Alternatively in Scheme 92b, intermediates bearing a methoxy group in place of a halogen may be converted to the corresponding alcohol in the presence of BBr₃. The alcohol can be converted to the triflate (41) using triflic anhydride in pyridine. Using the same palladium catalyzed conditions previously described, triflate (41) can be converted to the corresponding alkyne derivatives.

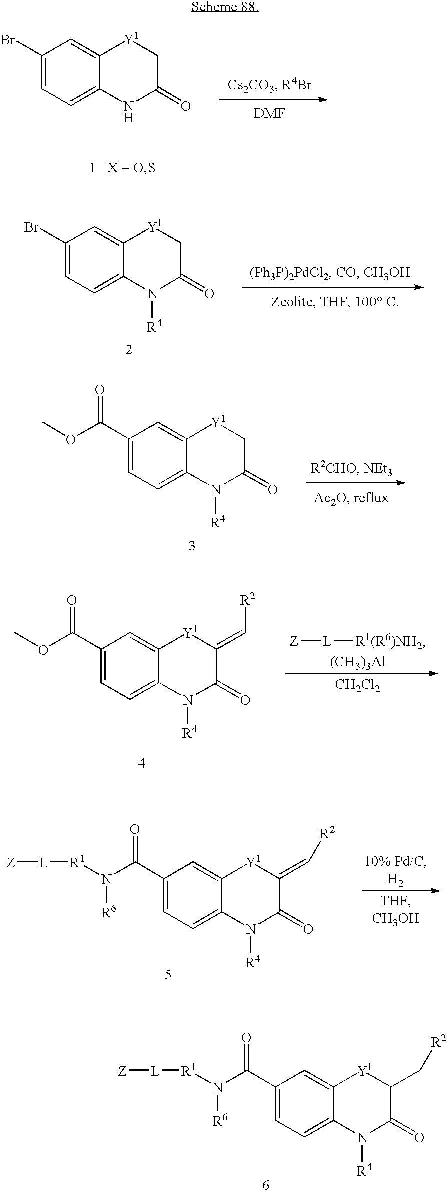

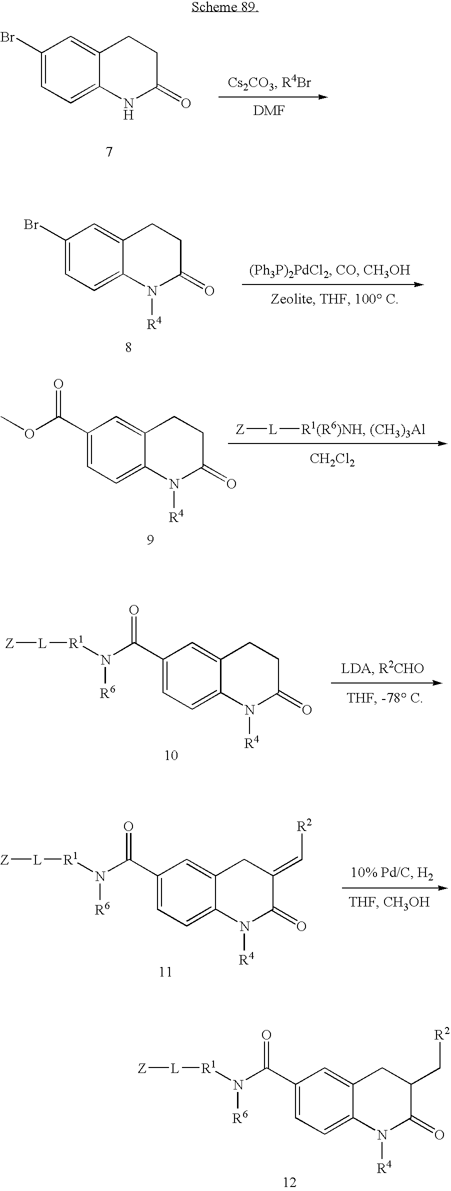

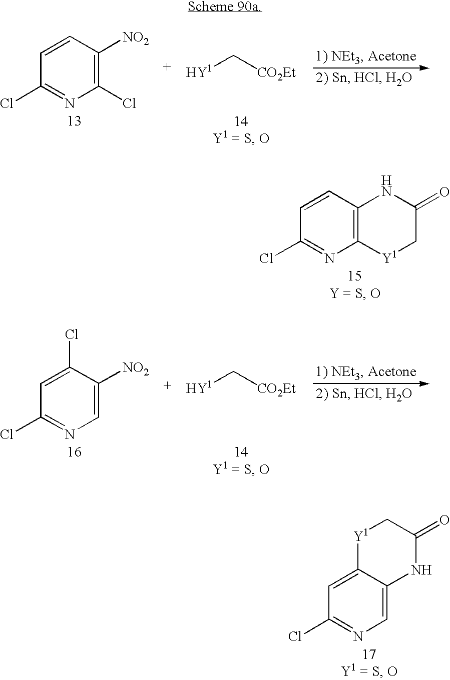

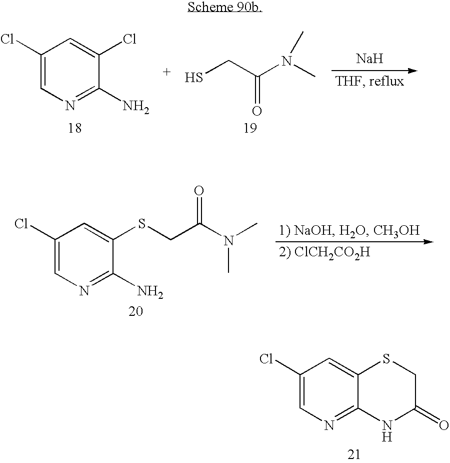

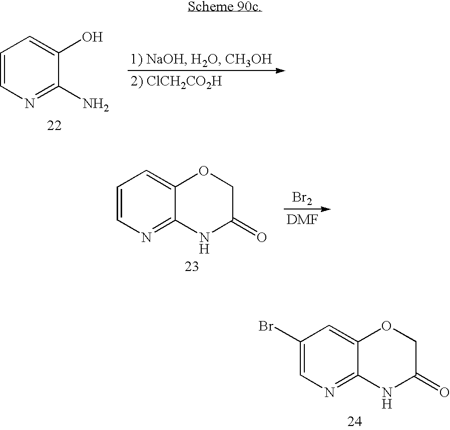

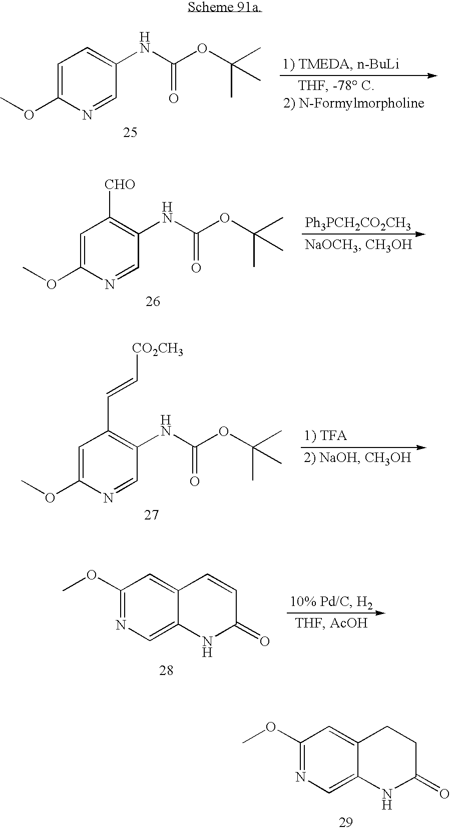

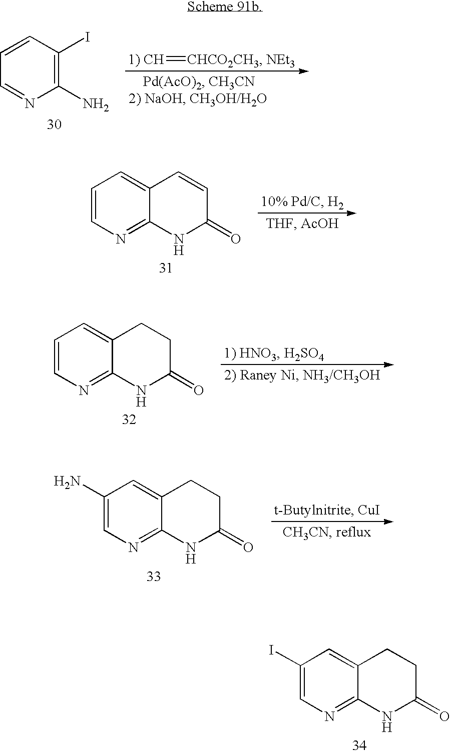

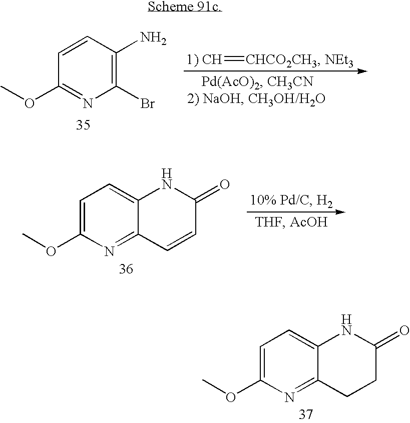

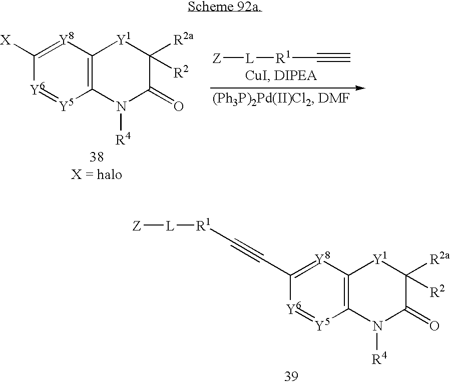

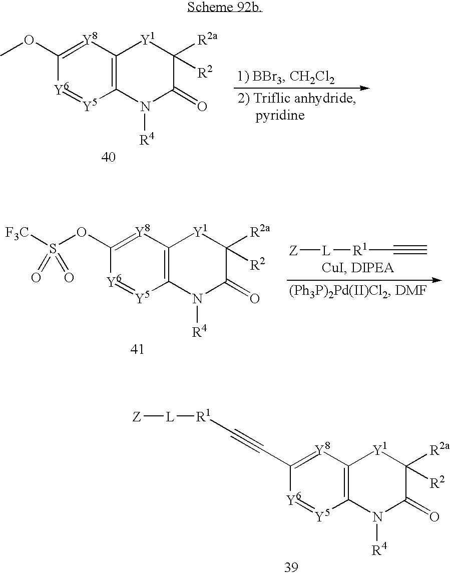

Alternatively, one of ordinary skill in the art of organic chemistry may prepare compounds of Formula I by adapting methods known in the art. For example, the skilled artisan may prepare compounds of Formula I wherein D is

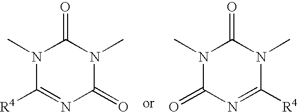

by adapting methods described or referenced in U.S. patent application Publication Ser. No. 2002049320 A1 and abstracted at Chemical Abstract number 2002:315486 (HCAPLUS file) and at Chemical Abstract number 136:340706 (CA file).

Further, the skilled artisan may prepare compounds of Formula I wherein D is

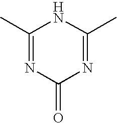

by adapting methods described or referenced in PCT International Application Publication number WO 2000/043369.

Further, the skilled artisan may prepare compounds of Formula I wherein D is

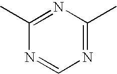

by adapting methods described or referenced in U.S. Pat. Nos. 3,770,717 and 3,507,864.

Further, the skilled artisan may prepare compounds of Formula I wherein D is

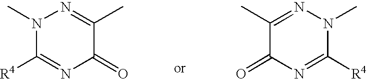

by adapting methods described or referenced in U.S. Pat. No. 6,294,668.

Further, the skilled artisan may prepare compounds of Formula I wherein D is

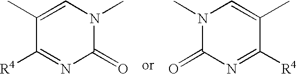

by adapting methods described or referenced in Canadian Patent number 2,228,875 AA and European Patent number 863 150 A1.

Further, the skilled artisan may prepare compounds of Formula I wherein D is

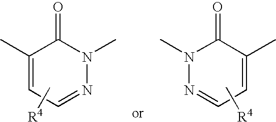

by adapting methods described or referenced in U.S. Pat. No. 5,019,587 A and European Patent Application Patent number 344 634 B1.

Further, the skilled artisan may prepare compounds of Formula I wherein D is

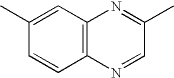

by adapting methods described or referenced in German Patent number 1,247320 and Great Britain Patent number 967890.

Further, the skilled artisan may prepare compounds of Formula I wherein D is

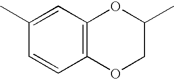

by adapting methods described or referenced in U.S. Pat. Nos. 5,965,607 and 6,020,361 and PCT International Patent Application Publication number WO 98/29405.

Further, the skilled artisan may prepare compounds of Formula I wherein D is

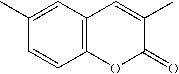

by adapting methods described or referenced in U.S. Pat. Nos. 4,317,774; 4,341,703; and 4,343,938.

Further, the skilled artisan may prepare compounds of Formula I wherein D is

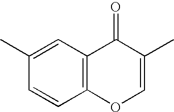

by adapting methods described or referenced in Japanese Patent numbers 57059884, 55111489, 62033235, and 54012392.

Further, the skilled artisan may prepare compounds of Formula I wherein D is

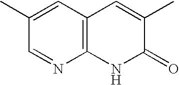

by adapting methods described or referenced in U.S. Pat. Nos. 4,324,893; and 4,350,817.

Further, the skilled artisan may prepare compounds of Formula I wherein D is

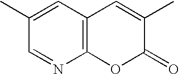

by adapting methods described or referenced in U.S. Pat. Nos. 5,510,319; 5,532,207; 5,534,485; 5,534,483; 5,534,484; 5,536,839; 5,622,913; 4,317,774; 4,341,703; and 4,343,938.

It should be appreciated that when Q is trans-(H)C═C(H), cis-(H)C═C(H), C≡C, CH₂C≡C, or CF₂C≡C and is bonded to a sp² carbon atom in D, a palladium catalyzed coupling of the corresponding terminal olefin or alkyne of formulas R¹-(trans-(H)C═CH₂), R¹-(cis-(H)C═CH₂), R¹—C≡CH, R¹—CH₂C≡CH, or R¹—CF₂C≡CH, wherein R¹ is as defined above, with a bromo- or iodo-substituted sp² carbon atom of formula:

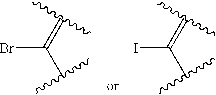

in the presence of a suitable base will yield a compound of Formula I wherein Q is trans-(H)C═C(H), cis-(H)C═C(H), C≡C, CH₂C≡C, or CF₂C≡C and D is a group that is bonded to Q at a sp² carbon atom, and R¹, V, and R² are as defined above for Formula I. Illustrative examples of the coupling reagents and catalysts include palladium tetrakis(triphenylphosphine) or palladium(II) acetate as catalyst, a tertiary organic amine base such as triethylamine or diisopropylethylamine, a suitable solvent such as dimethylformamide (“DMF”) or tetrahydrofuran (“THF”), and optionally a co-catalyst such as copper(I)iodide, at a suitable temperature such as from 0° C. to 100° C., for a suitable time such as from 30 minutes to 2 days, and under an inert atmosphere such as an atmosphere of nitrogen or argon gas.

Alternatively, a corresponding aldehyde of formula

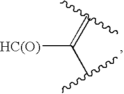

prepared as described below, may be coupled with a phosphonium ylide under Wittig olefination, or Horner-Emmons olefination, conditions to give a compound of Formula I wherein Q is trans-(H)C═C(H).

The bromo or iodo intermediates described above may be converted by conventional means to the corresponding carboxylic acid of formula

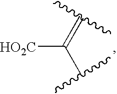

and the carboxylic acid converted by conventional means to compounds of Formula I wherein Q is OC(O), CH(R⁶)C(O), OC(NR⁶), CH(R⁶)C(NR⁶), N(R⁶)C(O), N(R⁶)C(S), N(R⁶)C(NR⁶), SC(O), CH(R⁶)C(S), or SC(NR⁶). Illustrative examples include coupling of the carboxylic acid with an amine to provide a compound of Formula I wherein Q is N(R⁶)C(O), and optionally sulfurating the resulting amide with, for example P₂S₅ to provide a compound of Formula I wherein Q is N(R⁶)C(S). Alternatively, the carboxylic acid may be coupled with an alcohol to provide a compound of Formula I wherein Q is OC(O).

Alternatively, the carboxylic acid may be reduced to the corresponding hydroxymethyl compound of formula

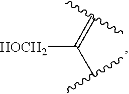

and the hydroxymethyl converted to a compound of Formula I wherein Q is OCH₂ or N(R⁶)CH₂ by conventional means.

Alternatively, the hydroxymethyl compound may be oxidized to the corresponding aldehyde of formula

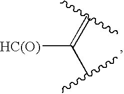

and the aldehyde coupled with hydroxylamine to give a corresponding oxime. The oxime may be chlorinated, and the chlorooxime cyclized with an olefin or alkyne to give a compound of Formula I wherein Q is a 5-membered heteroarylene.

Alternatively, the aldehyde may be prepared from the corresponding carboxylic acid by coupling the carboxylic acid with N,O-dimethylhydroxylamine and reducing the resulting dimethylhydroxamide with a suitable hydride reducing agent such as sodium borohydride or lithium aluminum hydride.

Alternatively, the above-described carboxylic acid intermediate may be converted by conventional means to the corresponding methyl ketone of formula

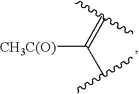

and the methyl ketone may be halogenated on methyl and coupled with various amines, alcohols, or other halogenated compounds to give a compound of Formula I wherein Q is CH(R⁶)C(O).

Alternatively, the above-described carboxylic acid intermediate or bromo- or iodo-intermediates may be converted by conventional means to the corresponding nitrile of formula

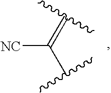

and the nitrile condensed with an amine or alcohol under non-nucleophilic basic conditions (e.g., 1,8-diazaundecane) to give a compound of Formula I wherein Q is N(R⁶)C(NR⁶) or OC(NR⁶), respectively.

Alternatively, compounds of Formula I wherein Q is a lactam diradical may be prepared by conventional means by cyclizing the corresponding gamma-amino acids.

In accordance with the above synthetic methods, the following invention compounds may be prepared.

### EXAMPLE 1

4-(3-{3-[3-(3,4-Difluoro-benzyl)-4-oxo-3,4-dihydro-quinazolin-6-yl]-prop-2-ynyl}-phenyl)-butyric acid

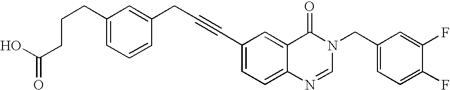

### EXAMPLE 2

5-(3,4-Difluoro-benzyl)-7-methyl-4,6-dioxo-3a,4,5,6-tetrahydro-thieno[3,2-c]pyridine-2-carboxylic acid [2-(3-mercapto-propoxy)-pyridin-4-ylmethyl]-amide

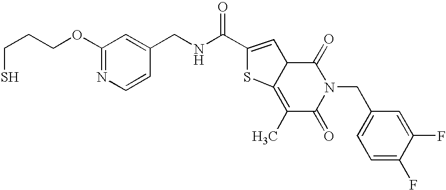

### EXAMPLE 3

4-(6-{2-[3-(2-Hydroxycarbamoyl-ethoxy)-phenyl]-oxazol-5-yl}-4-oxo-4H-quinazolin-3-ylmethyl)-benzoic acid

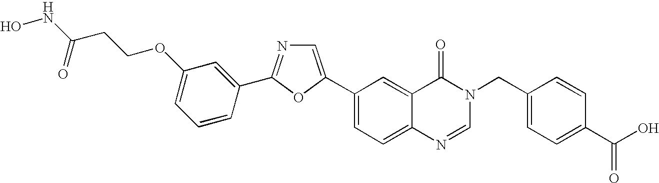

The compounds of Formula I can be evaluated in standard assays for their ability to inhibit the catalytic activity of MMP enzymes. The assays used to evaluate the MMP biological activity of the invention compounds are well-known and routinely used by those skilled in the study of MMP inhibitors and their use to treat clinical conditions. For example, compounds of Formula I may be readily identified by assaying a test compound for inhibition of MMP-13 according to Biological Methods 1 or 2, and further assaying the test compound for allosteric inhibition of MMP-13 according to Biological Methods 3 or 4, as described below.

The compounds of Formula I will be shown to be potent inhibitors of MMP-13 catalytic domain (“MMP-13CD”) or full length enzyme (“MMP-13FL”). Potencies, as measured by IC₅₀'s, with MMP-13 catalytic domain for the invention compounds will typically range from about 0.001 μM to about 30 μM.

Invention compounds can be further screened with full-length MMP-2, full-length MMP-7, full-length MMP-9, and MMP-14 catalytic domain to determine selectivity of the inhibitors with MMP-13 versus the other MMP enzymes also. Selectivities of the invention compounds for MMP-13 catalytic domain versus another MMP enzyme (full-length or catalytic domain), as determined by dividing the IC₅₀ for the inhibitor with a comparator MMP enzyme by the IC₅₀ of the inhibitor with MMP-13 catalytic domain, are expected to range from 5 to 50,000 fold.

To determine the inhibitory profiles, a compound of Formula I, or a pharmaceutically acceptable salt thereof, may be evaluated in standard assays for their ability to inhibit the catalytic activity of various MMP enzymes. The assays used to evaluate the MMP biological activity of the invention compounds are well-known and routinely used by those skilled in the study of MMP inhibitors and their use to treat clinical conditions. The compound of Formula I will be shown to be selective for inhibition of MMP-13CD versus MMP-1FL, MMP-2FL, MMP-3CD, MMP-7FL, MMP-9FL, MMP-12CD, and MMP-14CD with typical selectivity ranging between about 50 and about 500 fold, as measured by dividing the IC₅₀ of the compound of Formula I with MMP-1FL, MMP-2FL, MMP-3CD, MMP-7FL, MMP-9FL, MMP-12CD, or MMP-14CD by the IC₅₀ of the compound of Formula I with MMP-13CD.

The assays measure the amount by which a test compound reduces the hydrolysis of a thiopeptolide substrate catalyzed by a matrix metalloproteinase enzyme. Such assays are described in detail by Ye et al., in Biochemistry, 1992;31(45):11231-11235, which is incorporated herein by reference. One such assay is described below in Biological Method 1.

Some of the particular methods described below use the catalytic domain of the MMP-13 enzyme, namely matrix metalloproteinase-13 catalytic domain (“MMP-13CD”), rather than the corresponding full-length enzyme, MMP-13. It has been shown previously by Ye Qi-Zhuang, Hupe D., and Johnson L. (Current Medicinal Chemistry, 1996;3:407-418) that inhibitor activity against a catalytic domain of an MMP is predictive of the inhibitor activity against the respective full-length MMP enzyme.

### Biological Method 1

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of N-2-hydroxyethylpiperazine-N′-2-ethanesulfonic acid buffer (“HEPES,” pH 7.0), 10 mM CaCl₂, 100 μM thiopeptolide substrate, and 1 mM 5,5′-dithio-bis-(2-nitro-benzoic acid) (DTNB). The thiopeptolide substrate concentration may be varied, for example from 10 to 800 μM to obtain Kₘ and K_{cat} values. The change in absorbance at 405 nm is monitored on a Thermo Max microplate reader (molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Test compounds are evaluated at various concentrations in order to determine their respective IC₅₀ values, the micromolar concentration of compound required to cause a 50% inhibition of catalytic activity of the respective enzyme.

It should be appreciated that the assay buffer used with MMP-3CD was 50 mM N-morpholinoethane sulfonate (“MES”) at pH 6.0 rather than the HEPES buffer at pH 7.0 described above.

The test described above for the inhibition of MMP-13 may also be adapted and used to determine the ability of the compounds of Formula I to inhibit the matrix metalloproteases MMP-1, MMP-2, MMP-3, MMP-7, MMP-9, MMP-12, and MMP-14, or any other MMP enzyme.

### Biological Method 2

Some representative compounds of Formula I have been evaluated for their ability to inhibit MMP-13. Inhibitor activity versus other MMPs with the compounds may be determined using, for example, MMP-1FL, which refers to full length interstitial collagenase; MMP-2FL, which refers to full length Gelatinase A; MMP-3CD, which refers to the catalytic domain of stromelysin; MMP-7FL, which refers to full length matrilysin; MMP-9FL, which refers to full length Gelatinase B; MMP-13CD, which refers to the catalytic domain of collagenase 3; and MMP-14CD, which refers to the catalytic domain of MMP-14. Test compounds can be evaluated at various concentrations in order to determine their respective IC₅₀ values, the micromolar concentration of compound required to cause a 50% inhibition of the hydrolytic activity of the respective enzyme.

The results of the above assays with other MMPs will establish that the compounds of Formula I are potent inhibitors of MMP enzymes, and are especially useful due to their selective inhibition of MMP-13. Because of this potent and selective inhibitory activity, the compounds are especially useful to treat diseases mediated by the MMP enzymes.

Allosteric inhibitors of MMP-13 which are compounds of Formula I may be readily identified by assaying a test compound for inhibition of MMP-13 according to the methods described below in Biological Methods 3 and 4.

### Biological Method 3

Fluorigenic peptide-1 substrate based assay for identifying compounds of Formula I as allosteric inhibitors of MMP-13:

Final assay conditions:

50 mM HEPES buffer (pH 7.0)

10 mM CaCl₂

10 μM fluorigenic peptide-1 (“FP1”) substrate

0 or 15 mM acetohydroxamic acid (AcNHOH)=1 K_{d}

2% DMSO (with or without inhibitor test compound)

0.5 nM MMP-13CD enzyme

Stock solutions:

1) 10×assay buffer: 500 mM HEPES buffer (pH 7.0) plus 100 mM CaCl₂

2) 10 mM FP1 substrate: (Mca)-Pro-Leu-Gly-Leu-(Dnp)-Dpa-Ala-Arg-NH₂ (Bachem, M-1895; “A novel coumarin-labeled peptide for sensitive continuous assays of the matrix metalloproteinases,” Knight C. G., Willenbrock F., and Murphy, G., FEBS Lett., 1992;296:263-266). Is prepared 10 mM stock by dissolving 5 mg FP1 in 0.457 mL DMSO.

3) 3 M AcNHOH: Is prepared by adding 4 mL H₂O and 1 mL 10×assay buffer to 2.25 g AcNHOH (Aldrich 15,903-4). Adjusting pH to 7.0 with NaOH. Diluting volume to 10 mL with H₂O. Final solution will contain 3 M AcNHOH, 50 mM HEPES buffer (pH 7.0), and 10 mM CaCl₂.

4) AcNHOH dilution buffer: 50 mM HEPES buffer (pH 7.0) plus 10 mM CaCl₂

5) MMP-13CD enzyme: Stock concentration=250 nM.

6) Enzyme dilution buffer: 50 mM HEPES buffer (pH 7.0), 10 mM CaCl₂, and 0.005% BRIJ 35 detergent (Calbiochem 203728; Protein Grade, 10%)

Procedure (for one 96-well microplate):

A. Prepared assay mixture:

1100 μL 19×assay buffer

11 μL 10 mM FP1

55 μL 3 M AcNHOH or 55 μL AcNHOH dilution buffer

8500 μL H₂O

B. Diluted MMP-13CD to 5 nM working stock:

22 μL MMP-13CD (250 nM)

1078 μL enzyme dilution buffer

C. Ran kinetic assay:

1. Dispense 2 μL inhibitor test sample (in 100% DMSO) into well.

2. Add 88 μL assay mixture and mix well, avoiding bubbles.

3. Initiate reactions with 10 μL of 5 nM MMP-13CD; mix well, avoid bubbles.

4. Immediately measure the kinetics of the reactions at room temperature.

Fluorimeter: Fₘₐₓ Fluorescence Microplate Reader & SOFTMAX PRO Version 1.1 software (Molecular Devices Corporation; Sunnyvale, Calif. 94089).

Protocol menu:

excitation: 320 nm
run time: 15 mm
RFU min: −10
Vₘₐₓ points: 32/32
emission: 405 nm
interval: 29 sec
RFU max: 200

D. Compared % of control activity and/or IC₅₀ with inhibitor test compound ±AcNHOH.

Hydrolysis of the fluorigenic peptide-1 substrate, [(Mca)Pro-Leu-Gly-Leu-Dpa-Ala-Arg-NH₂; Bachem, catalog number M-1895], wherein “Mca” is (7-methoxy-coumarin-4-yl)acetyl and “Dpa” is (3-[2,4-dinitrophenyl]-L-2,3-diaminopropionyl), is used to screen for MMP-13 catalytic domain (CD) inhibitors. (Dpa may also be abbreviated as “Dnp”.) Reactions (100 μL) contain 0.05 M Hepes buffer (pH 7), 0.01 M calcium chloride, 0.005% polyoxyethylene (23) lauryl ether (“Brij 35”), 0 or 15 mM acetohydroxamic acid, 10 μM FP1, and 0.1 mM to 0.5 nM inhibitor in DMSO (2% final).

After recombinant human MMP-13CD (0.5 nM final) is added to initiate the reaction, the initial velocity of FP1 hydrolysis is determined by monitoring the increase in fluorescence at 405 nm (upon excitation at 320 nm) continuously for up to 30 minutes on a microplate reader at room temperature. Alternatively, an endpoint read can also be used to determine reaction velocity provided the initial fluorescence of the solution, as recorded before addition of enzyme, is subtracted from the final fluorescence of the reaction mixture. The inhibitor is assayed at different concentration values, such as, for example, 100 μM, 10 μM, 1 μM, 100 nM, 10 nM, and 1 nM. Then the inhibitor concentration is plotted on the X-axis against the percentage of control activity observed for inhibited experiments versus uninhibited experiments (i.e., (velocity with inhibitor) divided by (velocity without inhibitor)×100) on the Y-axis to determine IC₅₀ values. This determination is done for experiments done in the presence, and experiments done in the absence, of acetohydroxamic acid. Data are fit to the equation: percent control activity=100/[1+(([I]/IC₅₀)slope)], where [I] is the inhibitor concentration, IC₅₀ is the concentration of inhibitor where the reaction rate is 50% inhibited relative to the control, and slope is the slope of the IC₅₀ curve at the curve's inflection point, using nonlinear least-squares curve-fitting equation regression.

Results may be expressed as an IC₅₀ Ratio (+/−) ratio, which means a ratio of the IC₅₀ of the inhibitor with MMP-13 and an inhibitor to the catalytic zinc of MMP-13, divided by the IC₅₀ of the inhibitor with MMP-13 without the inhibitor to the catalytic zinc of MMP-13. Compounds of Formula I which are allosteric inhibitors of MMP-13 are expected to have an IC₅₀ Ratio (+/−) ratio of less than 1, and are expected to be synergistic with the inhibitor to the catalytic zinc of MMP-13 such as, for example, AcNHOH. Compounds of Formula I which are not allosteric inhibitors of MMP-13 will be inactive in the assay or will have an IC₅₀ Ratio (+/−) of greater than 1, unless otherwise indicated. Results can be confirmed by kinetics experiments which are well known in the biochemical art.

### Biological Method 4

Fluorigenic peptide-1 based assay for identifying allosteric compound inhibitors of matrix metalloproteinase-13 catalytic domain (“MMP-13CD”):

In a manner similar to Biological Method 3, an assay is run wherein 1,10-phenanthroline is substituted for acetohydroxamic acid to identify compounds of Formula I.

Animal models may be used to establish that the instant compounds of Formula I, or a pharmaceutically acceptable salt thereof, would be useful for preventing, treating, and inhibiting cartilage damage, and thus for treating osteoarthritis, for example. Examples of such animal models are described below in Biological Methods 5 and 6.

### Biological Method 5

Monosodium Iodoacetate-Induced Osteoarthritis in Rat Model of Cartilage Damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions.

Generally, In the MIA Rat model on Day 0, the hind-paw weight differential between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Then the rats are anesthetized with isofluorine, and the right, hind leg knee joint is injected with 1.0 mg of mono-iodoacetate (“MIA”) through the infrapatellar ligament. Injection of MIA into the joint results in the inhibition of glycolysis and eventual death of surrounding chondrocytes. The rats are further administered either an invention compound or vehicle (in the instant case, water) daily for 14 days or 28 days. The invention compound is typically administered at a dose of 30 mg per kilogram of rat per day (30 mg/kg/day), but the invention compound may be administered at other doses such as, for example, 10 mg/kg/day, 60 mg/kg/day, 90-mg/kg/day, or 100 mg/kg/day according to the requirements of the compound being studied. It is well within the level of ordinary skill in the pharmaceutical arts to determine a proper dosage of an invention compound in this model. Administration of the invention compound in this model is optionally by oral administration or intravenous administration via an osmotic pump. After 7 and 14 days for a two-week study, or 7, 14, and 28 days for a four-week study, the hind-paw weight distribution is again determined. Typically, the animals administered vehicle alone place greater weight on their unaffected left hind paw than on their right hind paw, while animals administered an invention compound show a more normal (i.e., more like a healthy animal) weight distribution between their hind paws. This change in weight distribution was proportional to the degree of joint cartilage damage. Percent inhibition of a change in hind paw joint function is calculated as the percent change in hind-paw weight distribution for treated animals versus control animals. For example, for a two week study,

$$Percent inhibition of a change in hind paw joint function = \{ 1 - [ \frac{( Δ {W}_{G} )}{( Δ {W}_{C} )} ] \} \times 100$$

wherein

ΔW_{C} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered an invention compound, as measured on Day 14.

In order to measure biochemical or histopathological end points in the MIA Rat model, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered an invention compound, and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as {[(proteoglycan loss from joint (%) with vehicle)−(proteoglycan loss from joint with an invention compound)]÷(proteoglycan loss from joint (%) with vehicle)}×100.

The MIA Rat data that are expected from the analysis of proteoglycan loss would establish that an invention compound is effective for inhibiting cartilage damage and inflammation and/or alleviating pain in mammalian patients, including human.

The results of these studies with oral dosing may be presented in tabular format in the columns labelled “IJFL (%+/−SEM)”, wherein IJFL means Inhibition of Joint Function Limitation, “SDCES”, wherein SDCES means Significant Decrease In Cartilage Erosion Severity, and “SIJWHLE”, wherein SIJWHLE means Significant Increase in Joints Without Hind Limb Erosion.

The proportion of subjects without hind limb erosions may be analyzed via an Exact Sequential Cochran-Armitage Trend test (SAS® Institute, 1999). The Cochran-Armitage Trend test is employed when one wishes to determine whether the proportion of positive or “Yes” responders increases or decreases with increasing levels of treatment. For the particular study, it is expected that the number of animals without joint erosions increased with increasing dose.

The ridit analysis may be used to determine differences in overall erosion severity. This parameter takes into account both the erosion grade (0=no erosion, I=erosion extending into the superficial or middle layers, or II=deep layer erosion), and area (small, medium and large, quantified by dividing the area of the largest erosion in each score into thirds) simultaneously. The analysis recognizes that each unit of severity is different, but does not assume a mathematical relationship between units.

Another animal model for measuring effects of an invention compound on cartilage damage and inflammation and/or pain is described below in Biological Method 6.

### Biological Method 6

Induction of Experimental Osteoarthritis in Rabbit (“EOA in Rabbit”):

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water) or an invention compound dosed three times per day with 30-mg/kg/dose or 10-mg/kg/dose. The invention compound may be administered at other doses such as, for example, 3 times 20 mg/kg/day or 3 times 60 mg/kg/day according to the requirements of the invention compound being studied. The rabbits are euthanized 8 weeks after surgery and the proximal end of the tibia and the distal end of the femur are removed from each animal.

Macroscopic Grading

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens may also be used for histologic grading (see below).

Histologic Grading

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe histologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average score is calculated and considered as a unit for the whole knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis

Mean values and SEM is calculated and statistical analysis was done using the Mann-Whitney U-test.

The results of these studies would be expected to show that an invention compound would reduce the size of the lesion on the tibial plateaus, and perhaps the damage in the tibia or on the femoral condyles. In conclusion, these results would show that an invention compound would have significant inhibition effects on the damage to cartilage.

The foregoing studies would establish that an invention compound is effective for the inhibition of cartilage damage and inflammation and/or alleviating pain, and thus useful for the treatment of osteoarthritis or rheumatoid arthritis in human, and other mammalian disorders. Such a treatment offers a distinct advantage over existing treatments that only modify pain or inflammation or and other secondary symptoms. The effectiveness of an invention compound in this model would indicate that the invention compound will have clinically useful effects in preventing and/or treating cartilage damage, pain and/or inflammation.

Administration according to the invention method of an invention compound to a mammal to treat the diseases listed above is preferably, although not necessarily, accomplished by administering the compound, or a salt thereof, in a pharmaceutical dosage form.

The compounds of Formula I, or a pharmaceutically acceptable salt thereof, can be prepared and administered according to the invention method in a wide variety of oral and parenteral pharmaceutical dosage forms. Thus, the compounds of Formula I, or a pharmaceutically acceptable salt thereof, can be administered by injection, that is, intravenously, intramuscularly, intracutaneously, subcutaneously, intraduodenally, or intraperitoneally. Also, the compounds of Formula I, or a pharmaceutically acceptable salt thereof, can be administered by inhalation, for example, intranasally. Additionally, the compounds of Formula I, or a pharmaceutically acceptable salt thereof, can be administered transdermally. It will be obvious to those skilled in the art that the following dosage forms may comprise as the active component an invention compound. The invention compounds generally are present in a concentration of about 5% to about 95% by weight of the formulation.

For preparing pharmaceutical compositions from the compounds of Formula I, or a pharmaceutically acceptable salt thereof, (i.e., the active component) pharmaceutically acceptable carriers can be either solid or liquid. Solid form preparations are preferred. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

The powders and tablets preferably contain from about 5% to about 70%, total, of the active component. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active component with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing an appropriate quantity of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 to 1000 mg, preferably 1 to 500 mg according to the particular application and the potency of the active components. The composition can, if desired, also contain other compatible therapeutic agents.

In therapeutic use as agents to treat the above-listed diseases, the compounds of Formula I, or a pharmaceutically acceptable salt thereof, are administered at a dose that is effective for treating at least one symptom of the disease or disorder being treated. The initial dosage of about 1 mg/kg to about 100 mg/kg daily of the active component will be effective. A daily dose range of about 25 mg/kg to about 75 mg/kg of the active component is preferred. The dosages, however, may be varied depending upon the requirements of the patient, the severity of the condition being treated, and the particular invention compound being employed in the invention combination. Determination of the proper dosage for a particular situation is within the skill of the art as described above. Typical dosages will be from about 0.1 mg/kg to about 500 mg/kg, and ideally about 25 mg/kg to about 250 mg/kg, such that it will be an amount that is effective to treat the particular disease or disorder being treated.

A preferred composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

A preferred composition provides delayed-, sustained- and/or controlled-release of an invention compound. Such preferred compositions include all such dosage forms which produce ≧40% inhibition of cartilage degradation, and result in a plasma concentration of the active component of at least 3 fold the active component's ED₄₀ for at least 2 hours; preferably for at least 4 hours; preferably for at least 8 hours; more preferably for at least 12 hours; more preferably still for at least 16 hours; even more preferably still for at least 20 hours; and most preferably for at least 24 hours. Preferably, there is included within the above-described dosage forms those which produce ≧40% inhibition of cartilage degradation, and result in a plasma concentration of the active component of at least 5 fold the active component's ED₄₀ for at least 2 hours, preferably for at least 2 hours, preferably for at least 8 hours, more preferably for at least 12 hours, still more preferably for at least 20 hours and most preferably for at least 24 hours. More preferably, there is included the above-described dosage forms which produce ≧50% inhibition of cartilage degradation, and result in a plasma concentration of the active component of at least 5 fold the active component's ED₄₀ for at least 2 hours, preferably for at least 4 hours, preferably for at least 8 hours, more preferably for at least 12 hours, still more preferably for at least 20 hours and most preferably for at least 24 hours.

The following Formulation Examples 1 to 8 illustrate the invention pharmaceutical compositions. When the formulations comprise the invention compound and a pharmaceutically acceptable carrier, diluent, or excipient, they contain a cartilage damage treating effective amount or a therapeutically effective amount such as, for example, an anti-osteoarthritic effective amount of the invention compound. The examples are representative only, and are not to be construed as limiting the invention in any respect.

FORMULATION EXAMPLE 1

| Ingredient | Amount (mg) |
|---|---|
| Tablet Formulation: | |
| An invention compound | 25 |
| Lactose | 50 |
| Cornstarch (for mix) | 10 |
| Cornstarch (paste) | 10 |
| Magnesium stearate (1%) | 5 |
| Total | 100 |

The invention compound, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for inhibiting cartilage damage or treating osteoarthritis.

### Formulation Example 2

Coated Tablets:

The tablets of Formulation Example 1 are coated in a customary manner with a coating of sucrose, potato starch, talc, tragacanth, and colorant.

### Formulation Example 3

Injection Vials:

The pH of a solution of 500 g of an invention compound and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of the invention compound.

### Formulation Example 4

Suppositories:

A mixture of 25 g of an invention compound, 100 g of soya lecithin, and 1400 g of cocoa butter is fused, poured into molds, and allowed to cool. Each suppository contains 25 mg of the invention compound.

### Formulation Example 5

Solution:

A solution is prepared from 1 g of an invention compound, 9.38 g of NaH₂PO₄.12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 25 mg of the invention compound.

### Formulation Example 6

Ointment:

500 mg of an invention compound is mixed with 99.5 g of petroleum jelly under aseptic conditions. A 5 g portion of the ointment contains 25 mg of the invention compound.

### Formulation Example 7

Capsules:

2 kg of an invention compound are filled into hard gelatin capsules in a customary manner such that each capsule contains 25 mg of the invention compound.

### Formulation Example 8

Ampoules:

A solution of 2.5 kg of an invention compound is dissolved in 60 L of double-distilled water. The solution is sterile filtered, and the filtrate is filled into ampoules. The ampoules are lyophilized under sterile conditions and aseptically sealed. Each ampoule contains 25 mg of the invention compound.

The following Formulation Examples 9 to 16 illustrate the invention pharmaceutical compositions containing an invention combination in a single formulation with a pharmaceutically acceptable carrier, diluent, or excipient. The examples are representative only, and are not to be construed as limiting the invention in any respect.

FORMULATION EXAMPLE 9

| Ingredient | Amount (mg) |
|---|---|
| Tablet Formulation: | |
| An invention compound | 25 |
| A COX-2 inhibitor | 20 |
| Lactose | 50 |
| Cornstarch (for mix) | 10 |
| Cornstarch (paste) | 10 |
| Magnesium stearate (1%) | 5 |
| Total | 120 |

The invention compound or COX-2 inhibitor, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of one of the above-listed diseases.

### Formulation Example 10

Coated Tablets:

The tablets of Formulation Example 9 are coated in a customary manner with a coating of sucrose, potato starch, talc, tragacanth, and colorant.

### Formulation Example 11

Injection Vials:

The pH of a solution of 250 g of a COX-2 inhibitor, 500 g of an invention compound, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 12.5 mg of COX-2 inhibitor and 25 mg of the invention compound.

### Formulation Example 12

Suppositories:

A mixture of 50 g of a COX-2 inhibitor, 25 g of an invention compound, 100 g of soya lecithin, and 1400 g of cocoa butter is fused, poured into molds, and allowed to cool. Each suppository contains 50 mg of the COX-2 inhibitor and 25 mg of the invention compound.

### Formulation Example 13

Solution:

A solution is prepared from 0.5 g of a COX-2 inhibitor, 1 g of an invention compound, 9.38 g of NaH₂PO₄.12H₂O, 28.48 g of Na₂HPO₄.12H₁₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 12.5 mg of the COX-2 inhibitor and 25 mg of the invention compound.

### Formulation Example 14

Ointment:

100 mg of a COX-2 inhibitor, 500 mg of an invention compound is mixed with 99.4 g of petroleum jelly under aseptic conditions. A 5 g portion of the. ointment contains 5 mg of the COX-2 inhibitor and 25 mg of the invention compound.

### Formulation Example 15

Capsules:

2 kg of a COX-2 inhibitor and 20 kg of an invention compound are filled into hard gelatin capsules in a customary manner such that each capsule contains 25 mg of the COX-2 inhibitor and 250 mg of the invention compound.

### Formulation Example 16

Ampoules:

A solution of 2.5 kg of a COX-2 inhibitor and 2.5 kg of an invention compound is dissolved in 60 L of double-distilled water. The solution is sterile filtered, and the filtrate is filled into ampoules. The ampoules are lyophilized under sterile conditions and aseptically sealed. Each ampoule contains 25 mg each of the COX-2 inhibitor and the invention compound.

While it may be desirable to formulate a COX-2 inhibitor and an invention compound together in one capsule, tablet, ampoule, solution, and the like, for simultaneous administration, it is not necessary for the purposes of practicing the invention methods. A COX-2 inhibitor and an invention compound alternatively can each be formulated independently in any form such as, for example, those of any one Formulation Examples 1 to 16, and administered to a patient either simultaneously or at different times.

The following examples illustrate the invention pharmaceutical compositions containing discrete formulations of the active components of an invention combination and a pharmaceutically acceptable carrier, diluent, or excipient. The examples are representative only, and are not to be construed as limiting the invention in any respect.

FORMULATION EXAMPLE 17

| Ingredient | Amount (mg) |
|---|---|
| Tablet Formulation of | |
| an invention compound: | |
| An invention compound | 25 |
| Lactose | 50 |
| Cornstarch (for mix) | 10 |
| Cornstarch (paste) | 10 |
| Magnesium stearate (1%) | 5 |
| Total | 100 |

An invention compound, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet.

Injection Vial Formulation of a COX-2 Inhibitor:

The pH of a solution of 500 g of a COX-2 inhibitor and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of the COX-2 inhibitor.

Such tablets containing the invention compound can be administered to a human from one to four times a day for treatment of the above-listed diseases, and the injection solutions containing the COX-2 inhibitor can be administered to a human 1 or 2 times per day, wherein the administration by injection is optionally simultaneous with administration of the tablets or at different times, for the treatment of one of the above-listed diseases.

### Formulation Example 18

Coated Tablets Containing an Invention Compound:

The tablets of Formulation Example 17 are coated in a customary manner with a coating of sucrose, potato starch, talc, tragacanth, and colorant.

Capsules Containing Valdecoxib or Celecoxib:

2 kg of a COX-2 inhibitor are filled into hard gelatin capsules in a customary manner such that each capsule contains 25 mg of the COX-2 inhibitor.

Such coated tablets containing the invention compound can be administered to a human from one to four times a day for treatment of the above-listed diseases, and the capsules containing the COX-2 inhibitor can be administered to a human 1 or 2 times per day, wherein the administration of the capsules is optionally simultaneous with administration of the tablets or at different times, for the treatment of one of the above-listed diseases.

Still further, it should be appreciated that the invention methods comprising administering an invention combination to a mammal to treat diseases or disorders listed above may be used to treat different diseases simultaneously. For example, administration of a COX-2 inhibitor in accordance with the invention combination may be carried out as described above to treat inflammation, arthritic pain, pain associated with menstrual cramping, and migraines, while an invention compound may be administered to treat OA or inhibit cartilage damage.

As shown above, the invention methods comprising administering an invention compound offer a distinct advantage over existing treatments for diseases such as OA that comprise cartilage damage, wherein the existing treatments modify pain or secondary symptoms, but do not show a disease modifying effect.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All references cited above are hereby incorporated by reference herein.

Having described the invention method, various embodiments of the invention are hereupon claimed.

What is claimed is:

1. A compound of Formula I
Z—L—R¹Q—D—(V¹)ₘ—R²
or a pharmaceutically acceptable salt thereof,
wherein:
Z is selected from:
HO₂C;
HO(H)N(O)C;
H(O)C—N(OH);
CH₃(O)C—N(OH);
CH₃(H)N(O)C—N(OH);
HS;
H₂N(O)₂S;
CH₃(H)N(O)₂S;
HO(O)P;
(HO)₂(O)P; 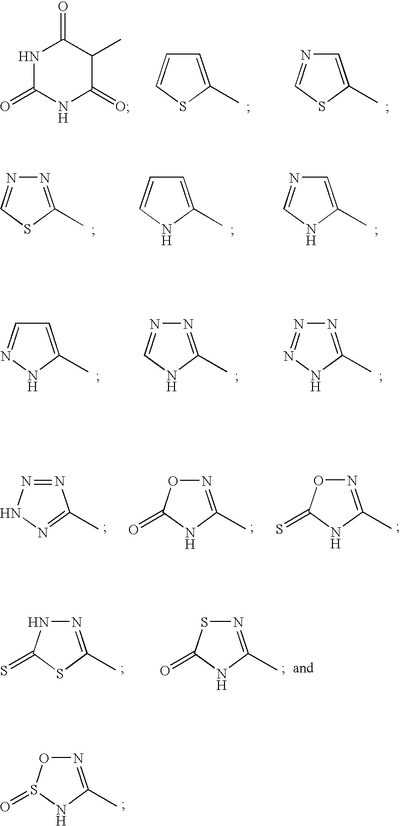
L is selected from:
C₃-C₅ alkylenyl;
Substituted C₃-C₅ alkylenyl;
3- to 5-membered heteroalkylenyl; and
Substituted 3- to 5-membered heteroalkylenyl;
Substituted L groups contain 1 or 2 substituents on a carbon atom or nitrogen atom independently selected from:
HO;
CN; and
CF₃;
wherein each substituent on a carbon atom may further be independently F, and wherein 2 substituents may be taken together with a carbon atom to which they are both bonded to form the group C═O;
R¹ is independently selected from:
C₅ or C₆ cycloalkylenyl-(C₁-C₈ alkylenyl);
Substituted C₅ or C₆ cycloalkylenyl-(C₁-C₈ alkylenyl);
5- or 6-membered heterocycloalkylenyl-(C₁-C₈ alkylenyl);
Substituted 5- or 6-membered heterocycloalkylenyl-(C₁-C₈ alkylenyl);
Phenylenyl-(C₁-C₈ alkylenyl);
Substituted phenylenyl-(C₁-C₈ alkylenyl);
5- or 6-membered heteroarylenyl-(C₁-C₈ alkylenyl);
Substituted 5- or 6-membered heteroarylenyl-(C₁-C₈ alkylenyl);
Phenyl;
Substituted phenyl;
Naphthyl;
Substituted naphthyl;
5- or 6-membered heteroaryl;
Substituted 5- or 6-membered heteroaryl;
8- to 10-membered heterobiaryl; and
Substituted 8- to 10-membered heterobiaryl;
R² is independently selected from:
H;
C₁-C₆ alkyl;
Phenyl-(C₁-C₈ alkylenyl);
Substituted phenyl-(C₁-C₈ alkylenyl);
Naphthyl-(C₁-C₈ alkylenyl);
Substituted naphthyl-(C₁-C₈ alkylenyl);
5- or 6-membered heteroaryl-(C₁-C₈ alkylenyl);
Substituted 5- or 6-membered heteroaryl-(C₁-C₈ alkylenyl);
8- to 10-membered heterobiaryl-(C₁-C₈ alkylenyl);
Substituted 8- to 10-membered heterobiaryl-(C₁-C₈ alkylenyl);
Phenyl-O—(C₁-C₈ alkylenyl);
Substituted phenyl-O—(C₁-C₈ alkylenyl);
Phenyl-S—(C₁-C₈ alkylenyl);
Substituted phenyl-S—(C₁-C₈ alkylenyl);
Phenyl-S(O)—(C₁-C₈ alkylenyl);
Substituted phenyl-S(O)—(C₁-C₈ alkylenyl);
Phenyl-S(O)₂—(C₁-C₈ alkylenyl); and
Substituted phenyl-S(O)₂—(C₁-C₈ alkylenyl);
Each substituted R¹ group contains from 1 to 3 substituents, and each substituted
R group contains from 1 to 4 substituents, wherein each substituent is independently on a carbon or nitrogen atom, independently selected from:
C₁-C₆ alkyl;
CN;
CF₃;
HO;
(C₁-C₆ alkyl)-O;
(C₁-C₆ alkyl)-S(O)₂;
H₂N;
(C₁-C₆ alkyl)-N(H);
(C₁-C₆ alkyl)₂-N;
(C₁-C₆ alkyl)-C(O)O—(C₁-C₈ alkylenyl)ₘ;
(C₁-C₆ alkyl)-C(O)O-(1- to 8-membered heteroalkylenyl)ₘ;
(C₁-C₆ alkyl)-C(O)N(H)—(C₁-C₈ alkylenyl)ₘ;
(C₁-C₆ alkyl)-C(O)N(H)-(1- to 8-membered heteroalkylenyl)ₘ;
H₂NS(O)₂_{13 (C}₁-C₈ alkylenyl);
(C₁-C₆ alkyl)-N(H)S(O)₂—(C₁-C₈ alkylenyl)ₘ;
(C₁-C₆ alkyl)₂-NS(O)₂—(C₁-C₈ alkylenyl)ₘ;
3- to 6-membered heterocycloalkyl-(G)ₘ;
Substituted 3- to 6-membered heterocycloalkyl-(G)ₘ;
5- or 6-membered heteroaryl-(G)ₘ;
Substituted 5- or 6-membered heteroaryl-(G)ₘ;
(C₁-C₆ alkyl)-S(O)₂—N(H)—C(O)—(C₁-C₈ alkylenyl)ₘ; and
(C₁-C₆ alkyl)-C(O)—N(H)—S(O)₂—(C₁-C₈ alkylenyl)ₘ;
wherein each substituent on a carbon atom may further be independently selected from:
Halo; and
HO₂C;
wherein 2 substituents may be taken together with a carbon atom to which they are both bonded to form the group C═O;
wherein two adjacent, substantially sp² carbon atoms may be taken together with a diradical substituent to form a cyclic diradical selected from: 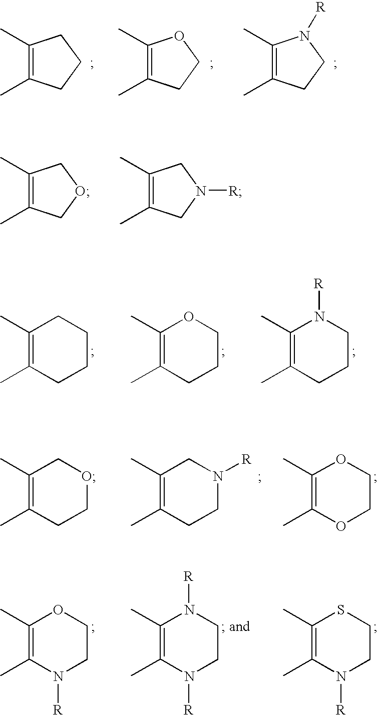
R is H or C₁-C₆ alkyl;
G is CH₂; O, S, S(O); or S(O)₂;
Each m is an integer of 0 or 1;
Q, when bonded to a nitrogen atom in group D, is selected from:
OC(O);
CH(R⁶)C(O);
OC(NR⁶);
CH(R⁶)C(NR⁶);
N(R⁶)C(O);
N(R⁶)C(S);
N(R⁶)C(NR⁶);
SC(O);
CH(R⁶)C(S);
SC(NR⁶);
C≡CCH₂; 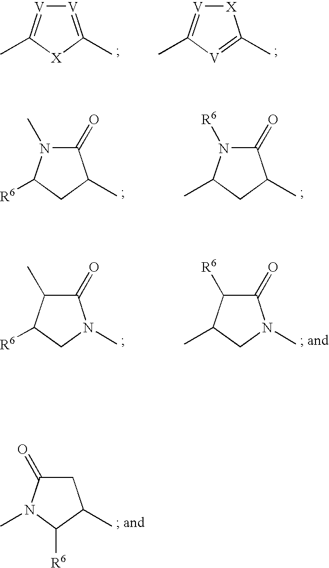
Q, when bonded to a carbon atom in group D, is as defined above and may further be selected from:
OCH₂;
N(R⁶)CH₂;
trans-(H)C═C(H);
cis-(H)C═C(H);
C≡C;
CH₂C≡C;
CF₂C≡C;
C≡CCF₂;
Each R⁶ independently is H, C₁-C₆ alkyl, C₃-C₆ cycloalkyl; 3- to 6-membered heterocycloalkyl; phenyl; benzyl; or 5- or 6-membered heteroaryl;
X is O, S, N(H), or N(C₁-C₆ alkyl);
Each V is independently C(H) or N;
D is a cyclic diradical group selected from: 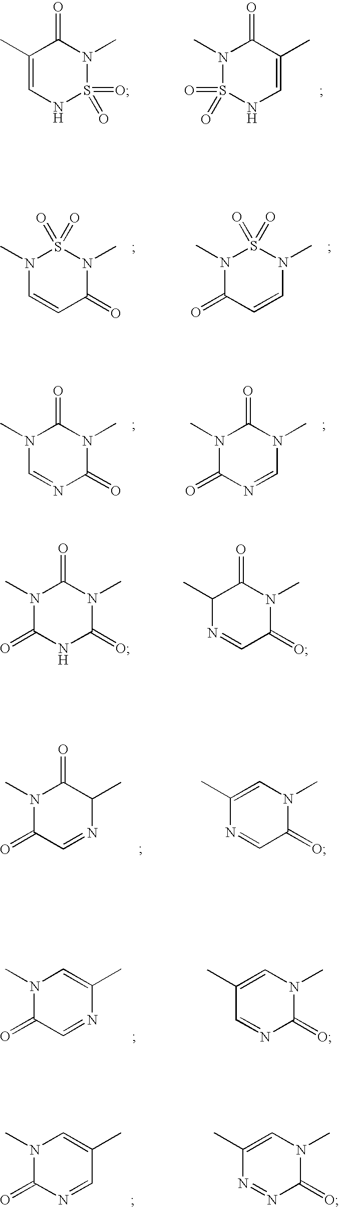 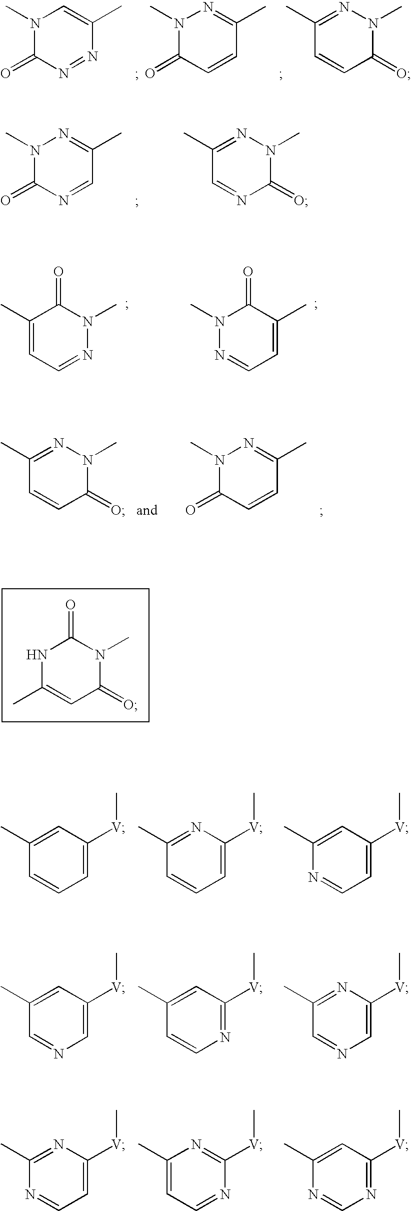 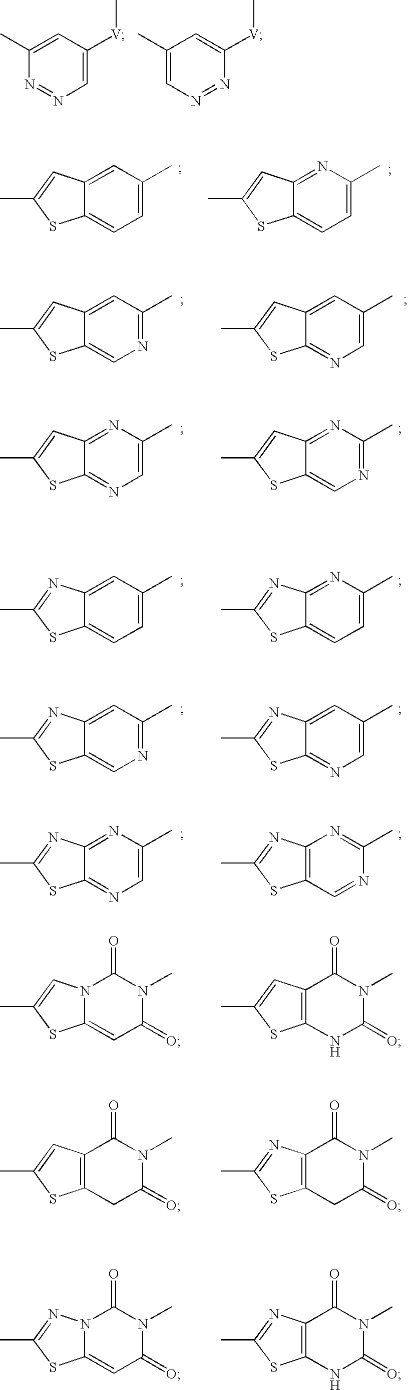 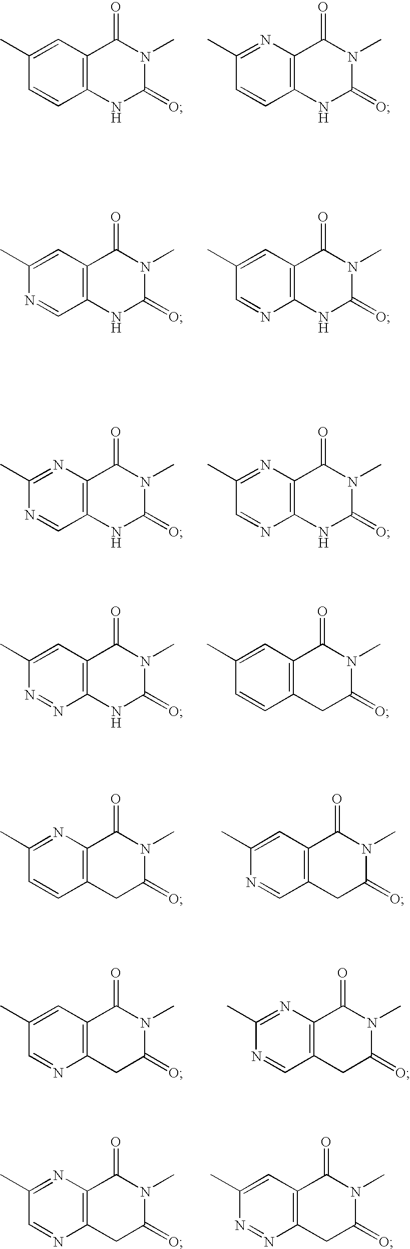 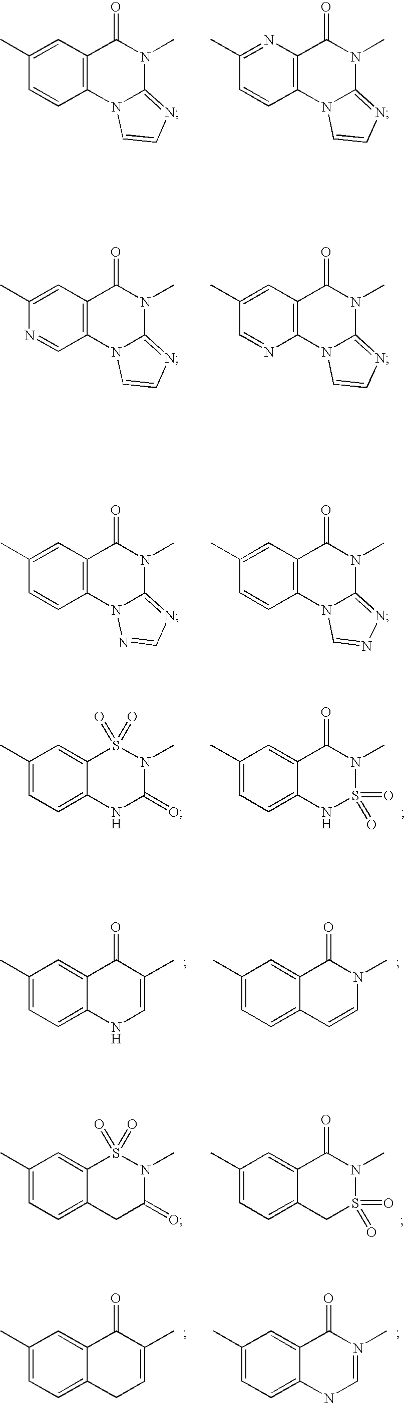 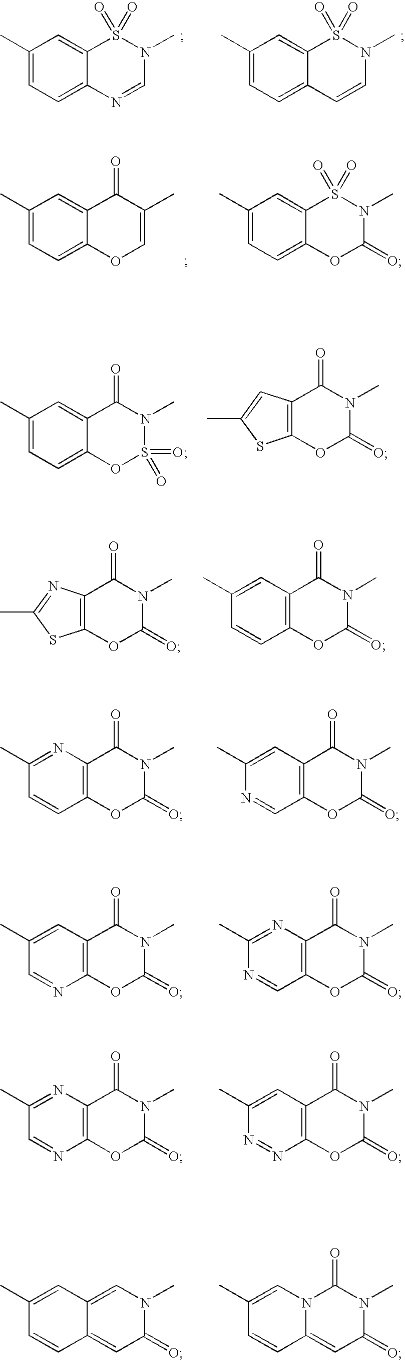 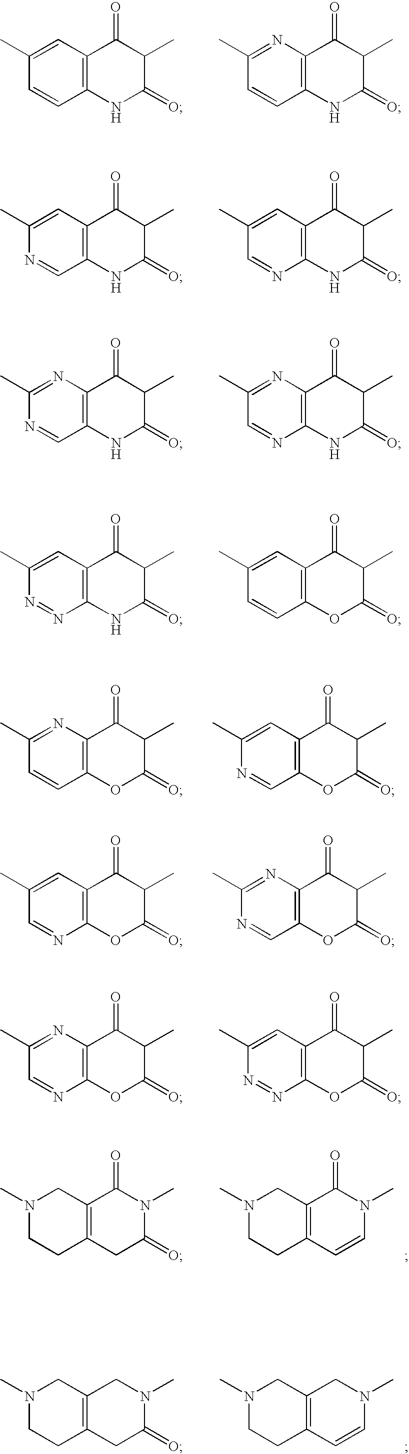 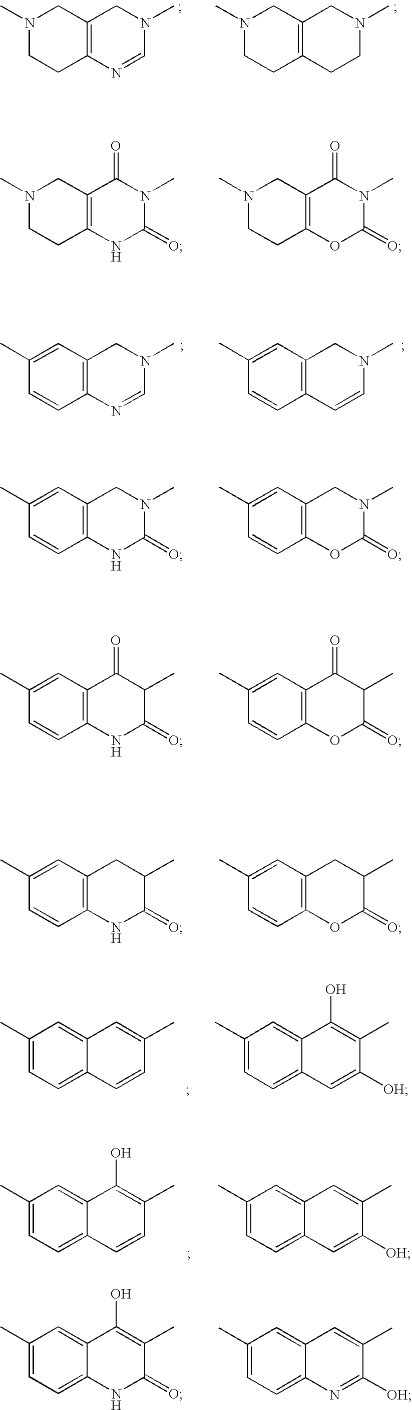 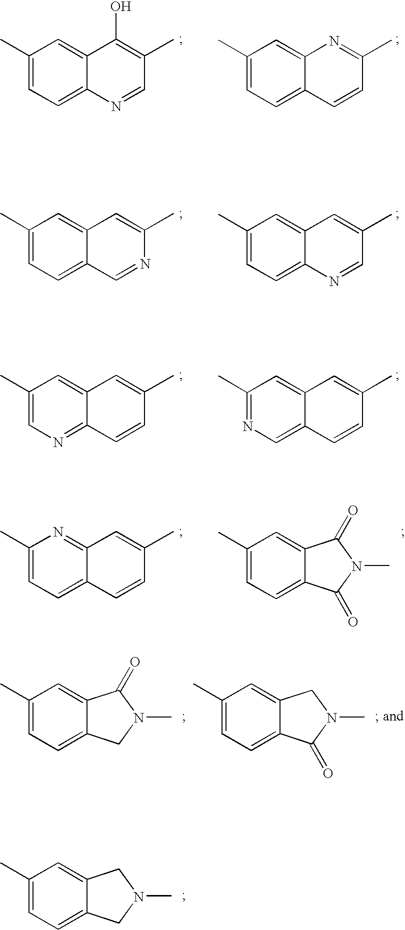
wherein the group D may be unsubstituted or substituted on a carbon atom or a nitrogen atom by replacement of a hydrogen atom with a group selected from:
CH₃;
CF₃;
C(O)H;
CN;
HO;
CH₃O;
C(F)H₂O;
C(H)F₂O; and
CF₃O;
wherein a carbon atom in the group D may further be substituted with F;
V¹ is a 5-membered heteroarylenyl containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;
wherein each C₈-C₁₀ bicycloalkyl is a bicyclic carbocyclic ring that contains 8-, 9-, or 10-member carbon atoms which are 5,5-fused, 6,5-fused, or 6,6-fused bicyclic rings, respectively, and wherein the ring is saturated or optionally contains one carbon-carbon double bond;
wherein each 8- to 10-membered heterobicycloalkyl is a bicyclic ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond, and wherein the heterobicycloalkyl is a 5,5-fused, 6,5-fused, or 6,6-fused bicyclic ring, respectively,
wherein each heterocycloalkyl is a ring that contains carbon atoms and from 1 to 4 heteroatoms independently selected from 2 O, 1 S, 1 S(O), 1 S(O)₂, 1 N, 4 N(H), and 4 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;
wherein each heterocycloalkylenyl is a ring diradical that contains carbon atoms and from 1 to 3 heteroatoms independently selected from 1 O, 1 S, 1 S(O), 1 S(O)₂, 1 N, 2 N(H), and 2 N(C₁-C₆ alkyl), and wherein when one O atom and one S atom are present, the one O atom and one S atom are not bonded to each other, and wherein the ring is saturated or optionally contains one carbon-carbon or carbon-nitrogen double bond;
wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings;
wherein a 5-membered heteroarylenyl is a 5-membered monocyclic diradical ring that contains carbon atoms and from 1 to 4 heteroatoms independently selected from 1 O, 1 S, 1 N(H), 1 N(C₁-C₆ alkyl), and 4 N, wherein the 1 O atom and 1 S atom are not both present, and 6-membered heteroarylenyl is a 6-membered monocyclic diradical ring that contains carbon atoms and 1 or 2 heteroatoms independently selected from 2 N;
wherein each heterobiaryl contains carbon atoms and from 1 to 4 heteroatoms independently selected from 1 O, 1 S, 1 N(H), 1 N(C₁-C₆ alkyl), and 4 N, and where the 8-, 9-, and 10-membered heterobiaryl are 5,5-fused, 6,5-fused, and 6,6-fused bicyclic rings, respectively, and wherein at least 1 of the 2 fused rings of a bicyclic ring is aromatic, and wherein when the O and S atoms both are present, the O and S atoms are not bonded to each other;
wherein with any (C₁-C₆ alkyl)₂-N group, the C₁-C₆ alkyl groups may be optionally taken together with the nitrogen atom to which they are attached to form a 5- or 6-membered heterocycloalkyl; and
wherein each group and each substituent recited above is independently selected.

2. The compound according to claim 1, or a pharmaceutically acceptable salt thereof, wherein Z is HO₂C.

3. The compound according to claim 1, or a pharmaceutically acceptable salt thereof, wherein Z is selected from:

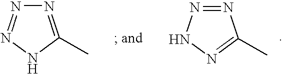

4. The compound according to claim 1, or a pharmaceutically acceptable salt thereof, wherein Z is selected from:

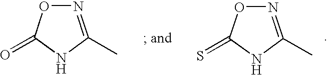

5. The compound according to any one of claims 1 to 4, or a pharmaceutically acceptable salt thereof, wherein Q is N(R⁶)C(O).

6. The compound according to any one of claims 1 to 4, or a pharmaceutically acceptable salt thereof, wherein Q is selected from:
C≡C;
CH₂C≡C;
C≡CCH₂;
CF₂C≡C; and
C≡CCF₂.

7. The compound according to claim 1, selected from:
4-(3-{3-[3-(3,4-Difluoro-benzyl)-4-oxo-3,4-dihydro-quinazolin-6-yl-]-prop-2-ynyl}-phenyl)-butyric acid; and
5-(3,4-Difluoro-benzyl)-7-methyl-4,6-dioxo-3a,4,5,6-tetrahydro-thieno[3,2-c]pyridine-2-carboxylic acid [2-(3-mercapto-propoxy)-pyridin-4-ylmethyl]-amide;
or a pharmaceutically acceptable salt thereof.

8. A pharmaceutical composition, comprising a compound according to claim 1, or a pharmaceutically acceptable salt thereof, admixed with a pharmaceutically acceptable carrier, excipient, or diluent.

9. The pharmaceutical composition according to claim 8, comprising a compound according to claim 7, or a pharmaceutically acceptable salt thereof, admixed with a pharmaceutically acceptable carrier, excipient, or diluent.

10. A method for treating osteoarthritis, comprising administering to a patient suffering from osteoarthritis or rheumatoid arthritis a nontoxic effective amount of a compound according to claim 1, or a pharmaceutically acceptable salt thereof.

11. The method according to claim 10, wherein the compound administered is a compound according to claim 7, or a pharmaceutically acceptable salt thereof.
